(19) United States
(12) Patent Application Publication
(10) Pub. No.: US 2024/0158810 A1
(43) Pub. Date: May 16, 2024

(54) COMPOSITIONS AND METHODS FOR AMELIORATING ANTERODORSAL THALAMUS HYPEREXCITABILITY

(71) Applicants: The Broad Institute, Inc., Cambridge, MA (US); Massachusetts Institute of Technology, Cambridge, MA (US)

(72) Inventors: Guoping FENG, Cambridge, MA (US); Dheeraj ROY, Cambridge, MA (US); Ying ZHANG, Cambridge, MA (US)

(73) Assignees: The Broad Institute, Inc., Cambridge, MA (US); Massachusetts Institute of Technology, Cambridge, MA (US)

(21) Appl. No.: 18/394,297

(22) Filed: Dec. 22, 2023

Related U.S. Application Data
(63) Continuation of application No. PCT/US2022/035072, filed on Jun. 27, 2022.
(60) Provisional application No. 63/216,463, filed on Jun. 29, 2021.

(51) Int. Cl. C12N 15/86; A61K 31/5513; A61K 48/00; A61P 25/00; C07K 14/72

(52) U.S. Cl. CPC C12N 15/86; A61K 31/5513; A61K 48/0058; A61P 25/00; C07K 14/723; C12N 2750/14143

(57) ABSTRACT

The disclosure features compositions and methods for ameliorating cognitive impairments associated with neuropsychiatric disorders, particularly those associated with anterodorsal (AD) thalamus hyperexcitability in the brain of a subject. Various embodiments of the disclosure provide for personalized and targeted therapeutic approaches for screening, diagnosing, preventing, and treating cognitive impairments and neuropsychiatric disorders.

### CROSS-REFERENCE TO RELATED APPLICATIONS

This application is a continuation under 35 U.S.C. § 111(a) of PCT International Patent Application No. PCT/US2022/035072, filed Jun. 27, 2022, designating the United States and published in English, which claims priority to and the benefit of U.S. Provisional Application No. 63/216,463, filed Jun. 29, 2021, the entire contents of each of which are incorporated by reference herein.

### STATEMENT REGARDING FEDERALLY SPONSORED RESEARCH OR DEVELOPMENT

This invention was made with government support under Grant No. MH114819 awarded by the National Institutes of Health. The government has certain rights in the invention.

### SEQUENCE LISTING

The present application contains a Sequence Listing which has been submitted electronically in XML format. The entire contents of the electronic XML Sequence Listing, (Date of creation: Dec. 22, 2023: Size: 89,834 bytes: Name: 167741-043002US-Sequence_Listing.xml), is incorporated by reference herein.

### BACKGROUND

Intellectual disability or cognitive impairment can be characterized by significant limitations in cognitive functions, including reasoning, learning, memory, and adaptive behaviors, which can co-occur with many neuropsychiatric disorders, including, e.g., autism spectrum disorder (ASD) and schizophrenia. Cognitive impairments in these disorders have been commonly linked to dysfunction within hippocampal and cortical circuits. However, whether converging neurobiological mechanisms underlie cognitive impairments across disorders has not been previously established. As such, the currently available treatments for cognitive impairment and neuropsychiatric diseases have many off-target effects on the brain that can cause unwanted side-effects that prevent the effective treatment of these conditions and reduce patient compliance. Thus, there is a significant need for personalized and targeted therapeutic approaches for screening, diagnosing, preventing, and treating cognitive impairments and neuropsychiatric disorders.

### SUMMARY

As described below, embodiments of the present invention feature compositions and methods for ameliorating cognitive impairments associated with neuropsychiatric disorders, particularly those associated with anterodorsal (AD) thalamus hyperexcitability in the brain of a subject.

Compositions and articles defined by embodiments of the invention were isolated or otherwise manufactured in connection with the examples provided below. Other features and advantages of the invention will be apparent from the description.

In accordance with the foregoing objectives, embodiments of the present disclosure satisfy the aforementioned needs and provide related advantages as well. The present disclosure provides in some aspects, a method for increasing cognitive performance in a subject. The method comprises administering to the subject a recombinant adeno-associated virus (rAAV), where the rAAV comprises a sequence encoding an engineered M4 muscarinic acetylcholine receptor. The method also comprises expressing the receptor in neurons of the subject that inhibit anterodorsal thalamus hyperexcitability, thereby increasing cognitive performance in the subject. Some objects of the disclosure are directed to the method, where the neurons project to the retrosplenial cortex. Other objects provide for neurons that are neurons of the anterodorsal (AD) thalamus, anteroventral (AV) thalamus, or entorhinal cortex neurons. In some objects, the receptor is expressed in neurons present in the anterodorsal thalamus to (→) retrosplenial cortex (RSC) circuit, thereby enhancing memory encoding. Additional objects are directed to the receptor that is expressed in neurons present in the anteroventral →RSC circuit, thereby enhancing memory specificity. In other objects, the subject has or is suspected of having or is at risk of developing a neuropsychiatric disorder. Further objects provide for a neuropsychiatric disorder that is autism, schizophrenia, or a disorder associated with an alteration in the sequence, activity, or expression of PTCHD1, YWHAG, or HERC1. In some objects, the method for increasing cognitive performance in a subject further comprises administering to the individual an agonist of the receptor. In some objects of the disclosure, the rAAV comprises a polynucleotide encoding a Cre-dependent inhibitory Designer Receptors Exclusively Activated by Designer Drugs (DREADD) hM4Di. Additional objects of the methods of the disclosure provide for a ligand, where the ligand is compound 21 (C21).

Another aspect of the disclosure provides a method for reducing cognitive impairment associated with a neuropsychiatric disorder in a subject. The method comprises administering to a subject a recombinant adeno-associated virus (rAAV), where the rAAV comprises a sequence encoding an engineered M4 muscarinic acetylcholine receptor. The method also comprises expressing the receptor in neurons of the subject that inhibit anterodorsal thalamus hyperexcitability, thereby reducing cognitive impairment in the subject. Some objects of the disclosure are directed to the method, where the neurons project to the retrosplenial cortex. Other objects provide for neurons that are neurons of the anterodorsal (AD) thalamus, anteroventral (AV) thalamus, or entorhinal cortex neurons. In some objects, the receptor is expressed in neurons present in the anterodorsal thalamus to (→) retrosplenial cortex (RSC) circuit, thereby enhancing memory encoding. Additional objects are directed to the receptor that is expressed in neurons present in the anteroventral →RSC circuit. thereby enhancing memory specificity. In other objects, the subject has or is suspected of having or is at risk of developing a neuropsychiatric disorder. Further objects provide for a neuropsychiatric disorder that is autism, schizophrenia, or a disorder associated with an alteration in the sequence, activity, or expression of PTCHD1, YWHAG, or HERC1. In some objects, the method for reducing cognitive impairment associated with a neuropsychiatric disorder in a subject further comprises administering to the individual an agonist of the receptor. In some objects of the disclosure, the rAAV comprises a polynucleotide encoding a Cre-dependent inhibitory Designer Receptors Exclusively Activated by Designer Drugs (DREADD) hM4Di. Additional objects of the methods of the disclosure provide for a ligand, where the ligand is compound 21 (C21).

In additional aspects of the disclosure, a method for increasing cognitive performance is provided. The method comprises administering to a subject a recombinant adeno-associated virus (rAAV), where the rAAV comprises a promoter expressed in a neuron selected from the group consisting of neurons of the anterodorsal (AD) thalamus. anteroventral (AV) thalamus, entorhinal cortex, neurons present in the anterodorsal thalamus →retrosplenial cortex (RSC) circuit, and neurons present in the anteroventral →RSC circuit. In some aspects, the promoter is operatively linked to a polynucleotide encoding an hM4Di polypeptide. Additional aspects are directed to the method for increasing cognitive performance comprising administering to the subject a ligand of hM4Di polypeptide. In further aspects, expression of the hM4Di polypeptide or the hM4Di polypeptide expression inhibits anterodorsal thalamus hyperexcitability, thereby increasing cognitive performance in the subject. In some objects of the disclosure, the rAAV comprises a polynucleotide encoding a Cre-dependent inhibitory Designer Receptors Exclusively Activated by Designer Drugs (DREADD) hM4Di. Additional objects of the methods of the disclosure provide for a ligand, where the ligand is compound 21 (C21).

Some aspects provide a method for reducing cognitive impairment associated with a neuropsychiatric disorder in a subject. The method comprises administering to a subject a recombinant adeno-associated virus (rAAV), where the rAAV comprises a promoter expressed in a neuron selected from the group consisting of neurons of the anterodorsal (AD) thalamus, anteroventral (AV) thalamus, entorhinal cortex, neurons present in the anterodorsal thalamus →retrosplenial cortex (RSC) circuit, and neurons present in the AV→RSC circuit. In some aspects, the promoter is operatively linked to a polynucleotide encoding an hM4Di polypeptide. Additional aspects are directed to the method for reducing cognitive impairment associated with a neuropsychiatric disorder in a subject comprising administering to the subject a ligand of hM4Di polypeptide, such that the hM4Di polypeptide expression inhibits anterodorsal thalamus hyperexcitability, thereby reducing cognitive impairment in the subject. In some objects of the disclosure, the rAAV comprises a polynucleotide encoding a Cre-dependent inhibitory Designer Receptors Exclusively Activated by Designer Drugs (DREADD) hM4Di. Additional objects of the methods of the disclosure provide for a ligand, where the ligand is compound 21 (C21).

### Definitions

Unless defined otherwise, all technical and scientific terms used herein have the meaning commonly understood by a person skilled in the art. The following references provide one of skill with a general definition of many of the terms used in embodiments of this invention: Kandel et al., “Principles of Neural Science.” McGraw-Hill Professional Pub: 6th edition (Mar. 29, 2021): Singleton et al., Dictionary of Microbiology and Molecular Biology (2nd ed. 1994): The Cambridge Dictionary of Science and Technology (Walker ed., 1988): The Glossary of Genetics, 5th Ed., R. Rieger et al. (eds.), Springer Verlag (1991): and Hale & Marham, The Harper Collins Dictionary of Biology (1991). As used herein, the following terms have the meanings ascribed to them below; unless specified otherwise.

By “hM4Di polypeptide” is meant a polypeptide having at least about 85% identity to GenBank Accession No. AID59732 or fragments thereof having ligand binding activity. An exemplary hM4Di amino acid sequence follows:

>AID59732.1 hM4Dnrxn [synthetic construct]
(SEQ ID NO: 1)
MANFTPVNGSSGNQSVRLVTSSSHNRYETVEMVFIATVTGSLSLVTVVGN
ILVMLSIKVNRQLQTVNNYFLFSLACADLIIGAFSMNLYTVYIIKGYWPL
GAVVCDLWLALDCVVSNASVMNLLIISFDRYFCVTKPLTYPARRTTKMAG
LMIAAAWVLSFVLWAPAILFWQFVVGKRTVPDNQCFIQFLSNPAVTFGTA
IAGFYLPVVIMTVLYIHISLASRSRVHKHRPEGPKEKKAKTLAFLKSPLM
KQSVKKPPPGEAAREELRNGKLEEAPPPALPPPPRPVADKDTSNESSSGS
ATQNTKERPATELSTTEATTPAMPAPPLQPRALNPASRWSKIQIVTKQTG
NECVTAIEIVPATPAGMRPAANVARKFASIARNQVRKKRQMAARERKVTR
TIFAILLAFILTWTPYNVMVLVNTFCQSCIPDTVWSIGYWLCYVNSTINP
ACYALCNATFKKTFRHLLLCQYRNIGTARYPYDVPDYAYPYDVPDYAYKY
RNRDEGSYHVDESRNYISNSAQSNGAVVKEKQPSSAKSANKNKKNKDKEY
YV

By “hM4Di polynucleotide” is meant a nucleic acid molecule encoding an hM4Di polypeptide. An exemplary sequence follows:

>KJ685217.1 Synthetic construct hM4Dnrxn
gene, complete cds
(SEQ ID NO: 2)
ATGGCGAACTTCACGCCTGTCAATGGGAGCTCCGGTAATCAGTCGGTACG
GCTCGTGACGTCGTCCTCGCACAATCGCTACGAAACAGTAGAAATGGTAT
TCATCGCCACAGTAACTGGAAGCCTTTCACTTGTGACTGTCGTGGGAAAC
ATTCTGGTCATGCTCTCGATTAAGGTCAACAGGCAACTCCAGACCGTAAA
CAACTATTTCTTGTTCTCCCTGGCTTGCGCGGATCTGATCATTGGGGCTT
TCTCGATGAACCTCTATACAGTATACATCATCAAAGGGTACTGGCCTCTC
GGAGCCGTCGTGTGTGACCTCTGGCTTGCACTTGACTGTGTGGTGAGCAA
TGCTTCAGTCATGAACCTTCTGATTATCTCATTTGATCGCTATTTCTGTG
TAACCAAACCCTTGACCTACCCCGCAAGGAGAACGACCAAAATGGCAGGG
CTGATGATCGCGGCTGCATGGGTCCTCTCGTTCGTACTTTGGGCTCCTGC
CATTCTGTTCTGGCAGTTTGTCGTGGGTAAACGCACAGTCCCGGACAACC
AATGTTTCATCCAATTCCTTTCGAACCCCGCTGTGACTTTCGGGACTGCG
ATTGCGGGATTCTACCTTCCGGTCGTCATTATGACCGTGTTGTACATTCA
CATCTCGCTTGCGTCAAGAAGCCGCGTCCACAAGCACAGGCCCGAGGGGC
CTAAGGAGAAGAAAGCCAAGACATTGGCTTTTCTCAAATCACCGCTTATG
AAACAGTCCGTGAAAAAGCCTCCTCCCGGTGAAGCAGCGCGCGAGGAGCT
GAGGAATGGTAAACTGGAAGAGGCGCCACCTCCGGCGCTGCCTCCTCCAC
CGCGACCCGTGGCGGATAAGGACACTTCCAACGAGTCGTCGTCGGGGAGC
GCGACCCAGAATACGAAAGAGCGACCTGCTACCGAGTTGAGCACAACAGA
AGCGACGACGCCGGCAATGCCGGCACCGCCTCTTCAGCCCAGGGCGCTGA
ACCCCGCGTCGCGATGGTCAAAGATTCAGATTGTGACGAAACAAACGGGT
AATGAGTGCGTAACAGCCATCGAGATCGTACCCGCAACGCCCGCTGGTAT
GAGGCCAGCCGCCAACGTCGCCAGAAAGTTCGCGTCAATCGCGCGGAATC
AGGTGCGGAAGAAAAGACAAATGGCCGCGAGAGAGAGAAAGGTGACAAGA
ACGATTTTCGCCATCCTCCTGGCGTTTATCTTGACGTGGACTCCGTATAA
TGTCATGGTGTTGGTGAATACATTTTGTCAATCGTGCATCCCAGACACGG
TCTGGTCGATCGGGTATTGGCTCTGCTACGTCAACTCCACTATCAATCCG
GCATGCTACGCATTGTGTAACGCCACGTTCAAGAAAACGTTCCGCCATCT
CCTGTTGTGCCAGTACCGAAATATCGGGACAGCGAGATACCCATATGATG
TCCCCGATTACGCCTATCCCTATGACGTCCCGGATTACGCTTACAAGTAT
CGGAACCGGGACGAAGGCTCGTATCATGTGGACGAGTCACGGAACTATAT
CTCGAACTCCGCCCAGTCAAACGGAGCGGTGGTGAAGGAAAAGCAGCCCT
CATCGGCGAAGTCAGCGAATAAGAACAAGAAGAATAAGGACAAAGAGTAC
TATGTGTGA

By “PTCHD1 polypeptide” is meant a polypeptide having at least 85% amino acid sequence identity to Uniprot Accession No. Q96NR3 or a fragment thereof that functions in neurodevelopment. An exemplary PTCHD1 amino acid sequence follows:

>sp|Q96NR3|PTHD1_HUMAN Patched domain-
containing protein 1 OS = Homo sapiens
OX = 9606 GN = PTCHD1 PE = 2 SV = 2
(SEQ ID NO: 3)
MLRQVLHRGLRTCFSRLGHFIASHPVFFASAPVLISILLGASFSRYQVEE
SVEHLLAPQHSLAKIERNLVNSLFPVNRSKHRLYSDLQTPGRYGRVIVTS
FQKANMLDQHHTDLILKLHAAVTKIQVPRPGFNYTFAHICILNNDKTCIV
DDIVHVLEELKNARATNRTNFAITYPITHLKDGRAVYNGHQLGGVTVHSK
DRVKSAEAIQLTYYLQSINSLNDMVAERWESSFCDTVRLFQKSNSKVKMY
PYTSSSLREDFQKTSRVSERYLVTSLILVVTMAILCCSMQDCVRSKPWLG
LLGLVTISLATLTAAGIINLTGGKYNSTFLGVPFVMLGHGLYGTFEMLSS
WRKTREDQHVKERTAAVYADSMLSFSLTTAMYLVTFGIGASPFTNIEAAR
IFCCNSCIAIFFNYLYVLSFYGSSLVFTGYIENNYQHSIFCRKVPKPEAL
QEKPAWYRFLLTARFSEDTAEGEEANTYESHLLVCFLKRYYCDWITNTYV
KPFVVLFYLIYISFALMGYLQVSEGSDLSNIVATATQTIEYTTAQQKYFS
NYSPVIGFYIYESIEYWNTSVQEDVLEYTKGFVRISWFESYLNYLRKLNV
STGLPKKNFTDMLRNSFLKAPQFSHFQEDIIFSKKYNDEVDVVASRMFLV
AKTMETNREELYDLLETLRRLSVTSKVKFIVFNPSFVYMDRYASSLGAPL
HNSCISALFLLFFSAFLVADSLINVWITLTVVSVEFGVIGFMTLWKVELD
CISVLCLIYGINYTIDNCAPMLSTFVLGKDFTRTKWVKNALEVHGVAILQ
SYLCYIVGLIPLAAVPSNLTCTLFRCLFLIAFVTFFHCFAILPVILTFLP
PSKKKRKEKKNPENREEIECVEMVDIDSTRVVDQITTV

By “PTCHD1 polynucleotide” is meant a polynucleotide encoding a PTCHD1 polypeptide. An exemplary PTCHD1 polynucleotide sequence provided at GenBank Accession No. NM_173495.3 follows:

(SEQ ID NO: 4)

| | |
|---|---|
| 1 | ctcctcccgc gcccgctctg ctctaggatg ctgcggcagg ttctgcacag gggcttgagg |
| 61 | acgtgtttct cccggctcgg ccacttcatt gccagtcacc ctgtcttctt cgcctcggcg |
| 121 | ccggtgctca tctccatcct gctcggcgcc agcttcagcc gctaccaggt cgaggagagc |
| 181 | gtggagcacc tgctgggcgc ccagcacagc ctggccaaga tcgagcgcaa cctcgttaac |
| 241 | agcctcttcc cggtcaaccg ctccaagcac cgtctctact cggacctgca gacccccggg |
| 301 | cgctacggcc gggtcatcgt cacctccttc cagaaagcca acatgctgga ccagcatcac |
| 361 | accgacctga tcttaaagtt gcatgctgct gtcaccaaga tccaggttcc aaggcctggt |
| 421 | tttaattaca cgtttgccca tatatgtatc ctgaataatg ataagacttg catcgtggat |
| 481 | gacatagtgc acgtcctgga agagctaaag aatgctcggg ccaccaatcg gaccaatttt |
| 541 | gctatcacat acccaatcac tcacttaaag gacgggaggg ctgtgtacaa tgggcaccag |
| 601 | cttgggggcg tcactgtgca cagcaaagac cgggtgaaat ctgcagaggc catccagctc |
| 661 | acctactacc tgcagtcaat caacagtctc aatgacatgg tggctgagag gtgggagtcc |
| 721 | agcttctgcg acactgtcag actgtttcag aaatccaaca gcaaagtcaa aatgtaccct |
| 781 | tacacgtcct cctcactgag ggaagatttc cagaagacca gccgcgtatc agaacgttac |
| 841 | ctggtcacca gcctgattct ggtggttacc atggccatcc tgtgttgctc tatgcaggac |
| 901 | tgcgtccgca gcaaaccctg gctaggcctg ctcggattgg tgaccataag cctggccact |
| 961 | ctcactgcag ccgggatcat caatcttact ggtgggaaat ataattccac cttcctggga |
| 1021 | gtccctttcg tcatgctagg tcatggatta tatgggactt ttgaaatgtt atcctcctgg |
| 1081 | aggaaaacta gagaagacca acatgttaaa gagagaactg cagcagtcta tgcagactcc |
| 1141 | atgctctcct tttctctcac cactgccatg tacctggtca cctttggcat aggggccagc |
| 1201 | cctttcacga acattgaggc agccaggatt ttctgctgca attcctgtat tgcaatcttc |
| 1261 | ttcaactacc tctatgtact ctcgttttat ggttccagcc tagtgttcac tggctacata |
| 1321 | gaaaacaatt accagcatag tatcttctgt agaaaagtcc caaagcctga ggcattgcag |
| 1381 | gagaagccgg catggtacag gtttctcctg acggccagat tcagtgagga cacagctgaa |
| 1441 | ggcgaggaag cgaacactta cgagagtcac ctattggtat gtttcctcaa acgctattac |
| 1501 | tgtgactgga taaccaacac ctatgtcaag ccttttgtag ttctctttta ccttatttat |
| 1561 | atttcctttg ccttaatggg ctatctgcag gtcagtgaag ggtcagacct tagtaacatt |
| 1621 | gtagcaaccg cgacacaaac cattgagtac actactgccc agcaaaagta cttcagcaac |
| 1681 | tacagtcctg tgattgggtt ttacatatat gagtctatag aatactggaa cactagtgtc |
| 1741 | caagaagatg ttctagaata caccaagggg tttgtgcgga tatcctggtt tgagagctat |
| 1801 | ttaaattacc ttcggaaact caatgtatcc actggcttgc ctaagaaaaa tttcacagac |
| 1861 | atgttgagga attcctttct gaaagcccct caattttcac attttcaaga ggacatcatc |
| 1921 | ttctctaaaa aatacaatga tgaggtcgat gtagtggcct ccagaatgtt tttggtggcc |
| 1981 | aagaccatgg aaacaaacag agaagaactc tatgatctct tggaaaccct gaggagactt |
| 2041 | tctgtcacct ccaaggtgaa gttcatcgtc ttcaatccgt cctttgtata catggatcga |
| 2101 | tatgcctcct ctctgggagc ccccctgcac aactcctgca tcagtgcttt gttcctgctc |
| 2161 | ttcttctcgg cattcctggt ggcagattca ctgattaacg tctggatcac tctcacagtt |
| 2221 | gtgtccgtgg agtttggagt gataggtttc atgacattat ggaaagtaga actggactgc |
| 2281 | atttctgtgc tatgcttaat ttatggaatt aattacacaa ttgacaattg tgctccaatg |
| 2341 | ttatccacat ttgttctggg caaggatttc acaagaacta aatgggtaaa aaatgccctg |
| 2401 | gaagtgcatg gggtagctat tttacagagt tacctctgct atattgttgg tctgattcct |
| 2461 | cttgcagctg tgccttcaaa tctgacctgt acactgttca agtgcttgtt tttaatagca |
| 2521 | tttgtcacct tctttcactg ctttgccatt ttacctgtga tactgacttt cctgccaccc |
| 2581 | tctaagaaaa aaaggaaaga gaagaaaaat cctgagaacc gggaggaaat tgagtgtgta |
| 2641 | gaaatggtag atatcgatag tacccgtgtg gttgaccaaa ttacaacagt gtgataatgt |
| 2701 | ctgcttggca tattttcacc ttaggtctta tcaagaccaa agagattatg ttaatgaaac |
| 2761 | aattaaattc aaagttcttc ccttttttaa agataggaaa caggcattgc caaaaaaaaa |
| 2821 | aaaaaaaaaa aaaggaaagg acagtgggga gaaatgggcc tggcatattt tcagtcttta |
| 2881 | aaacaaagga gttgttatga gaattcacac acacatagac acacacacac acacacacac |
| 2941 | acacacacac acacacacac cctgggagac ctatagtctc ttaaactaag atcaagtaga |
| 3001 | agaaagctta ttaacaagca ggatcctgcc ttatccaaac tgcagatgtt gctggcattg |
| 3061 | tgacaaaacc cactgattga aaggtcaact gccaaggcag aaacaccttt aagcattgtt |
| 3121 | caaacaataa ggcttccaga acttctgtag agcagtagct ccagtcatgg tctgtggttt |
| 3181 | gaggttttag ctgtctcacc tagctcccta acactgaagg agatacttgt gaaagttctg |
| 3241 | accagcaaaa gcaagccaga gccttggaaa ctgatatgtg gtagagtggc catcactcat |
| 3301 | ggactaaaat tgattcaccg ctaaatttac ccaggtgaag cagtttcgtt gtctagaatg |
| 3361 | aaattatcat attccgccat tggtatgcct ttaacatttg tatagtttgg tttgcttaaa |
| 3421 | acaccttaaa accaatgaca gctccagcac tgcagaattg gtgtgattct actttggaat |
| 3481 | agcttgtcac ttgtcaccaa atgggtctgc tttattagtt acagctcttg gcaggaggat |
| 3541 | ccagggaccc aaaaccacag ggccaaaccc aaatacctgg catgatggag caaaagcagg |
| 3601 | tgtctacttg gacccagata tagtgtctcc attttaacaa caacaacaaa atagccagct |
| 3661 | ggtacagctg tttgcattgg ccctacatgc attttttgca tggatatcca gaaacatctg |
| 3721 | cccacacaaa actgcgggga aaaaaaatga acactgaaat agttatttgc tgttgcttcc |
| 3781 | aacttgtagt gccagtctgc ctttgctgtg aaacacacct gctcagagac agagagggga |
| 3841 | agaagatctt tggtaagtct aagtcctgac gctgagaagc tttgtaaaag tgcagggaga |
| 3901 | taaagggcca aaagggagat agatggaaaa cactggaaaa agtattcact gatacaaatc |
| 3961 | tatcaatgat ggcagtccaa ttctcttgct aaagtggctg cacctcacct tgctggtccc |
| 4021 | ccccacacct tttttgatgt ccttctgcgt catcatagca aggcccttct gtaaattaac |
| 4081 | aagcctagat atttatactc ttgacttcca gtatctacag aagaatggtt catagatcta |
| 4141 | aacagaaatg gtttagatct aaaaaggctg tatacgttgc ccaggcccct gcatttcttt |
| 4201 | aaatttataa aaatgaagct aaaacctggt tacatttgaa gcaaatatct acagtatttt |
| 4261 | tcccttttag agatgtagct tccttagaca tctgtagtgg taagcatttc ccaaaagcat |
| 4321 | cttacctttc tgaaccttag cagacatact gtgcagctta cctatcttct gcagaggagg |
| 4381 | aaactgagac ctaggagaat aaagtgactc actcaggtca caccactaaa gggttttcat |
| 4441 | catttcagca tacctaagac agggcagtcc aattttcagt attctcataa gatggctatt |
| 4501 | actcctctca aaatgcattt ccaaagtagg aacataggac ttcgttggcc acagggcaga |
| 4561 | cattttttta gtgtctggaa ttaaaatgtt tgaggtttag gtttgccatt gtctttccaa |
| 4621 | aaggccaaat aattcagatg taaccacacc aagtgcaaac ctgtgctttc tatttcacgt |
| 4681 | actgttgtcc atacagttct aaatacatgt gcaggggatt gtagctaatg cattacacag |
| 4741 | tcgttcagtc ttctctgcag acacactaag tgatcatacc aacgtgttat acactcaact |
| 4801 | agaagataat aagctttaat ctgagggcaa gtacagtcct gacaaaaggg caagtttgca |
| 4861 | taatagatct tcgatcaatt ctctctccaa ggggcccgca actaggctat tattcataaa |
| 4921 | acacaactga agaggggatt ggttttactg ttaaatcatg tgttgctaaa tcattttctg |
| 4981 | aacagtgtgt tctaaatcag tcattgattt agtgtcagcc acgtggagca cctcggctta |
| 5041 | aagcagctcc acaaaacctg acacaacaca cacaccaatt aaatggattt tgttgagaat |
| 5101 | ttaatcattc aatttggtca accagaatga cttcctgtgg aactctgttt tatgacagat |
| 5161 | aatagttttc caacttgatt gagtctctgt ataccctggg atattgtatt ttttaatgaa |
| 5221 | gggcattttc aaacttgtca acttctcttt tcagcacttg aaatgaaggc ttatggaatt |
| 5281 | ctgactgtga aatgaatttt tctattggga gattatgcat gccaagattt attatttatt |
| 5341 | gttagataat tattatttat ttgttaatac tgattattga ttattgctct gctgcattgg |
| 5401 | tttagccccg ctatggagaa gacactttag aagtaactca gaatcacatc cccataaaat |
| 5461 | ctaagtcttt acttcatcaa gttttacttt ttgaattgct ataaagacgg aaaaattgat |
| 5521 | ccctgcaaac acttgaagaa gctgtgtttt ttttctttca attaattcca accaagacat |
| 5581 | taacgtccac tttaccaaag aaacttactt ggttggtttt tagttggtgt ttgttgtttg |
| 5641 | tttaccaaag aaaatattcg attcatcaaa aatattgctc ctggtcaact aaaaaactgt |
| 5701 | gatactggga cttttttggc cattcataag gaaaattttt aaaagtcaag gtaagggtga |
| 5761 | tattctctca tttagtggaa gaagaagatg tcaattcaca gggaatgtgg tgttcgcaag |
| 5821 | aaagaaaatg atacttgaaa tagggcatta ttgtttagcc tacagcaaca tttcaacaag |
| 5881 | cagcatcagg gctcctcagg ttaaaagaag tacagtttgt ttcagagaaa aaggaagaca |
| 5941 | aaccctcgtg aaatgtttct ccattgttcc agctcataag ttttactttg ttttgggttt |
| 6001 | tggagttttt agtttctttg atgtttgttt tgctttaatt tggtttgtgg tttgtgtttg |
| 6061 | cttgtgtttg cttctgcctt tcctaccttc tgccttttac tccctcagtt ttcagtcaga |
| 6121 | agtgtgataa tcagatgcca tcctccatgg ccacgcttat gtatgcaata taccgctcca |
| 6181 | aatgaggacc ttggtaaccc ttgacccttt aattctaact aaccatcacc caatcgtaag |
| 6241 | tggcattatg ggaccatgat accacttttc tccagtgtca atgacagagc tcagctgtgt |
| 6301 | ccatcacaag aaaaggtgga atgggcagct gtcttggggg tgtggcaaga ggcagaacct |
| 6361 | cttggacaca aatgttatct tcctcataat ggctttattc ttttggatcc tattaaagta |
| 6421 | cccctgtagc ttatagtagt gtttgacctc tgtgactctg ttttctcatc tgtaaaatgg |
| 6481 | ggataatatc agtaaatgtc tttaaattca gcaaatactt atcgagtgcc aaatatcgcc |
| 6541 | cagatactgt tcgagaggct ggggacacaa cagtcaacaa atcagatttt ttaaaatctg |
| 6601 | ctcttgtaga gtctaccttt gagtgacatt gcaggtatat tgtggggatt gaatgagatg |
| 6661 | agagatgaca tttggaaagc actcttggaa aggcctggca aagaaaagag actcaagaca |
| 6721 | ttatttgtaa ttcagcaatc tcgcacagta gcattagggc tcgggatgta aagctactga |
| 6781 | gaagtagatt tgagcctgag aattagggct acaaatacaa tgagttgcca cagaaggtag |
| 6841 | ttaattacca gtcattttag gacatcaaaa tgaagctgat caccacatag tggggccact |
| 6901 | gtaaaggggc tgcaagtcct ggatgttggc atcatactat gatctttgaa aaaaacagat |
| 6961 | acaggcactt gcttttgggg atggggaagg gacaccactg attactgaat aactgtggct |
| 7021 | ggcttgtctg agggaatgta aacattccct tgaggtaatt tgcttttaat tttgtgtaaa |
| 7081 | aagcaccagg tatttagaga cataaagttc ccgtttttta aaagaatagt aaatctgcaa |
| 7141 | tacaaagaaa aatattaagt tagtggattc gctggcaaag aactaagaat ggctgcagcc |
| 7201 | aacacactgc agagttcctc cctgtggtgg ttacaactgt tatttctgtt tcatgccagt |
| 7261 | ccaggagaaa gcaaagagga aagtggatga gctataatcc gttcacctga ataggcagtg |
| 7321 | ccaggatgtc ccccccagct tgcctctcac ccgccccgct gctgcccagc tgatttgttt |
| 7381 | gaagtcctct ttgatatcta tgagagcacc gtggaaggaa gaagaaggga ggcgaccgga |
| 7441 | gagctatggc ctctgtgcag atttttgagt tagattaaaa ttagtaccac ctgattagga |
| 7501 | gcctacctgg tgaggaaaaa agccttcttt tcaaagtaca aaaaaaagcg atgtgtacca |
| 7561 | aggagaaaac cagcagagaa agcttggtgg aagaagtgat gttcttgtgg aatgtgccag |
| 7621 | cagcttctcc ttccagaatg ctgacaccag atgaatcttt ggttagaatt aagcctaccc |
| 7681 | ctttgttttt cacatgagaa aactgaaccc tcgagtaatg aaattatatt cacagagcta |
| 7741 | attagaaaca ggagactata attgaaatct agtttttagc ctcaattctt tctccatttt |
| 7801 | gccactttac tgccaaattc tattcatcta aattattggg gtcttaacct gggggccaaa |
| 7861 | tgagttcatg ggaggggtct ctgagtttcc tacaattaag ttacatgcaa aaatgtgtgt |
| 7921 | atatgttcaa catgtgcatt ttcctaaact gagggctcag attccaaaag aggtctatga |
| 7981 | ccgaaaaagg gtgaagaacc actgtcctaa attaatacag acctcattgg gagtgtgatc |
| 8041 | ctggtgtcct cattttggct aatgagcaat caagaacttt tgaactatct tttctctaag |
| 8101 | gatccaaaaa tgcttcaaaa tattggtttc ttcatcccaa gccaagtgcc tacttctatt |
| 8161 | catttattct tcattcattc tttcattcat tcatttcttt attctgcaaa tgtattttca |
| 8221 | gtgccaattc atgttctagc agcagctaaa tatgccctcc acagcaggac caggaaagga |
| 8281 | gccctctgta ctgtgctctt gttggcaaga ggaacaccag gttgctacca cattctcact |
| 8341 | gttgcaacaa attatatata gcctcaaatt ttcagtaacc atcccacaca cacatacaca |
| 8401 | cataaaggca gcagccaaca aagccaacat atagaagtgt gccctgtcaa agaaccagga |
| 8461 | agagcagcct atttccaggt tatatacagt gatatgctgt ttacaactgc ataccacatc |
| 8521 | cttagaaggc ctgctcccaa atgagcccta ctctgagatg tctacaccag gcatgctttc |
| 8581 | acacacgtgc agtaccctac tctcaaaaca agtatggaga gaataagtct cattcatctc |
| 8641 | gttaatattt ttagtgggca aagaagcaac cgtggccatg gacagatggg tgcatttaag |
| 8701 | gacaggtgtg gccgcgcaca gtggctcaag cctgtaatcc cagcactttg ggaggcggag |
| 8761 | gcgggcagat cacgaggtta agagattgag accatcctgg ccagcatggt gaaaccccgc |
| 8821 | ctctactaaa aaatacaaaa aaattagctg ggcgtggtgg cacgtgcctg tagtcccagc |
| 8881 | tactcaggag gcaggagaat cccttgaacc cgggaggtag aagttgcagt gagccgagat |
| 8941 | ttcaccactg ccctccagac tggcaacaga gtgagacttc gtctcaaaaa aataaaggac |
| 9001 | aagtgtgttt tatacacaaa tatgcagaag cccagttctc ttgactccaa gtcagaagct |
| 9061 | agataaaata gaggttgctg ggggaagttg gaaaataaat gactatactg tacttgttgg |
| 9121 | ctctgctgct gctgatataa cttcaggcac aacggaagcc gcctcggttg gcctatggga |
| 9181 | agggctgggt gaatcactgc ttggccttgc atgaagcaca aaggacacac caatggctga |
| 9241 | tcagtggccc ttccatcatc ttctgcccag ccatgataag gaaagccttc tattgagatg |
| 9301 | gaggaatatt agctcaactt tgtagcatct gatgtaaagt ttgaggcgtt tgtctttttt |
| 9361 | gtttgtttgt ttgttttttt ttgagatgga gtctcactct gtcacccagg ctggagtgca |
| 9421 | gtggcgccat ctcagctcac tgcaagctcc acctcccagg ttcacgacat tctcctgcct |
| 9481 | cagcctcctg agtagctggg actacaggtg cccgccaccg cgcccggcta attgtgtgtg |
| 9541 | tgtgtgtgtg tgtgtgtgtg tgtgtgttta gtagagatgg ggtttcaccg tgttacccat |
| 9601 | gatagtctcg atctcctaac ctcgtgatcc acccgcctcg gcctcccaaa gtgctgggat |
| 9661 | tacaggcgtg agccaccgtg cccggcaagg gctttgttca ttaggatcaa caaagtgctt |
| 9721 | tgtctaattc ctgcaaaatg gcaaagaccc agcccatttc cattactcat tccaggctca |
| 9781 | tgctcaggaa aactaataac gttctctttg acaaaacaat ttccttaatt ttctaggatg |
| 9841 | aatcttgaac atgtttagct cagcctttca gatatttatt ctttaaatta ccatcctgtc |
| 9901 | aaaattgcct ttacgcactg atccagaatc accaaggcag ttgccacgat gcctcccagg |
| 9961 | actgtgggtg actgggcaaa ctggtaatga cccgttggct ctgcttcaca cacacacata |
| 10021 | cacattgaca cacataaatg tacacatgca tatatgtgtg tacaacacaa ctataaaaca |
| 10081 | tagaaacata tacctgtaca tatgcacatg cttacataca cacatatcac agatgtgcat |
| 10141 | gcacacatac acatgcacac caccttttac aatatgcctg acacatttga ggcacattaa |
| 10201 | aatgccttgg caggcccagc cctatagttc tgtaaggttt ggcaggccaa aggcttcgga |
| 10261 | tgtctgcagg ataccattga aaaaatgtta tcttcccctt ctatatgttt cttatgcacg |
| 10321 | agcacagcta agagcagcca aatagtaagc tttaaagaaa agaaacctgg aaaagtgaat |
| 10381 | gtgccttgca atttaaaaat tgctaacagt ttcacagtgg gaaaattgca tttgttggaa |
| 10441 | ttttccaagt cataagcaag aagaatagca tagacccaaa acgacagaga tctcttccaa |
| 10501 | ggtatggaat tgtggaaatt tatttagaat atttgcttgg tgatccagag aagggggaaa |
| 10561 | gaagtggtga ggaagaatat atggggtgat ccctccaaat tgccaactcc tcgtcttagg |
| 10621 | aagaagtggg cagtttccat tgatccctag catcagtttg acgggaaaac ccgtgcattt |
| 10681 | catcaacctc aaggaaagga agtgccatgc atggaggcct ggtgataggc aacatccatt |
| 10741 | tagaagaaca gtcacacaag caacagaaga actatccaag actagaagct tagaagctta |
| 10801 | taccaaactc tctgtctctc tctctctctc tcacacacac acacacagag tggatgggag |
| 10861 | gggacattaa gaggaaccct agtgcagttc ccccaaaaaa ccttggcata gagtataaca |
| 10921 | gggaaaaagt aattactggg aagctgaaaa caatcaaata tgtgaggaat gactgaatat |
| 10981 | gagctgagga taatgtaaat tgttctggat tgtctataag ataaagatac agagcctgaa |
| 11041 | tctatgtttt tctgaataaa gagaccccat attctctttt gaattattga cctataattc |
| 11101 | ctcttcagca gctttgaata cactcaagtt ctcttcctat tgtttatgat cccataatac |
| 11161 | aaccacccac tatttgttgt ggtttacaat ccagcttaga acccttgagc aaaattattg |
| 11221 | aattattgaa tgctaggccc aaacaaaaga aatttcagct tggttttagt aatttcttga |
| 11281 | tcaaacttct ctcattcctc taaatgtaca aaggtttcaa agttgtaaaa aatatgtcta |
| 11341 | catagccaat gctcttagta tttttaacag caaagataat ctctgccaat aattattaat |
| 11401 | gacaaaatag tgataactgt tcaatgttgc tttaacagtc caattgtagt aggtgagaaa |
| 11461 | acaaaaaaga agagaagcta tgttgaatat ttagtaatgt tggaaaagat cctcacccta |
| 11521 | gacataaaat agtttaaaca tttaaacatg gcccctctct tattgtcatt gctagtgaaa |
| 11581 | gaagttcctg aaattgcata ttacattgta aagttttggg ctaaatcaca atacattttt |
| 11641 | aagttgcttc catggcaata gatcgtaaga ccacagcttg tcaaagtaac cactaattca |
| 11701 | gtattcagaa accttatcat ctgttcctcc aaaaagaccc catagttaat gttgtgtttc |
| 11761 | ccagtatttt agttgcgttt cttcacactt aggcaagtgg gtgattctgg tcataataaa |
| 11821 | ctattttcaa actaggaaaa agtatccaaa tgctagagca cataatgagt tcagcttggt |
| 11881 | cagttttgag acagataagc ataaaaccct actagaaaaa aataatagtc aaacctaaag |
| 11941 | ctttaaaaaa tgtgagtcac atcacttttc tccaggaaag gcagaacgtg ctcagggtgg |
| 12001 | aatttaatca aaaatgtacc cagaacaatt tcccacaaag agctgtaata ctttgcagca |
| 12061 | aggggcatta ataaactcta atttttaccg aaagtcaaga gacacacaga tcttttttgg |
| 12121 | taccctctac atcatcatag caaggccctt ctgaacatta acaagcctag atgtttctac |
| 12181 | tgcttatttg cagtatctac aggggaatgg ttcgtggata taaacagaaa tgttttatct |
| 12241 | gaagaggctg tgtatgccac cccagacccc tgcatttctt aatatctata aaaatatgac |
| 12301 | taaacttgtt acatttgagg caaatatgta cagtattttt cccttttaaa gatagtttaa |
| 12361 | aaaatcaagg taataaaaaa cggccaactc tttcaggaaa aaaaaaatcc ggtattttgt |
| 12421 | actacttgag tttctctaat gcctaaaata tgcatttaaa tcatcccaca cctacacaga |
| 12481 | aacaaaatca gcagggaaaa ccatcccact gatgattggc aattccaccg ttcctctcca |
| 12541 | tctgaccagc tttcaccaga gggcgatctt tcatgcttgc acatcgagtt tatacaatgt |
| 12601 | agaagccccc aagtcacctg ggaaatggct catgccaaat acatggagtc cttagttgac |
| 12661 | tatcttttca tattaaaatc ccatcttcca atgaagcatc ctgttttcaa tactgatatc |
| 12721 | aaatttcgaa tgtaaaggag atttcaaatg tattgctttc accttttctt ttttattcat |
| 12781 | ttatttttaa ttgacataaa aattgtatat atttatggga tacaatgtga tgttttgata |
| 12841 | tatgtatgca ttgtggaatg attaaatcaa gctaattaac ata |

By “YWHAG polypeptide” is meant a polypeptide having at least 85% amino acid sequence identity to NCBI Accession No. NP_036611 or a fragment thereof that functions in neurodevelopment. An exemplary YWHAG amino acid sequence follows:

(SEQ ID NO: 5)

| | |
|---|---|
| 1 | mydreqlvqk arlaeqaery ddmaaamknv telneplsne |
| | ernllsvayk nvygarrssw |
| 61 | rvissieqkt sadgnekkie myrayrekie keleaveqdy |
| | lslldnylik ncsetqyesk |
| 121 | vfylkmkgdy yrylaevatg ekratvvess ekayseahei |
| | skehmgpthp irlglalnys |
| 181 | vfyyeiqnap eqachlakta fddaiaeldt lnedsykdst |
| | limgllrdnl tlwtsdqqdd |
| 241 | dggegnn |

By “YWHAG polynucleotide” is meant a polynucleotide encoding a YWHAG polypeptide. An exemplary YWHAG polynucleotide sequence is provided at GenBank Accession No. CR541925.1, which follows:

| (SEQ ID NO: 6) | |
|---|---|
| 1 | gcagccgcct cgcgcccggt cccgcggtcg cagctccagc cgcctcctcc gcgcagccgc |
| 61 | cgcctcagct gctcgctctg tgggtcggtc ctctccggca cttgggctcc agtcgcgccc |
| 121 | tccaagccct tcaggccgcc ccagtgtcct cctccttctc cggccagacc cagccccgcg |
| 181 | aagatggtgg accgcgagca actggtgcag aaagcccggc tggccgagca ggcggagcgc |
| 241 | tacgacgaca tggccgcggc catgaagaac gtgacagagc tgaatgagcc actgtcgaat |
| 301 | gaggaacgaa accttctgtc tgtggcctac aagaacgttg tgggggcacg ccgctcttcc |
| 361 | tggagggtca tcagtagcat tgagcagaag acatctgcag acggcaatga gaagaagatt |
| 421 | gagatggtcc gtgcgtaccg ggagaagata gagaaggagt tggaggctgt gtgccaggat |
| 481 | gtgctgagcc tgctggataa ctacctgatc aagaattgca gcgagaccca gtacgagagc |
| 541 | aaagtgttct acctgaagat gaaaggggac tactaccgct acctggctga agtggccacc |
| 601 | ggagagaaaa gggcgacggt ggtggagtcc tccgagaagg cctacagcga agcccacgag |
| 661 | atcagcaaag agcacatgca gcccacccac cccatccgat taggcctggc tcttaactac |
| 721 | tccgtcttct actatgagat ccagaacgcc ccagagcaag cgtgccactt ggccaagacc |
| 781 | gcgttcgacg acgccatcgc cgagcttgac accctcaacg aggactccta caaggactcc |
| 841 | acgctcatca tgcagctcct ccgcgacaac ctcacgctct ggacgagcga ccagcaggac |
| 901 | gacgatggcg gcgaaggcaa caattaaggc cccaggggaa ctggcagcgc acgcggatgc |
| 961 | tactactgca gtctttattt ttttcccatg agttgggggt cgggtguggg agggaaaggg |
| 1021 | agggatgacc ttcccaggga gaaacccacg acctgtcctg tctttgatcg cctctttgac |
| 1081 | atttttgcca aaataccact agtggaaagt caggctagct gtgctggtat tggaatagca |
| 1141 | gcctcacact ggcgtctgga ctgttctgta gattcatgca agtggagctg tctgtctcta |
| 1201 | atttaactta ttgctagata atagggtttt cagatgaaaa gaaaacttaa agaggaatgg |
| 1261 | ccctcattca gtaagttctg tggttccagt aaggattttt atgtacatac gctctcgtct |
| 1321 | ctcgttttgg gtactttcta tctcatctgt ctcggctctg catgttttcc agggtgtagc |
| 1381 | ctacagacat ggaacagtgt aaatcccaga ctgacagact tagaacctga ggtctcattc |
| 1441 | atccttatgg tttaggcctt gccagttttc cgaagtctct gattagttga cagtattaac |
| 1501 | actaaattgc agtttacagt atttctacat tacagccata tgtaacatca agccatcgat |
| 1561 | tgtgtacttt tcctttgcta gttgtttggg ctttaacatc cttattcagc cttatccagg |
| 1621 | ttggttttgc tgttgatcgg tctcctaggc taaatgagaa tgaaagcgac ttcaggtcag |
| 1681 | gtggctgtgg gatttttttt ttttggtcct tctttcctct taacgtaaat ccaccaccaa |
| 1741 | aattattaat cctcttgaga gaaacgtgaa acgccacaaa aatagagaaa attcaggtct |
| 1801 | gtatgtcatg gatcgtgttg gtattttcag agaacatccc gcttctgaag ctgctgcagc |
| 1861 | tccctcctca gggatcacac tgccgtcacc cactctgcac tggggcgttt cctactgcgc |
| 1921 | ctcgtgctgg cggacgcagc tgggtgcaga agctgtgggg tcggagaggc gtttggagaa |
| 1981 | ggtctgtggt gcagtgtgtg aaaattcagg tgctagaagc ctactggtag aaaaacccaa |
| 2041 | aaggaagagc tatatcctta accattctgt ccaatttcgg gagccttgtc agtgtgtcag |
| 2101 | tttttcctcc ccgaagacac tccttcccca agtaattgta ggaagataaa aaaactgtta |
| 2161 | ccagataaca aacactgaac tcctatttga ccagaacttt ttcctctcga gatagttttt |
| 2221 | tctttttaat gaaaaaagca taggaattgg agattggctt gtctcacgca gccagtgcac |
| 2281 | atttggaatt gacggaaaca acgttgctat ttccacccat ttgttttcgg cagccttaag |
| 2341 | gccctcattc tcatttcggg tgaatctgtc tatctgtgaa cgtggcccgc atgtgcattc |
| 2401 | ttttttttat atatataaag tcagtgacga ggaactcccg agacgtgtaa tgacaccaca |
| 2461 | cttgttttct ttgtttcttt gttttattta ggcaagaaga ggtgtgagta attgaggaaa |
| 2521 | aactgacaga tgcttttgct aataccaaaa ttgagcttac aattaggaac tgagtatgtg |
| 2581 | taacaggata caggtgacag tgaagataga agaaccacga tgaccacaga ctcaatgtgc |
| 2641 | tctgtaacat cgcacagttt acccagcatg actttcctta ggaggccccc tcctcacgct |
| 2701 | agagtaaaag tcccagttaa gtgaagccta ccagaagaac tagtagaaga agctttgccg |
| 2761 | cttttgtgcc tctcacaggc gcctaaagtc attgccatgg gaggaagacg atttgggggg |
| 2821 | gggagggggg gggggcaggg taggtggggc tttccctaat ttatcttcat gtccagtgag |
| 2881 | cagtgttgcg tttttccttg tagcatttgg aaatgattta ctggaattac aaaacctatt |
| 2941 | tttcctttaa atttcagctt tggctctggc tgctttttag aataatgcaa gataaaaatc |
| 3001 | acacctgagg gctgaaaacg gagagggaat gggagacttg atatttaagc agcttgaatg |
| 3061 | gtttttcttt tctttatttt taaagaaatg cacttgccta tgatactgtc tctccagtga |
| 3121 | aatgattact cctccattac tctattgata caatattgtg catgctagtg ttgtatttct |
| 3181 | atacagtagc ttgaaattga ttaacttata ctgtaggtgt tatgtattcc tatgacaaaa |
| 3241 | aaaattaagt cttcaaattt tttaaaggtt tttttttttt aatttaattt ttccttttgg |
| 3301 | gggtaaagtt tgctctacca aatagtgatt gtaacaaatt gatctgtttt ggatgttgct |
| 3361 | atagtgacat gcagttatat attttgtttt taaaaggggg ggagcaaaag aaacaccagt |
| 3421 | gttagcttaa tcttaatgtc tcgtgtttgt catggtgaaa ttataactat tacagtgttg |
| 3481 | gagaacaaca aatatgttct ctgaatgagc ctttgtgctt tttgtcatgt tatgcagtga |
| 3541 | actattttta aggtctaatc agtgattatt tttccagctc cgtgtttctc taaggaatta |
| 3601 | tttcacacac ggaccatctt tagcagtttc ctcagtgatg gaatatcatg aatgtgagtc |
| 3661 | attatgtagc tgtcgtacat tgagcaaata aacttacaga tctga |

By “HERC1 polypeptide” is meant a polypeptide having at least 85% amino acid sequence identity to GenBank Accession No. NP_003913.3 or a fragment thereof having ubiquitin-protein ligase acitivity. An exemplary HERC1 amino acid sequence follows:

(SEQ ID NO: 7)
MATMIPPVKLKWLEHLNSSWITEDSESIATREGVAVLYSKLVSNKEVVPL
PQQVLCLKGPQLPDFERESLSSDEQDHYLDALLSSQLALAKMVCSDSPFA
GALRKRLLVLQRVFYALSNKYHDKGKVKQQQHSPESSSGSADVHSVSERP
RSSTDALIEMGVRTGLSLLFALLRQSWMMPVSGPGLSLCNDVIHTAIEVV
SSLPPLSLANESKIPPMGLDCLSQVTTFLKGVTIPNSGADTLGRRLASEL
LLGLAAQRGSLRYLLEWIEMALGASAVVHTMEKGKLLSSQEGMISFDCFM
TILMQMRRSLGSSADRSQWREPTRTSDGLCSLYEAALCLFEEVCRMASDY
SRTCASPDSIQTGDAPIVSETCEVYVWGSNSSHQLVEGTQEKILQPKLAP
SFSDAQTIEAGQYCTFVISTDGSVRACGKGSYGRLGLGDSNNQSTLKKLT
FEPHRSIKKVSSSKGSDGHTLAFTTEGEVFSWGDGDYGKLGHGNSSTQKY
PKLIQGPLQGKVVVCVSAGYRHSAAVTEDGELYTWGEGDFGRLGHGDSNS
RNIPTLVKDISNVGEVSCGSSHTIALSKDGRTVWSFGGGDNGKLGHGDTN
RVYKPKVIEALQGMFIRKVCAGSQSSLALTSTGQVYAWGCGACLGCGSSE
ATALRPKLIEELAATRIVDVSIGDSHCLALSHDNEVYAWGNNSMGQCGQG
NSTGPITKPKKVSGLDGIAIQQISAGTSHSLAWTALPRDRQVVAWHRPYC
VDLEESTFSHLRSFLERYCDKINSEIPPLPFPSSREHHSFLKLCLKLLSN
HLALALAGGVATSILGRQAGPLRNLLFRLMDSTVPDEIQEVVIETLSVGA
TMLLPPLRERMELLHSLLPQGPDRWESLSKGQRMQLDIILTSLQDHTHVA
SLLGYSSPSDAADLSSVCTGYGNLSDQPYGTQSCHPDTHLAEILMKTLLR
NLGFYTDQAFGELEKNSDKFLLGTSSSENSQPAHLHELLCSLQKQLLAFC
HINNISENSSSVALLHKHLQLLLPHATDIYSRSANLLKESPWNGSVGEKL
RDVIYVSAAGSMLCQIVNSLLLLPVSVARPLLSYLLDLLPPLDCLNRLLP
AADLLEDQELQWPLHGGPELIDPAGLPLPQPAQSWVWLVDLERTIALLIG
RCLGGMLQGSPVSPEEQDTAYWMKTPLFSDGVEMDTPQLDKCMSCLLEVA
LSGNEEQKPFDYKLRPEIAVYVDLALGCSKEPARSLWISMQDYAVSKDWD
SATLSNESLLDTVSRFVLAALLKHTNLLSQACGESRYQPGKHLSEVYRCV
YKVRSRLLACKNLELIQTRSSSRDRWISENQDSADVDPQEHSFTRTIDEE
AEMEEQAERDREEGHPEPEDEEEEREHEVMTAGKIFQCFLSAREVARSRD
RDRMNSGAGSGARADDPPPQSQQERRVSTDLPEGQDVYTAACNSVIHRCA
LLILGVSPVIDELQKRREEGQLQQPSTSASEGGGLMTRSESLTAESRLVH
TSPNYRLIKSRSESDLSQPESDEEGYALSGRRNVDLDLAASHRKRGPMHS
QLESLSDSWARLKHSRDWLCNSSYSFESDFDLTKSLGVHTLIENVVSFVS
GDVGNAPGFKEPEESMSTSPQASIIAMEQQQLRAELRLEALHQILVLLSG
MEEKGSISLAGSRLSSGFQSSTLLTSVRLQFLAGCFGLGTVGHTGGKGES
GRLHHYQDGIRAAKRNIQIEIQVAVHKIYQQLSATLERALQANKHHIEAQ
QRLLLVTVFALSVHYQPVDVSLAISTGLLNVLSQLCGTDTMLGQPLQLLP
KTGVSQLSTALKVASTRLLQILAITTGTYADKLSPKVVQSLLDLLCSQLK
NLLSQTGVLHMASFGEGEQEDGEEEEKKVDSSGETEKKDFRAALRKQHAA
ELHLGDFLVFLRRVVSSKAIQSKMASPKWTEVLLNIASQKCSSGIPLVGN
LRTRLLALHVLEAVLPACESGVEDDQMAQIVERLFSLLSDCMWETPIAQA
KHAIQIKEKEQEIKLQKQGELEEEDENLPIQEVSFDPEKAQCCLVENGQI
LTHGSGGKGYGLASTGVTSGCYQWKFYIVKENRGNEGTCVGVSRWPVHDF
NHRTTSDMWLYRAYSGNLYHNGEQTLTLSSFTQGDFITCVLDMEARTISF
GKNGEEPKLAFEDVDAAELYPCVMFYSSNPGEKVKICDMQMRGTPRDLLP
GDPICSPVAAVLAEATIQLIRILHRTDRWTYCINKKMMERLHKIKICIKE
SGQKLKKSRSVQSREENEMREEKESKEEEKGKHTRHGLADLSELQLRTLC
IEVWPVLAVIGGVDAGLRVGGRCVHKQTGRHATLLGVVKEGSTSAKVQWD
EAEITISFPTFWSPSDTPLYNLEPCEPLPFDVARFRGLTASVLLDLTYLT
GVHEDMGKQSTKRHEKKHRHESEEKGDVEQKPESESALDMRTGLTSDDVK
SQSTTSSKSENEIASFSLDPTLPSVESQHQITEGKRKNHEHMSKNHDVAQ
SEIRAVQLSYLYLGAMKSLSALLGCSKYAELLLIPKVLAENGHNSDCASS
PVVHEDVEMRAALQFLMRHMVKRAVMRSPIKRALGLADLERAQAMIYKLV
VHGLLEDQFGGKIKQEIDQQAEESDPAQQAQTPVTTSPSASSTTSFMSSS
LEDTTTATTPVTDTETVPASESPGVMPLSLLRQMFSSYPTTTVLPTRRAQ
TPPISSLPTSPSDEVGRRQSLTSPDSQSARPANRTALSDPSSRLSTSPPP
PAIAVPLLEMGFSLRQIAKAMEATGARGEADAQNITVLAMWMIEHPGHED
EEEPQSGSTADSRPGAAVLGSGGKSNDPCYLQSPGDIPSADAAEMEEGFS
ESPDNLDHTENAASGSGPSARGRSAVTRRHKFDLAARTLLARAAGLYRSV
QAHRNQSRREGISLQQDPGALYDFNLDEELEIDLDDEAMEAMFGQDLTSD
NDILGMWIPEVLDWPTWHVCESEDREEVVVCELCECSVVSFNQHMKRNHP
GCGRSANRQGYRSNGSYVDGWFGGECGSGNPYYLLCGTCREKYLAMKTKS
KSTSSERYKGQAPDLIGKQDSVYEEDWDMLDVDEDEKLTGEEEFELLAGP
LGLNDRRIVPEPVQFPDSDPLGASVAMVTATNSMEETLMQIGCHGSVEKS
SSGRITLGEQAAALANPHDRVVALRRVTAAAQVLLARTMVMRALSLLSVS
GSSCSLAAGLESLGLTDIRTLVRLMCLAAAGRAGLSTSPSAMASTSERSR
GGHSKANKPISCLAYLSTAVGCLASNAPSAAKLLYQLCTQNLISAATGVN
LTTVDDSIQRKFLPSFLRGIAEENKLVTSPNFVVTQALVALLADKGAKLR
PNYDKSEVEKKGPLELANALAACCLSSRLSSQHRQWAAQQLVRTLAAHDR
DNQTTLQTLADMGGDLRKCSFIKLEAHQNRVMTCVWCNKKGLLATSGNDG
TIRVWNVTKKQYSLQQTCVFNRLEGDAEESLGSPSDPSFSPVSWSISGKY
LAGALEKMVNIWQVNGGKGLVDIQPHWVSALAWPEEGPATAWSGESPELL
LVGRMDGSLGLIEVVDVSTMHRRELEHCYRKDVSVTCIAWFSEDRPFAVG
YFDGKLLLGTKEPLEKGGIVLIDAHKDTLISMKWDPTGHILMTCAKEDSV
KLWGSISGCWCCLHSLCHPSIVNGIAWCRLPGKGSKLQLLMATGCQSGLV
CVWRIPQDTTQTNVTSAEGWWEQESNCQDGYRKSSGAKCVYQLRGHITPV
RTVAFSSDGLALVSGGLGGLMNIWSLRDGSVLQTVVIGSGAIQTTVWIPE
VGVAACSNRSKDVLVVNCTAEWAAANHVLATCRTALKQQGVLGLNMAPCM
RAFLERLPMMLQEQYAYEKPHVVCGDQLVHSPYMQCLASLAVGLHLDQLL
CNPPVPPHHQNCLPDPASWNPNEWAWLECFSTTIKAAEALTNGAQFPESF
TVPDLEPVPEDELVFLMDNSKWINGMDEQIMSWATSRPEDWHLGGKCDVY
LWGAGRHGQLAEAGRNVMVPAAAPSFSQAQQVICGQNCTFVIQANGTVLA
CGEGSYGRLGQGNSDDLHVLTVISALQGFVVTQLVTSCGSDGHSMALTES
GEVFSWGDGDYGKLGHGNSDRQRRPRQIEALQGEEVVQMSCGFKHSAVVT
SDGKLFTFGNGDYGRLGLGNTSNKKLPERVTALEGYQIGQVACGLNHTLA
VSADGSMVWAFGDGDYGKLGLGNSTAKSSPQKIDVLCGIGIKKVACGTQF
SVALTKDGHVYTFGQDRLIGLPEGRARNHNRPQQIPVLAGVIIEDVAVGA
EHTLALASNGDVYAWGSNSEGQLGLGHTNHVREPTLVTGLQGKNVRQISA
GRCHSAAWTAPPVPPRAPGVSVPLQLGLPDTVPPQYGALREVSIHTVRAR
LRLLYHFSDLMYSSWRLLNLSPNNQNSTSHYNAGTWGIVQGQLRPLLAPR
VYTLPMVRSIGKTMVQGKNYGPQITVKRISTRGRKCKPIFVQIARQVVKL
NASDLRLPSRAWKVKLVGEGADDAGGVFDDTITEMCQELETGIVDLLIPS
PNATAEVGYNRDRFLFNPSACLDEHLMQFKFLGILMGVAIRTKKPLDLHL
APLVWKQLCCVPLTLEDLEEVDLLYVQTLNSILHIEDSGITEESFHEMIP
LDSFVGQSADGKMVPIIPGGNSIPLTFSNRKEYVERAIEYRLHEMDRQVA
AVREGMSWIVPVPLLSLLTAKQLEQMVCGMPEISVEVLKKVVRYREVDEQ
HQLVQWFWHTLEEFSNEERVLFMRFVSGRSRLPANTADISQRFQIMKVDR
PYDSLPTSQTCFFQLRLPPYSSQLVMAERLRYAINNCRSIDMDNYMLSRN
VDNAEGSDTDY

By “HERC1 polynucleotide” is meant a polynucleotide encoding a HERC1 polypeptide. An exemplary HERC1 polynucleotide sequence follows:

>NM_003922.4 Homo sapiens HECT and RLD domain
containing E3 ubiquitin protein ligase family
member 1 (HERC1), mRNA
(SEQ ID NO: 8)
GTCTTTCCCTTTGACAAGTCGCTGCTGCTGCAGCAAAAATAAAGGACGCGACCGCCGCAGCCTCTGCGGA
GCCGGGCTCGGGTCGCCGGAGCCGCGCCCCACCCCGCCAGCTCCAGAGCCACGACTAATGGCTGAAGGAT
AAATCAACATGGCAACTATGATTCCACCAGTGAAGCTGAAATGGCTTGAACACTTGAACAGCTCCTGGAT
TACAGAGGACAGTGAATCTATTGCTACAAGAGAGGGAGTTGCTGTTCTGTATTCTAAACTGGTTAGCAAT
AAGGAAGTAGTACCTTTGCCCCAACAAGTTTTATGCCTCAAAGGACCACAGTTGCCAGACTTTGAACGTG
AGTCTCTTTCAAGTGATGAGCAGGACCACTATTTGGATGCCCTTCTTAGCAGCCAGCTAGCATTGGCAAA
GATGGTATGTTCAGATTCCCCATTTGCCGGGGCACTTAGAAAACGACTGCTTGTACTCCAGCGTGTCTTT
TATGCACTTTCTAATAAATACCATCACAAAGGCAAGGTGAAGCAGCAGCAGCATTCTCCGGACAGCAGTT
CTGGTTCAGCAGATGTCCATTCTGTTAGTGAACGCCCCCGGTCAAGCACTGATGCACTTATAGAAATGGG
TGTTCGAACTGGTCTAAGTTTATTATTTGCGCTTCTAAGACAAAGTTGGATGATGCCTGTGTCAGGACCT
GGTCTCAGTCTTTGCAACGATGTCATTCATACTGCAATTGAAGTTGTGAGCTCTTTGCCACCATTATCAT
TAGCAAATGAAAGCAAGATTCCTCCTATGGGCTTGGACTGCTTATCGCAAGTAACAACATTTCTTAAAGG
AGTCACTATTCCTAATTCTGGGGCAGACACTTTAGGTCGTAGATTAGCTTCTGAGTTGCTGCTTGGTTTG
GCAGCTCAACGAGGCTCATTGCGATATCTTCTTGAATGGATAGAAATGGCTTTGGGGGCTTCGGCAGTTG
TACACACCATGGAGAAAGGCAAACTACTCTCAAGCCAGCAAGGAATGATCAGCTTTGACTGCTTTATGAC
CATATTAATGCAGATGAGGCGTTCTTTGGGTTCATCTGCTGATCGGAGTCAGTGCAACAACCAACCACGA
ACATCGGATGGCTTGTGCTCCCTTTACGAGGCAGCATTATGTCTCTTTGAAGAGGTTTGCAGAATGGCTT
CTGATTATTCGAGAACATGTGCTAGCCCAGATAGCATTCAGACTGGTGATGCTCCCATTGTCTCCGAAAC
CTGTGAGGTTTATGTTTGGGGGAGCAATAGCAGCCATCAGTTGGTAGAAGGTACACAGCACAAAATACTG
CAACCCAAACTGGCTCCTAGTTTCTCTGATGCACACACCATTGAAGCTGGACAGTACTGCACTTTTGTCA
TTTCTACGGATGGCTCTGTTAGAGCTTGCGGAAAGGCAGCTATGGGACGACTGGGCCTTGGACACTCCAA
TAATCAGTCAACTTTAAAAAAGTTAACATTCGAGCCTCACAGATCCATTAAAAAGGTTTCATCTTCTAAA
GGATCTGATGGTCACACTTTAGCCTTTACGACAGAAGGAGAAGTCTTCAGTTGGGGAGATGGTGATTATG
GGAAACTGGGGCATGGAAATAGTTCAACACAGAAATATCCCAAGCTTATTCAGGGACCTCTACAAGGAAA
GGTAGTTGTTTGTGTGTCAGCTGGATACAGACATAGTGCTGCTGTCACAGAGGATGGGCAATTATACACA
TGGGGTGAAGGAGACTTTGGAAGATTAGGTCATGGTGACAGCAATAGTCGTAACATTCCAACATTAGTAA
AAGACATCAGCAATGTAGGAGAGGTTTCTTGTGGCAGTTCACATACTATTGCTCTGTCTAAAGATGGGAG
AACTGTATGGTCTTTTGGAGGAGACGACAATGGTAAACTTGGTCATGGTGATACCAACACAGTGTATAAA
CCTAAAGTTATTGAAGCTTTACAAGGAATGTTCATTCGCAAAGTTTGTGCTGGGAGCCAGTCTTCACTTG
CTTTGACATCAACAGGGCAGGTCTATGCTTGGGGCTGTGGAGCTTGTCTAGGTTGTGGTTCTTCAGAAGC
TACTGCTTTGAGACCCAAGCTTATTGAAGAACTGGCTGCCACAACAATAGTTGATGTTTCTATTGGAGAC
AGTCATTGTTTGGCTCTTTCTCATGATAATGAAGTTTATGCCTGGGGCAATAACTCAATGGGGCAATGTG
GTCAGGGAAATTCCACAGGTCCTATTACTAAACCAAAGAAAGTGAGTGGCTTAGATGGCATAGCTATTCA
GCAGATTTCGGCTGGAACATCACATAGTCTGGCATGGACTGCTCTTCCTAGGGACAGACAAGTTGTTGCA
TGGCACCGACCTTATTGTGTAGATCTTGAAGAGAGTACCTTCTCACACCTGCGTTCTTTTCTTGAGAGAT
ACTGTGATAAAATAAACAGTGAGATTCCCCCACTCCCTTTCCCTTCATCAAGACAACACCACAGTTTTCT
CAAGCTGTGCCTGAAGCTACTTTCAAATCACCTTGCTCTTGCACTTGCGGGAGGGGTAGCTACCAGCATT
CTCGGGAGGCAGGCAGGTCCACTTCGAAATTTGCTCTTCAGACTGATGGACTCAACTGTCCCAGATCAAA
TCCAAGAGGTGGTAATTGAAACTTTATCAGTGGGAGCAACCATGCTGTTACCTCCATTACGACAACGGAT
GGAATTACTTCATTCTCTTTTACCTCAAGGACCTGATAGATGGCAAAGCTTATCTAAAGCACACACAATG
CAACTGGATATCATCCTGACAAGTTTGCAAGATCATACCCACGTAGCCTCCCTACTTGGCTATAGTTCAC
CCTCTGATGCTGCTGACCTATCTTCTGTGTGTACTGGCTACGGAAATCTGTCAGATCAACCTTACGGCAC
TCAGAGCTGCCATCCAGATACCCACCTGGCTCAAATTTTGATGAAGACCCTCTTAACAAATTTAGGATTT
TATACAGATCAAGCATTTGGAGAGCTAGAAAACAATACTGATAAATTTCTACTTGGAACATCATCATCAG
AAAACAGTCAGCCTGCTCATCTTCATGAACTGCTATGTTCACTACAGAAACAGCTGCTGGCATTTTGCCA
TATCAATAACATTAGTGAGAACTCAAGCAGTGTGGCATTGCTTCATAAACATCTTCAGCTTTTGTTGCCT
CATGCCACAGATATTTATTCACGTTCTGCAAATTTGCTCAAAGAAAGTCCTTGGAATGGCAGTGTTGGAG
AAAAATTAAGAGATGTGATATACGTCTCAGCTGCTGGCAGTATGCTCTGCCAGATTGTTAACTCCCTGCT
GTTACTCCCTGTGTCAGTGGCTCGGCCTTTATTGAGTTACCTCCTCGACTTGTTGCCACCTCTTGATTGC
CTTAATAGACTCCTGCCAGCTGCTGATCTTTTAGAAGACCAGGAGTTACAGTGGCCTCTTCATGGAGGGC
CAGAACTAATTGATCCTGCTGGTCTGCCATTACCTCAGCCAGCTCAGTCCTGGGTATGGCTTGTGGATCT
AGAAAGAACAATTGCTCTCCTTATTGGGCGGTGTCTTGGTGGCATGCTTCAGGGCTCCCCTGTGTCTCCA
GAGGAACAGGACACTGCATATTGGATCAAAACGCCACTGTTCAGTGACGGTGTAGAAATGGACACTCCTC
AATTGGATAAATGTATGAGTTGCCTGTTAGAAGTAGCACTTTCTGGAAATGAACAACAGAAGCCTTTTGA
TTATAAATTGCGGCCTGAAATTGCTGTCTATGTAGACTTGGCATTGGGTTGTTCTAAAGAGCCTGCCCGA
AGCCTTTGGATCAGCATGCAGGACTATGCTGTTAGTAAAGATTGGGACAGTGCAACTTTAAGTAATCAGT
CACTCTTGGACACTGTGTCTAGATTTGTTCTTGCAGCTCTTCTGAAACACACAAATTTACTTAGTCAAGC
ATGTGGAGAAAGCCGATATCAACCTGGTAAACACTTATCAGAAGTGTACCGTTGTGTATACAAAGTTCGA
AGTCGTTTACTTGCTTGCAAGAACCTTGAACTTATTCAAACAAGGTCATCATCACGGCACACATGGATAT
CAGAAAACCAGGACTCTGCAGATGTTGATCCTCAGGAGCATTCATTTACTCGAACTATTGATGAAGAAGC
TGAAATGGAAGAACAGGCTGAGAGAGACCGGGAAGAGGGGCATCCGGAGCCAGAGGATCAAGAGGAGGAA
CGGGAACATGAAGTGATGACAGCTGGCAAAATCTTTCAGTGTTTCCTCTCAGCCCGTGAAGTAGCTCGTA
GCCGAGACCGAGATACAATGAACAGTGGGGCAGGGTCTGGGGCTCGAGCTGATGATCCACCTCCTCAGTC
TCAGCAAGAGCGAAGGGTCAGCACAGACCTTCCTGAGGGTCAGGATGTGTACACTGCTGCATGCAACTCC
GTGATCCATCGGTGTGCCCTGTTAATATTAGGAGTAAGTCCTGTGATAGATGAGCTTCAGAAGCGAAGAG
AAGAAGGACAGTTGCAGCAACCTTCAACAAGTGCCTCTGAAGGGGGTGGACTTATGACCAGGAGTGAAAG
TCTTACTGCAGAGAGCCGGCTAGTCCACACAAGCCCAAATTATAGACTGATCAAATCGAGGACTGAATCT
GATTTGTCTCAGCCTGAATCAGATGAAGAGGGTTACGCACTGAGTGGCAGACGAAATGTTGATTTGGATT
TGGCAGCATCTCACAGAAAGAGAGGTCCTATGCACAGTCAATTGGAATCCCTGAGTGACTCTTGGGCTCG
CCTGAAACATAGCAGACACTGGTTATGCAACTCCTCCTATTCCTTTGAGTCAGATTTTGATCTTACCAAG
TCTTTGGGAGTTCACACTTTGATTGAAAATGTTGTAAGCTTTGTGAGTGGAGATGTGGGGAATGCCCCAG
GTTTTAAAGAGCCAGAGGAAAGTATGTCTACAAGTCCCCAGGCCTCCATCATTGCAATGGAACAGCAGCA
GTTAAGGGCAGAACTTCGTTTAGAGGCACTTCATCAGATCCTCGTTCTATTGTCTGGGATGCAACAAAAA
GGTAGCATCTCACTGGCAGGAAGCAGATTGAGTTCAGGCTTCCAGTCCTCCACACTACTCACGTCTGTGA
GGCTGCAGTTCCTAGCAGGGTGTTTTGGTTTAGGCACTGTTGGACACACAGGAGGCAAGGGAGAGAGTGG
CCGATTGCATCACTATCAGGATGGGATCAGAGCAGCTAAGAGAAATATTCAGATTGAAATCCAGGTAGCT
GTGCATAAAATTTATCAACAGTTGTCTGCTACCCTGGAAAGAGCCCTGCAAGCAAACAAGCATCACATTG
AAGCCCAGCAACGTCTGCTTCTGGTTACAGTTTTTGCCCTAAGTGTTCATTATCAACCAGTAGATGTTTC
TTTGGCAATTTCCACTGGTCTGCTAAACGTATTGTCACAGTTGTGTGGTACAGACACCATGCTAGGACAG
CCCCTGCAGTTGTTGCCAAAGACGGGTGTTTCCCAGCTTAGCACAGCTTTGAAAGTGGCCAGTACAAGGT
TGCTCCAGATTCTAGCCATCACTACTGGGACCTATGCTGATAAACTGAGTCCCAAAGTAGTTCAATCCTT
GTTGGATCTACTCTGTAGTCAGTTGAAGAATTTATTGTCCCAAACTGGTGTACTACATATGGCCTCTTTC
GGAGAAGGGGAGCAAGAAGACGGTGAAGAAGAAGAAAAAAAAGTTGACTCCAGTGGAGAAACTGAGAAGA
AAGATTTCAGAGCTGCTCTTAGCAAACAACATGCAGCCGAACTCCATCTAGGGGATTTTTTAGTTTTTCT
TCGCAGAGTTGTATCTTCAAAAGCAATTCAATCAAAAATGGCTTCCCCAAAGTGGACCGAAGTGCTTCTA
AATATAGCATCTCAGAAATGTTCTTCAGGTATCCCTCTGGTTGGTAACTTAAGAACAAGGCTCCTTGCAC
TTCATGTCCTTGAAGCTGTGCTGCCAGCTTGTGAATCTGGTGTAGAAGATGATCAAATGGCCCAGATTGT
TGAGCGCTTATTTTCCCTTCTCTCTGATTGTATGTGGGAGACACCCATTGCTCAGGCCAAACATGCTATT
CAGATAAAGGAAAAAGAACAACAAATAAAACTACAGAAGCAGGGCGACTTGGAAGAAGAAGATGAGAATC
TTCTATCCAAGAAGTATCCTTTGACCCGGAGAAAGCTCAGTGTTGCCTAGTGGAGGAATGGACAGATTTT
AACTCACGGCAGTGGAGGAAAGGATATGGATTGGCATCTACAGCGAGTAACTTCTGGGTGCTATCAGTGG
AAGTTTTATATTGTGAAGGAAAACAGAGGTAATGAAGGCACGTGTGTTGGAGTTTCTCGCTGGCCAGTAC
ATGACTTTAATCACCGCACTACCTCGGATATGTGGCTCTATAGGGCCTACAGTGGTAACCTCTATCACAA
TGGAGAACAGACTCTCACATTGTCCAGCTTTACTCAAGGAGATTTCATTACCTGTGTGTTAGACATGGAA
GCCAGGACCATTTCTTTTGGGAAAAATGGAGAGAACCCAAATTAGCTTTTGCAAGATGTGGATGCAGCAG
AGTTGTACCCATGTGTGATGTTCTATAGTAGCAATCCAGGGGAAAAGCTCAAAATTTGTGATATGCAGAT
GCGTGGCACACCCCGAGACTTACTTCCAGGAGACCCTATTTGTAGTCCAGTAGCAGCAGTGCTGGCTGAG
GCCACTATTCAGCTCATCCGTATCCTTCACCGAACAGACCGTTGGACTTACTGCATTAACAAAAAAATGA
TGGAAAGGCTTCACAAAATTAAGATATGTATTAAAGAGTCAGGTCAGAAGCTAAAGAAAAGCCGCTCGGT
TCAGAGCCGAGAGGAAAATGAAATGAGAGAGGAGAAGGAGAGCAAAGAGGAAGAGAAAGGTAAACATACT
AGGCATGGCCTCGCTGACCTCTCAGAGCTGCAGCTGAGGACTCTTTGCATAGAGGTGTGGCCCGTGCTGG
CTGTGATAGGAGGAGTTGATGCTGGTCTTAGAGTTGGAGGTCGGTGTGTTCACAAGCAAACTGGGCGCCA
TGCCACGCTGCTGGGAGTGGTCAAAGAGGGCAGCACGTCTGCCAAGGTCCAATGGCATCAAGCACAAATT
ACTATCAGCTTCCCAACTTTTTGGTCGCCTAGTGATACTCCATTGTATAATCTGGAACCCTGTGAACCAT
TGCCGTTTGATGTGGCGCGATTCCGAGGCCTGACGGCTTCTGTGCTGCTGGACCTAACATATCTCACTGG
CGTTCATGAACACATGGGCAAACAGAGACCAAACCACATCAAAAGAAACACCCGACATGAATCCGAGGAG
AAAGGGGATGTTGAGCAGAAACCTGAGAGTGAATCCGCTTTAGATATGCGAACAGGCCTAACATCTGATG
ACGTCAAAAGTCAGAGTACCACAAGCTCCAAATCACAAAATCAAATCGCTTCATTTTCTTTAGATCCAAC
ACTGCCAAGTGTGGAATCCCAACATCAAATAACACAAGGGAAAAGAAAAAATCATGAACACATGTCCAAA
AACCATGATGTAGCCCAGTCAGAAATCAGAGCAGTCCAGCTGTCCTATCTTTACCTCGGTGCTATGAAGT
CACTTAGTGCCCTTCTTGGCTGTAGTAAATATGCTGAGCTGTTGCTGATACCAAAAGTTCTGGCTGAAAA
TGGCCACAACTCAGACTGTGCAAGTTCTCCAGTTGTTCATGAAGACGTGGAGATGCGAGCAGCCCTGCAG
TTCTTGATGCGACACATGGTGAAGCGAGCAGTCATGCGGTCACCCATAAAGAGAGCATTGGGATTAGCTG
ATCTGGAACGAGCGCAAGCCATGATCTATAAATTAGTGGTTCATGGGCTTTTGGAAGACCAGTTTGGGGG
CAAAATTAAGCAAGAGATTGATCAACAAGCTGAAGAAAGTGACCCTGCCCAGCAGGCACAGACACCAGTT
ACTACTAGCCCATCAGCCTCAAGCACGACCTCCTTTATGAGCAGCTCTCTGGAGGACACCACAACTGCCA
CCACTCCAGTCACTGACACAGAAACAGTGCCTGCATCCGAGTCCCCGGGAGTGATGCCTCTTAGTCTTCT
CAGGCAAATGTTCTCTAGTTACCCAACTACCACTGTACTTCCCACACGTCGGGCACAGACTCCTCCAATA
TCTTCGTTACCAACCTCTCCTTCTGATGAAGTAGAAGCGAGGCAAAGTTTAACTTCTCCTGATTCCCAGT
CAGCAAGGCCAGCTAACCGCACAGCCTTGTCAGACCCAAGCAGTAGACTTTCAACTTCTCCTCCTCCTCC
AGCAATTGCAGTTCCCTTGCTGGAAATGGGGTTCTCTCTTCGGCAGATTGCCAAAGCCATGGAAGCTACA
GGTGCTAGGGGAGAGGCTGATGCCCACAATATCACTGTCCTTGCCATGTGGATGATAGAGCACCCTGGGC
ATGAGGATGAAGAGGAGCCCCAGTCGGGCAGCACAGCAGACTCTAGGCCTGGAGCAGCCGTTCTAGGCAG
TGGCGGGAAGTCAAATGATCCCTGTTATTTGCAGTCACCTGGACACATACCATCAGCTGATGCTGCTGAA
ATGGAGGAAGGTTTTAGTGAAAGCCCTGATAATTTGGATCATACAGACAATGCAGCTTCTGGAAGTGGAC
CATCAGCTAGAGGTCGCTCAGCGGTAACAACAAGACACAAGTTTGACTTAGCTGCTCGCACACTGCTAGC
AAGAGCAGCGGGATTATACCGCTCTGTGCAGGCCCACAGCAATCAAAGTCGCACACAAGCAATATCTTTG
CAGCAAGACCCAGGGGCGTTGTATGACTTTAATTTAGATGAGGAATTGGAAATTGATCTTGATGATGAGG
CGATGGAAGCTATGTTTGGACAAGACCTGACCAGTGACAATGATATTCTGGGAATGTGGATCCCAGAGGT
ACTGGATTGGCCTACCTGGCATGTTTGTGAGTCTGAAGACAGGGAAGAAGTGGTGGTGTGTGAACTGTGT
GAATGCAGCGTCGTCAGCTTCAATCAGCACATGAAGAGAAACCATCCAGGCTGTGGGCGCAGTGCAAACC
GCCAGGGCTATCGCAGCAATGGTTCCTATGTGGATGGCTGGTTTGGCGGTGAATGTGGGAGTGGAAATCC
GTACTACCTGTTATGTGGCACCTGCAGGCACAACTACTTAGCCATCAAGACCAAATCTAAGTCAACAAGT
TCTGAAAGGTACAAGGGACAAGCTCCAGATCTAATTGGCAAGCAAGACAGTGTGTATGAAGAAGACTGGG
ACATGTTGGATGTTGATGAAGATCAAAAGCTAACTGGTGAACAAGAATTTGAATTACTTGCTGGACCGCT
TGGTTTAAATGACCGGCGCATTGTACCAGAACCAGTTCAGTTCCCTGACAGCGATCCACTGGGAGCATCA
GTAGCAATGGTCACAGCCACCAACAGTATGCAACAGACTCTGATGCAAATAGGTTGCCATGGCTCCGTAG
AAAAGAGCTCCTCTGGGAGAATAACGTTAGGAGAGCAGGCAGCTGCCCTAGCAAACCCTCATGACCGTGT
GGTGGCTTTAAGGAGAGTGACTGCTGCTGCTCAGGTTCTTCTGGCCAGAACCATGGTCATGAGAGCGCTG
TCTCTTCTCTCAGTCAGTGGTTCCAGTTGTAGCCTGGCTGCTGGTCTTGAGTCTCTGGGGCTAACAGATA
TCCGAACGCTAGTTCGATTAATGTGCTTGGCAGCAGCAGGGAGAGCTGGCCTCTCCACCAGCCCTTCTGC
CATGGCTAGCACCTCAGAACGATCACGAGGTGGGCATAGCAAGGCTAACAAGCCTATCTCTTGCCTGGCC
TATTTGAGCACAGCAGTGGGATGTCTGGCATCAAATGCTCCTAGTGCTGCCAAACTGCTTGTACAGTTGT
GTACACAGAACTTGATTTCTGCTGCAACAGGTGTAAATCTAACCACAGTTGATGACTCAATTCAGCGAAA
GTTTCTACCCAGCTTTCTCCGAGGAATTGCTGAACAGAACAAGCTTGTGACCTCCCCAAACTTTGTTGTA
ACACAGGCCCTTGTGGCATTGCTAGCAGACAAAGGGGCCAAACTAACACCTAACTATGATAAGTCAGAAG
TTGAAAAGAAAGGCCCTCTGGAGTTGGCTAATGCCCTGGCAGCCTGCTGCCTCTCCTCCAGGCTGTCCTC
ACAGCATCGGCAATGGGCAGCTCAGCAACTCGTGCGCACTCTTGCTGCACACGACCGTGACAACCAAACT
ACTCTGCAGACACTTGCTGATATGGCAGCACATCTTAGAAAATGCTCCTTTATCAAATTGGAGGCTCATC
AGAACAGAGTAATGACATGTGTTTGGTGTAATAAAAAAGGTCTTTTGGCTACAAGTGGCAATGATGGCAC
CATCCGCGTATGGAATGTTACCAAGAAGCAATATTCACTGCAACAGACCTGTGTGTTCAACAGATTGGAA
GGGGATGCTGAGGAAAGCCTGGGATCACCCAGTGATCCAAGTTTCTCACCAGTTTCCTGGAGTATCAGTG
GCAAATATCTAGCAGGCGCTTTGGAAAAGATGGTGAATATCTGGCAAGTTAATGGAGAAAAGCGATTAGT
AGATATTCAGCCTCATTGGGTATCTGCCCTGGCTTGGCCAGAAGAGGGTCCGGCTACAGCCTGGTCAGGA
GAGTCTCCAGAATTGTTGTTGGTGGGACGATGCGATGGATCTCTGGGACTGATTGAAGTTGTTGATGTGT
CCACCATGCACCGTCGAGAATTGGAGCATTGCTATCGAAAGGATGTGTCTGTTACTTGCATTGCATGGTT
CAGTGAAGACAGACCATTTGCAGTGGGATATTTTGATGGAAAACTGTTACTGGGAACAAAGGAACCACTT
GAGAAAGGAGGCATTGTTCTAATTGATGCACATAAGGATACTCTTATTAGCATGAAGTGGGACCCTACAG
GTCATATTCTTATGACATGTGCCAAACAAGACACTGTGAAACTCTGGGGCTCTATTTCGGGATGCTGGTG
CTGTCTACATTCACTCTGCCATCCATCTATTGTAAATGGCATTGCTTGGTGCCGCCTTCCAGGDAAAGGA
TCCAAGTTGCAGTTACTGATGGCTACTGGCTGTCAGAGTGGCTTAGTATGTGTTTGGCGCATTCCTCAAG
ATACTACACACGACCAATGTGACTAGTGCAGAAGGATGGTGGAGCAGAATCAAATTGCCAGGATGCGATA
TAGGAAATCATCAGGAGCCAAGTGTGTTTATCAGCTGCGGGGACACATCACTCCTGTTCGGACTGTTGCC
TTTAGTTCTGATGGGTTGGCCCTGGTGTCTGGTGGACTAGGTGGGCTCATGAACATTTGGTCTTTAAGGG
ATGGCTCTGTCTTGCAAACTGTTGTGATAGGCTCTGGAGCTATTCAGACCACAGTATGGATTCCAGAAGT
TGGAGTAGCTGCTTGCTCAAATAGATCAAAGCATGTTTTGGTCGTGAATTGTACAGCAGAATGGGCAGCT
GCCAATCATGTTTTGGCAACCTGTAGGACAGCATTGAAACAGCAGGGTGTTCTGGGATTGAACATGGCTC
CCTGCATGAGAGCATTTTTGGAGCGGCTCCCCATGATGCTTCAGGAGCAGTATGCCTATGAAAAGCCTCA
TGTGGTTTGTGGTGACCAACTTGTTCATAGCCCCTATATGCAATGCTTGGCTTCCCTTGCTGTGGGACTT
CATCTGGATCAGCTGTTGTGTAACCCTCCAGTGCCACCACACCACCACAACTGTCTCCCTGACCCTGCAT
CCTGGAATCCAAATGAATGGGCCTGGTTAGAATGTTTCTCAACCACTATAAAAGCTGCCGAAGCCCTGAC
CAATGGAGCCCAGTTTCCAGAATCTTTTACCGTTCCAGATCTAGAACCTGTTCCAGAGGATGAACTTGTA
TTTCTAATGGATAACAGTAAATGGATTAACGGCATGGATCAACAAATTATGTCTTGGGCAACTTCCAGAC
CTGAGGACTGGCACCTGGGAGGTAAATGTGATGTCTACTTATGGGGTGCTGGTAGGCATGGACAGCTGGC
AGAAGCTGGAAGAAATGTAATGGTACCTGCAGCAGCTCCCTCATTCTCACAGGCCCAACAGGTCATTTGT
GGTCAGAATTGTACCTTTGTCATCCAGGCCAATGGCACAGTGTTGGCTTGTGGGGAAGCAAGTTATGGCA
GATTAGGACAAGGAAATTCAGATGACCTTCATGTGCTGACAGTTATTTCAGCCTTACAAGGCTTTGTGGT
GACCCAGCTGGTGACTTCCTGTGGTTCTGATGGGCACTCTATGGCCCTAACTGAAAGTGGTGAGGTCTTT
AGCTGGGGAGATGGTGACTATGGTAAACTTGGCCATGGCAACAGCGACAGGCAGCGGCGGCCCAGGCAGA
TCGAGGCCTTACAAGGAGAAGAAGTGGTGCAGATGTCTTGTGGCTTCAAGCACTCAGCAGTGGTCACTTC
AGATGGCAAACTGTTCACCTTTGGGAATGGTGACTATGGTCGTCTGGGTCTTGGAAATACCTCTAACAAA
AAACTTCCAGAGAGAGTGACTGCACTGGAGGGATATCAGATTGGACAGGTGGCCTGTGGATTAAACCACA
CTTTGGCAGTGTCAGCAGATGGTTCCATGGTGTGGGCTTTTGGAGATGGAGATATGGCAAAACTAGGCTT
AGGAAATTCCACTGCAAAATCTTCACCTCAGAAAATTGACGTCCTTTGTGGAATTGGAATAAAAAAGGTT
GCTTGTGGAACTCAGTTTTCTGTTGCTTTGACCAAAGATGGTCATCTGTATACCTTTGGTCAAGATCGCC
TGATAGGCTTGCCAGAGGGGCGTGCTCGCAATCACAATCGACCGCAACAAATCCCTGTCCTGGCTGGAGT
AATCATAGAAGATGTGGCAGTTGGAGCTGAACACACACTTGCTTTGGCATCAAATGGAGATGTGTATGCC
TGGGGGAGCAATTCAGAAGGGCAGCTCGGCTTAGGCCATACCAACCATGTTCGAGAACCAACCCTGGTAA
CAGGTCTGCAAGGCAAAAATGTTCGGCAGATCTCGGCTGGCCGCTGCCACAGTGCTGCATGGACAGCACC
ACCTGTCCCACCAAGAGCACCAGGTGTGTCAGTACCTCTGCAGCTGGGCCTGCCTGACACAGTGCCCCCC
CAGTATGGGGCGCTGACAGAAGTCAGCATTCACACGGTGCGGGCCAGGCTCCGGCTGCTCTACCACTTCT
CTGACCTCATGTACTCATCCTGGAGACTGCTGAACCTTAGCCCCAACAACCACAACAGCACATCCCATTA
TAATGCTGGAACTTGGGGCATTGTACAGGGACAACTTCGGCCTTTGTTAGCCCCAAGAGTCTACACTCTG
CCAATGGTGCGCTCCATAGCAAAAACCATGGTTCAAGGCAAAAACTATGGACCTCAGATAACTGTAAAGA
GGATATCAACCAGAGGACGGAAGTGTAAGCCTATTTTTGTCCAAATAGCGAGAGAAGTAGTTAAGCTGAA
TGCTTCAGACCTCCGCCTGCCTTCCCGAGCGTGGAAGGTTAAGCTGGTTGGAGAAGGGGCTGATGATGCT
GGAGGAGTGTTTGATGACACCATCAGAGAGATGTGCCAGCAACTTCAAACTGGTATTGTTGACCTTCTTA
TACCCTCTCCCAATGCCACCGCAGAAGTGGGTTACAATAGGGACAGGTTCCTTTTTAACCCTTCTGCCTG
CCTCGATGAACACTTAATGCAGTTTAAGTTTTTAGGAATTTTAATGGGGGTTGCCATTCGCACAAACAAG
CCTCTGGACCTCCACTTGGCCCCTCTGGTGTGGAAGCAGCTGTGCTGTGTCCCACTCACCCTAGAGCACC
TGGAGGAGGTGGATCTGCTCTACGTGCAGACTCTCAACAGCATTCTTCACATTGAACACAGTGGGATTAC
CGAGGAGAGTTTCCATGAGATGATTCCTCTTGATTCTTTTGTTGGCCAGAGTGCTGATGGCAAAATGGTT
CCTATAATCCCTGGTGGAAATAGTATCCCACTCACATTTTCCAACAGCAAGGAATATGTGGAGAGGGCCA
TTGAATATCGACTTCATGAGATGGACAGACAGGTGGCTGCAGTCCGAGAAGGGATGTCCTGGATTGTTCC
TGTGCCGCTGCTGTCCCTCCTCACAGCAAAACAACTGGAGCAGATGGTGTGTGGGATGCCCGAGATCTCT
GTGGAAGTCTTGAAGAAAGTGGTGCGGTACCGTGAGGTGGATGAGCAGCATCAGCTGGTGCAGTGGTTCT
GGCACACGCTGGAACAGTTCTCCAATGAGGAGCGGGTGCTTTTCATGAGGTTTGTGTCAGGAAGATCTCG
ACTACCAGCCAACACTGCTGACATTTCTCAGAGATTTCAAATCATGAAGGTTGATAGGCCTTACGACAGT
CTGCCTACCTCACAGACCTGCTTCTTCCAGCTGAGGCTGCCCCCGTACTCCAGCCAGCTGGTCATGGCCG
AGCGCCTGCGCTATGCCATCAACAACTGCCGCTCAATCGACATGCACAACTACATGCTCTCGAGAAACGT
GGACAACGCGAGGGCTCCGACACTGACTACTGACCGTGCGGCGTGCTCTCACCCTCCCTTCTCTCCCTCA
ATAATGCTCACTTCTGATTTGATGTTGATATACTTTTATGGTAACTACATAGATCTTATAACAACATAAA
CCAACATTATAAACAATGGCCACATTTAGTTACTTAAATGTAACAAACAAATTAGCATGTTTTTATTTTT
CTGTGATTGTACAAAAACAACAAAAACGAAGTGCTCTCAGTCAGGTTTTTCCCTCCATATTTTTGGTCAC
TTTTGATAAGTTTGCATGAAACCATTTTGGTGCATTTTTAGTTGGGAATGGTACATTTTTGTAAATCCAC
CCAGTGAACATGAAATTGTACATTGTGTATAATTGTTCATTAGAAAGCACAGTTTTACATGAATATTCAT
ATATTTATTTTGTTTTAATTTGAATTGCCTGTTCAGGGTTCCTTATGCACACAAATAAAGCAGATTCAGG
AATTGGA

By “ameliorate” is meant decrease, suppress, attenuate, diminish, arrest, or stabilize the development or progression of a disease or symptom thereof.

By “agent” is meant any small molecule chemical compound, antibody, nucleic acid molecule, or polypeptide, or fragments thereof.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “ includes,” “including,” and the like: “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. Examples of diseases include cognitive impairments, including cognitive impairments associated with neuropsychiatric disorders (e.g., autism and schizophrenia).

By “effective amount” is meant the amount of a required to ameliorate the symptoms of a disease or symptom thereof relative to an untreated patient. The effective amount of active compound(s) used to practice embodiments of the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

Embodiments of the invention provide a number of targets that are useful for the development of highly specific drugs to treat a disorder characterized by the methods delineated herein. In addition, the methods of the disclosure provide a facile means to identify therapies that are safe for use in subjects. In addition, the methods of the disclosure provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The terms “isolated,” “purified,” or “biologically pure” refer to material that is free to varying degrees from components which normally accompany it as found in its native state. “Isolate” denotes a degree of separation from original source or surroundings. “Purify.” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example. phosphory lation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects, the disclosure encompasses sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V: (b) F, Y, W: (c) K. R. H: (d) A, G: (e) S, T: (f) Q, N: and (g) E. D. In various embodiments, conservative amino acid substitutions can be made to the amino acid sequence of the proteins and polypeptides disclosed herein.

By “reduces” is meant a negative alteration of at least 10%, 25%, 50%, 75%, or 100%

By “reference” is meant a standard or control condition.

A “reference sequence” is a defined sequence used as a basis for sequence comparison. A reference sequence may be a subset of or the entirety of a specified sequence: for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the disclosure, but which does not substantially recognize and bind other molecules in a sample, for example, a biological sample, which naturally includes a polypeptide of the disclosure.

Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See. e.g., Wahl. G. M. and S. L. Berger (1987) Methods Enzymol. 152:399: Kimmel. A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C. more preferably of at least about 37° C. and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment. hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate. 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975): Ausubel et al. (Current Protocols in Molecular Biology, Wiley Interscience, New York, 2001): Berger and Kimmel (Guide to Molecular Cloning Techniques, 1987, Academic Press, New York): and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions. deletions, and/or other modifications. Conservative substitutions typically include substitutions within the following groups: glycine, alanine; valine, isoleucine, leucine: aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43. 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### A BRIEF DESCRIPTION OF THE DRAWINGS

FIGS. 1A-1J show memory impairments in AD thalamus-specific PTCHD1 knockdown mice. FIGS. 1A-1J include micrographs and graphs. FISH staining of ASD (A), schizophrenia risk genes (B), in ATN. Anterodorsal (AD), anteroventral (AV). (C) 11 excitatory neuron clusters in mouse thalamus from DropViz (89.027 cells, n=6 mice) (left). top differentially expressed (DE) genes from the highlighted cluster (right). Rspo3 (R-spondin 3), Col27a1 (collagen type XXVII alpha I chain). Syndig1 (synapse differentiation inducing 1). egf11 (multiple EGF like domains 11). Hs3st4 (heparan sulfate-glucosamine 3-sulfotransferase 4), (D) FISH staining in ATN, parvalbumin (PV) neurons in TRN. DAPI staining (blue).(E) antibody staining in ATN. (F) FISH staining in marmoset ATN. (G) retrograde CTB labeling from PreSub or RSC in ATN. Average of 296 CTB555⁺ and 271 CTB488⁺ cells were observed in AD. 84% of all PreSub-projecting neurons send collaterals to RSC (n=3 mice). (H) circuit-based PTCHD1 knockdown (KD) strategy (left). FISH staining after KD (right). Ptchd1 expression is decreased by 96% (fluorescence intensity) in KD mice as compared to mCh controls in FIG. 9H (n=3 mice per group). (I) CFC behavior. mCh control mice received an AAV expressing mCherry in AD in place of the AAV expressing sgRNAs. Long-term memory (LTM) recall test (mCh n=9, KD n=10 mice). (J) T-maze behavior (mCh n=9, KD n=10 mice). Dashed line indicates chance level (50% correct). Dashed line indicates the border between AD and AV. Two-tailed unpaired t test (I, J). For statistical comparisons, ** p<0.01; NS, not significant. Data are presented as mean ±SEM.

FIGS. 2A-2L show that knockdown of several ASD and Schizophrenia risk genes from AD thalamus leads to memory impairments. FIGS. 2A, 2D, 2G, and 2J are micrographs. FIGS. 2B, 2C, 23, 2F, 2H, 2I, 2K, and 2L are graphs. (A-C) FISH staining (A). CFC behavior (B). T-maze behavior (C) (n=9) mice per group). Ywhag expression is decreased by 94% (fluorescence intensity) in KD mice as compared to mCh controls (n=3 mice per group).(D-F) FISH staining (D). CFC behavior (E). T-maze behavior (F) (n=9) mice per group). Gria3 expression is decreased by 92% (fluorescence intensity) in KD mice as compared to mCh controls (n=3 mice per group).(G-I) FISH staining (G). CFC behavior (H). T-maze behavior (I) (n=9 mice per group). Cacna1g expression is decreased by 90% (fluorescence intensity) in KD mice as compared to mCh controls (n=3 mice per group). (J-L) FISH staining (J). CFC behavior (K). T-maze behavior (L) (n=9) mice per group). Herc1 expression is decreased by 97% (fluorescence intensity) in KD mice as compared to mCh controls (n=3 mice per group). Dashed line indicates the border between AD and AV. Control FISH staining (A, D, G, J) from mCh mice. Dashed line in T-maze (C, F, I, L) indicates chance level (50% correct). Two-tailed unpaired t test (B-C, E-F, H-I, K-L). For statistical comparisons, * p<0.05, ** p<0.01; NS, not significant. Data are presented as mean ±SEM.

FIGS. 3A-3K provide staining, graphs, and electrophysiological traces showing the inputs and electrophysiological properties of AD and AV thalamus. (A) FISH staining in ATN. (B-D) Mapping brain-wide inputs to AD or AV, RV starters (vellow) in AD (B) or AV (C), average RV-positive cell counts (D) (n=3 mice for AD, n=4 mice for AV. normalized starters across groups). PrL (prelimbic cortex). Cg1 (cingulate cortex area 1). Cg2 (cingulate cortex area 2), M2 (secondary motor cortex). SIBF (primary somatosensory cortex barrel field), RSA (retrosplenial agranular cortex). RSG (retrosplenial granular cortex). Dashed line in panel C indicates the border between AD and AV. see also FIGS. 10E-10F.(E-I) RV-GFP labeling (E) of AD neurons (staining), recorded neurons (staining) (F), after-depolarization potential (ADP) amplitude (G). In current-induced sag (H), excitability (I) (22 AD RV⁺, 17 AD RV⁻, 18 AV neurons, n=3 mice). (J-K) Terminals of ChR2-eYFP injected into PreSub (left) or ChR2-mCherry injected into RSC (right) (J), connectivity between AD, AV. PreSub, and RSC (K). One-way ANOVA followed by Bonferroni post-hoc test (G-H), and two-way ANOVA with repeated measures followed by Bonferroni post-hoc test (I). For statistical comparisons, *** p<0.001; NS, not significant. Data are presented as mean ±SEM.

FIGS. 4A-4M provide graphs and electrophysiological recordings showing that the AD→RSC→EC circuit is necessary for contextual memory encoding. (A) hM4Di expression in AD. (B) CFC behavior (n=9 mice per group), mCherry control (mCh) mice received a Cre-dependent mCherry virus in place of the hM4Di virus. (C) mEPSCs of AD neurons from home cage (16 neurons) or CFC training (18 neurons) groups (n=3 mice per group). (D) Activity of AD neurons using Fos-TRAP mice (n=6 mice per group). Immediate shock (Imm. Shk.). AD neurons revealed by C1QL2 staining. (E-F) LFP traces before (Pre) vs. after (Post) CFC training. change in LFP power after training (E), change in power for individual frequency bands (F) (n=15 mice). (G-H) AMPA/NMDA ratio recordings of AD circuits. representative traces (G), quantification (H) (AD→PreSub: 29 neurons per group. AD→RSC: 27 home cage and 26 training neurons, n=3 mice per group). (I) Optogenetic terminal inhibition (eArch-eYFP, n=12 mice) or activation (ChR2-eYFP, n=7 mice) during CFC training. Control (eYFP, n=14 mice). LTM test is plotted. (J-K) cFos staining in RSC using home cage (n=7 mice), training control (mCherry or mCh. n=7 mice), training AD hM4Di-mCh (n=8 mice) groups (J), cFos staining in hippocampal CAI (K) (n=6 mice per group). Both mCh and hM4Di-mCh groups received C21 injections prior to training. Dentate gyrus (DG).(L) Two-step RV tracing showing AD. AV inputs to entorhinal cortex (EC)-projecting RSC neurons. Starters (yellow) in RSC (left image), upstream ATN labeling (right image).(M) Optogenetic terminal inhibition of EC-projecting RSC neurons, which receive ATN input, during training (eYFP n=9 mice, eArch-eYFP n=11 mice). Two-tailed unpaired t test (B-C. H. M), paired t test (F), and one-way ANOVA followed by Bonferroni post-hoc test (D. I-K). For statistical comparisons, * p<0.05, ** p<0.01, *** p<0.001; NS, not significant. Data are presented as mean ±SEM.

FIGS. 5A-5L provide staining, graphs, and electrophysiological recordings showing that the AV→RSC circuit regulates memory specificity. (A) Halorhodopsin (NpHR) expression in AV. C1QL2 staining (red).(B) AV cell bodies or AV→RSC terminal inhibition during CFC training (day 1) followed by LTM recall and neutral context tests (control eGFP n=8 mice. AV NpHR n=10 mice. AV→RSC NpHR n=8 mice).(C) cFos staining in RSC using home cage (n=4 mice). training control (eGFP, n=5 mice), training AV→RSC NpHR-eYFP (n=5 mice) groups. (D) Retrograde RV tracing in PV-Cre. somatostatin (SST)-Cre, or VIP-Cre mice. Images show RV labeling in AV thalamus (left). quantification of RV cells in AV (n=4 mice per group) (right). Normalized starters across groups.(E-G) cFos activation of PV, VIP cell types in RSC during CFC training. representative images (E-F), overlap quantification (G) (PV-Cre: home cage n=7 and training n=8 mice. VIP-Cre: home cage n=5 and training n=7 mice). Cre mice were prepared by injecting a Cre-dependent eYFP virus in RSC.(H) Fold change plotted relative to average home cage counts (n=8 PV-Cre training mice, n=7 VIP-Cre training mice). (I-J) AV→RSC inhibition with PV or VIP activation in RSC during training. viral injection schematic (I), neutral context test (J) (PV-Cre: C21 n=8 and C21+light n=6 mice. VIP-Cre: C21 n=7 and C21+light n=6 mice). (K-L) AD→RSC or AV→RSC terminal inhibition during training in the cocaine-induced conditioned place preference behavior. Preference for the cocaine (Coc) vs. the saline (Sal) side is plotted within animal for the recall test (K), and the modified chamber test (L) (n=12 mice per group). One-way ANOVA followed by Bonferroni post-hoc test (B-D). two-tailed unpaired t test (G-H. J). and paired t test (K-L). For statistical comparisons, * p<0.05. ** p<0.01, *** p<0.001; NS, not significant. Data are presented as mean ±SEM.

FIGS. 6A-6H provide staining, electrophysiological recordings, and graphs showing that normalizing neuronal hyperexcitability rescues memory deficits in PTCHD1 KD mice. (A) Ex vivo recordings from control (mCherry or mCh) vs. KD mice showing action potential (AP) threshold. AP half width, and neuronal excitability (24 mCh neurons, 23 KD neurons. n=3 mice per group). (B-C) Viral injection schematic for electrophysiological recordings (B). AMPA/NMDA ratio recordings of the AD→RSC circuit (C) in wild type (data from FIG. 4H) or KD (17 neurons per group, n=3 mice each) animals.(D-F) Viral approach to chemogenetically normalize excitability in KD mice (D). AD neuronal excitability rescue ex vivo (E) (mCh control data from panel A, 14 neurons each for KD C21 low dose and KD C21 regular dose from n=3 mice per group). AMPA/NMDA (A/N) ratio rescue in the AD→RSC circuit of KD mice (F) (PTCHD1 KD home cage and training data from panel C, 18 neurons for training low dose and 19 neurons for training regular dose from n=3 mice per group). (G-H) clos activation in RSC during CFC training for KD and rescue groups (G) (mCh controls n=4 mice per group. KD home cage and training n=4 mice per group. KD hM4Di groups n=8 mice per group). CFC LTM test in KD and rescue groups (H) (mCh control and PTCHI)I KD data from FIG. 1I. KD low n=9 mice. KD regular n=8 mice). Two-way ANOVA with repeated measures followed by Bonferroni post-hoc test (excitability data in A, E), two-tailed unpaired t test (AP threshold/half width in A, C, mCh control in G), and one-way ANOVA followed by Bonferroni post-hoc test (F. PTCHD1 KD in G. H). For statistical comparisons, * p<0.05, ** p<0.01; NS, not significant. Data are presented as mean ±SEM.

FIGS. 7A-7M provide electrophysiological recordings and graphs showing that normalizing neuronal hyperexcitability rescues memory deficits in YWHAG and HERC1 KD mice (A-B) Ex vivo recordings from control (mCherry or mCh) vs. YWHAG KD mice showing AP threshold and AP half width (A), neuronal excitability (B) (15 mCh neurons. 16 KD neurons, n=3 mice per group). (C) AMPA/NMDA ratio recordings of the AD→RSC circuit in YWHAG KD mice (14 neurons per group, n=3 mice each). (D) Viral approach to 25 chemogenetically normalize excitability in YWHAG KD mice. (E) AD neuronal excitability rescue ex vivo (mCh control and YWHAG KD data from panel B, 15 neurons for KD C21 low dose group from n=3 mice). (F) CFC training and LTM recall test in KD and rescue groups (mCh control and YWHAG KD data from FIG. 2B, KD low n=9 mice). (G-H) Ex vivo 30 recordings from mCh control vs. HERC1 KD mice showing AP threshold and AP half width (G). neuronal excitability (H) (15 mCh neurons, 23 KD neurons, n=3 mCh mice, n=4 KD mice). (I) AMPA/NMDA ratio recordings of the AD→RSC circuit in HERC1 KD mice (12 home cage, 13 training neurons, n=3 mice each). (J) CFC training and LTM recall test in KD and rescue groups (mCh control and HER('I KD data from FIG. 2K. KD low n=9 mice). (K-M) KIR2.2 (K) (11 mCh neurons from 5 mice, 10 PTCHD1 KD neurons from 5 mice, 11 YWHAG KD neurons from 5 mice, 12 HERC1 KD neurons from 6 mice). CAV2.1 (L) (9 mCh neurons from 6 mice. 8 PTCHD1 KD neurons from 5 mice. 8 YWHAG KD neurons from 5 mice. 8 HERC1 KD neurons from 6 mice), and CAV2.2 (M) (9 mCh neurons from 6 mice, 8 PTCHD1 KD neurons from 5 mice. 8 YWHAG KD neurons from 5 mice. 8 HERC1 KD neurons from 6 mice) ex vivo current recordings. Current-voltage plotted for KIR2.2, current density-voltage plotted for CAV2.1 and CAV2.2. Two-tailed unpaired t test (A, C, G, I), two-way ANOVA with repeated measures followed by Bonferroni post-hoc test (B, E, H, K-M), and one-way ANOVA followed by Bonferroni post-hoc test (F, J). For statistical comparisons, * p<0.05, ** p<0.01, *** p<0.001; NS, not significant. Data are presented as mean ±SEM.

FIGS. 8A-8N provide staining that shows that C1QL2 Expression in human ATN, AD/AV outputs, and risk gene KD in AD, (Related to FIGS. 1 and 2). (A) C1ql2 FISH staining in human anterior thalamus tissue. Reference atlas image from the Allen Institute showing human AD. DAPI staining (blue), and C1ql2 staining (green). (B) C1ql2. Slc17a6 FISH staining in mouse AD. Over 95% of all Slc17a6+ neurons in AD expressed C1ql2 (n=3 mice). Slc17a6 is also known as Vglut2. a marker of excitatory neurons. (C-D) RSC (C) and PreSub (D) CTB injection sites. (E) Retrograde rabies virus (RV), which was injected into PreSub or RSC, and C1ql2 FISH staining in mouse ATN. Over 89% of all PreSub-projecting RV+ neurons were C1ql2+. and over 92% of all RSC-projecting RV+ neurons were C1ql2+(n=3 mice per group). (F-G) Retrograde tracing using excitatory CaMKII-Cre and inhibitory GAD2-Cre mice by injecting Cre-dependent RV into PreSub or RSC. AD/AV retrograde labeling from PreSub (F) or RSC (G). An average of 192 RV+ and 14 RV+ cells were observed in AD from CaMKII+ and GAD2+ PreSub cells, respectively (n=3 mice). An average of 152 RV+and 114 RV+ cells were observed in AD from CaMKII+ and GAD2+ RSC cells, respectively (n=3 mice). (H) Ptchd1 FISH staining in a mCh control virus injected mouse, which is related to the in vivo KD experiments. (I-K) Using the circuit-based approach described in FIG. 1H. CNTNAP2 KD (I), MTOR KD (J), and ATP1A3 KD (K) was performed in AD neurons, as revealed by FISH staining. Cntnap2, Mtor, and Atpla3 expression is decreased by 94%. 98%, and 96% (fluorescence intensity) respectively in KD mice as compared to mCh control mice (n=3 mice per group). (L-M) CFC training (L) and LTM recall tests (M) in CNTNAP2, MTOR, and ATP1A3 KD mice (n=8 mice per group). (N) ASD and schizophrenia risk gene expression in mediodorsal (MD) thalamus. AD thalamus, and hippocampal CA1. Counts are based on 428 ASD and schizophrenia risk genes. Plot shows risk gene counts that are clearly expressed in each of these brain regions. One-way ANOVA followed by Bonferroni post-hoc test (L-M). NS, not significant. Data are presented as mean ±SEM.

FIGS. 9A-9I provide staining and graphs showing risk gene KD in hippocampal CA1, COL25A1 expression in marmosets, and brain-wide input patterns of AD/AV thalamus, related to FIGS. 2 and 3. (A) FISH staining demonstrates successful KD of various risk genes from hippocampal CAI using a virus cocktail expressing target guide RNAs and constitutive SpCas9. In the control group (referred to as mCh control). a mCherry virus replaced the guide RNA virus and the SpCas9 virus was identical. The top row shows staining in mCh control tissue, whereas the bottom row shows 4 staining in individual KD mice, Ywhag, Gria3, Cacnalg, Herc1, Cntnap2, Mtor, and Atpla3 expression is decreased by 90%, 88%. 91%. 89%. 88%, 94%, and 95% (fluorescence intensity) respectively in KD mice as compared to mCh control mice (n=3 mice per group). (B) KD of YWHAG. GRIA3, CACNA1G, and HERC1 from CAI followed by CFC training and LTM recall tests (n=8 mice per group). The KD of these four genes individually from AD neurons led to CFC deficits. (C) KD of CNTNAP2. MTOR, and ATP1A3 from CAI followed by CFC training and LTM recall tests (n=7 mice per group). The KD of these three genes individually from AD neurons did not alter CFC memory behavior. (D) FISH staining in marmoset ATN showing Col25al expression. Dashed line indicates the border between AD and AV. (E-F) Individual helper (GFP, green) (E) and RV (mCherry, red) (F) channels showing restriction of RV labeling (i.e., starter cells) to mouse AV thalamus. Dashed line indicates the border between AD and AV. Surgery information is provided in the legend of FIG. 3C. (G-H) Inputs to AD (G) or AV (H) rank-ordered from highest to lowest average RV-positive cell counts (n=3 mice for AD, n=4 mice for AV, normalized starter cells across groups). Regions with an asterisk are among those reported in the literature previously (Jankowski et al., 2013), A1 (primary auditory cortex). AcbC (accumbens nucleus core), AcbSh (accumbens nucleus shell), APTD (anterior pretectal nucleus dorsal part), Cg1 (cingulate cortex area 1), Cg2 (cingulate cortex area 2), Cg/RS (cingulate retrosplenial), CM (central medial thalamic nucleus), CPu (caudate putamen, dorsal striatum), DM (dorsomedial hypothalamic nucleus), DpG (deep gray layer of the superior colliculus), DpMe (deep mesencephalic nucleus), DpWh (deep white layer of the superior colliculus), FrA (frontal association cortex), InG (intermediate gray layer of the superior colliculus), InWh (intermediate white layer of the superior colliculus), IPF (interpeduncular fossa), LH (lateral hypothalamic area), LHb (lateral habenular nucleus), LO (lateral orbital cortex), LPAG (lateral periaqueductal gray), LPtA (lateral parietal association cortex), MI (primary motor cortex), M2 (secondary motor cortex), MCPC (magnocellular nucleus of the posterior commissure), MDc (mediodorsal thalamic nucleus central part), MDI (mediodorsal thalamic nucleus lateral part), MHb (medial habenular nucleus), ML (medial mammillary nucleus. lateral part), MM (medial mammillary nucleus, medial part), MO (medial orbital cortex), MPtA (medial parietal association cortex), PAG (periaqueductal gray), PH (posterior hypothalamic area), Pir (piriform cortex), PPT (posterior pretectal nucleus), PrL (prelimbic cortex), RSA (retrosplenial agranular cortex), RSG (retrosplenial granular cortex), Rt (reticular thalamic nucleus), S1BF (primary somatosensory cortex barrel field), SIHL (primary somatosensory cortex hindlimb region), S1TR (primary somatosensory cortex trunk region), Sub (submedius thalamic nucleus). Subl (subincertal nucleus), V2L (secondary visual cortex lateral area), V2MM (secondary visual cortex mediomedial area), VL (ventrolateral thalamic nucleus), VM (ventromedial thalamic nucleus), VO (ventral orbital cortex), VPM (ventral posteromedial thalamic nucleus), VPL (ventral posterolateral thalamic nucleus), ZID (zona incerta dorsal part), ZIV (zona incerta ventral part), (I) RV labeling in AD or AV tracing mice showing PrL, SIBF, and RSG brain regions. One-way ANOVA followed by Bonferroni post-hoc test (B-C). For statistical comparisons. ** p<0.01; NS, not significant. Data are presented as mean ±SEM.

FIGS. 10A-10V provides staining, electrophysiological recordings, and graphs showing the electrophysiological properties of AD/AV neurons and AD Circuits, and role of AD thalamus in various behavioral paradigms, related to FIGS. 3 and 4. (A-H) Resting membrane potential or RMP (A). input resistance or Rin (B), membrane time constant or tau (C), membrane capacitance or Cm (D), action potential (AP) threshold (E), AP amplitude (F). AP half width (G), after-hyperpolarization potential (AHP) (H) (22 AD RV+. 17 AD RV−, 18 AV neurons, n=3 mice). Surgery information is provided in the legend of FIG. 3F. (I) Channelrhodopsin-2 (ChR2) expression in mouse AD by injecting a retrograde RV expressing Cre into PreSub and Cre-dependent ChR2-eYFP in AD, current clamp (right top) or voltage clamp (right bottom) traces show light responses. (J) Demonstration of monosynaptic connectivity in ex vivo optogenetic mouse brain slice recordings from AD circuits. TTX (tetrodotoxin), 4AP (4 aminopyridine). CNQX (cyanquixaline), (K-L) Light-induced current traces, onset latency, amplitude of AD circuits (K), traces, pairedpulse ratio of AD circuits (L) (26/27 AD PreSub and 28/35 AD→RSC neurons had responses, n=3 mice). (M) Anterograde labeling experiments, PreSub and RSC injection sites. (N) C21-induced AD neuronal inhibition ex vivo using a 50 pA step current injection protocol. (O-V) C21 was injected prior to the open field test session (15 min duration) (O), rotarod test (3 trials) (P), and inhibitory avoidance training. which was followed by a recall test 24 hr later (Q) (n=9 mice per group). C21 was injected immediately after CFC training to inhibit AD during the cellular consolidation phase followed by a LTM test 24 hr later (R) (n=8 mice per group). C21 was injected prior to the CFC LTM recall test (S), innate avoidance test (T), tone fear conditioning training, which was followed by a recall test 24 hr later (U), and prior to the T-maze spatial working memory training, 10 s delay tests. and 60 s delay tests (V) (n=9 mice per group), mCherry control (mCh). Surgery information is provided in the legend of FIG. 4A. One-way ANOVA followed by Bonferroni post-hoc test (A-H). and two-tailed unpaired t test (KL, O-V). For statistical comparisons. * p<0.05, ** p<0.01, *** p<0.001; NS, not significant. Data are presented as mean ±SEM.

FIGS. 11A-11M provide staining, electrophysiological recordings, and graphs showing that mEPSC and LFP Recordings from AD During CFC, and Chemogenetic Inhibition of PreSub or RSC Excitatory Neurons During CFC, Related to FIG. 4. (A) AD mEPSC traces and cumulative (cum.) probability plots (16 home cage neurons. 18 CFC training neurons, n=3 mice per group). (B) Representative images of cFos+neurons in AD from the home cage group (Home), immediate shock group (Im. Shk.), and CFC training group (Training). Related to FIG. 4D. (C-D) Dil555 (red) electrode localization in AD, DAPI staining (blue) for LFP recordings from AD thalamus (C), AD inhibition using the chemogenetic strategy in FIG. 4A during CFC training prevented the training-induced increases in theta and gamma rhythms (D) (n=9 mCh mice. n=7 hM4Di-mCh mice). Related to FIGS. 4E-4F. (E) Di1555 (red) electrode localization in RSC and PreSub, DAPI staining (blue). (F-G) AD→PreSub (F) and AD→RSC (G) circuit coherence Pre vs. Post CFC training (n=7 mice per group). (H) In vivo coupling between the theta rhythm phase in AD and gamma rhythm amplitude in RSC was enhanced by CFC training, which was blocked by AD inhibition during training (n=7 mice per group). AD inhibition used the chemogenetic strategy in FIG. 4A. Modulation index (MI). (I-K) CaMKII-hM4Di-mCherry labeling in PreSub and RSC (I), C21-induced inhibition of excitatory neurons during CFC training in PreSub (J) or RSC (K) (n=6 mCh mice, n=8 hM4DimCh mice per brain region). LTM recall tests were performed 24 hr after training. (L) Optogenetic AD→RSC circuit manipulation. CFC training data (n=14 eYFP mice, n=12 eArch-eYFP mice, n=7 ChR2-eYFP mice). Surgery information is provided in the legend of FIG. 4I. (M) Various optogenetic groups of mice were used in a 10 min light-on open field session. eYFP, AD→RSC eArch. and AD→RSC ChR2 mice from FIG. 4I. AD→PreSub eArch and AD→RSC eArch using C1q12-Cre mice from FIG. S5A. AD→RSC→EC eArch mice from FIG. 4M, and AV NpHR and AV→RSC NpHR mice from FIG. 5B. Two-tailed unpaired t test (D. J-K), paired t test (F-G), and one-way ANOVA followed by Bonferroni post-hoc test (L-M). For statistical comparisons, * p<0.05, ** p<0.01; NS, not significant. Data are presented as mean ±SEM.

FIGS. 12A-120 provides staining, electrophysiological recordings, and graphs showing that AD Circuit Manipulations in C1q12-Cre Mice, RSC→EC Circuit Tracing, and AV mEPSC Recordings After CFC Training, Related to FIGS. 4 and 5. (A) Injection of a Cre-dependent eYFP virus in the ATN region of C1q12-Cre mice showing anterior and posterior AD labeling, and eYFP+ neurons accounted for over 85% of the CIQL2+ neurons (via antibody staining) in AD thalamus (n=3 mice). Cre mice injected with a Credependent eYFP control virus in the ATN region and two groups of Cre mice injected with a Credependent eArch-eYFP virus in the ATN region along with optic fibers targeting bilateral PreSub or RSC were used in the CFC behavioral paradigm (n=7 mice per group). Terminal inhibition was performed during CFC training, which was followed by a LTM recall test 24 hr later. (B-C) Representative images of cFos+ neurons in RSC (B) and hippocampal CAI (C) from home cage, mCh, and hM4Di-mCh groups. Related to FIGS. 4J-4K. (D-E) Anterograde labeling using CaMKII-ChR2-eYFP injected into RSC (D) and retrograde tracing by injecting CTB555+ into EC (E) validated the RSC→EC circuit. EC layer 5 (EC5). (F) Quantification of RV mCherry+ cells in AD and AV from the ATN→RSC→EC tracing experiment in FIG. 4L (n=4 mice). (G) cFos activation of EC-projecting RSC neurons labeled with CTB647 using home cage, training control (mCh), and training AD hM4Di groups (n=4 mice per group). CTB647, red pseudocolor. Surgery information identical to FIG. 4J with the addition of a CTB647 injection into EC. (H-I) 30) ATN RSC EC circuit manipulation experiment. RSC labeling (H). CFC training data (I) (n=9) eYFP mice. n=11 eArch-eYFP mice). Surgery information is provided in the legend of FIG. 4M. (J) Light-induced AV neuronal inhibition ex vivo. (K) AV cell bodies or AV→RSC terminal manipulation experiment. CFC training data (eGFP n=8 mice, AV NpHR n=10 mice, AV→RSC NpHR n=8 mice). Surgery information is provided in the legend of FIG. 5A. (L-M) AV mEPSC traces and cumulative (cum.) probability plots (L). AV mEPSC amplitude and frequency (M) (15 home cage neurons, 14 CFC training neurons, n=3 mice per group). (N) Representative images of cFos+ neurons in RSC from home cage, eGFP, and NpHR-eYFP groups. Related to FIG. 5C. (O) cFos activation of EC-projecting RSC neurons labeled with CTB555 using home cage. training control (eGFP), training AV→RSC NpHR-eYFP groups (n=4 mice per group). Surgery information for AV manipulation is provided in the legend of FIG. 5C. One-way ANOVA followed by Bonferroni post-hoc test (A, G, K, O), paired t test (F). and twotailed unpaired t test (I, M). For statistical comparisons. * p<0.05, ** p<0.01; NS, not significant. Data are presented as mean ±SEM.

FIGS. 13A-13O provides staining, electrophysiological recordings, and graphs showing that AV Inputs to Inhibitory Neuron Subtypes in RSC, and Electrophysiological Recordings in PTCHD1, GRIA3, and CACNA1G KD Mice, Related to FIGS. 5, 6, and 7. (A) Cre-dependent RV starter cells (yellow) in PV-Cre, SST-Cre, and VIP-Cre mice from RSC. (B-C) AV→RSC inhibition with simultaneous PV or VIP activation in RSC during training from FIG. 5I. CFC training data (B). LTM recall test (C) (PV-Cre: C21 n=8 and C21+light n=6 mice. VIP-Cre: C21 n=7 and C21+light n=6 mice). Surgery information is provided in the legend of FIG. 5I. (D) AD→RSC and AV→RSC terminal inhibition during training in the cocaine-induced conditioned place preference behavior test. Pre-exposure data plotted (n=12 mice per group). Related to FIGS. 5K-5L. (E) Ex vivo recordings from mCh control and PTCHD1 KD AD neurons showing RMP and Cm (24 mCh neurons, 23 KD neurons, n=3 mice per group). Surgery information is provided in the legend of FIG. 1H. (F) AD→RSC AMPA/NMDA ratio traces from PTCHD1 KD experiment in FIG. 6C. (G) Neuronal excitability of AD neurons in home cage (before CFC training) and post-CFC training conditions, using mCh control and PTCHD1 KD groups (mCh: 14 home cage and 13 training neurons. KD: 14 home cage and 16 training neurons, n=3 mice per group). (H) Representative images of cFos+neurons in RSC from home cage, training, training hM4Di low, and training hM4Di reg groups. Related to FIG. 6G. (I) CFC behavioral experiment using PTCHD1 KD and rescue groups. Training data (mCh n=9,KD n=10 mice. KD low n=9 mice. KD regular n=8 mice). Related to FIG. 6H. (J) Neuronal excitability of AD neurons in mCh control, GRIA3 KD, and CACNA1G KD groups (GRIA3: 15 mCh and 18 KD neurons. CACNA1G: 15 mCh and 14 KD neurons, n=3 mice per group). (K-M) KD rescue mice injected with saline (Sal group) or C21 low dose (Low group) prior to the spatial working memory T-maze test (60 s delay between sample and choice), for PTCHD1 KD (K), YWHAG KD (L), and HERC1 KD (M) (n=9 mice per group). Surgeries used the strategy described in FIG. 6D. Dashed line indicates chance level of performance (i.e., 50%). Two-tailed unpaired t test (B-C, E, K-M), paired t test (D), two-way ANOVA with repeated measures followed by Bonferroni post-hoc test (G, J), and one-way ANOVA followed by Bonferroni post-hoc test (I). For statistical comparisons,* p<0.05, *** p<0.001; NS, not significant. Data are presented as mean ±SEM.

FIGS. 14A-14E provides staining, electrophysiological recordings, and graphs showing that Channel Expression in KD AD Neurons, PFC Input to AV Thalamus, and RSC Neurons Receiving Both AD/AV Inputs, Related to FIG. 7. (A-B) FISH staining in AD thalamus in mCh control. PTCHD1 KD. YWHAG KD, and HERC1 KD mice using probes for Kcnj12, Kcnq2, Kcna1, Cacna1a, and Cacna1b (A), quantification (B) (n=6 mice per group). Fluorescence intensity (Fluores. intensity) is plotted in arbitrary units (a.u.). (C) A retrograde Cre-expressing virus injected into nucleus reuniens (RE) with Cre-dependent ChR2-eYFP injected in PFC shows terminal labeling of RE-projecting PFC neurons in AV thalamus. (D-E) Using retrograde RV expressing Cre from PreSub. AD neurons were labeled with Cre-On (DIO) C1V1-eYFP. AV neurons were labeled with Cre-Off (DO) ChETA-tdT. RSC active neurons were labeled using a cFos-CreERT2 virus (Ye et al., 2016) mixed with Cre-dependent eYFP. Dashed line indicates the border between AD and AV (D). AD terminals in RSC were activated using 570 nm light with simultaneous 4-OHT-induced tagging of cFos+ RSC neurons in the home cage, one week later again in the home cage AV terminals in RSC were activated using 410 nm light followed by cFos staining for activated ensembles in RSC. Overlap between the 4-OHT-tagged RSC ensembles and cFos stained ensembles indicated that not all RSC neurons receive inputs from both AD and AV (i.e., ˜25% receive input from AD or AV but not both). Sensitivity of this approach is demonstrated by high degree of overlap when both optogenetic proteins were expressed in AD (AD-AD) rather than one in AD and the other in AV (n=7 mice per group). Immunohistochemistry (IHC) (E). One-way ANOVA followed by Bonferroni post-hoc test (B). For statistical comparisons, ** p<0.01, *** p<0.001; NS, not significant. Data are presented as mean ±SEM.

### DETAILED DESCRIPTION

The disclosure features compositions and methods for ameliorating cognitive impairments associated with neuropsychiatric disorders, particularly those associated with anterodorsal (AD) thalamus hyperexcitability in the brain of a subject.

Embodiments of the disclosure are based, at least in part, on the discovery that many autism and schizophrenia risk genes are expressed in the anterodorsal (AD) subdivision of anterior thalamic nuclei, which has reciprocal connectivity with learning and memory structures. CRISPR-Cas9 knockdown of multiple risk genes selectively in AD thalamus led to memory deficits. While AD is necessary for contextual memory encoding, the neighboring anteroventral (AV) subdivision regulates memory specificity. These distinct functions of AD and AV are mediated through their projections to retrosplenial cortex, using differential mechanisms. Furthermore, knockdown of autism and schizophrenia risk genes PTCHD1, YWHAG, or HERC1 from AD led to neuronal hyperexcitability, and normalization of hyperexcitability rescued memory deficits in these models. This study identifies converging cellular to circuit mechanisms underlying cognitive deficits in a subset of neuropsychiatric disease models.

### Cognitive Impairment

Intellectual disability (ID)/cognitive impairment is characterized by significant limitations in cognitive functions, including reasoning, learning, memory, and adaptive behaviors, which co-occur with many neuropsychiatric disorders, including autism spectrum disorder (ASD) and schizophrenia (Morgan et al., 2008: Matson and Shoemaker, 2009). Cognitive impairments in these disorders have been commonly linked to dysfunction within hippocampal and cortical circuits (O'Tuathaigh et al., 2007; Kvajo et al., 2008; Golden et al., 2018), however whether converging neurobiological mechanisms underlie cognitive impairments across disorders has not been established. This issue has an important implication: if common mechanisms can be identified, therapeutic approaches capable of treating cognitive impairments in a subset of neuropsychiatric disorders may be developed.

PTCHD1 is mutated in some ASD patients with ID (Chaudhry et al., 2015). These patients have multiple symptoms including attention deficits, hyperactivity, sleep abnormality, and memory deficits. Our previous study in mice showed that the selective deletion of PTCHD1 from the thalamic reticular nucleus (TRN) was responsible for attention deficits, hyperactivity, and sleep abnormality, but not memory deficits (Wells et al., 2016). Interestingly, in addition to TRN, PTCHD1 exhibits strong expression in one other brain region, the anterodorsal (AD) thalamus, but not in well-known memory structures such as hippocampus, entorhinal cortex, or amygdala (Lein et al., 2007).

AD thalamus is part of the understudied anterior thalamic nuclei (ATN) complex, which also contains anteroventral (AV) and anteromedial (AM) subdivisions. ATN has reciprocal connectivity with frontal cortical areas, hippocampal subregions, and hypothalamic nuclei involved in memory functions (Jankowski et al., 2013). Lesion studies have suggested a potential role for ATN in spatial navigation (Winter et al., 2015) and cognitive tasks (Aggleton et al., 1991: Mitchell and Dalrymple-Alford, 2006; Savage et al., 2011: Warburton and Aggleton, 1999). Recent work has indicated that ATN are necessary for fear memory encoding and remote memory retrieval (Yamawaki et al., 2019; Vetere et al., 2021). Robust reductions in the number of ATN neurons was reported in tissue from patients (Young et al., 2000), suggesting a potential role for ATN dysfunction in schizophrenia. For these reasons, the inventors hypothesized that AD thalamus dysfunction underlies memory deficits in PTCHD1 mutant mice (Wells et al., 2016), which may extend to other ASD and schizophrenia models.

PTCHD1 led us to focus on AD thalamus, whose precise role in memory remains unclear. In this study, using wild type mice the inventors showed that the AD→retrosplenial cortex (RSC) circuit is necessary for memory encoding, whereas the neighboring AV→RSC circuit regulates memory specificity. The inventors observed that AD thalamus shows a high percentage of ASD and schizophrenia risk gene expression. The knockdown (KD) of different risk genes from AD leads to cognitive deficits. Several KD models had AD neuronal hyperexcitability that correlated with an impairment in learning-induced synaptic strengthening. The inventors demonstrated that rescuing AD hyperexcitability in KD models is sufficient to restore multiple memory functions. Together, this study identifies cellular, circuit, and behavioral convergence underlying cognitive deficits in a subset of neuropsychiatric disease models.

### Compositions and Methods of Treating Anterodorsal (AD) Hyperexcitability

Provided herein are compositions, assays, and methods of screening, diagnosing, and treating a subject with cognitive dysfunction, anterodorsal (AD) hyperexcitability, and/or a neuropsychiatric disease.

In one aspect, provided herein is a method of ameliorating anterodorsal (AD) thalamus hyperexcitability in a subject, the method comprising: administering to the subject an agent that reduces and/or normalizes AD thalamus hyperexcitability.

In another aspect, provided herein is a method of ameliorating anterodorsal (AD) thalamus hyperexcitability in a subject, the method comprising: administering to the subject a chemogenetic composition that reduces and/or normalizes AD thalamus hyperexcitability.

In another aspect, provided herein is a method of screening for an agent that reduces and/or normalizes AD thalamus hyperexcitability, the method comprising: contacting a neuron or population thereof comprising an alteration in a PTCHD1. YWHAG. or HERC1 polynucleotides and/or polypeptides with a test agent: and detecting an biopotential in the neuron.

In some embodiments of any of the aspects, the agent or test agent provided herein is selected from the group consisting of: an NMDA receptor agonist, an ion-channel blocker, an ion channel modulator, an ion channel activator, a chemogenetic system, and a gene-editing system.

Non-limiting examples of chemogenetic compositions that can be used in the disclosure are described, e.g., in U.S. Pat. Nos. 8,435,762 B2, 10,538,571 B2, and 10,961,296 B2;US Pg. US2019/ 0175763A1: WO 2017/049252A1, the teachings of each of which are incorporated herein by reference in their entireties.

In some embodiments, the chemogenetic composition or gene-editing system alters the level or activity of one or more of PTCHD1, YWHAG, and HERC1 polynucleotides and/or polypeptides in a neuron.

In some embodiments, the chemogenetic composition or gene-editing system alters the level or activity of the NMDA receptor in the AD thalamus. In some embodiments, the agent or test agent increases the level or activity of KIR2.2, CAV2.1, and CAV2.2 in the AD thalamus.

### Chemogenetic Receptors and Ion Channels

In another aspect, provided herein is a chemogenetic composition comprising: an engineered ligand-gated receptor comprising a drug-binding domain. In another aspect, provided herein is a chemogenetic composition comprising: an engineered ligand-gated ion channel comprising a drug-binding domain. In some embodiments, the composition further comprises an agent that specifically binds to the drug-binding domain of the engineered ligand-gated receptor or the engineered ligand-gated ion channel. In some embodiments, the ligand-gated receptor or ion channel is selected from the group consisting of: hM4Di (inhibitory), hM3Dq (activatory), hM3Ds (activatory), KORD (activatory), PSAM/PSEM ligand activated ion channels (both inhibitory and activatory versions), GluCl (inhibitory), Tetracycline transactivator (changes in gene expression, inhibition), reverse transactivator (changes in gene expression, activation).

In some embodiments, chemogenetics may involve the use of Designer Receptors Exclusively Activated by Designer Drugs (DREADDS). DREADD receptors can be introduced into neural tissue through a range of gene transfer strategies, allowing for transient and repeatable interventions in brain dynamics upon application of otherwise inert exogenous ligands, for example clozapine-n-oxide (CNO). In brief, DREADDs involve the use of receptor proteins derived from targeted mutagenesis of endogenous G-protein coupled receptor DNA to yield synthetic receptors. These receptors are readily expressed in neuronal membranes, but lack an endogenous ligand to activate them. However, they are sensitive to the otherwise inert drug CNO, which can be delivered systemically and binds to DREADD receptors. One popular variant is hM4Di, which is an engineered version of the M4 muscarinic acetylcholine receptor. When bound by CNO, membrane hyperpolarization results through a decrease in cAMP signaling and increased activation of inward rectifying potassium channels (Armbruster et al., Proc Natl Acad Sci U S A. 2007:104(12):5163-5168: Rogan & Roth, Pharmacol Rev. 2011:63(2):291-315), each of which is incorporated herein by reference in its entirety. This yields a temporary suppression of neuronal activity similar to that seen after endogenous activation of the M4 receptor. Compositions and methods for using DREADDs to treat disorders affecting the nervous system are described for example in US Patent Publication No. 20210179676. 20210077635, 20200323863, 20200316217, 20200208201, 20190194287, 20190175763, 20190134155, 20190083652. 20190046662, 20180193414, 20180078658, 20160375097, and 20160354330, each of which is incorporated herein by reference in its entirety.

In some embodiments of any of the aspects, the agent is an exogenous ligand of the engineered ligand-gated receptor or the engineered ligand-gated ion channel provided herein.

A ligand that can bind to and activate engineered receptors or ion channels described herein can have selective binding (e.g., enhanced binding or increased potency) for the engineered receptor or ion channel described herein (e.g., relative to an unmodified receptor or ion channel). In some cases, a ligand that can bind to and activate engineered receptors described herein does not bind to and activate endogenous receptors (e.g., endogenous receptors). In some cases, a ligand that can bind to and inhibit engineered ion channels described herein does not bind to and inhibit endogenous ion channels. A ligand that selectively binds to and activates or inhibits a modified engineered receptor or ion channel provided herein (e.g., a chemogenetic receptor or channel having at least one amino acid modification that confers pharmacological selectivity to the engineered ion channel or receptor) described herein over an unmodified ligand can also be described as having enhanced potency for a modified engineered receptor or ion channel.

The methods and compositions provided herein are directed to a polynucleotide encoding an engineered receptor and ion channels as described herein. In some aspects of the disclosure, the present disclosure is directed to a vector comprising a polynucleotide encoding an engineered receptor or ion channel as described herein. In some aspects of the disclosure, the present disclosure is directed to a pharmaceutical composition comprising a polynucleotide encoding an engineered receptor or ion channel as described herein or a vector comprising the polynucleotide encoding an engineered receptor or ion channel as described herein and a delivery vehicle.

### Delivery Vehicles and Vectors

In some embodiments of any of the aspects, the delivery vehicle is a vector. In some embodiments of any of the aspects, the delivery vehicle is a lipid, a liposome, or a nanoparticle. In some embodiments of any of the aspects, the vector is a viral vector comprising a polynucleotide encoding an engineered receptor or ion channel described herein. In some embodiments, the viral vector is an adenoviral vector, a retroviral vector, an adeno-associated viral (AAV) vector, or a herpes simplex-1 viral vector (HSV-1). In some embodiments, the AAV vectors is selected from the group consisting of AAV1, AAV2, AAV3b, AAV4, AAV5, AAV6, AAV7, AAV8, AAV9, and AAVrh10. In some embodiments, the AAV vector is selected from AAV5, AAV6, and AAV9. In some embodiments, the vector is derived from a vector selected from the group consisting of AAVI, AAV2, AAV3b, AAV4, AAV5, AAV6, AAV7, AAV8, AAV9, and AAVrh10. In some embodiments, the AAV vector is derived from AAV-2 or AAV-9.

AAV vectors have been shown to transduce neurons, with no evidence of cytotoxicity (Freese et al., Epilepsia, 38(7):759-766, 1997). AAV vectors are reviewed in general in Monahan et al., Gene Therapy, 7:24-30, 2000. Furthermore, U.S. Pat. No, 5,677,158 describes methods of making AAV vectors. AAV vectors carrying transgenes have been described, for example, in Kaplitt et al., Nat. Genet., 8:148-154, 1994: Alexander et al., Hum. Gene Ther., 1996, 7:841-850; Bartlett et al., Hum. Gene Ther., 1998, 9:1181-1186; Mandel et al., Proc. Natl. Acad. Sci., U.S.A., 94:14083-14088, 1997; Lo et al., Hum. Gene Ther., 10:201-21, 1999; Bankiewicz et al., Exp. Neurol., 164:2-14, 2000; Peel et al., J Neurosci Methods., 98:95-104, 2000; Bueler H., Biol. Chem., 380:613-22, 1999; Rabinowitz et al., Curr Opin Biotechnol., 9:470-5, 1998: Monahan et al., Mol. Med. Today 2000, 11:433-440. The teachings of each of the above references are incorporated herein by reference in their entireties.

Exemplary formulations for non-viral delivery of polypeptides are also disclosed, e.g., in U.S. Pat. Nos. 5,981,505; 6,217,900; 6,383,512; 5,783,565; 7,202,227; 6,379,965; 6,127,170; 5,837,533; and WO 03/093449, , the teachings of each of which are incorporated herein by reference in their entireties.

Furthermore, physical methods for introducing a polynucleotide (e.g., encoding the chemogenetic receptor or channel provided herein) into a host cell include calcium phosphate precipitation, DEAE-dextran, lipofection, particle bombardment, microinjection, electroporation, cell sonication, receptor-mediated transfection, and the like. See, e.g., Shigekawa et al., BioTechniques, 6:742-751, 1988: Mannino et al., BioTechniques, 6:682 690, 1988: and Huang, Q., et al., Effective Gene Transfer into Central Nervous System Following Ultrasound-Microbubbles-Induced Opening of the Blood-Brain Barrier. Ultrasound in Medicine & Biology, 2012. 38(7): p. 1234-1243, the teachings of each of which are incorporated herein by reference in their entireties.

Methods of targeting a composition to a neuron in a subject are known in the art, e.g., Kügler et al., “Neuron-Specific Expression of Therapeutic Proteins: Evaluation of Different Cellular Promoters in Recombinant Adenoviral Vectors,” Molecular and Cellular Neuroscience, vol. 17, Issue 1, January 2001, pp. 78-96. For example, a promoter specific to the brain region to be targeted can be used.

### Promoters

In some embodiments of any of the aspects, the method provided herein comprises expressing the engineered receptor in an excitable cell. In some embodiments, the excitable cell is a neuron. In some embodiments, the neuron is a thalamic neuron.

The methods and compositions provided herein can comprise a promoter specific to excitable cell expression, e.g., neurons in the CNS.

In some embodiments, the promoter is selected from the group consisting of: c-fos, human synapsin-1, myelin basic protein (MBP), glial fibrillary acid protein (GFAP), neuron specific enolase (NSE), CMV promotor, Thy1, calcium/calmodulin-dependent protein kinase II promoter, a tubulin alpha I promoter, a neuron-specific enolase promoter, a platelet-derived growth factor beta chain promoter, an hSYN1 promoter, a TRPV1 promoter, a Nav1.7 promoter, a Nav 1.8 promoter, a Nav1.9 promoter, Advillin promoter, somatostatin, parvalbumin, GABAα6, L7, and calbindin, promoters for kinases such as PKC. PKA, and CaMKII: promoters for other ligand receptors such as NMDAR1, NMDAR2B, GluR2; promoters for ion channels including calcium channels, potassium channels, chloride channels, and sodium channels: and promoters for other markers that label classical mature and dividing cell types, such as calretinin, nestin, and beta3-tubulin.

In some embodiments, the promoter is an inducible promoter. For instance, the promoter can be inducible by a trans-acting factor which responds to an exogenously administered drug. The promoters could be, but are not limited to tetracycline-on or tetracycline-off, or tamoxifen-inducible Cre-ER.

Exemplary promoters are further described, e.g., in Gordon et al. Cell 50:445 (1987), Feng et al., Neuron 28:41 (2000), Li L, Suzuki T, Mori N, Greengard P. Identification of a functional silencer element involved in neuron-specific expression of the synapsin I gene. Proc Natl Acad Sci USA 1993: 90: 1460-1464, Gloster A, Wu W, Speelman A, Weiss S, Causing C, Pozniak C et al. The T alpha 1 alpha-tubulin promoter specifies gene expression as a function of neuronal growth and regeneration in transgenic mice. J Neurosci 1994: 14: 7319-7330., Mayford M, Baranes D, Podsypanina K, Kandel E R. The 3′-untranslated region of CaMKII alpha is a cis-acting signal for the localization and translation of mRNA in dendrites. Proc Natl Acad Sci USA 1996: 93: 13250-13255, Sasahara M, Fries J W. Raines E W, Gown A M, Westrum L E, Frosch M P et al. PDGF B-chain in neurons of the central nervous system, posterior pituitary, and in a transgenic model. Cell 1991: 64: 217-227, and Hioki, H., Kameda, H., Nakamura, H. et al. Efficient gene transduction of neurons by lentivirus with enhanced neuron-specific promoters. Gene Ther 14, 872-882 (2007), the teachings of which are incorporated herein by reference in their entireties.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Expression of ASD and Schizophrenia Risk Genes in AD Thalamus

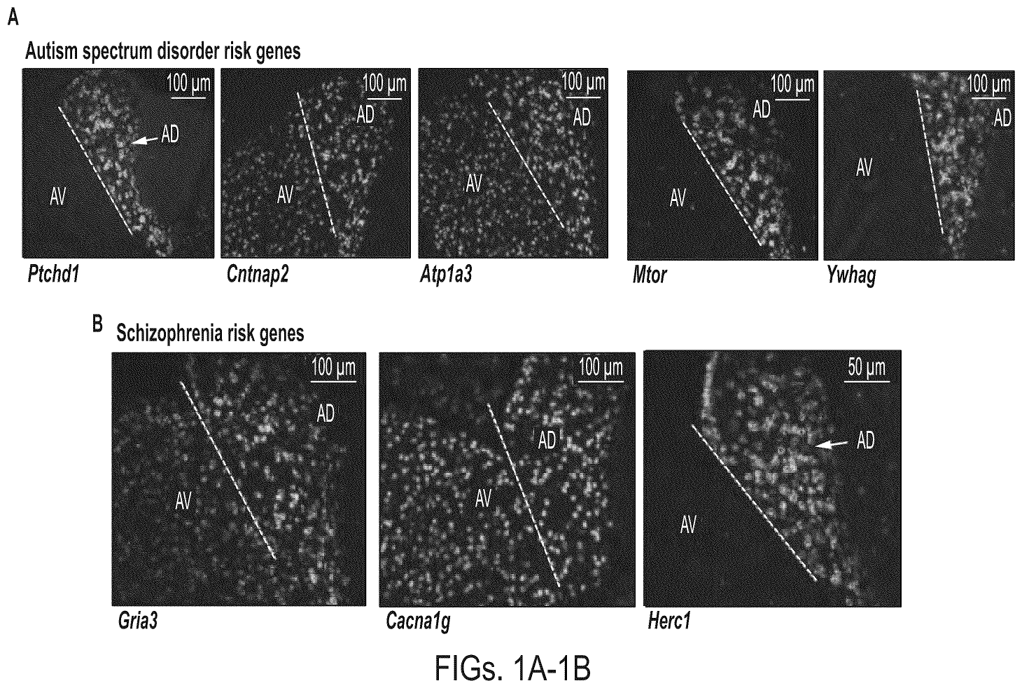

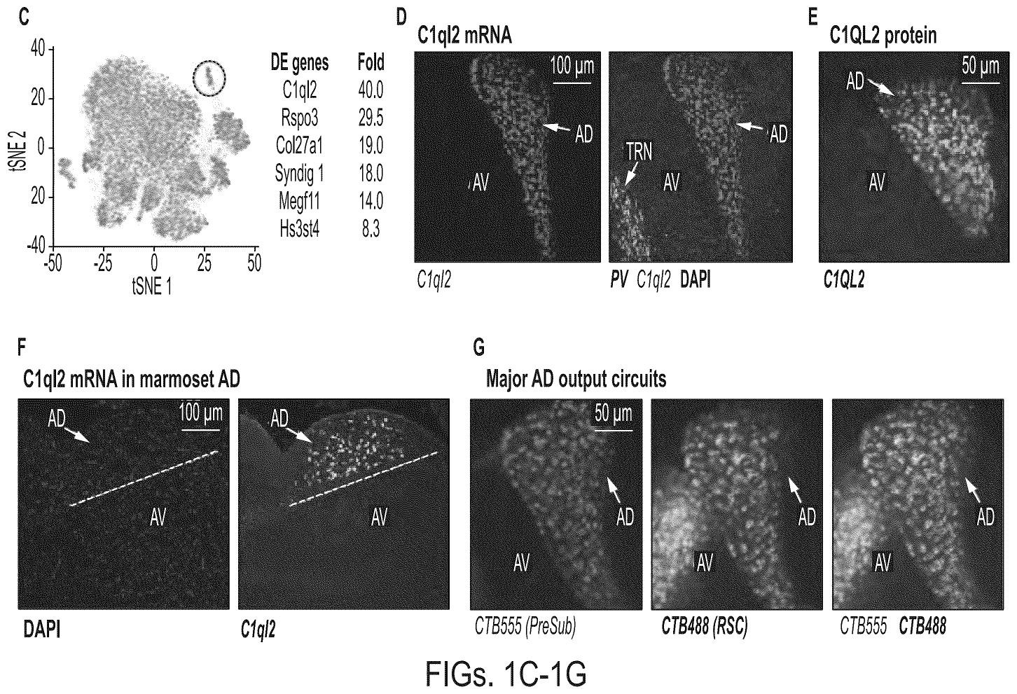

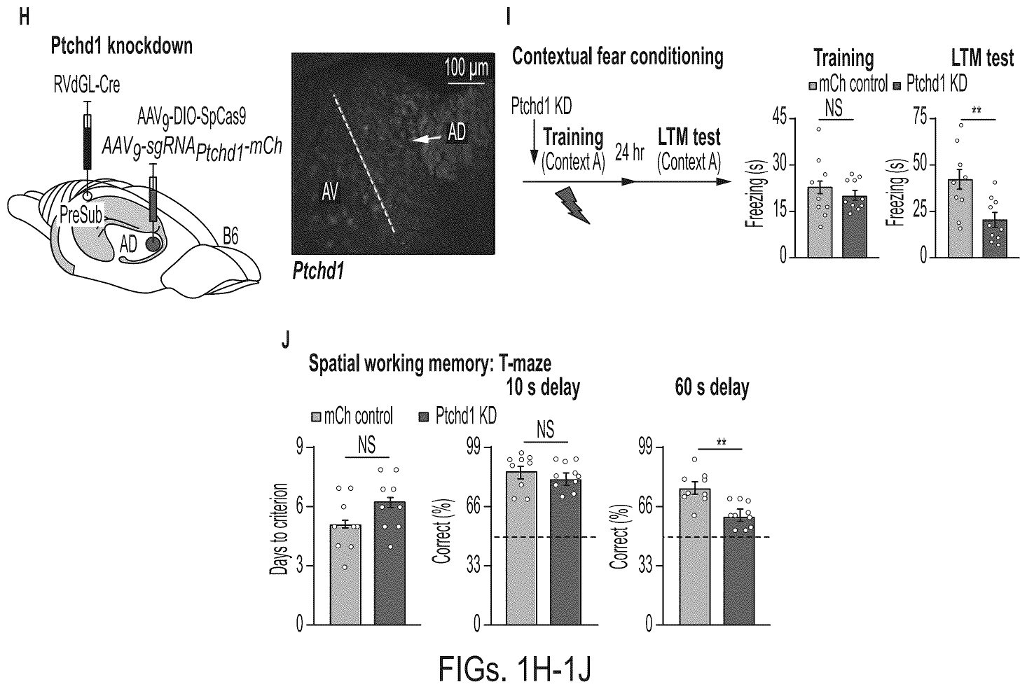

Fluorescent in situ hybridization (FISH) revealed that the ASD/ID gene, PTCHD1, is selectively expressed in AD thalamus within ATN (FIG. 1A). By examining gene expression in the Allen Brain Atlas (Lein et al., 2007) using 45 syndromic ASD risk genes from the SFARI database, the inventors noticed that 10 risk genes had clear AD expression. Their expression pattern could be divided into three groups, namely expression only in AD thalamus within ATN, higher expression in AD thalamus relative to other ATN subdivisions, and high expression in the entire ATN. The inventors focused on four of these risk genes, two of which had higher expression in AD vs. other ATN subdivisions (contactin associated protein 2 or CNTNAP2, ATPase Na⁺/K⁺ transporting subunit alpha 3 or ATP1A3), while the other two showed expression only in AD within ATN (mechanistic target of rapamycin kinase or MTOR, tyrosine 3-monooxygenase/tryptophan 5-monooxygenase activation protein gamma or YWHAG) (FIG. 1A). Following a similar approach but this time using the top ten schizophrenia risk genes (Singh et al.1 2020), the inventors noticed that three risk genes exhibited high expression in AD thalamus, namely glutamate ionotropic receptor AMPA type subunit 3 (GRIA3), calcium voltage-gated channel subunit alpha 1G (CACNA1G), and HECT and RLD domain containing E3 ubiquitin protein ligase family member 1 (HERC1) (FIG. 1B). Given that AD thalamus was the only ATN subdivision to exhibit expression of many risk genes, it is possible that AD thalamus-specific dysfunction contributes to disease phenotypes in a subset of different disorders.

### Example 2: Molecular Marker and Outputs of AD Thalamus

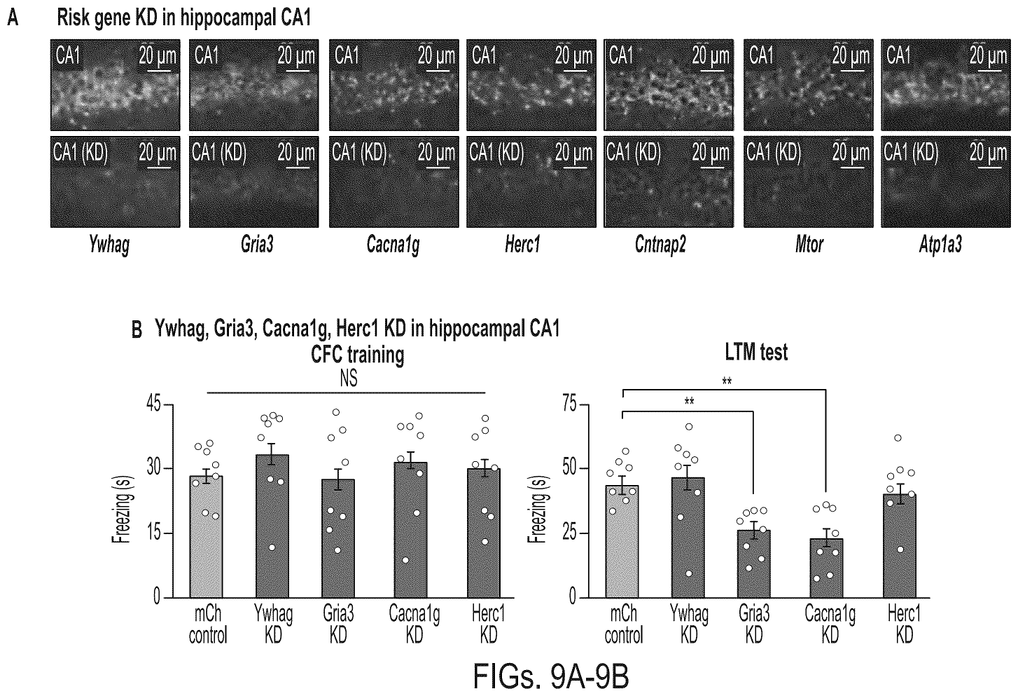

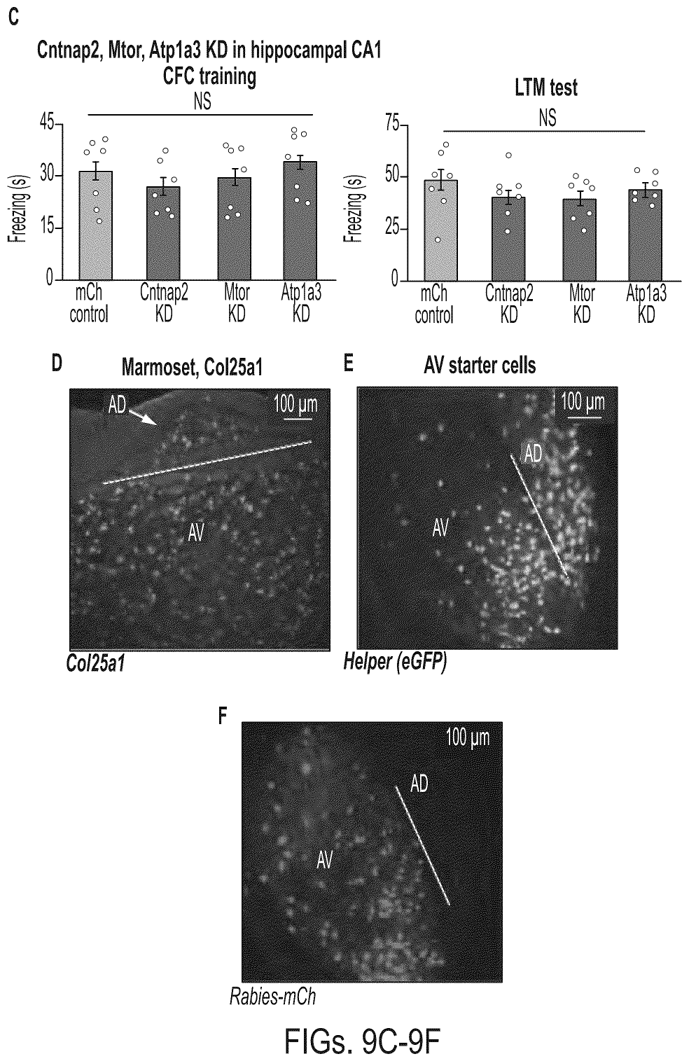

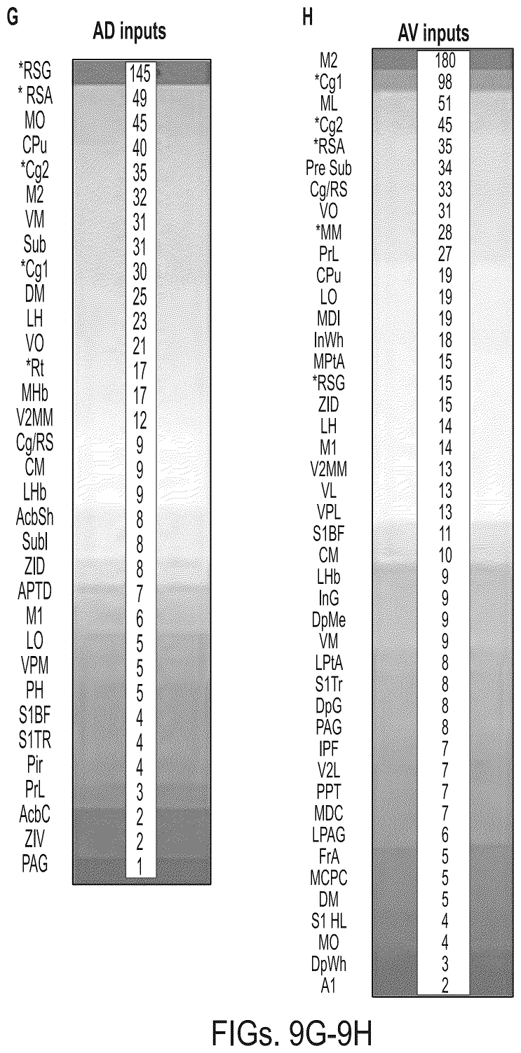

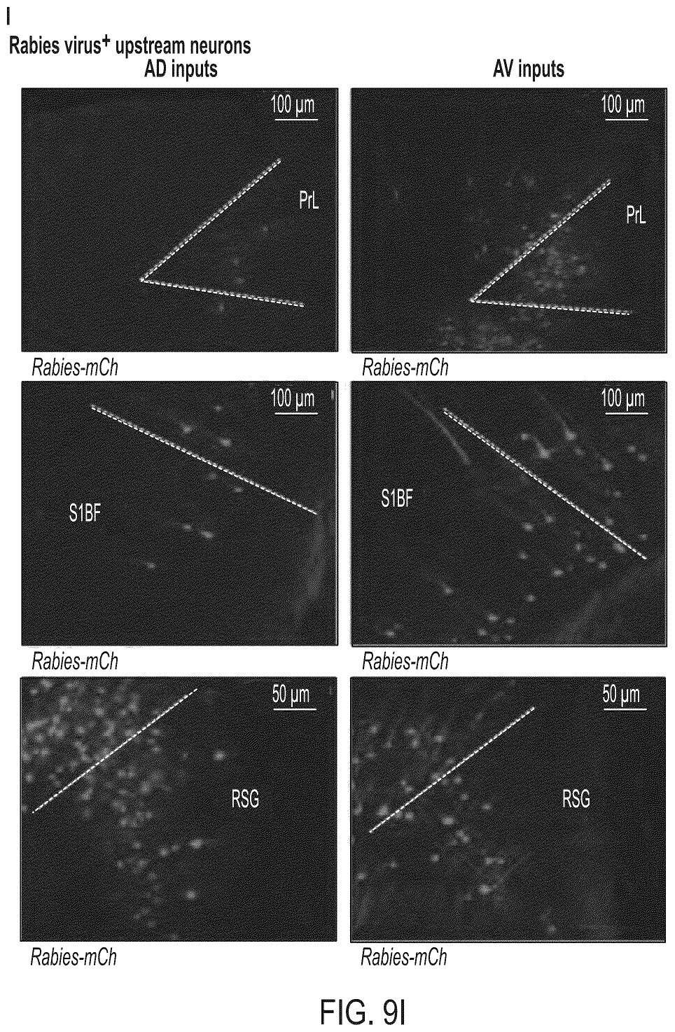

To test this hypothesis, the inventors needed to develop an approach to selectively manipulate risk genes in AD thalamus within ATN. The inventors started by determining whether specific molecular markers could be identified within ATN. Taking advantage of the DropViz RNA-sequencing dataset (Saunders et al., 2018), the inventors focused on 11 excitatory neuron clusters in mouse thalamus (FIG. 1C). One of these clusters had the highest levels of complement Clq like 2 (C1QL2) gene expression. Staining experiments showed that C1QL2 is selectively expressed in AD thalamus within ATN (FIGs. 1D-1E) (Vertes et al., 2015). Similar to mice. (1QL.2 mRNA was restricted to AD thalamus in the ATN of marmosets (FIG. IF), and the inventors also observed C1QL2 expression in human tissue containing anterior thalamus (FIG. 9). Thus. C1QL2 is an AD thalamus-specific molecular marker conserved from rodents to primates.

C1QL2⁺ AD neurons are excitatory (FIG. 9B). It is known that AD neurons primarily project to pre-subiculum (PreSub) and retrosplenial cortex (RSC) (Jankowski et al., 2013) (FIG. 1G, and see FIGS. 9C-9D). These retrograde tracing experiments also showed that AV thalamus projects to RSC but not PreSub, indicating that both AD and AV subdivisions converge on RSC. By examining the overlap of two different tracers in AD, the inventors found that the majority of AD neurons send collaterals to both PreSub and RSC (FIG. 1G). Further, using FISH the inventors directly demonstrated that C1QL2⁺ AD neurons have a high degree of overlap with AD neurons projecting to either downstream target (FIG. 9E). Using calcium/calmodulin-dependent protein kinase II (CAMKII)-Cre mice, glutamate decarboxylase 2 (GAD)2)-Cre mice, and Cre-dependent, monosynaptic retrograde tracing (Wickersham et al., 2007), the inventors found that most AD neurons project to excitatory CaMKII⁺ neurons in PreSub (FIG. 9F). Applying a similar strategy to RSC, the inventors observed that AD and AV project to both excitatory and inhibitory neurons (FIG. 9G).

### Example 3: Memory Impairments in Multiple AD Thalamus-Specific Risk Gene Knockdown Mice

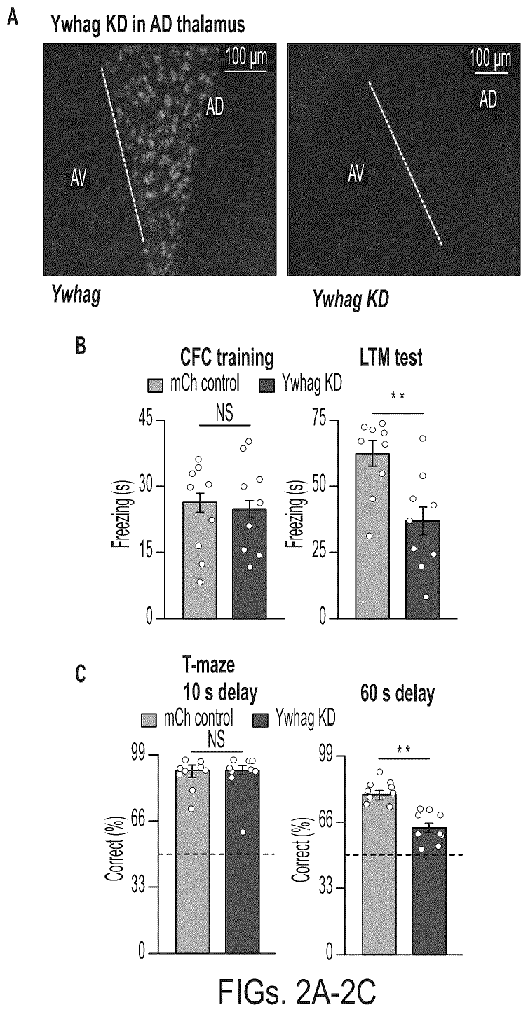

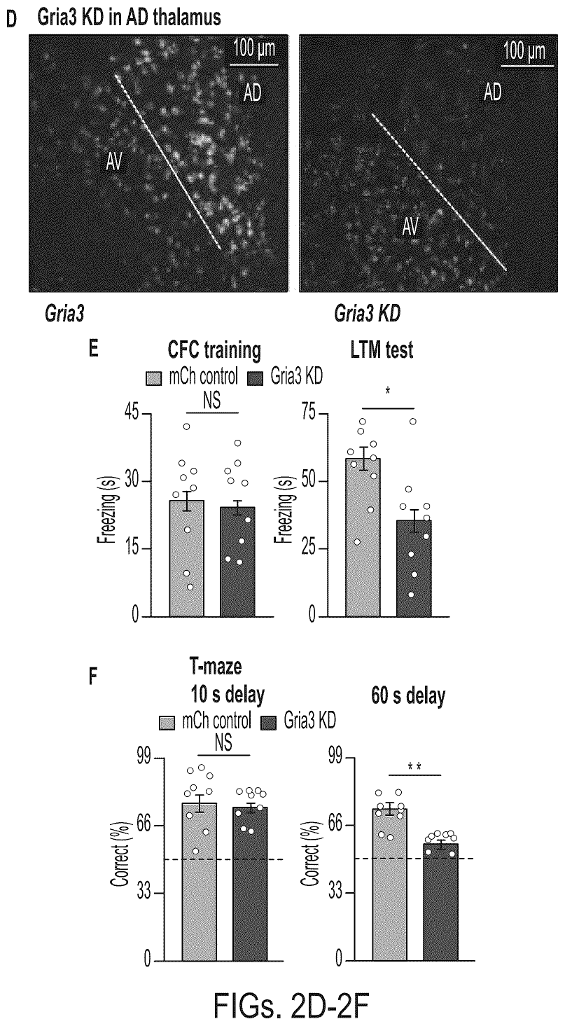

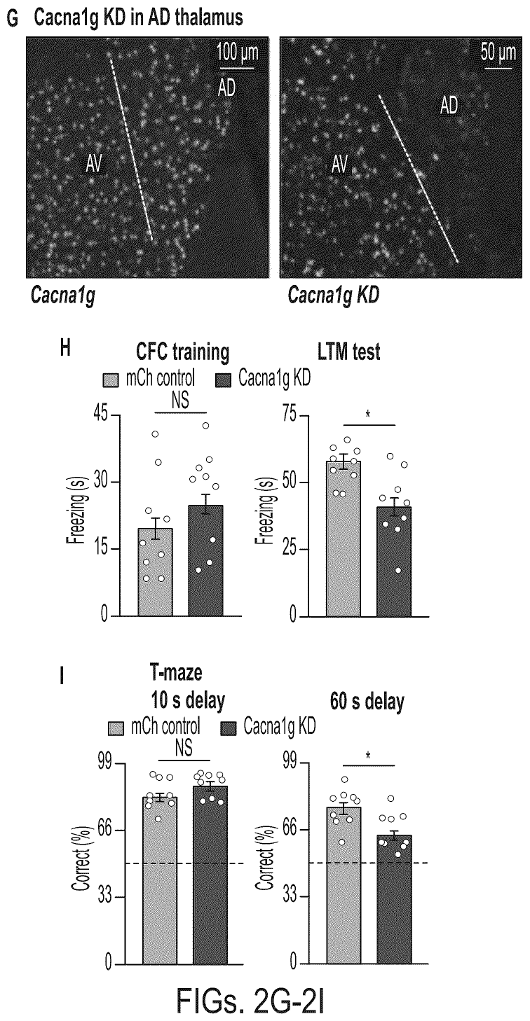

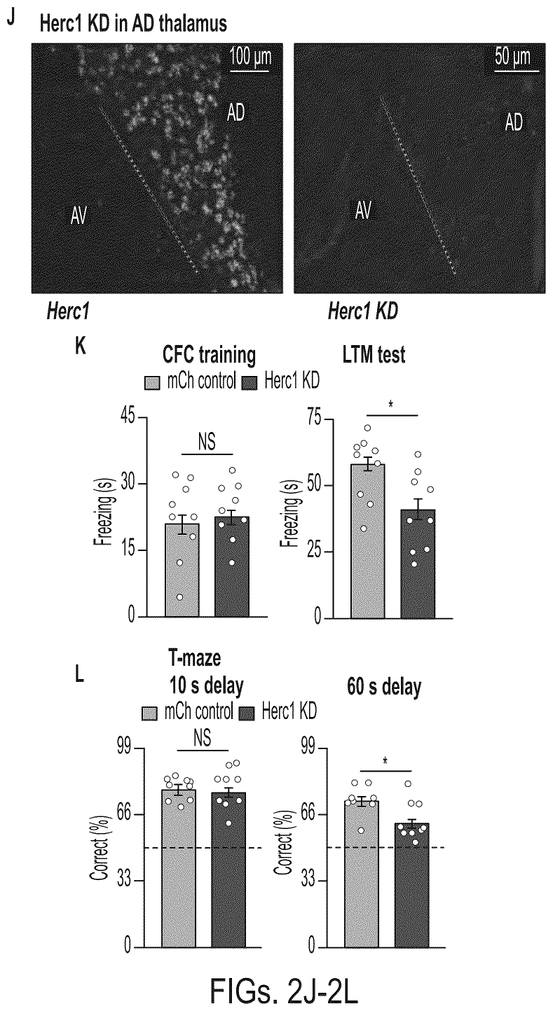

To test whether AD dysfunction plays a role in memory deficits in a PTCHD1 model, the inventors took advantage of our finding that AD but not AV projects to PreSub. The inventors optimized a circuit-based CRISPR-Cas9 viral approach, which included a retrograde rabies virus (RV)-expressing Cre (Chatterjee et al., 2018) injected in PreSub and a virus expressing target guide RNAs combined with a Cre-dependent SpCas9 virus (Xu et al., 2018) injected in AD, to knockdown (KD) PTCHD1 in AD (FIG. 1H. and see FIG. 9H). Using the contextual fear conditioning (CFC) memory paradigm, the inventors found that PTCHD1 KD in AD did not alter foot shock-induced freezing during CFC training,but led to a long-term memory (LTM) recall deficit (FIG. 1I). Using a spatial working memory paradigm, the inventors found that PTCHD1 KD did not alter days to criterion during training or performance when the delay between sample and choice phases was short (10 s), but led to a working memory impairment when the inventors used a more demanding long delay (60) s) (FIG. 1J). These observations provide evidence linking an ASD/ID risk gene, PTCHD1, to behaviorally relevant AD circuit dysfunction. The inventors performed KD of another ASD risk gene YWHAG (FIG. 2A). YWHAG KD mice exhibited significant CFC memory deficits (FIG. 2B, and see FIGS. 9I-9M). Strikingly. AD thalamus-specific KD of schizophrenia risk genes GRIA3 (FIGS. 2D-2E), CACNA1G (FIGS. 2G-2H), or HERC1 (FIGS. 2J-2K) all led to CFC memory deficits. Furthermore, YWHAG, GRIA3, CACNA1G, and HERC1 KD mice were impaired in the long delay working memory test (FIGS. 2C, 2F, 2I, 2L), indicating that AD dysfunction induces cognitive impairments in a subset of different disease models.

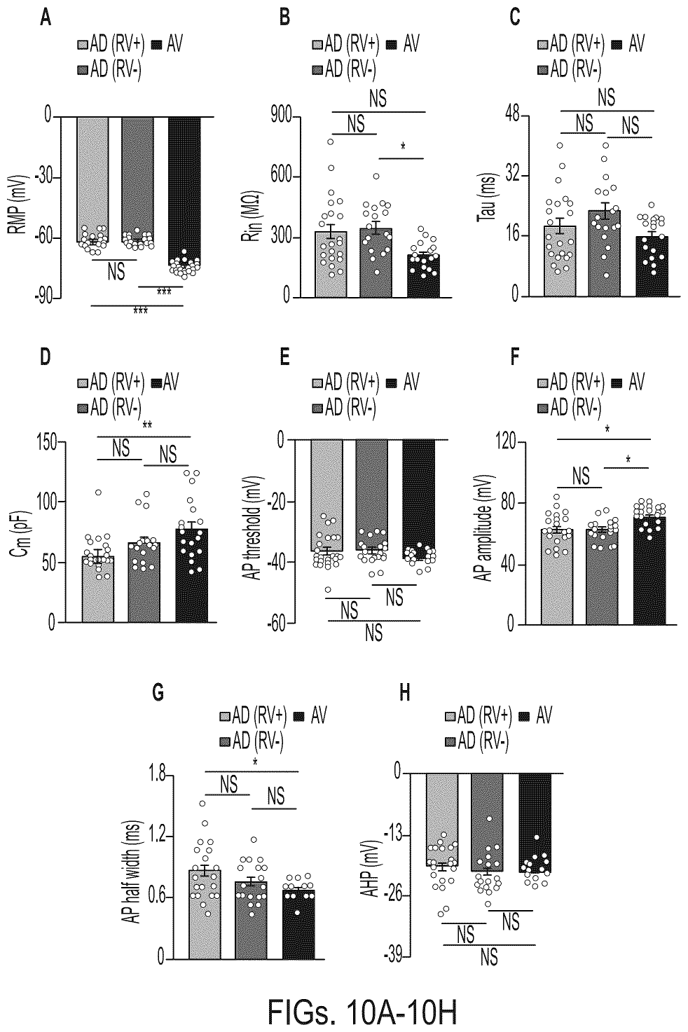

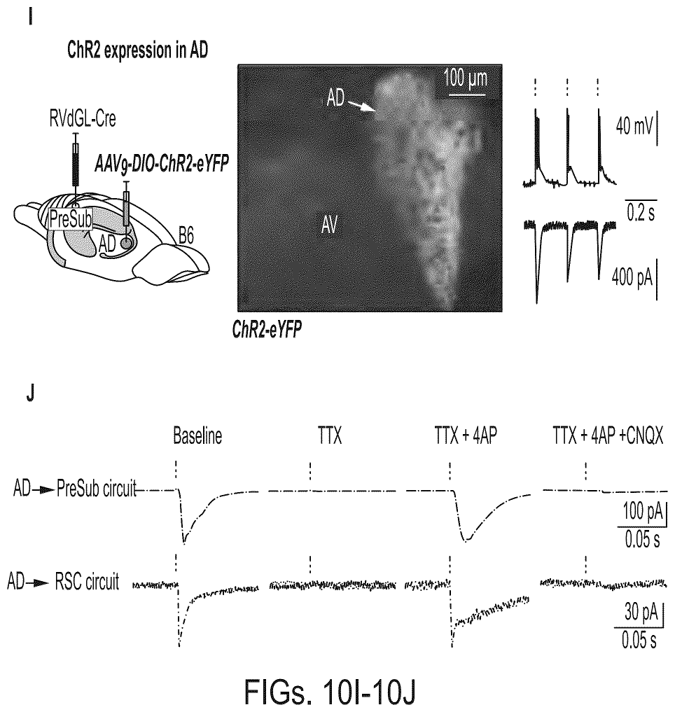

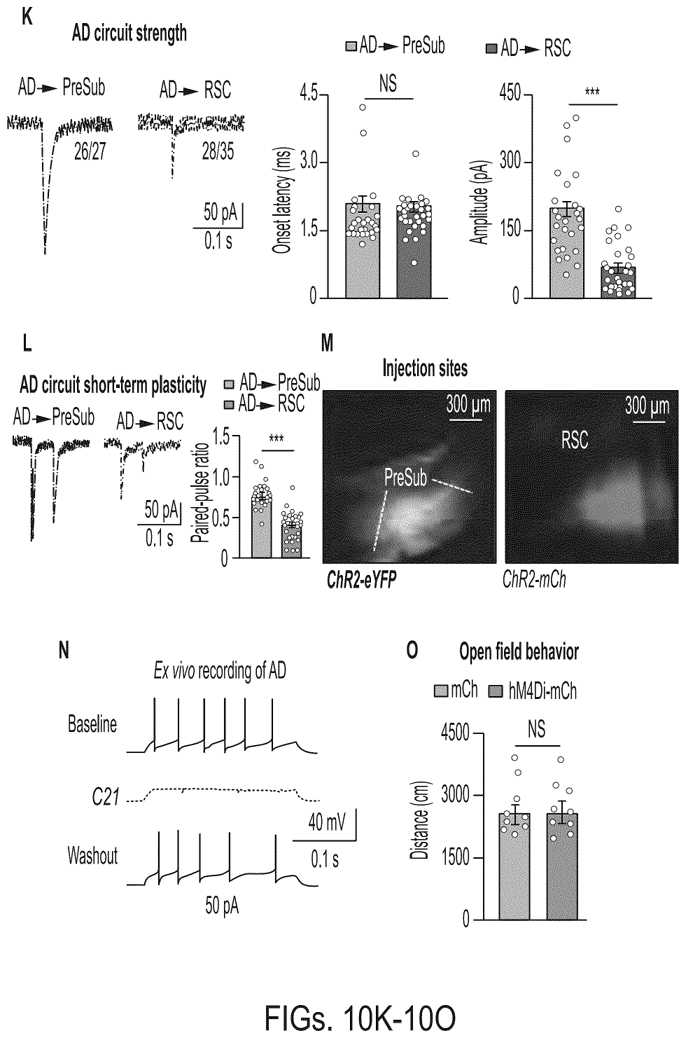

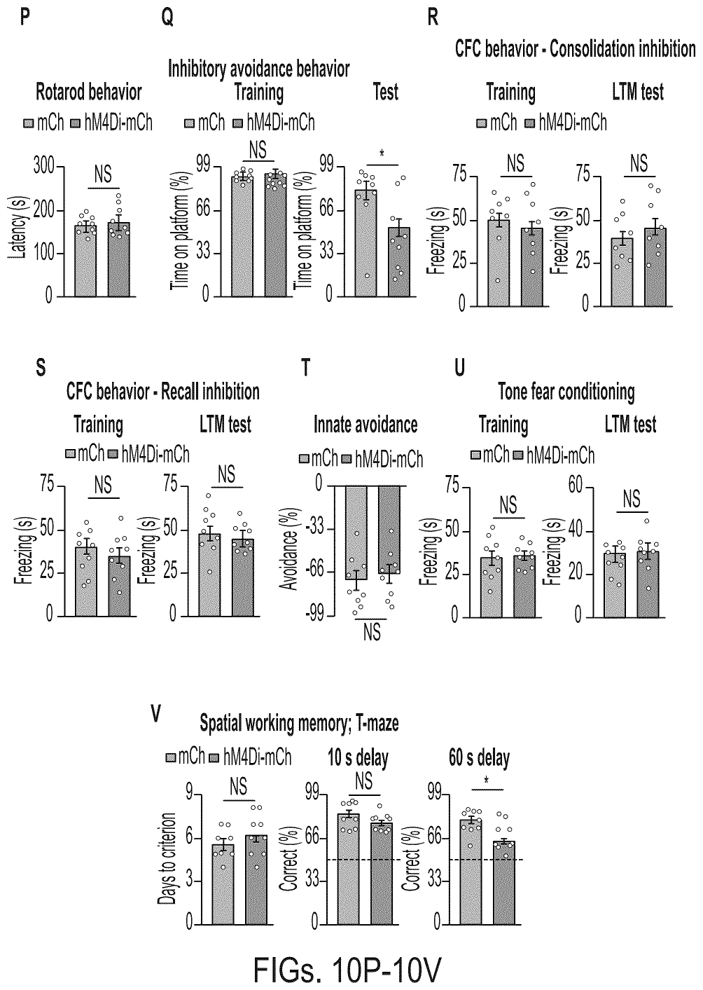

Because many ASD and schizophrenia risk genes are not only highly expressed in AD thalamus but the KD of several risk genes selectively from AD lead to cognitive deficits, the inventors wanted to know how this convergence compared to well-known cognitive brain regions. The inventors examined the expression of 428 ASD (category S, 1, and 2 from the SFARI database) and schizophrenia (FDR <5%) (Singh et al., 2020) risk genes using the Allen Brain Atlas (Lein et al., 2007) with a focus on AD thalamus and two other memory brain regions, hippocampal CA1 and mediodorsal thalamus (MD). While 21% of these risk genes were robustly expressed in MD, 48% and 57% were expressed in AD and CCA1 respectively (FIG. 9N). Among the risk genes that the inventors functionally tested in AD thalamus (FIGS. 1A-1B), other than PTCHIDI all risk genes are also expressed in CA1 (Lein et al., 2007). To determine whether the KD of these risk genes from CA1 alters memory, the inventors injected a virus expressing target guide RNAs combined with a constitutive SpCas9 virus (FIG. 10A). While the KD of YWHAG. GRIA3, CACNA1G, and HERC1 from AD thalamus led to CFC memory deficits, in CA1 only GRIA3 and CACNA1G KD mice showed comparable memory deficits (FIGS. 10B-10C). These experiments indicate that for a subset of risk genes expressed in both AD and CA1, risk gene KD shows a greater functional convergence in AD thalamus.

### Example 4: Inputs and Electrophysiological Properties of AD and AV Thalamus

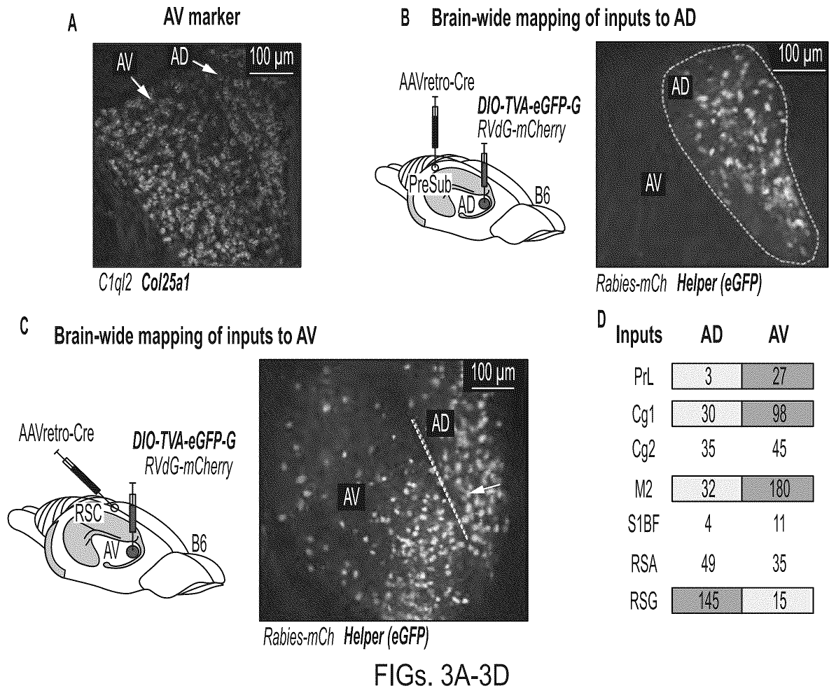

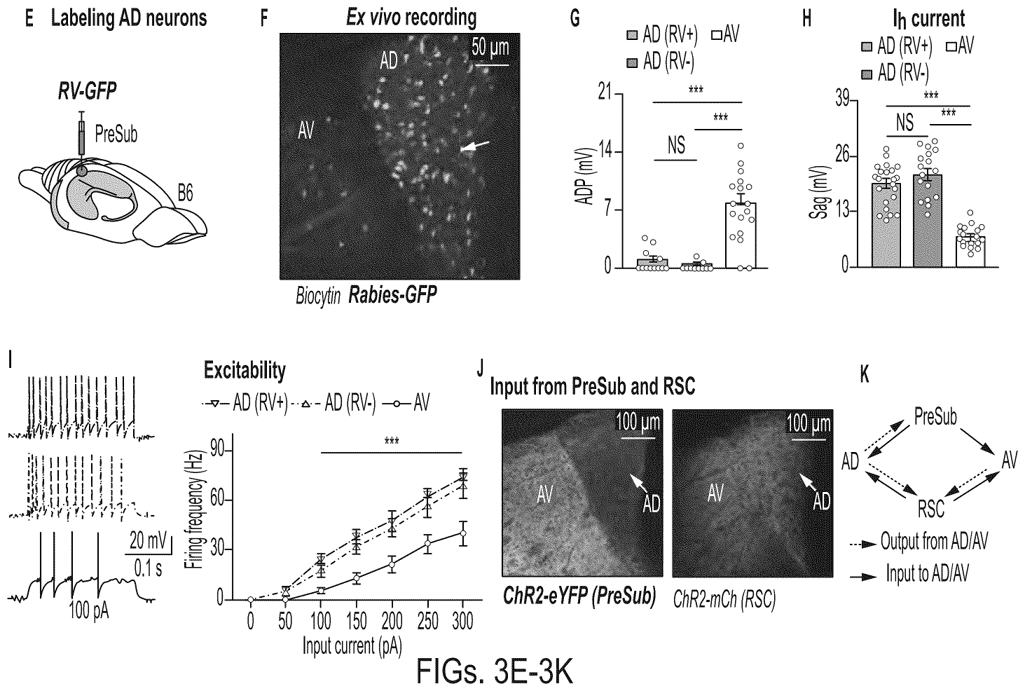

Because some risk genes are not only expressed in AD but also in neighboring AV thalamus (FIGS. 1A-1B), it is important to understand the cellular/circuit properties and behavioral contributions of these two ATN subdivisions in wild type mice. By examining highly expressed genes in other thalamic clusters (FIG 1C), the inventors found that collagen type XXV alpha 1 chain (COL25A1) mRNA is selectively expressed in AV thalamus within ATN in mice (FIG. 3A, and see FIG. 10D for marmosets). The inventors next wanted to map brain-wide inputs to AD and AV. By injecting a retrograde Cre virus (Tervo et al., 2016) in PreSub combined with Cre-dependent RV-mCherry injection in ATN, the inventors characterized inputs to AD thalamus with high specificity (FIG. 3B). For selective AV labeling, the inventors injected the retrograde Cre virus in RSC combined with Cre-dependent RV-mCherry injection targeting AV (FIG. 3C, and see FIGS. 10E-10F). Given that the starter cells in AV are less dense than COL25A1⁺ AV neurons, it is likely that these experiments underestimate input cell numbers to this subdivision. Nevertheless, by normalizing inputs to each ATN subdivision to their respective starter cell counts, the inventors found that most structures projected to both AD and AV (FIG. 3D, and see FIGS. 10G-10I), however prelimbic cortex input was observed for AV but not AD. Interestingly, most inputs had more neurons projecting to AV than AD. The granular division of RSC did not fit this pattern as it sent a larger input to AD.

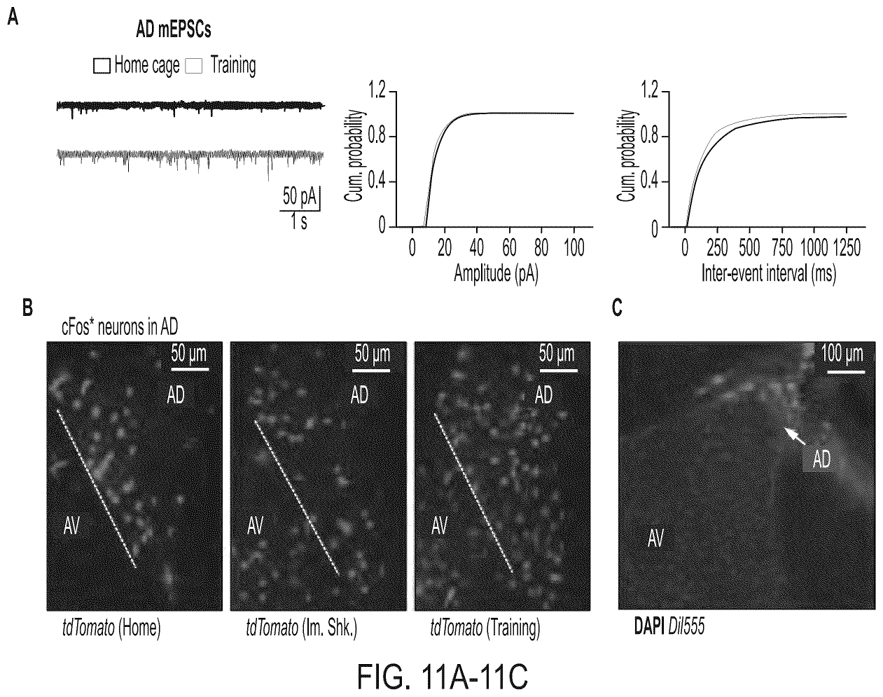

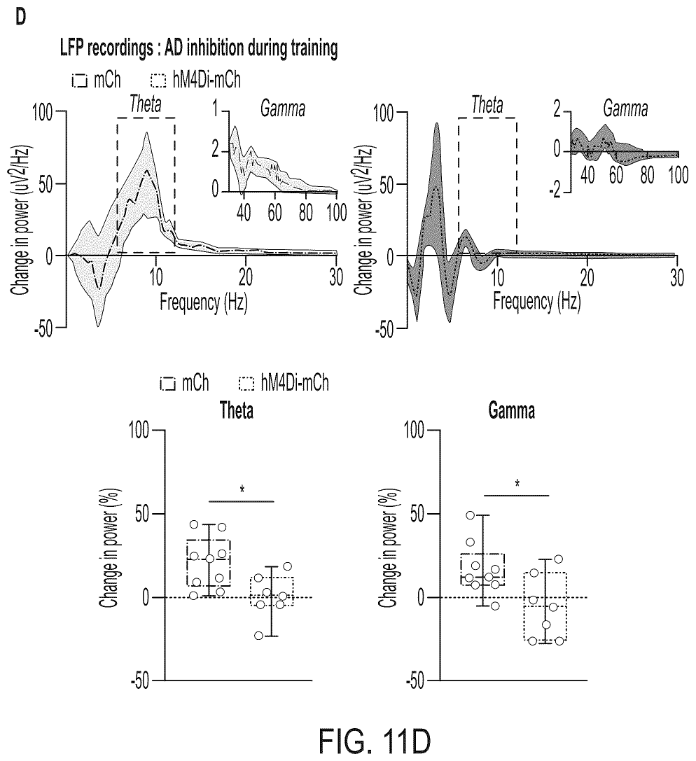

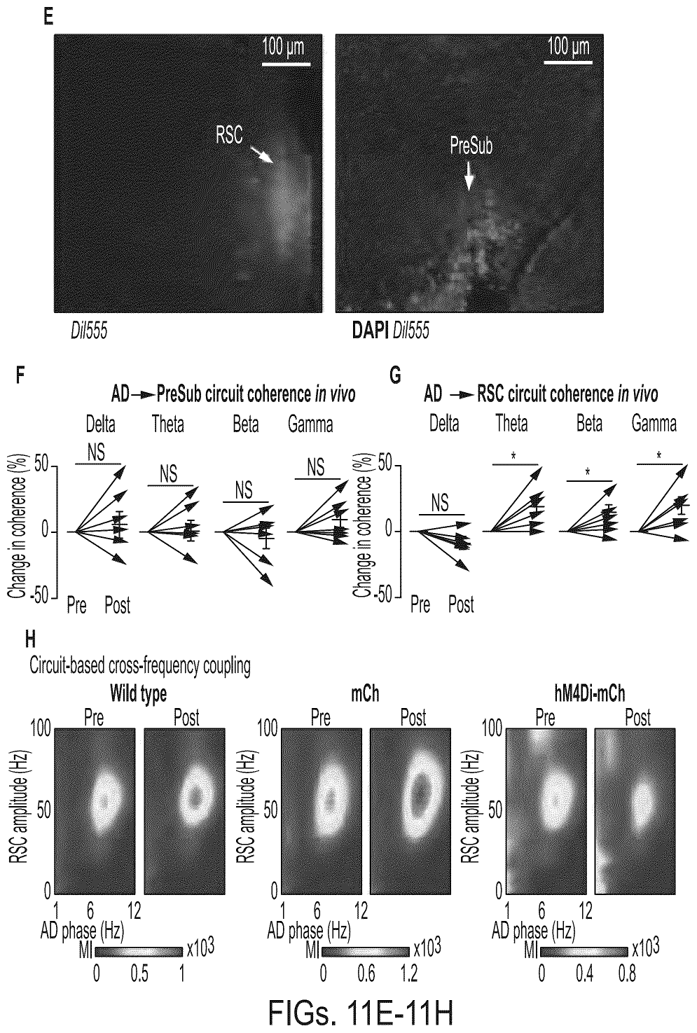

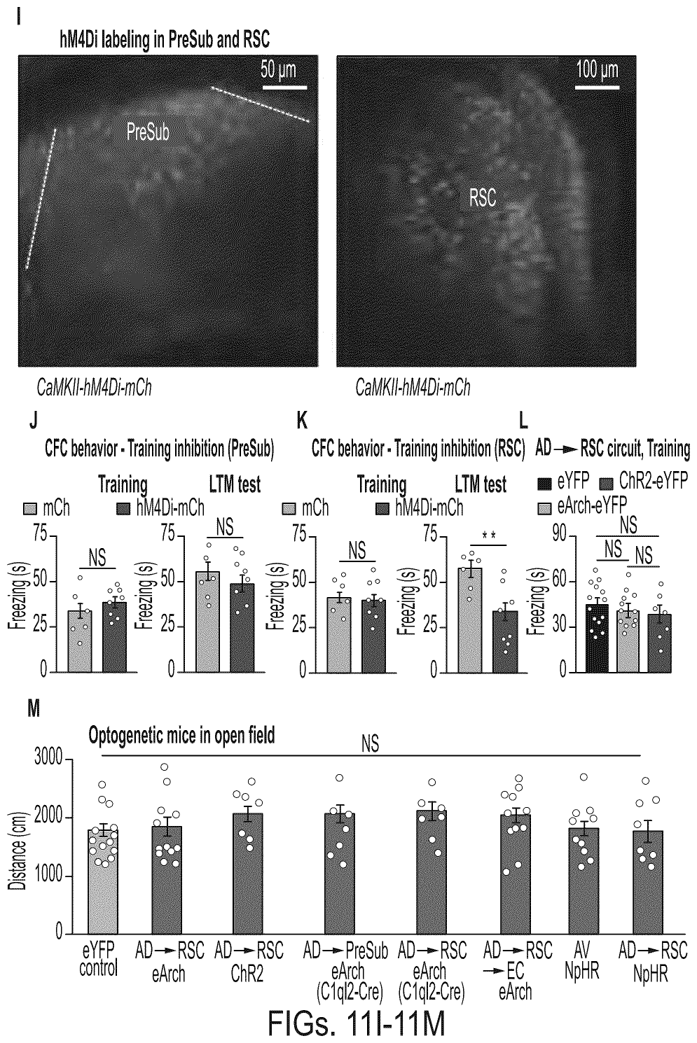

Given that AD and AV have distinct molecular markers and connectivity patterns, the inventors used ex vivo electrophysiology in ATN slices to compare these subdivisions. AD neurons projecting to PreSub were labeled by a retrograde RV expressing green fluorescent protein (GFP) (FIGS. 3E-3F). Within AD. GFP and GFP neurons had similar properties (FIGS. 3G-3I). However, the inventors observed striking differences between AD and AV (FIGS. 3G-3I, and see FIGS. 11A-11H). The inventors next characterized the two major AD output circuits (FIG. 11I). Ex vivo electrophysiological recordings showed that optogenetic stimulation of AD neurons resulted in larger excitatory post-synaptic currents in PreSub as compared to RSC neurons (FIGS. 11J-11K). In addition, these two circuits were different in their short-term plasticity (FIG. 11L). Interestingly, when the inventors injected a ChR2-eYFP virus in the PreSub region and a ChR2-mCherry virus in RSC, the inventors observed that their axonal terminals showed distinct patterns of projections back to ATN: AV received stronger input from the PreSub region as compared to AD, whereas both AD and AV received strong input from RSC (FIGS. 3J-3K, and see FIG. 11M). These experiments revealed distinct properties between AD and AV thalamus.

### Example 5: The AD→RSC Circuit is Necessary for Contextual Memory Encoding

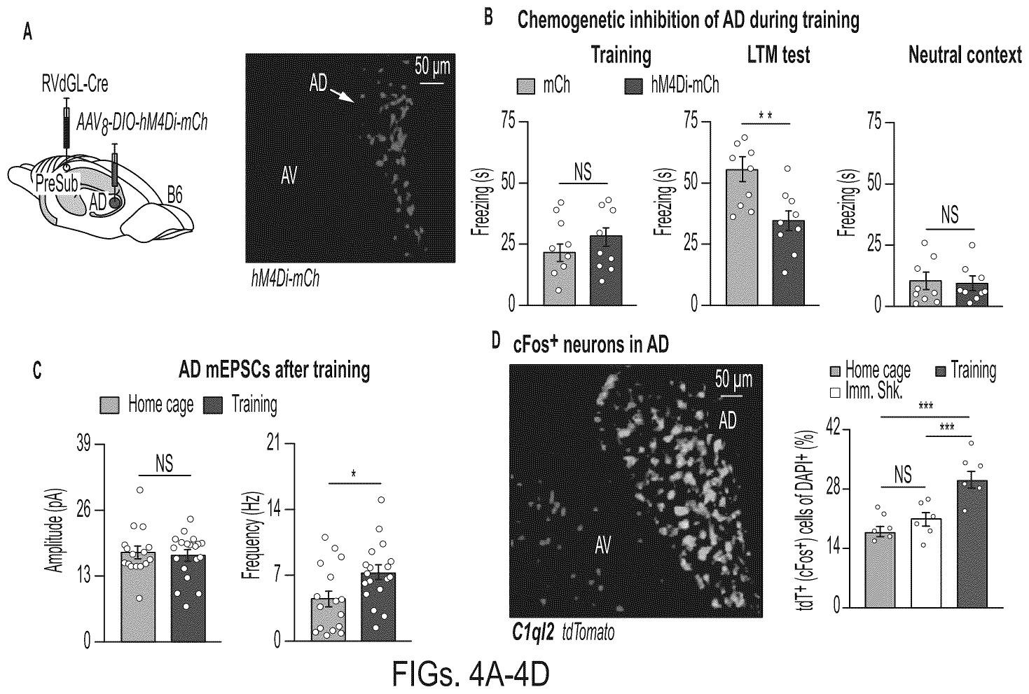

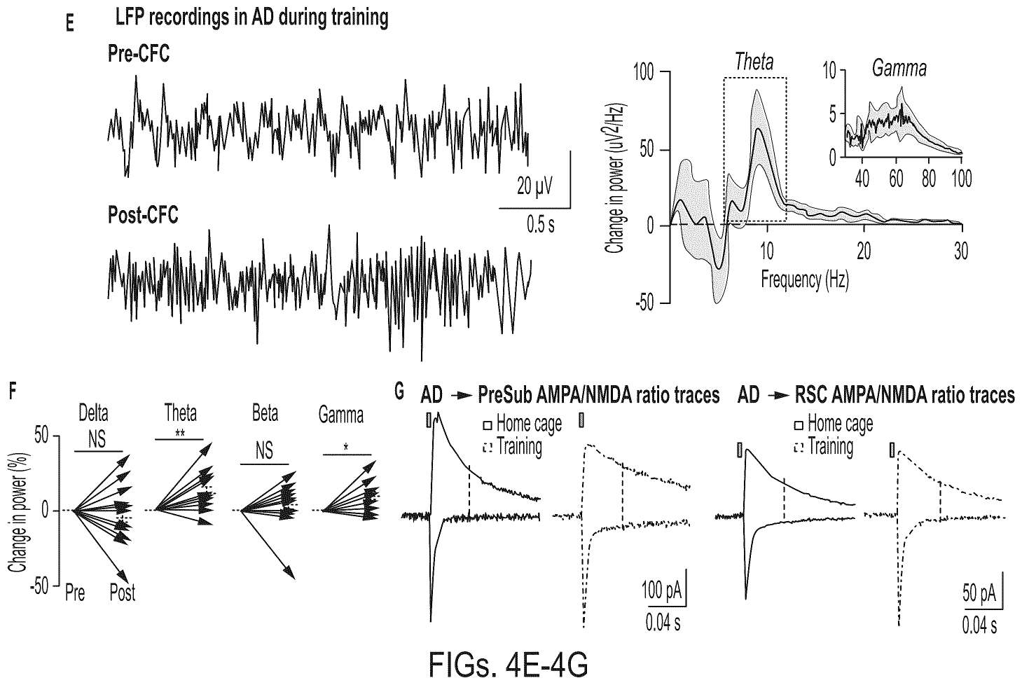

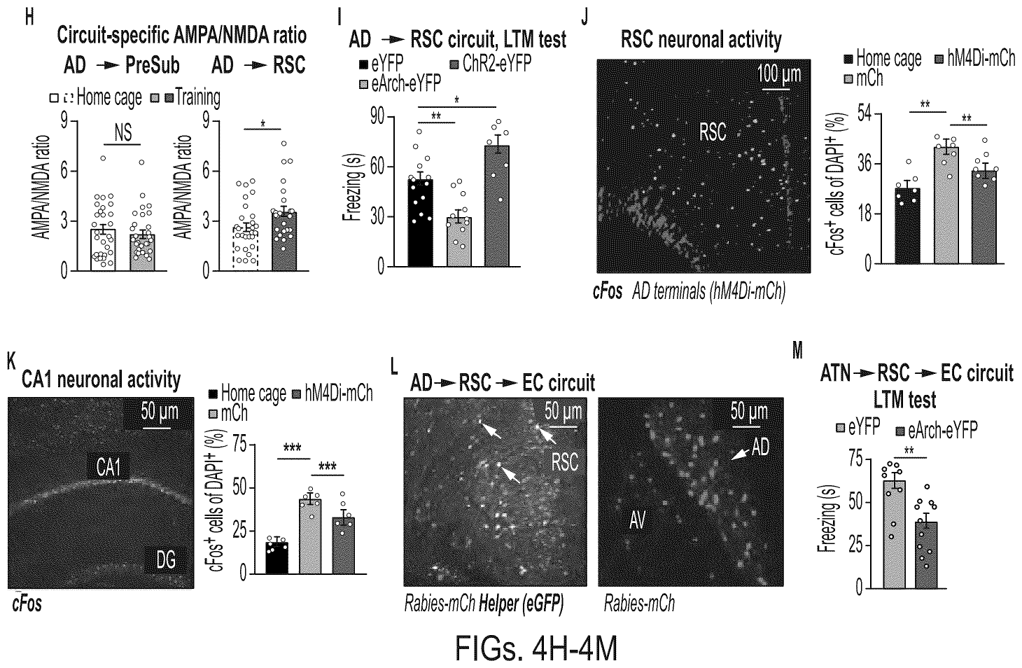

Although our risk gene KD experiments clearly link AD thalamus to CFC memory, the precise role of this ATN subdivision in wild type mouse behavior remains unclear. The inventors first injected a retrograde RV expressing Cre in PreSub and a Cre-dependent inhibitory DREADDs hM4Di-mCherry virus in AD (FIG. 4A), and subsequently validated that the chemogenetic ligand compound 21 (C21) reversibly decreased AD neuronal firing (FIG. 11N). In the CFC paradigm, inhibiting AD during training did not alter foot shock-induced freezing, however LTM recall was impaired (FIG. 4B). Neither control nor AD inhibited mice displayed increased freezing behavior in a neutral context (FIG. 4B), and motor behaviors were normal in these mice (FIGS. 11O-11P). To determine whether our observation that AD plays an important role in contextual memory encoding extended to another memory paradigm, the inventors performed the inhibitory avoidance (IA) task. AD inhibition during encoding also impaired performance in the IA memory task (FIG. 11Q). In contrast, inhibition of AD immediately after CFC encoding (referred to as the cellular consolidation phase) or during CFC LTM recall did not affect performance (FIGS. 11R-11S), and AD was not necessary for innate avoidance or tone fear encoding (FIGS. 11T-11U). The inventors also noted that AD plays an important role in a demanding version of the spatial working memory paradigm (FIG. 11V).

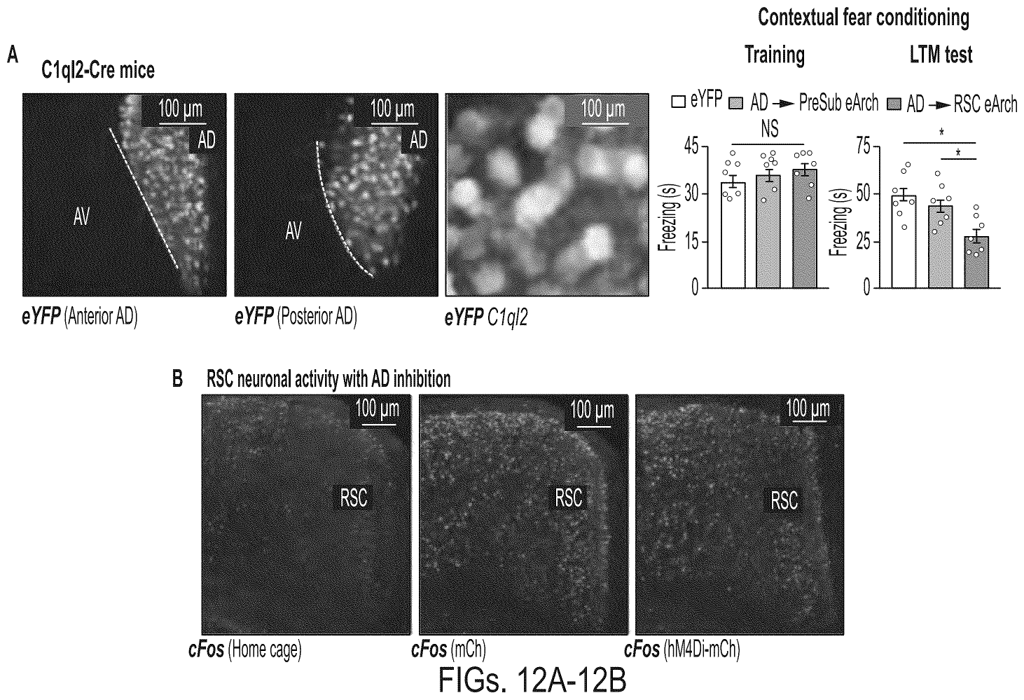

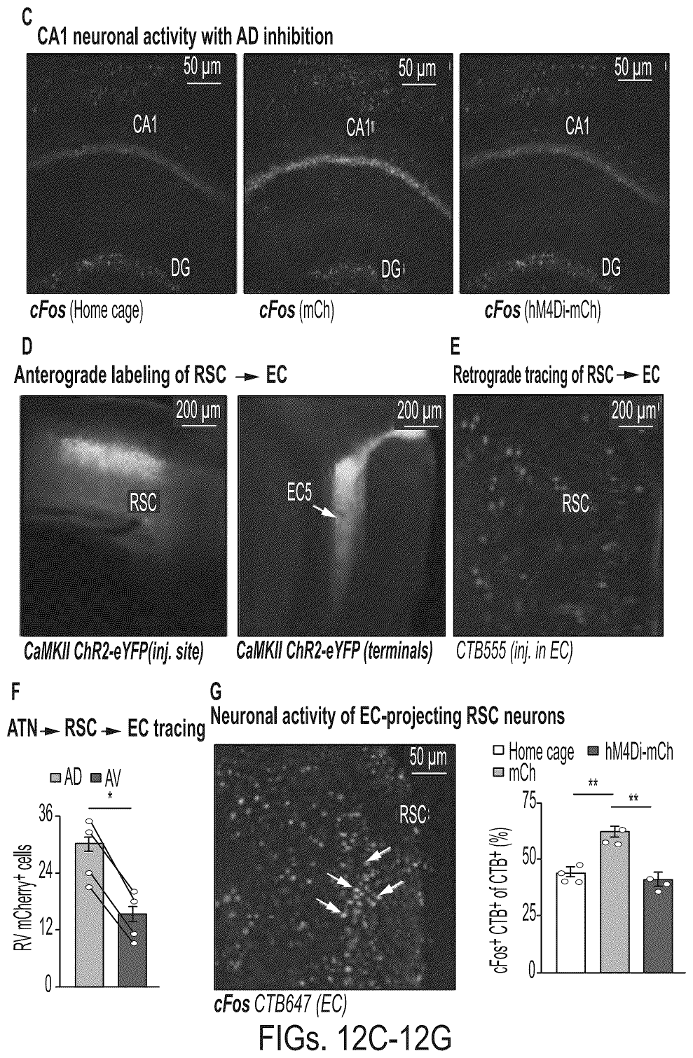

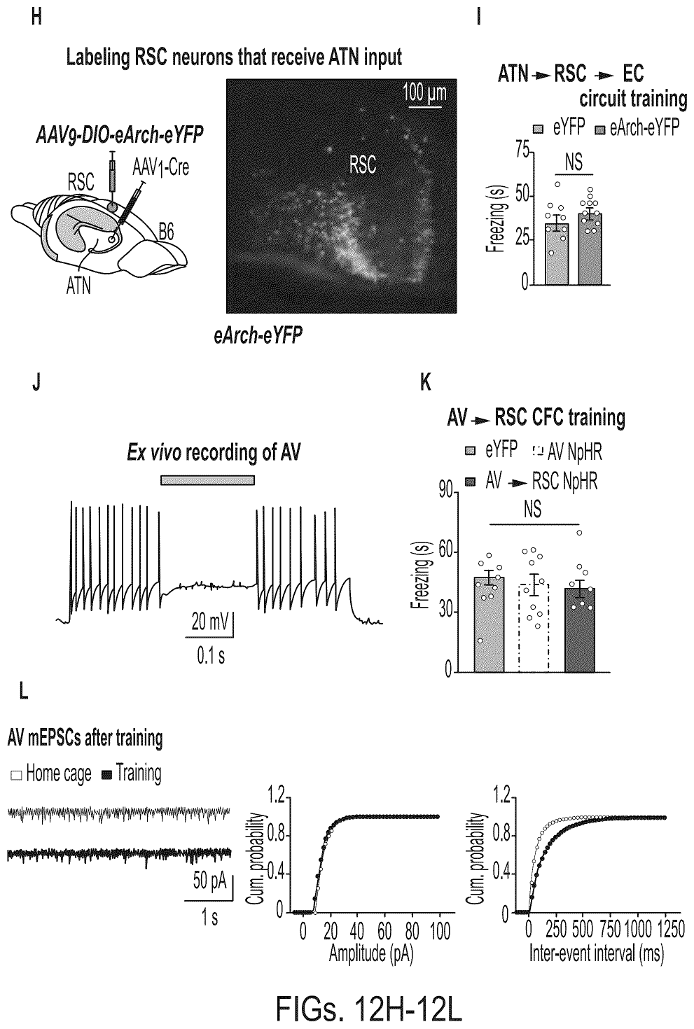

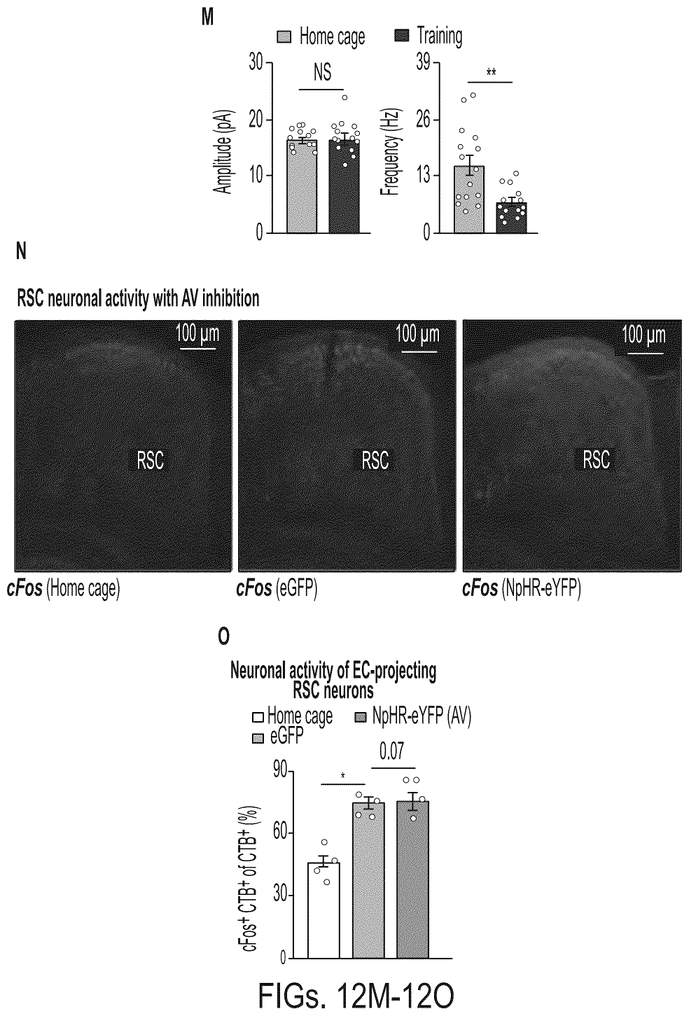

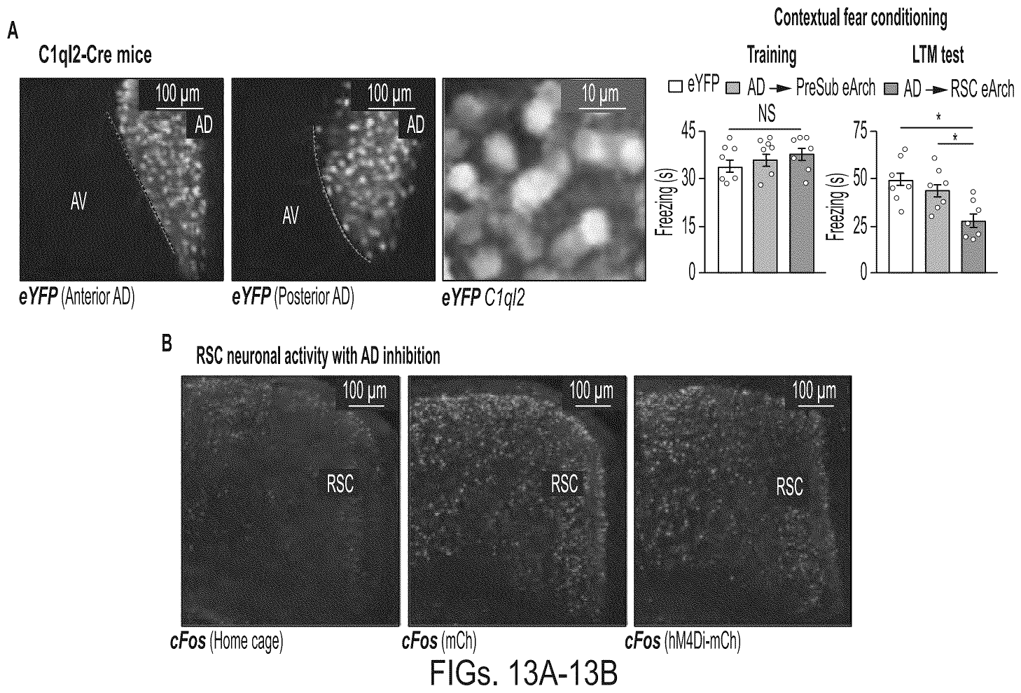

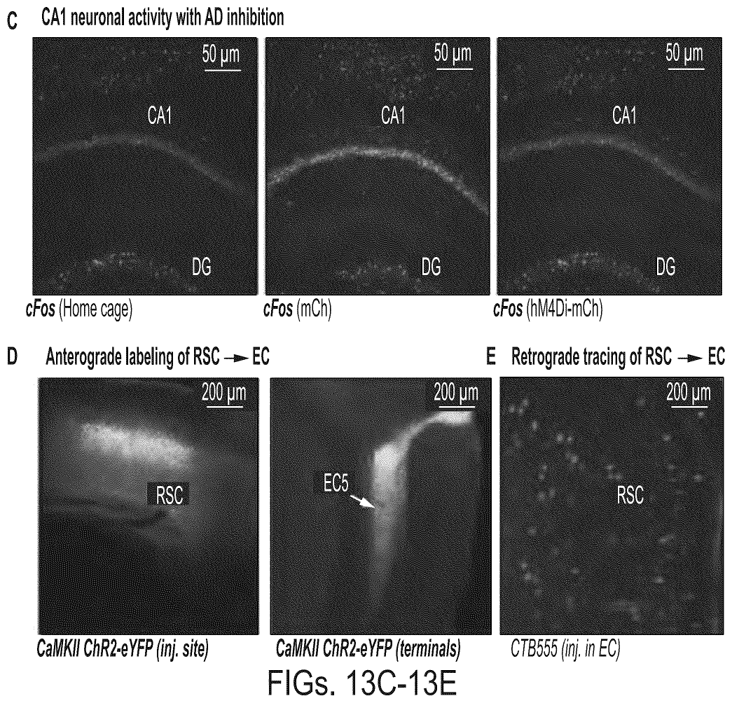

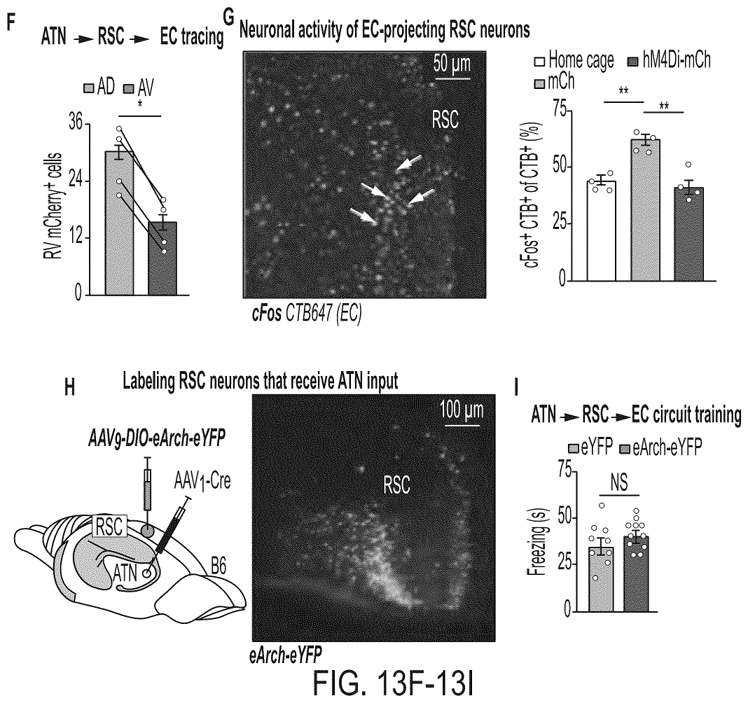

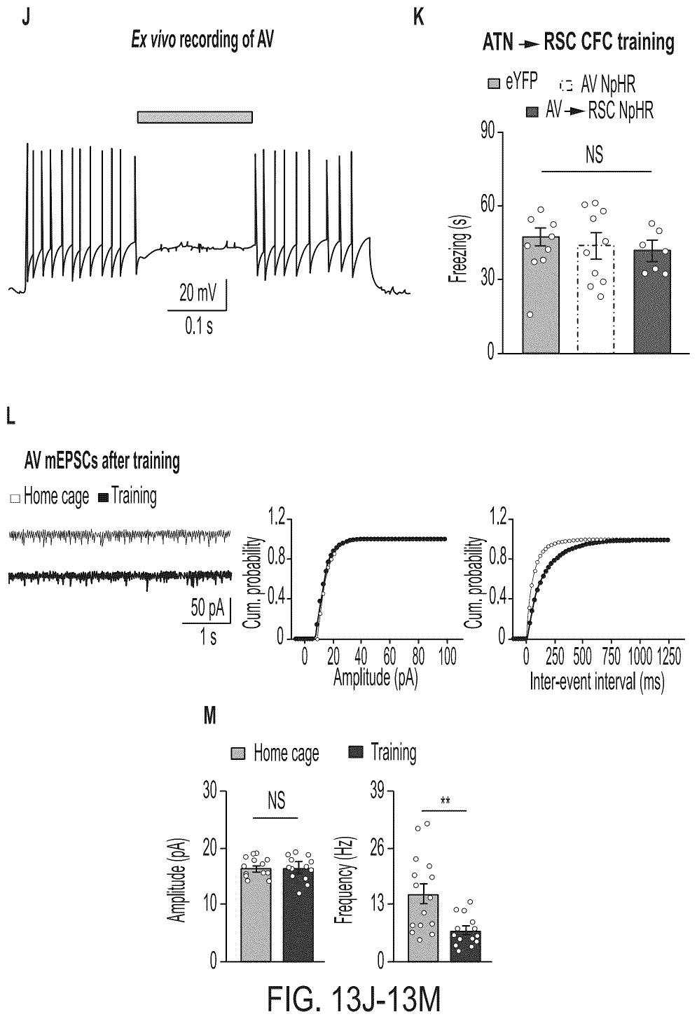

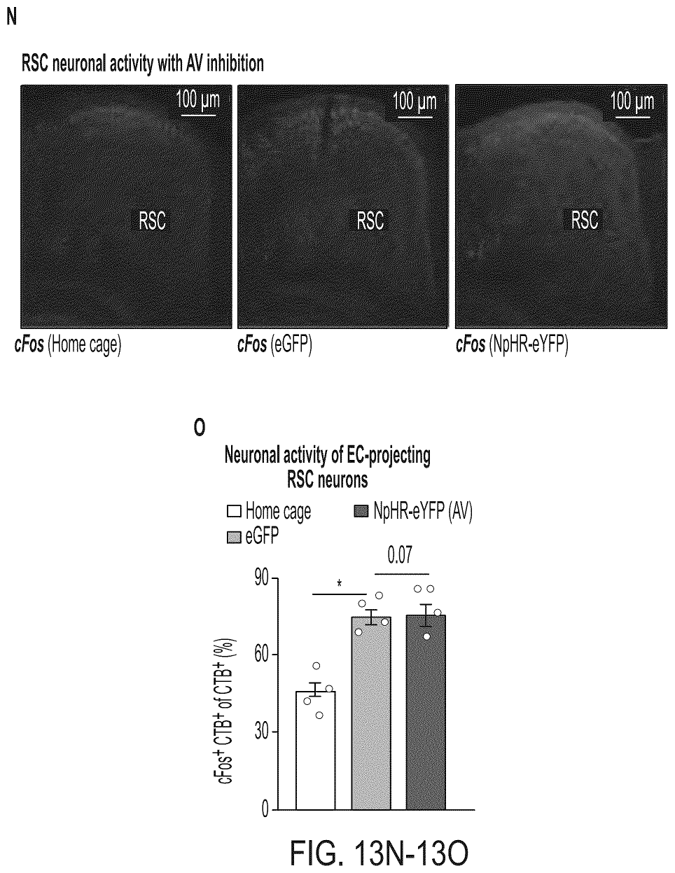

In search of cellular correlates of memory encoding in AD, the inventors found that the frequency of miniature excitatory post-synaptic currents (mEPSCs) was increased post-CFC training (FIG. 4C, and see FIG. 12A). This increase correlated with an increase in the active cFos⁺ ensemble size in AD (FIG. 4D, and see FIG. 12B). In vivo local field potential (LFP) recordings from AD showed significant increases in the power of theta and gamma oscillations following encoding (FIGS. 4E-4F, and see FIG. 12C), which was not observed when AD was chemogenetically inhibited during encoding (FIG. 12D). To determine if one or both of the major AD outputs play a role in memory encoding, the inventors measured synaptic strengthening in these circuits post-encoding. Encoding increased the AMPA/NMDA ratio of the AD→RSC circuit, but not the AD→PreSub circuit (FIGS. 4G-4H). Consistently, in vivo LFP coherence between AD and RSC, but not AD and PreSub. exhibited enhancements post-encoding (FIGS. 12E-12G), and enhanced theta-gamma cross-frequency coupling (FIG. 12H). Since these in vivo electrophysiological correlates have been consistently linked to cognitive processes (Colgin. 2015), these data support the idea that AD neurons and their projections to RSC in particular play an important role in memory encoding. To directly test this idea, the inventors performed chemogenetic inhibition of RSC or PreSub excitatory neurons during CFC encoding. Inhibition of RSC neurons, but not PreSub neurons, led to a recall deficit, which mimicked the effect of AD inhibition (FIGS. 121-12K). Further, optogenetic terminal inhibition in RSC directly demonstrated that the AD→RSC circuit is necessary for encoding, and optogenetic activation of this circuit during encoding is sufficient to enhance LTM recall (FIG. 4I, and see FIGS. 12L-12M). These observations are further supported by optogenetic terminal inhibition experiments using C1q12-Cre mice, which revealed that the AD→RSC circuit, but not the AD→PreSub circuit. is necessary for encoding (FIG. 13A, and see FIG. 12M).

The inventors next examined the effect of chemogenetic AD inhibition during encoding on neural activity in downstream structures such as RSC and hippocampal CA1. The inventors found that AD inhibition impaired the learning-induced enhancement of CFOS⁺ ensembles in both RSC and CA1 (FIGS. 4J-4K, and see FIGS. 13B-13C). Because this suggested that manipulating AD contributed to changes in hippocampal activity, the inventors wanted to identify the circuit basis for this observation. The inventors started by confirming that RSC projects to entorhinal cortex (EC) (Witter et al., 2017) (FIGS. 13D-13E), which serves as the major input to the hippocampus. The inventors hypothesized that AD→RSC→EC may underlie the important contribution of AD→RSC in encoding. To directly visualize connectivity between AD. RSC, and EC, the inventors injected a retrograde Cre virus in EC and Cre-dependent RV in RSC. In support of this idea, the inventors showed that more AD neurons, in comparison to AV, project to the EC-projecting RSC neurons (FIG. 4L, and see FIG. 13F). The finding that AD inhibition during encoding decreased CFOS activation of EC-projecting RSC neurons (FIG. 13G) further strengthened this idea. To link these tracing and neural activity data to behavior, the inventors injected an anterograde virus expressing Cre (Zingg et al., 2017) in ATN, a Cre-dependent eArch-eYFP virus in RSC, and implanted optic fibers in EC (FIG. 13H). Optogenetic terminal inhibition in EC of the ATN→RSC→EC circuit during encoding impaired CFC LTM recall (FIG. 4M, and see FIGS. 12M and 13I). These experiments uncovered the neural circuit mechanism by which AD thalamus contributes to the cortico-hippocampal memory network.

### Example 6: The AV→RSC Circuit Regulates Memory Specificity

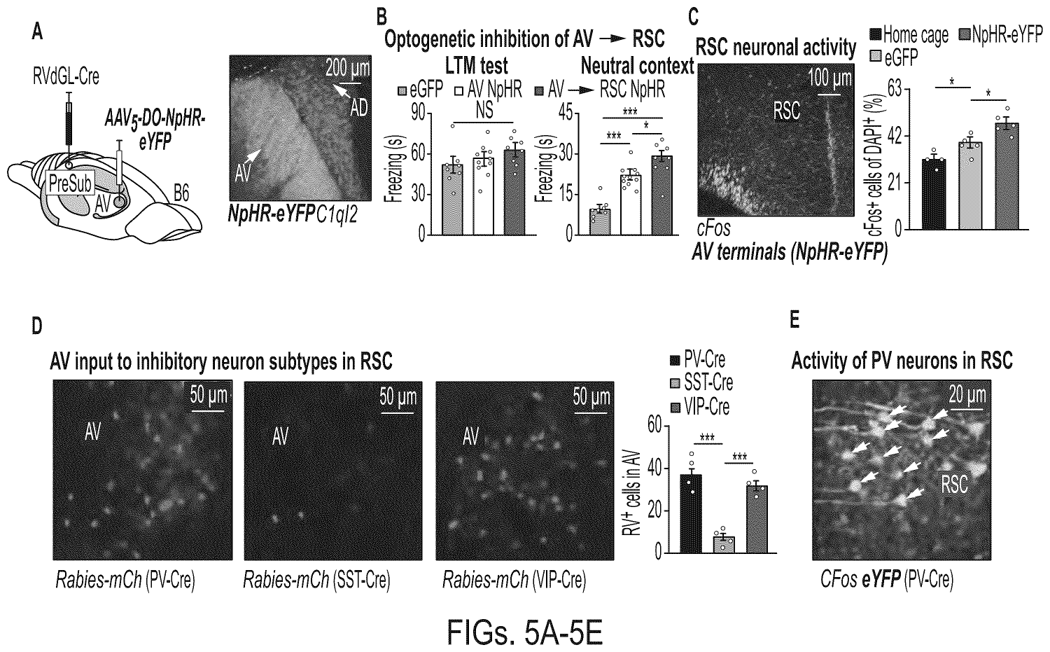

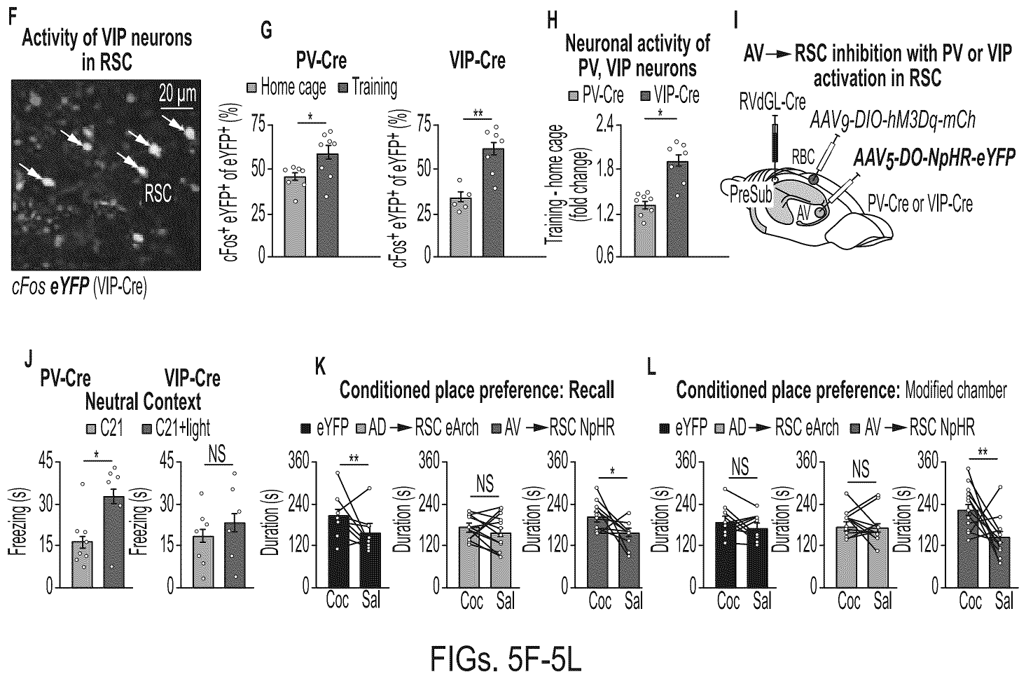

Since AV thalamus also projects to RSC, the inventors wanted to investigate their role in CFC memory. By expressing Cre in AD through injection of a retrograde RV expressing Cre in PreSub and a Cre-Off halorhodopsin (NpHR-eYFP) virus (Saunders et al., 2012) in ATN, the inventors confirmed specific AV thalamus labeling and light-induced neuronal inhibition (FIG. 5A, and see FIG. 13J). In contrast to AD thalamus, inhibition of AV cell bodies or AV→RSC terminals during CFC training had no effect on LTM recall, however these mice displayed robust generalization in the neutral context test (FIG. 5B, and see FIGS. 12M and 13K). This phenotype correlated with a post-training decrease in the frequency of mEPSCs on AV neurons (FIGS. 13L-13M).

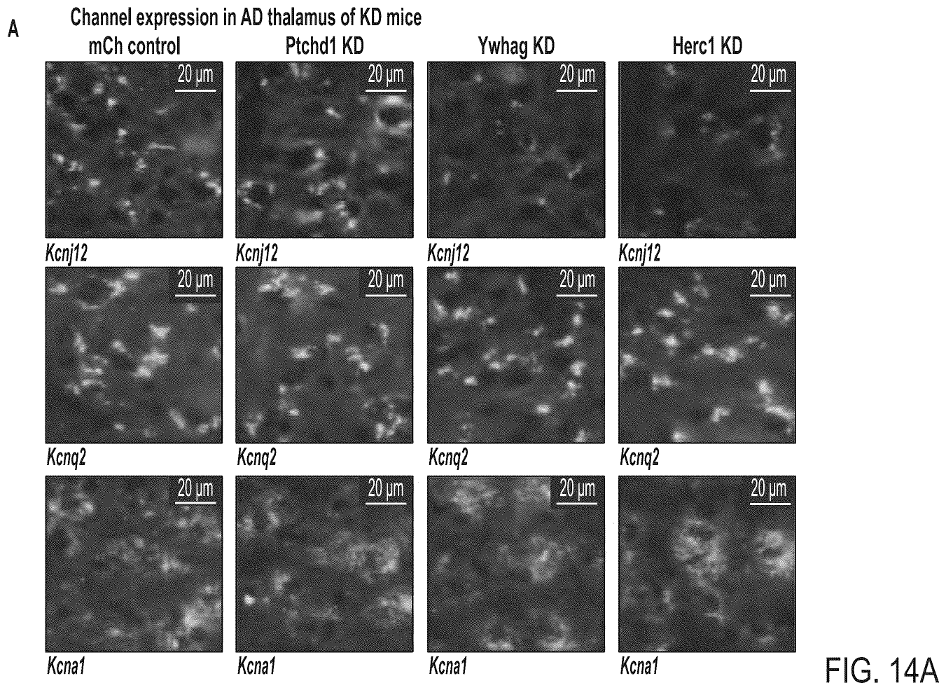

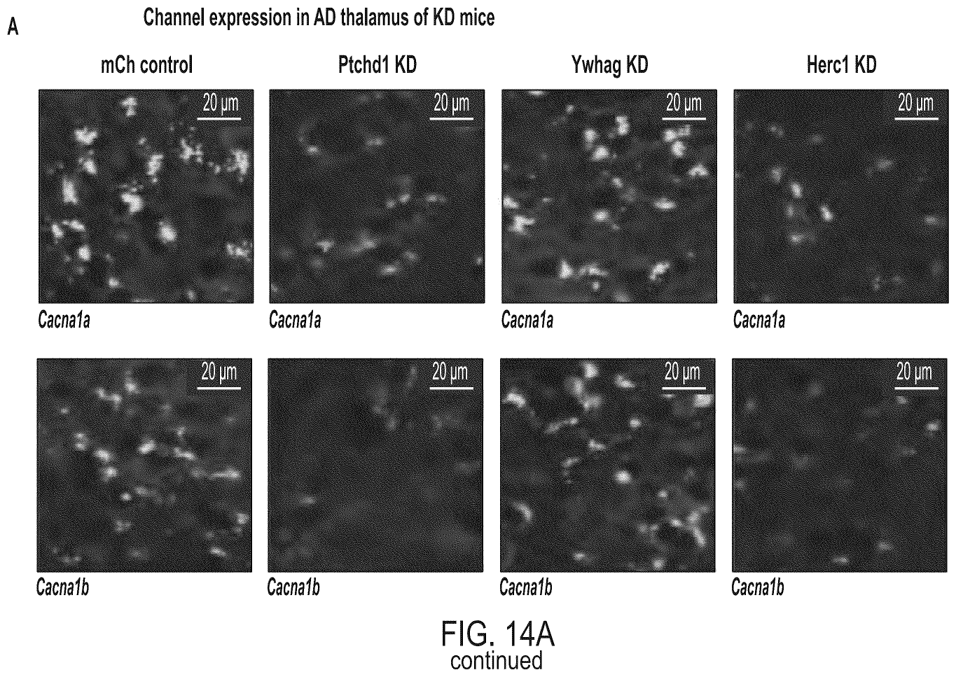

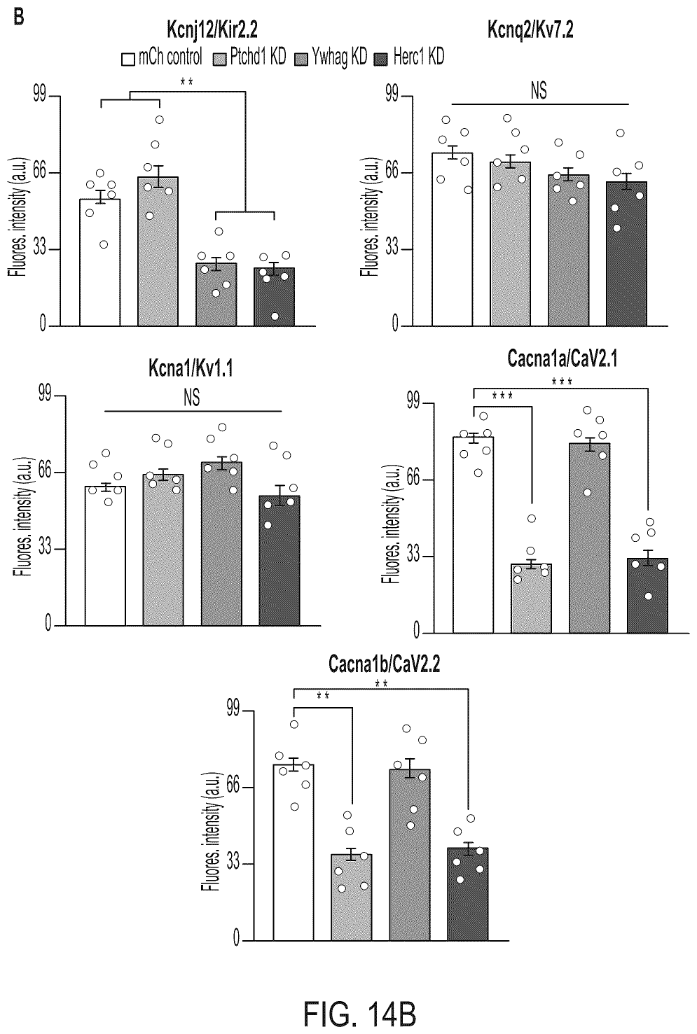

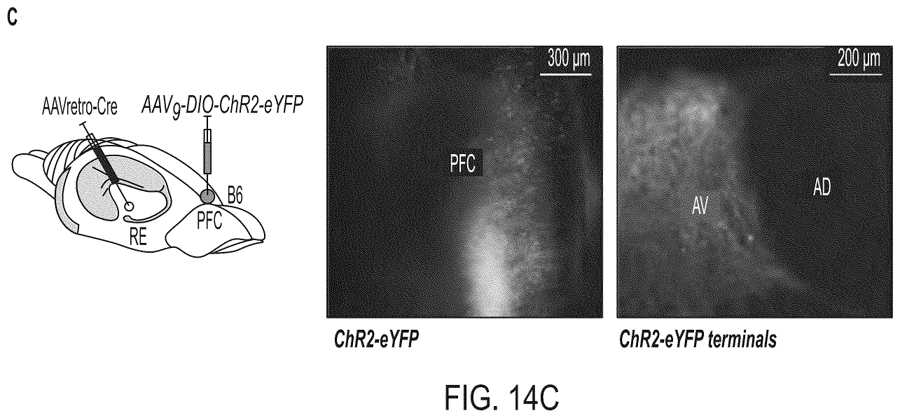

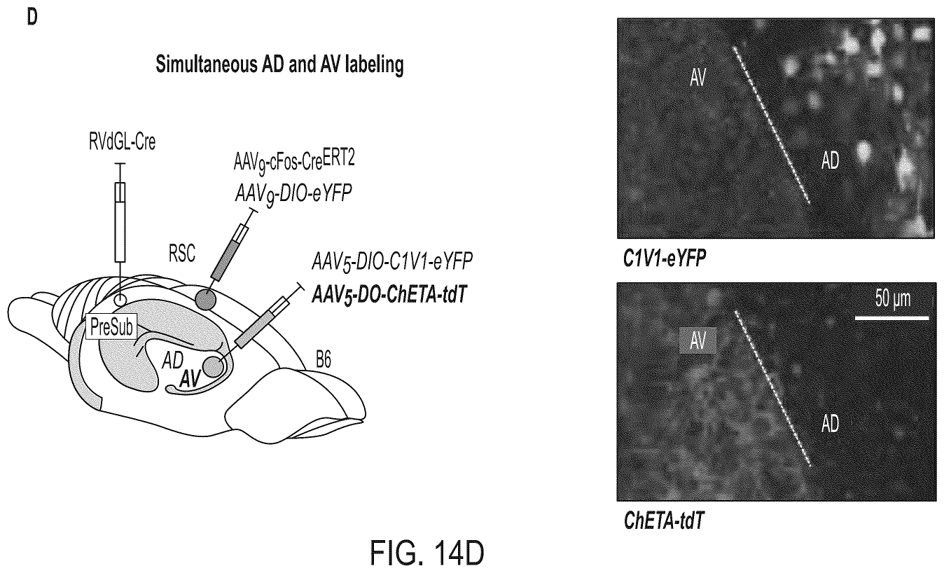

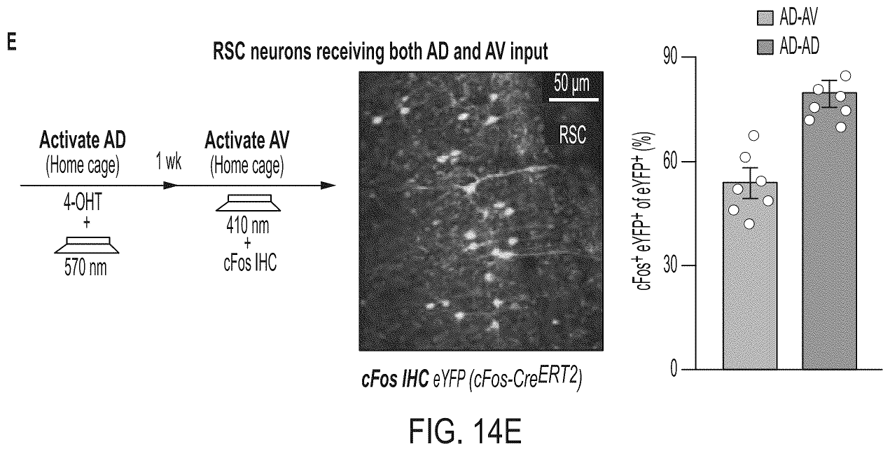

To further examine this generalization phenotype, the inventors optogenetically inhibited AV→RSC terminals during encoding and quantified activated ensembles in RSC (FIG. 5C, and see FIGS. 13N-13O). Strikingly, AV→RSC inhibited mice showed increased levels of learning-induced CFOS⁺ ensembles in RSC, which hinted at the possibility that the role of AV during encoding requires inhibitory neurons in RSC. Pursuing this possibility. using Cre-dependent RV injected in RSC of different inhibitory neuron-specific Cre mouse lines the inventors found that AV neurons primarily project to parvalbumin (PV) and vasoactive intestinal polypeptide (VIP) inhibitory neurons (FIG. 5D, and see FIG. 14A). Though both PV and VIP populations, which were labeled using a Cre-dependent eYFP virus in PV-Cre and VIP-Cre mice, exhibited an increase in CFOS activation post-training (FIGS. 5E-5G). VIP neurons had a greater fold change (FIG. 5H). The inventors next prepared mice in which AV→RSC terminals could be inhibited optogenetically with simultaneous activation of either PV or VIP neurons in RSC chemogenetically (FIG. 5I). AV→RSC inhibition with VIP, but not PV activation, during encoding prevented the generalization phenotype in AV inhibited mice (FIG. 5J, and see FIGS. 14B-14 C). In addition, using the cocaine-induced conditioned place preference (CPP) paradigm. the inventors showed that the AD→RSC circuit is necessary for effective memory encoding (FIG. 5K, and see FIG. 14D). However, in a modified CPP chamber, although the control eYFP group no longer exhibited any behavioral preference, the AV→RSC inhibited group showed significant preference (i.e., generalization behavior) (FIG. 5L), which demonstrated that the differential roles of AD (encoding) and AV (specificity) inputs to RSC in a negative-valence CFC memory task extends to a positive-valence CPP memory task.

### Example 7: Normalizing Hyperexcitability of AD Neurons Rescues Memory Deficits in ASD and Schizophrenia Models

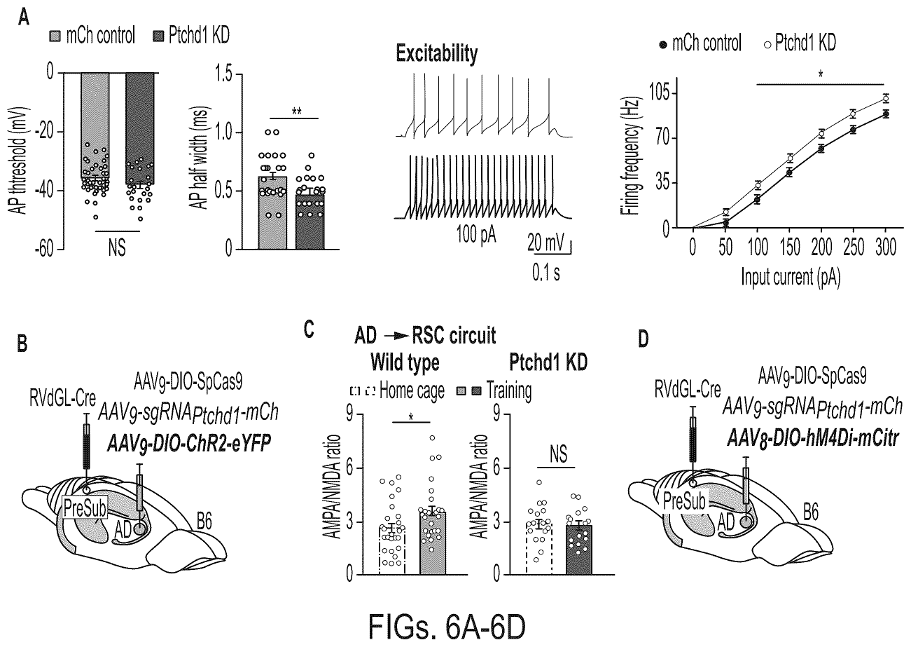

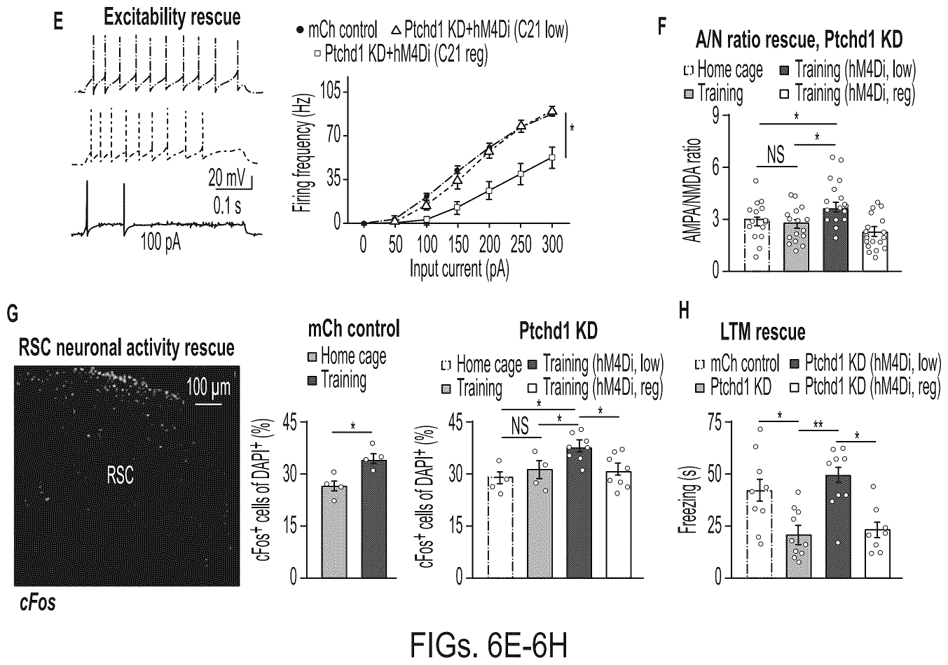

With a better understanding of AD circuits underlying memory in wild type mice, the inventors wanted to examine how PTCHD1 KD alters AD neuronal properties. Using ex vivo electrophysiology. PTCHD1 KD revealed a decrease in action potential (AP) half width, which correlated with an increase in the excitability of AD neurons (FIG. 6A, and see FIG. 14E), consistent with our previous findings in the TRN (Nakajima et al., 2019). To determine whether PTCHD1 KD has any impact on CFC training-induced AMPA/NMDA ratio increases in the AD→RSC circuit, the inventors prepared KD mice that included a Cre-dependent ChR2-eYFP virus in AD for recordings (FIG. 6B). The inventors observed a lack of CFC training-induced synaptic strengthening (AMPA/NMDA ratio) in the AD→RSC circuit of KD mice (FIG. 6C, and see FIG. 14F).

The inventors hypothesized that the increased excitability of AD neurons in KD mice may prevent synaptic strengthening during CFC training, which is necessary for efficient encoding. Specifically, in control mice the excitability of AD neurons would increase during training, which leads to strengthening of the AD→RSC circuit, but in KD mice due to the increased excitability of AD neurons before training there will not be the important training-induced increase in excitability and corresponding synaptic strengthening. By recording from AD neurons before and after CFC training in control and KD groups, the inventors obtained experimental evidence to support this idea (FIG. 14G). Without intending to be bound by theory, these findings support a correlation between neuronal hyperexcitability and impairments in long-term potentiation (Speca et al., 2014: Gruter et al., 2015). The inventors next developed a dose-dependent chemogenetic approach to normalize the excitability of AD neurons in KD mice (FIG. 6D). When the excitability was returned to physiological levels (i.e., using a low dose of C21) (FIG. 6E), training-induced strengthening of the AD→RSC circuit (FIG. 6F). training-induced CFOS ensemble size in RSC (FIG. 6G, and see FIG. 14H), and LTM recall were all rescued (FIG. 6H, and see FIG. 14I).

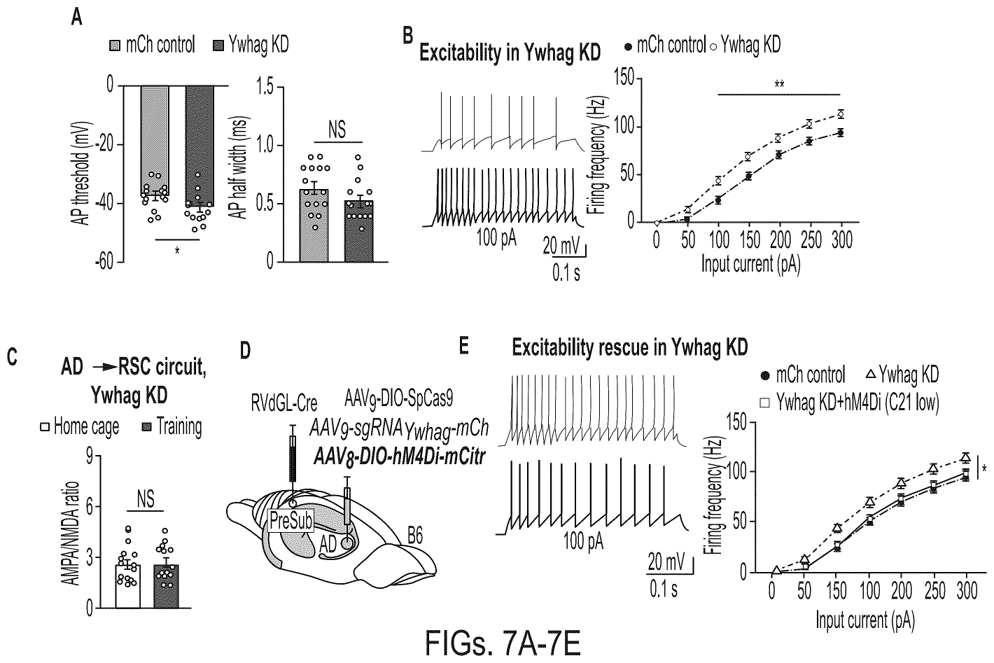

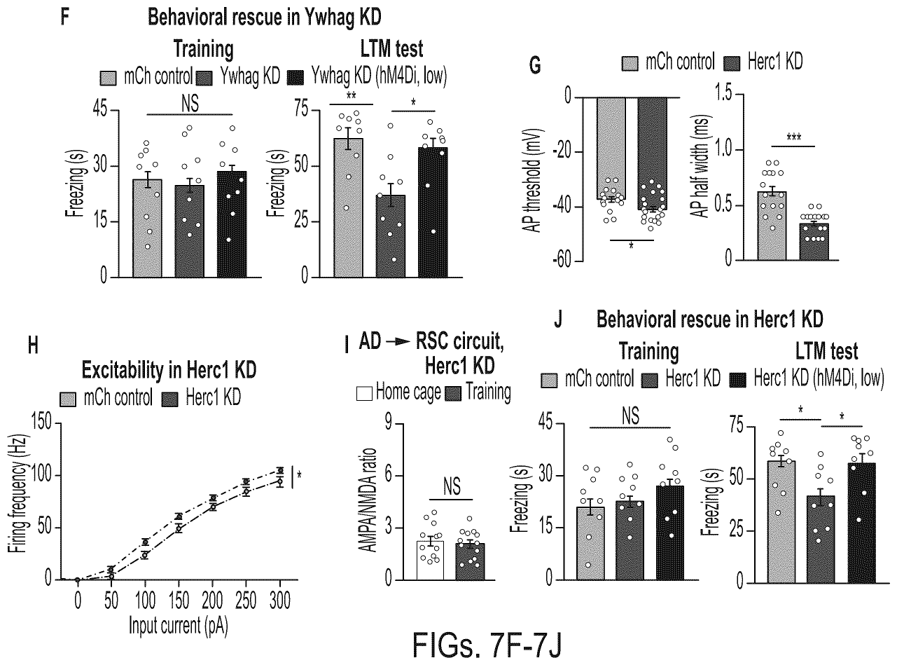

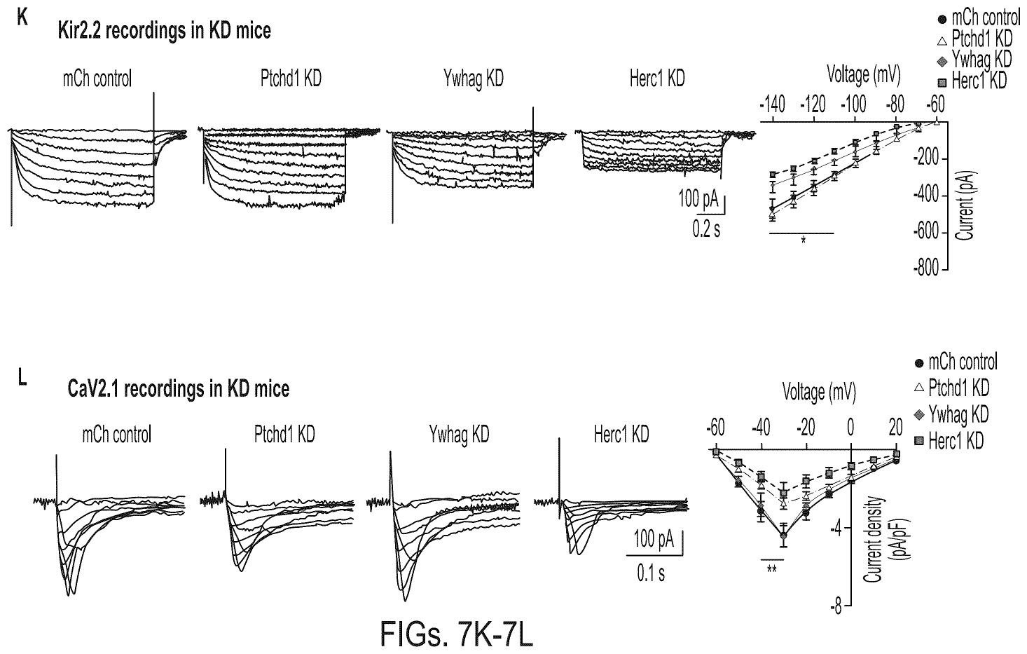

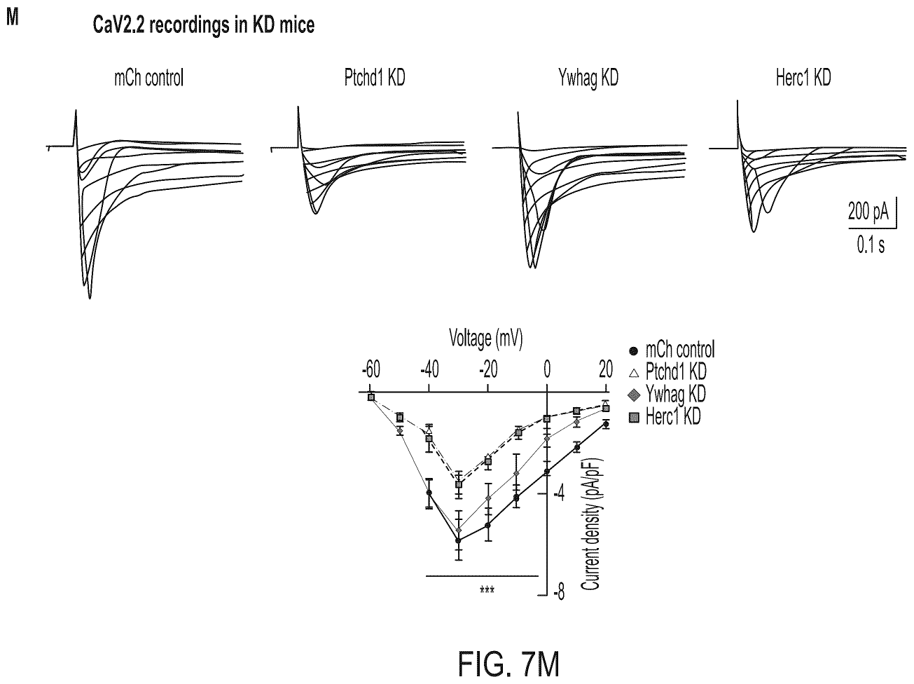

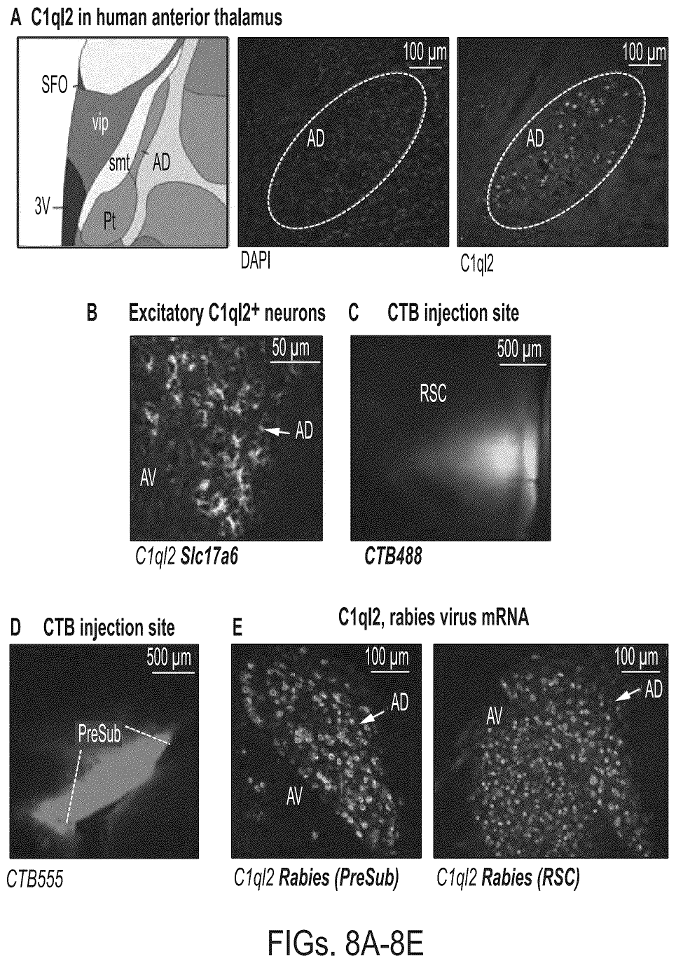

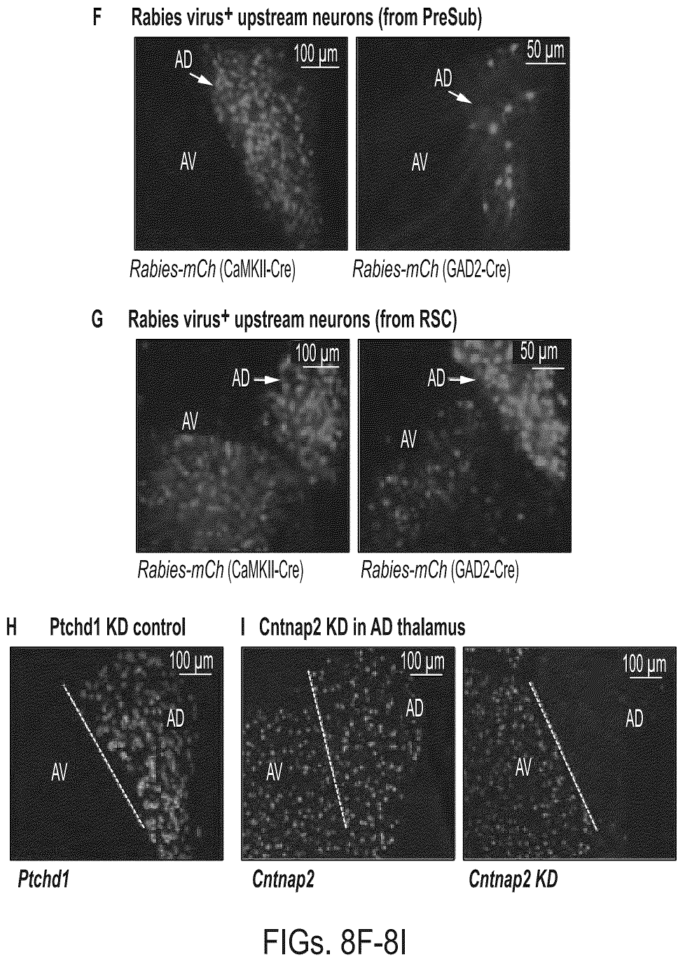

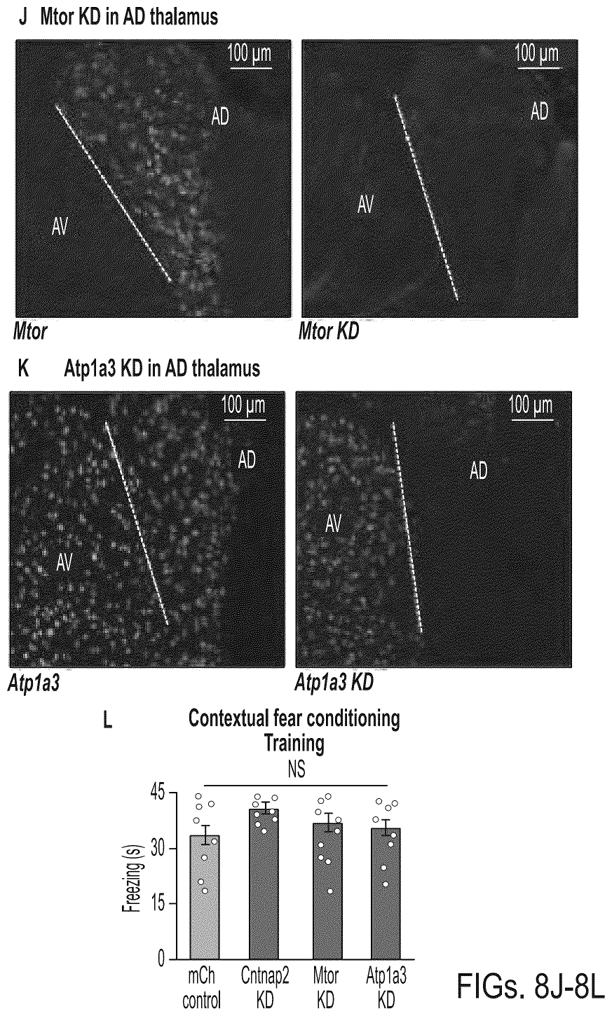

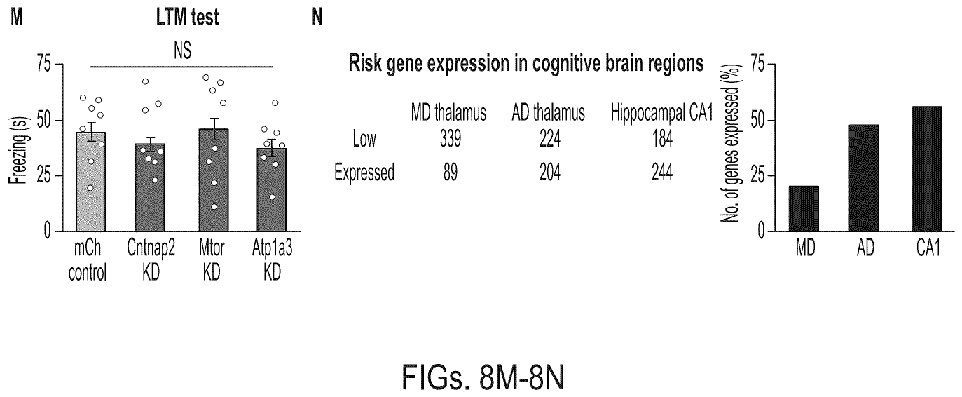

The inventors wanted to know whether the KD of risk genes other than PTCHD1 might also lead to neuronal excitability alterations in AD (FIG. 14J). In contrast to PTCHD1 KD. YWHAG KD in AD neurons did not have an effect on AP half width, but resulted in a decreased AP threshold (FIG. 7A). Similar to PTCHD1 KD. YWHAG KD neurons also showed hyperexcitability (FIG. 7B), which prevented training-induced strengthening of the AD→RSC circuit (FIG. 7C). Therefore, the inventors applied the excitability normalization strategy (FIG. 7D) and found that the hyperexcitability of YWHAG KD neurons could be returned to physiological levels (FIG. 7E). YWHAG KD mice with normalized AD excitability showed control levels of behavioral performance in the CFC paradigm (FIG. 7F). HERC1 KD mice also exhibited AD neuronal hyperexcitability (FIGS. 7G-7H), and lacked training-induced strengthening of the AD→RSC circuit (FIG. 71). Normalizing the excitability of AD neurons in HERC1 KD mice rescued their CFC memory (FIG. 7J). The inventors further demonstrated that normalizing the excitability of AD rescues performance of PTCHD1, YWHAG, and HERC1 KD mice in the spatial working memory task (FIGS. 14K-14M). These experiments show that the KD of different disease risk genes from AD thalamus leads to a common alteration in neuronal excitability, which if treated is sufficient to rescue memory deficits.

The inventors wanted to identify molecular alterations underlying hyperexcitability in AD neurons of PTCHD1, YWHAG, and HERC1 KD mice. The inventors focused on channels that are necessary for maintaining AP threshold and AP half width in thalamic neurons (Kasten et al., 2007), and among these, ones that are robustly expressed in AD (Lein et al., 2007). The inventors narrowed down to two channels that may underlie AP threshold changes (potassium voltage-gated channel subfamily A member 1 or KV1.1, potassium inwardly rectifying channel subfamily J member 12 or KIR2.2) and three channels that may underlie AP half width changes (potassium voltage-gated channel subfamily Q member 2 or KV7.2. calcium voltage-gated channel subunit alpha-1A or (AV2.1, calcium voltage-gated channel subunit alpha-1B or (AV2.2). FISH staining revealed that three out of the five candidate channels, specifically KIR2.2, CAV2.1, and CAV2.2, are decreased in at least one KD mouse model (FIGS. S7A-S7B). To directly measure these individual currents in KD mice, the inventors performed ex vivo recordings. The inventors found that the KIR2.2 current amplitude is decreased in YWHAG and HERC1 KD mice. whereas the (AV2.1 and ('AV2.2 current amplitudes are decreased in PTCHD1 and HERC1 KD mice (FIGS. 7K-7M). These studies identified individual channel subtypes that may underlie AD neuronal hyperexcitability in different KD models.

As reported above, anterior thalamic dysfunction, in particular impairments in the AD subdivision, is a shared feature across a subset of ASD and schizophrenia models that exhibit ID-like memory defects. At the cellular level, three different ASD and schizophrenia KD models exhibited hyperexcitability of AD neurons, through different mechanisms. Furthermore, neuronal hyperexcitability was causally related to cognitive deficits in these KD mice because normalization of this physiological property rescued memory deficits in all three models. These observations suggest that a subset of different human disorders with ID may involve anterior thalamic dysfunction.

The inventor's interest in understanding the role of PTCHD1 in the context of cognitive impairments led to the discovery that AD thalamus underlies memory phenotypes in a subset of different neuropsychiatric models. For two reasons, the inventors xamined the role of AD and neighboring AV in wild type mice. First, in the literature, the precise role of these two ATN subdivisions has not been reported, primarily due to the lack of precise manipulation strategies. This is important to help explain how dysfunction in these nuclei contribute to disease phenotypes. Second, in addition to AD, several ASD and schizophrenia risk genes are expressed in AV thalamus. Therefore, the inventors anted to know whether these two nuclei support the same or different cognitive processes. The inventors ound that the AD→RSC circuit is necessary for memory encoding, whereas the AV→RSC circuit regulates memory specificity. These findings indicate that neighboring ATN subdivisions differentially contribute to a cognitive task.

AD thalamus is specifically important for contextual encoding processes, as evidenced by loss of function phenotypes observed in contextual fear conditioning and inhibitory avoidance paradigms, but not in tone fear encoding. Further support for this role of AD comes from the fact that it is the only ATN subdivision that directly receives visual input (Jankowski et al., 2013). Regarding the AD→PreSub circuit, since the inventors did not observe a significant contribution to our memory behavioral paradigm, it is likely that this circuit plays a bigger role in head direction coding (Winter et al., 2015). The function of AV thalamus in memory specificity is strengthened by the findings that AV but not AD receives prefrontal cortex (PFC) inputs (FIG. 3D), and that PFC is important for generalization behavior (Xu and Sudhof, 2013). Interestingly, their study showed that the PFC→nucleus reuniens (RE) circuit is important for memory specificity, based on which the inventors found that PFC neurons projecting to RE also send collaterals to AV but not AD thalamus (FIG. S7C).

Given that AD and AV converge on the same cortical region, it is important to understand how these two excitatory inputs give rise to distinct behavioral phenotypes at the level of RSC neurons. Without intending to be bound by theory, one mechanism is that distinct RSC ensembles receive input from AD or AV neurons, for which the inventors have obtained some cellular-level evidence (FIGs. S7D-S7E). Another mechanism is that AD and AV together control the level of activation of EC-projecting RSC (i.e., RSC→EC) neurons during encoding within a physiological range. Specifically, if the neural activity of RSC→EC neurons were below a minimal threshold, memory encoding would be impaired, whereas if their activity level exceeded an upper limit, memory encoding would be unaffected but there would be a decrease in specificity. Our data supports this second mechanism because the inventors found that AD but not AV provides the major excitatory drive to RSC→EC neurons, and AV provides important excitatory drive to VIP⁺ inhibitory neurons in RSC that are capable of regulating the overall activity of RSC→EC neurons.

The work described herein provides a better understanding of how anterior thalamus regulates cortico-entorhinal-hippocampal circuits during memory formation. These studies also reveal an important link between anterior thalamic dysfunction and cognitive impairments in a subset of ASD and schizophrenia models, which provide for the development of therapeutic strategies capable of treating cognitive impairments in multiple disorders.

The results described above were obtained using the following methods and materials.

### EXPERIMENTAL MODEL AND SUBJECT DETAILS

Mice. C57BL/6J wild type male mice were obtained from Jackson Laboratory. Experiments using CaMKII-Cre mice employed the T29-1 transgenic line (Stock No. 005359, Jackson Laboratory). Experiments using GAD2-Cre mice employed the GAD2-IRES-Cre knock-in line (Stock No. 028867, Jackson Laboratory). Experiments using PV-Cre mice employed the B6 PV^{Cre} knock-in line (Stock No. 017320, Jackson Laboratory). Experiments using SST-Cre mice employed the SST-IRES-Cre knock-in line (Stock No. 028864, Jackson Laboratory). Experiments using VIP-Cre mice employed the VIP-IRES-Cre knock-in line (Stock No. 031628. Jackson Laboratory). For AD neural activity labeling based on the c-fos promoter, the inventors used the previously described c-fos-Cre^{ERT2} mouse line (Guenthner et al., 2013). These mice are also known as Fos^{CreER} or Fos-TRAP mice in which cFos-positive neurons can be labeled by the intraperitoneal injection of 4-hydroxytamoxifen (4-OHT) within a user-defined time-window. For our experiments. Fos-TRAP mice were crossed with the Cre-dependent tdTomato reporter mouse line Ai14. which were obtained from Jackson Laboratory (Stock No. 007908). All transgenic and knock-in mouse lines were maintained as hemizygotes. Mice had access to food and water ad libitum and were socially housed in numbers of two to five littermates until surgery. Following surgery, mice were single housed. For behavioral experiments, all mice were male and 3-5 months old. All experiments were conducted in accordance with U.S. National Institutes of Health (NIH) guidelines and the Massachusetts Institute of Technology Department of Comparative Medicine and Committee on Animal Care.

Generation of C1ql2-Cre mice. C1ql2-IRES-Cre knock-in mice were generated using cloning-free CRISPR as previously described (Aida et al., 2015). Briefly, a C1ql2-IRES-Cre targeting vector was constructed by Gibson assembly (NEB E2621X) using IRES-Cre-pA cassette (from PL450-IRES-Cre-pA plasmid, a kind gift from Z. Josh Huang at Cold Spring Harbor Laboratory). PCR amplified 2 kb C1q12 homology arms, and a pBluescript plasmid backbone. Synthetic crRNA and tracrRNA were purchased from IDT. Synthego, and Fasmac, Injection mixtures were prepared by mixing crRNA (CGCCCUCUAGGCCCCUAAUC (SEQ ID NO: 9) for protospacer sequence, final concentration 1.22 μM) and tracrRNA (final concentration 1.22 μM) in nuclease-free water and Tris-HCI pH 7.39 (final concentration 10) mM). The mixture was heat denatured at 94° C. for 5 min, followed by re-annealing at room temperature for 10 min. EnGen Cas9 NLS. S. pyogenes (New England Biolabs, final concentration 60 ng μl⁻¹) was added and the mixture was incubated at 37° C. for 15 min, then mixed with the C1ql2-IRES-Cre targeting vector (final concentration 5 ng μl⁻¹) and RAD51 protein (Abcam ab63808. final concentration 10 ng μl⁻¹). The injection mixture was kept on ice and briefly heated to 37° C. prior to injections. Female mice (4-5 weeks old, C57BL/6NTac) were super-ovulated by intraperitoneal injection of PMS (5 IU per mouse, three days prior to microinjections) and hCG (5 IU per mouse. 47 hr after PMS injections) and then paired with males. Pregnant females were sacrificed by cervical dislocation at day 0.5 pcd, and zygotes were collected into 0.1% hyaluronidase/FHM (Sigma). Zygotes were washed in drops of FHM, and cumulus cells were removed. Zygotes were cultured in KSOM-AA for one hour and then used for microinjections. Pronuclear microinjections were performed using a Narishige micromanipulator, Nikon Eclipse TE2000-S microscope, and Eppendorf 5242 microinjector. Individual zygotes were injected with 1-2 pl of the injection mixture using an automatic injection mode set according to needle size and adjusted for a visible increase in pronuclear volume. Following injections, cells were cultured in KSOM-AA overnight, then embryos were surgically implanted into pseudopregnant CD-1 females (Charles River Laboratories, strain code 022) 24 hr post-injection, and allowed to develop normally until natural birth. Genomic DNA was purified from tail samples and PCR genotyped. Cre activity and specificity were tested by injection of AAV₉-EF1α-DIO-eYFP into ATN and eYFP fluorescence localized to CIQL2⁺ AD neurons.

Marmosets. Common marmoset (Callithrix jacchus) monkeys were used for fluorescent in situ hybridization (FISH) experiments. Marmosets had access to food and water ad libitum and were socially housed in numbers of two to three cage mates. Male marmosets ranging from 4-6 years old were used for all experiments. All experiments were conducted in accordance with U.S. National Institutes of Health (NIH) guidelines and the Massachusetts Institute of Technology Department of Comparative Medicine and Committee on Animal Care.

### METHOD DETAILS

### DropViz RNA-Sequencing Dataset

Single-cell suspensions were generated from adult male C57BL/6J mice (60-70 days old). Mouse thalamic excitatory (VGLUT2⁺) neuron single-cell RNA-sequencing data is based on 89,027 cells (n=6 mice). Detailed information regarding cell suspensions, cell recovery rates, cell type and subtype acquisition, Drop-seq library preparation and sequencing, and quantitative analyses has been previously described (Saunders et al., 2018).

Fluorescent in situ hybridization. Experiments used C57BL/6J mouse brain samples, virus-injected mouse brain samples, or common marmoset brain samples. These mouse and marmoset brain samples were extracted, embedded in OCT compound (Tissue-Tek), and flash frozen in liquid nitrogen. A normal human donor thalamus brain sample containing ATN was obtained from Cureline Inc. This human sample was also embedded in OCT compound and flash frozen in liquid nitrogen. Coronal sections (16 μm thickness) were prepared on a cryostat (Leica) and stored at −80° C. FISH mRNA staining was performed using the ACD RNAScope multiplex fluorescent protocol for fresh frozen tissue. Briefly, charged slides with mouse, marmoset, or human tissue sections were fixed in pre-chilled paraformaldehyde (PFA) for 30 min, followed by a series of dehydration steps using 50%, 70%, and 100% ethanol. Sections were then permeabilized with ACD protease IV for 30 min, followed by probe hybridization for 2 hr at 40° C. Fluorescent labeling of up to 3 probes per section was performed using four steps of Amp 1-FL to Amp 4-FL. Sections were stained with DAPI and stored at 4° C. Mouse ACD probes for Ontnap2 (Cat. No. 449381), Atpla3 (Cat. No. 432511), Gria3 (Cat. No. 426251), Mtor (Cat. No. 451651), Ywhag (Cat. No. 812981), Hercl (Cat. No. 871341), Cacna1g (Cat. No. 459761), C1ql2 (Cat. No. 480871), PV (Cat. No. 421931), Col25a1 (Cat. No. 538511), rabies virus (Cat. No. 456781), Ptchd1 (Cat. No. 489651), Sle17a6 (Cat. No. 319171), Konj12 (Cat. No. 525171), Kenq2 (Cat. No. 444251), Kcnal (Cat. No. 481921), Cacna1a (Cat. No. 493141), and Cacna1b (Cat. No. 468811) were used. Marmoset ACD probes for C1ql2 (Cat. No. 525821) and Col25a1 (Cat. No. 557651) were used. Human ACD probe for C1ql2 (Cat. No. 478011) was used. Stained sections were imaged with a 20× magnification objective on a Leica confocal microscope. Images were processed using ImageJ, and quantifications were performed manually from 3-5 sections per animal. All counting experiments were conducted blind to experimental group.

Viral constructs. The following viruses were acquired from Addgene: AAVretro-Cre (specifically AAVretro-hSyn-Cre, catalog #105553-AAVrg, 7×10¹² GC ml⁻¹ titer), AAV₉-EF1α-DIO-ChR2-eYFP (catalog #20298-AAV9, 7×10¹² GC ml⁻¹ titer), AAV₉-CaMKIIα-ChR2-eYFP (catalog #26969-AAV9, 1×10¹³ GC ml⁻¹ titer), AAV₉-CaMKIIα-ChR2-mCherry (catalog #26975-AAV9), 7×10¹² GC ml⁻¹ titer), AAV₈-hSyn-DIO-hM4Di-mCherry (catalog #44362-AAV8, 1×10¹³ GC ml⁻¹ titer), AAV₈-hSyn-DIO-mCherry (catalog #50459-AAV8, 7×10¹² GC ml⁻¹ titer), AAV₁-hSyn-Cre (anterograde virus, catalog #105553-AAV1, 1×10¹³ GC ml⁻¹ titer), AAV₉-hSyn-DIO-hM3Dq-mCherry (catalog #44361-AAV9, 1×10¹³ GC ml⁻¹ titer), AAV₈-hSyn-mCherry (catalog #114472-AAV8, 1×10¹³ GC ml⁻¹ titer), AAV₈-hSyn-DIO-hM4Di-mCitrine (catalog #50455-AAV8, 1×10¹³ GC ml⁻¹ titer), and AAV₈-CaMKIIα-hM4Di-mCherry (catalog #50477-AAV8, 2×10¹² GC ml⁻¹ titer). The following Cre-Off (DO) AAV constructs were acquired from Addgene: AAV-EF1α-DO-NpHR3.0-eYFP (plasmid #37087), AAV-EF1α-DO-eGFP (plasmid #37085), and AAV-EF1α-DO-ChETA-tdTomato (plasmid #37756). The AAV-EF1α-DIO-C1V1-eYFP construct (plasmid #35497) was also acquired from Addgene. All these plasmids were serotyped with AAV₅ coat proteins and packaged by the Viral Core at Boston Children's Hospital (2×10¹³ GC ml⁻¹ viral titers). The AAV-CaMKIIα-mCherry construct (plasmid #114469) was obtained from Addgene, serotyped with AAV& coat proteins, and packaged by the Viral Core at Boston Children's Hospital (4×10¹² GC ml⁻¹ viral titer). The AAV-cFos-Cre^{ERT2} construct was a gift from Karl Deisseroth, which was serotyped with AAV coat proteins and packaged by the Viral Core at Boston Children's Hospital (5×10^{12 12} GC ml⁻¹ viral titer). The AAV₉-EF1α-DIO-eYFP (1.2×10^{12 13} GC ml⁻¹ viral titer) and AAV₉-EF1α-DIO-eArch3.0-eYFP (1.6×10^{12 13} GC ml⁻¹ viral titer) viruses were acquired from the University of North Carolina (UNC) at Chapel Hill Vector Core.

Cholera toxin subunit B. To characterize neuronal populations in AD, AV, and RSC based on their projection targets, the inventors used cholera toxin subunit B (CTB) conjugated to Alexa-488, Alexa-555, or Alexa-647 diluted in phosphate buffered saline (PBS) solution at a final concentration of 1% wt vol⁻¹. Diluted CTB was aliquoted and stored at −20° C. For mouse circuit tracing experiments, 80-300 nl CTB was unilaterally injected into target sites. Six days after injections, mice were perfused for histology followed by coronal/sagittal sectioning (50 μm thickness) using a vibratome (Leica). For circuit-specific neuronal activity (i.e., cFos) experiments using mice, CTB only-, CTB and AD hM4Di-mCh virus-, or CTB and AV NpHR-eYFP virus-injected animals went through the contextual fear conditioning (CFC) behavior protocol 30 days after injections followed by timed perfusions 60 min after behavior. For AD and AV manipulation mice, details are provided in the rabies virus sub-heading. CTB sections were imaged with a 20× magnification objective on a Leica confocal microscope. Images were processed using ImageJ, and quantifications were performed manually from 3-5 sections per animal. All counting experiments were conducted blind to experimental group.

Rabies virus. To label ATN inputs to PreSub and RSC, 150 nl first generation rabies virus (RV) expressing GFP was injected into each of these downstream targets. Five days after injections, these mice were used for FISH staining as described above. For mouse ex vivo electrophysiological recordings from AD vs. AV thalamic neurons, RV-GFP was injected into PreSub followed by recordings five days later. For recordings, details are provided in the “Ex vivo electrophysiology” sub-heading. To identify inputs to Cre neurons, the inventors used a monosynaptic retrograde tracing approach via a Cre-dependent helper virus combined with RV technology. The first component was an AAV vector that allowed simultaneous expression of three genes: TVA, eGFP, and RV glycoprotein (G). Briefly, this vector was constructed by deleting the sequence between the inverse terminal repeats of pAAV-MCS (Stratagene), and replacing it with a cassette containing the following: human synapsin-1 promoter (Syn, Genbank NG_008437); the Kozak sequence: a FLEX cassette containing the transmembrane isoform of TVA (lacking a start codon), eGFP, and G separated by the highly efficient porcine teschovirus self-cleaving 2A element: the woodchuck post-transcriptional regulatory element (WPRE) and a bovine growth hormone polyadenylation site. This vector was termed pAAV-synP-FLEX-sTpEpB (i.e., the helper virus) and serotyped with AAVᵣₕ₈ coat proteins. The second component was a deletion-mutant RV produced by replacing the eGFP gene in cSPBN-4GFP with the mCherry gene (i.e., the RVΔG-mCherry virus, also known as the Rabies-mCh virus), which was packaged with the ASLV-A envelope protein. For tracing experiments using different Cre mouse lines, 100 nl of the Cre-dependent helper virus was unilaterally injected into PreSub or RSC. One week later, 100 nl of RVAG-mCherry virus was unilaterally injected into the same PreSub or RSC. Six days after the second viral injection, mice were perfused for histology and imaging. To map brain-wide inputs to AD vs. AV, 150 nl AAVᵣₑₜᵣₒ-Cre virus was unilaterally injected into PreSub (for AD) or RSC (for AV) combined with 100 nl Cre-dependent helper virus injections into ATN. Three weeks later, 100 nl of RVΔG-mCherry virus was unilaterally injected targeting AD (PreSub injected mice) or AV (RSC injected mice). One week after the second viral injection. mice were perfused for histology and imaging. To identify ATN neurons that project to EC-projecting RSC neurons. 250 nl AAVᵣₑₜᵣₒ-Cre virus was unilaterally injected into EC combined with 100 nl Cre-dependent helper virus injections into RSC. Three weeks later. 100 nl of RVAG-mCherry virus was unilaterally injected into the same RSC. One week after the second viral injection. mice were perfused for histology and imaging. RV⁺ coronal sections (50 μm) were imaged with a 10× or 20× magnification objective on an Olympus epifluorescent microscope. Images were processed using ImageJ, and quantifications were performed manually from 3-5 sections per animal.

For brain-wide inputs to AD vs. AV, tiled images were taken for entire coronal sections (every 4^{th} section from each brain sample), which were needed for manual atlas alignment using an electronic version of the Franklin and Paxinos Mouse Brain in Stereotaxic Coordinates' (3^{rd} edition). Quantifications for these brain-wide input mapping experiments were performed manually. For each RV experiment, starter cell counts across mice were normalized, which has also been indicated in the respective FIG. legends. All counting experiments were conducted blind to experimental group. A third type of RV. referred to as the second generation RV, has been used for ex vivo electrophysiology and behavioral experiments. Specifically, this RV expresses Cre recombinase (i.e., RVdGL-Cre) in upstream neurons. For cell body electrophysiology. RVdGL-Cre was injected into PreSub combined with a Cre-dependent ChR2-eYFP virus in ATN, which allowed labeling of only AD neurons within ATN with high specificity. This strategy to label AD neurons was employed for CFC behavioral manipulations with a Cre-dependent hM4Di-mCherry virus. AD circuit electrophysiology with a Cre-dependent ChR2-eYFP virus, AD→RSC circuit manipulations during behavior with either a Cre-dependent ChR2-eYFP virus or a Cre-dependent eArch-eYFP virus. AD manipulations during behavior with a Cre-dependent hM4Di-mCherry virus for cFos analyses. AD manipulations during behavior with a Cre-dependent hM4Di virus for cFos analyses in EC-projecting RSC neurons that have been labeled with CTB. AD-specific gene knockdown (KD) experiments. AD circuit electrophysiology with a Cre-dependent ChR2-eYFP virus in KD mice, rescue experiments in KD mice. AD behavioral manipulations with a Cre-dependent hM4Di virus for in vivo local field potential (LFP) recordings, and simultaneous AD and AV labeling experiments. The RVdGL-Cre virus injected into PreSub combined with a Cre-Off (DO) NpHR-eYFP virus injected in ATN allowed labeling of only AV neurons within ATN with high specificity (i.e., because AD but not AV projects to PreSub. RVdGL-Cre in AD neurons turns off viral expression). This strategy to label AV neurons was employed for behavioral manipulations. AV manipulations during behavior for cFos analyses in RSC neurons. AV→RSC inhibition with PV or VIP activation in RSC during behavior. AV manipulations during behavior for cFos analyses in EC-projecting RSC neurons that have been labeled with CTB, and simultaneous AD and AV labeling experiments.

In vivo genome editing. In vivo knockdown experiments targeting AD thalamus or hippocampal CA1 employed an AAV CRISPR/Cas9 approach. Single guide RNA (sgRNA) candidates targeting Ptchd1, Ywhag, Gria3, Herc1, Atpla3, Mtor, and Ontnap2 with high specificity and high efficiency were computationally identified from sgRNA libraries for genome-wide CRISPR knockout screening (Doench et al., 2016). Three U6-sgRNA(FE) gene fragments with the F+E tracrRNA backbone were synthesized by Integrated DNA

Technologies (sequences are provided below, spacer sequences are capitalized). These fragments were cloned into the pX552-mCherry plasmid (EGFP in pX552 plasmid was replaced with mCherry, pX552 was obtained from Addgene, plasmid #60958) by Gibson assembly (NEB E2621X) to construct pX552-3xsgRNA(FE)-mCherry. The inventors used a previously reported sgRNA plasmid targeting Caena1g (Li et al., 2020). These constructs were functionally validated in Neuro2A cells. The AAV vectors were serotyped with AAV₉ coat proteins and packaged in-house or by the Viral Core at Boston Children's Hospital (8×10¹² genome copy (GC) ml⁻¹ viral titers for Ptchd1, Cacna1g).

In-house AAV production followed a previously described method (Challis et al., 2019). Briefly, sgRNA plasmids, pAdDeltaF6 (Addgene, plasmid #112867), and pAAV2/9 (Addgene, plasmid #112865) were co-transfected into HEK293T cells using polyethylenimine (Cat. No. 23966-1, Polysciences). Cells were cultured in Dulbecco's modified essential medium (DMEM, Invitrogen) containing 10% fetal bovine serum (Gibco) and 1% penicillin-streptomycin (Gibco) at 37° C. with 5% CO₂. Cells were harvested 72 hr post transfection by 4,000× g centrifugation at 4° C. for 10 min. Virus in media was precipitated by 8% PEG8000 (Sigma). Cell pellets and virus precipitated from media were re-suspended in digestion buffer containing 500 mM NaCl, 40 mM Tris base, and 10 mM MgCl₂. Benzonas nuclease (100U, Sigma) was added in the digestion buffer and incubated at 37° C. water bath for 1 hr. Next, the inventors performed centrifugation at 2,000xg for 15 min, and the supernatant was used on a discontinuous gradient of 15%, 25%, 40%, and 60% iodixanol in a 36.2 ml ultracentrifuge tube (Optiseal Seal, Cat. No. 362183, Beckman). Ultracentrifugation was performed at 350,000×g. 18° C. for 2.5 hr. 5 ml fractions in 40% layer and 40%-60% interface was collected. These fractions were desalted using a 100 kDa cutoff ultrafiltration tube (15 ml, Millipore). Buffer was exchanged 4 times with 1× PBS with 0.001% Pluronic F-68. AAV titers were determined by real-time quantitative PCR (qPCR) using the primers of mCherry. Forward primer: 5′ GAGTTCATCTACAAGGTGAAGCTGCGC 3′ (SEQ ID NO: 10), reverse primer: 5′ CTTGTAGGTGGTCTTGACCTCAGCGTC 3′ (SEQ ID NO: 11) (1-2.5×10¹² GC ml⁻¹ for Ywhag, Gria3, Herc1, Atpla3, Mtor, Cntnap2).

For AD targeting, these sgRNA AAVs were combined with a Cre-dependent SpCas9 AAV, which was developed by Jie Xu and Dong Kong. The AAV-DIO-SpCas9 plasmid was serotyped with AAV9 coat proteins and packaged by the Viral Core at Boston Children's Hospital (2 × 1013 GC ml⁻¹ viral titer). For these in vivo experiments, RVdGL-Cre was injected into PreSub and a 1:1 mix of AAV₉-sgRNA-mCherry:AAVs-DIO-SpCas9 was injected into ATN, which allowed for AD-specific knockdown of target genes. For CA1 targeting, sgRNA AAVs were combined 1:1 with a constitutive AAV₉-CMV-SpCas9 virus (4×10¹² GC ml⁻¹ viral titer, Vector Biolabs). FISH was used for in vivo knockdown validation.

U6-sgPitchd1-1(FE) sequence
(SEQ ID NO: 12)
agtggccaactccatcactaggggttcctgcggccggcacgcgtaaggtcgggcaggaagagggcctattt
cccatgattccttcatatttgcatatacgatacaagcctgttagagagataattagaattaatttgactgt
aaacacaaagatattagtacaaaatacgtgacgtagaaagtaataatttcttgggtagtttgcagttttaa
aattatgttttaaaatggactatcatatgcttaccgtaacttgaaagtatttcgatttcttggctttatat
atcttgtggaaaggacgaaacaccgcTACCAGGTCGAAGAGAGCGgtttaagagctatgctggaaacagca
tagcaagtttaaataaggctagtccgttatcaacttgaaaaagtcgggcaccgagtcggtgctttttttcc
cagtcacgacgttgtaaaacgacggccagtgagcgcgcgtaatacgactcactatagg
U6-sgPitchd1-2(FE) sequence
(SEQ ID NO: 13)
cccagtcacgacgttgtaaaacgacggccagtgagcgcgcgtaatacgactcactataggaaggtcgggca
ggaagagggcctatttcccatgattccttcatatttgcatatacgatacaagcctcttagagagataatta
gaattaatttgactgtaaacacaaagatattagtacaaaatacgtgacgtagaaagtaataatttcttgcg
gtagtttgcagttttaaaattatgttttaaaatggactatcatatgcttaccgtaacttgaaagtatttcg
atttcttgcctttatatatcttgtcggaaaggacgaaacacccggGTGCTTGGAGCGGTTGACCgtttaag
agctatgctggaaacagcatagcaagtttaaataaggctagtccgttatcaacttgaaaaagtcggcaccc
gagtcggtcctttttttagcggataacaatttcacacaggaaacagctatgaccatgattacgccaagcgc
gc
U6-sgPitchd1-3(FE) sequence
(SEQ ID NO: 14)
agcggataacaatttcacacaggaaacagctatgaccatgattacgccaaggcgggcaaggtcgggcagga
agagggcctatttcccatgattccttcatatttgcatatacgatacaaggctgttagagagataattagaa
ttaatttgactgtaaacacaaagatattagtacaaaatacctgacgtagaaagtaataatttcttgggtag
tttgcagttttaaaattatgttttaaaatggactatcatatgcttaccgtaacttgaaagtatttcgattt
cttgcctttatatatcttgtggaaaggacgaaacaccgTATAATGGGCACCAACTCGGgtttaagagctat
gctggaaacagcatagcaagtttaaataaggctagtcggttatcaacttgaaaaagtggcaccgagtcggt
gcttttttttctagactgcagagggccctgcgtatgagtgcaagtgggttttaggaccaggatgaggcggg
ctg
U6-sgYwhag-1(FE) sequence
(SEQ ID NO: 15)
agtggccaactccatcactaggggttcctgcggccgcacgcgtaaggtcgggcaggaagagggcctatttc
ccatgattccttcatatttgcatatacgatacaaggctgttagagagataattagaattaatttgactgta
aacacaaagatattagtacaaaatacgtgacgtagaaagtaataatttcttgggtagtttgcagttttaaa
attatgttttaaaatggactatcatatgcttaccgtaacttgaaagtatttcgatttcttggctttatata
tcttgtggaaaggacgaaacaccgGATGGTGGACCGCGAGCAACgtttaagagctatgctggaaacagcat
agcaagtttaaataaggctagtccgttatcaacttgaaaaagtggcaccgagtcggtgcttttittcccag
tcacgacgttgtaaaacgacggccagtgagcgcgcgtaatacgactcactatagg
U6-sgYwhag-2(FE) sequence
(SEQ ID NO: 16)
cccagtcacgacgttgtaaaacgacggccagtgagcgcgcgtaatacgactcactataggaaggtcgggca
ggaagagggcctatttcccatgattccttcatatttgcatatacgatacaaggctgttagagagataatta
gaattaatttgactgtaaacacaaagatattagtacaaaatacgtgacgtagaaagtaataatttcttggg
tagtttgcagttttaaaattatgttttaaaatggactatcatatgcttaccgtaacttgaaagtatttcga
tttcttggctttatatatcttgtggaaaggacgaaacaccgCAGGAGGTTCCGTTCCTCATgtttaagagc
tatgctggaaacagcatagcaagtttaaataaggctagtccgttatcaacttgaaaaagtcggcacccgag
tcggtgctttttttagcggataacaatttcacacaggaaacagctatgaccatgattacgccaagcgcgc
U6-sgYwhag-3(FE) sequence
(SEQ ID NO: 17)
agcggataacaatttcacacaggaaacagctatgaccatgattacgccaagcgcgcaaggtcgggcaggaa
gagggcctatttcccatgattccttcatatttgcatatacgatacaaggctgttagagagataattagaat
taatttgactgtaaacacaaagatattagtacaaaatacctgacgtagaaagtaataatttcttgggtagt
ttgcagttttaaaattatgttttaaaatggactatcatatgcttaccgtaacttgaaagtatttcgatttc
ttgcctttatatatcttgtggaaaggacgaaacaccgGAGCAGAAGACGTCTGCGGAgtttaagagctatg
ctggaaacagcatagcaagtttaaataaggctagtccgttatcaacttgaaaaagtggcaccgagtcggtg
cttttttttctagactgcagagggccctgcgtatgagtgcaagtgggttttaggaccaggatgaggcgggg
tg
U6-sgGria3-1(FE) sequence
(SEQ ID NO: 18)
agtggccaactccatcactaggggttcctgcggccggcacgcgtaaggtcgggcaggaagagggcctattt
cccatgattccttcatatttgcatatacgatacaaggctgttagagagataattagaattaatttgactgt
aaacacaaagatattagtacaaaatacgtgacgtagaaagtaataatttcttgggtagtttgcagttttaa
aattatgttttaaaatggactatcatatgcttaccgtaacttgaaagtatttcgatttcttggctttatat
atcttgtgggaaaggacgaaacaccgACGTGGTAGTTCAAATGGAAgtttaagagctatgctggaaacagc
atagcaagtttaaataaggctagtccgttatcaacttgaaaaagtggcaccgagtcggtgctttttttccc
agtcacgacgttgtaaaacgacggccagtgagcgcgcgtaatacgactcactatagg
U6-sgGria3-2(FE) sequence
(SEQ ID NO: 19)
cccagtcacgacgttgtaaaacgacggccagtgagcgcgcgtaatacgactcactataggaaggtcgggca
ggaagagggcctatttcccatgattccttcatatttgcatatacgatacaaggctgttagagagataatta
gaattaatttgactgtaaacacaaagatattagtacaaaatacgtgacgtagaaagtaataatttcttggg
tagtttgcagttttaaaattatgttttaaaatggactatcatatgcttaccgtaacttgaaagtatttcga
tttcttggctttatatatcttgtggaaaggacgaaacaccgCCTTTAAGGCTGGGCGCATCgtttaagagc
tatgctggaaacagcatagcaagtttaaataaggctagtccgttatcaacttgaaaaagtggcacccgagt
cggtgctttttttagcggataacaatttcacacaggaaacagctatgaccatgattacgccaagcgcgc
U6-sgGria3-3(FE) sequence
(SEQ ID NO: 20)
agcggataacaatttcacacaggaaacagctatgaccatgattacgccaagcgcgcaaggtcgggcaggaa
gagggcctatttcccatgattccttcatatttgcatatacgatacaaggctgttagagagataattagaat
taatttgactgtaaacacaaagatattagtacaaaatacgtgacgtagaaagtaataatttcttgggtagt
ttgcagttttaaaattatgttttaaaatggactatcatatgcttaccgtaacttgaaagtatttcgatttc
ttgcctttatatatcttgtggaaaggacgaaacaccgCAAAGCCATTTATGAGCCTGgtttaagagctatg
ctggaaacagcatagcaagtttaaataaggctagtccgttatcaacttgaaaaagtggcaccgagtcggtg
cttttttttctagactgcagagggccctgcgtatgagtgcaagtgggttttaggaccaggatgaggcggcg
ctg
U6-sgHerc1-1(FE) sequence
(SEQ ID NO: 21)
agtggccaactccatcactaggggttcctgcggccggcacgcgtaaggtcgggcaggaagagggcctattt
cccatgattccttcatatttgcatatacgatacaaggctgttagagagataattagaattaatttgactgt
aaacacaaagatattagtacaaaatacgtgacgtagaaagtaataatttcttgggtagtttgcagttttaa
aattatgttttaaaatggactatcatatgcttaccgtaacttgaaagtatttcgatttcttggctttatat
atcttgtggaaaggacgaaacaccgCATCTGCTGATCGGAGTCAGgtttaagagctatgctggaaacagca
tagcaagtttaaataaggctagtccgttatcaacttgaaaaagtggcaccgagtcggtgctttttttccca
gtcacgacgttgtaaaacgacggccagtgagcgcgcgtaatacgactcactatagg
U6-sgHerc1-2(FE) sequence
(SEQ ID NO: 22)
cccagtcacgacgttgtaaaacgacggccagtgagcgcgcgtaatacgactcactatagcAaggtcgggca
ggaagagggcctatttcccatgattccttcatatttgcatatacgatacaaggctgttagagagataatta
gaattaatttgactgtaaacacaaagatattagtacaaaatacgtgacgtagaaagtaataatttcttggg
tagtttgcagttttaaaattatgttttaaaatggactatcatatgcttaccgtaacttgaaagtatttcga
tttcttggctttatatatcttgtggaaaggacgaaacaccgACTATGGGAAACTAGGACATgtttaagagc
tatgctggaaacagcatagcaagtttaaataaggctagtccgttatcaacttgaaaaagtggcaccgagtc
ggtgctttttttagcggataacaatttcacacaggaaacagctatgaccatgattacgccaagcgcgc
U6-sgHerc1-3(FE) sequence
(SEQ ID NO: 23)
agcggataacaatttcacacaggaaacagctatgaccatgattacgccaagcgcgcaaggtcgggcaggaa
gagggcctatttcccatgattccttcatatttgcatatacgatacaaggctgttagagagataattagaat
taatttgactgtaaacacaaagatattagtacaaaatacgtgacgtagaaagtaataatttcttgggtagt
ttgcagttttaaaattatgttttaaaatggactatcatatgcttaccgtaacttgaaagtatttcgatttc
ttggctttatatatcttgtggaaaggacgaaacaccgCCGAGATAGAATGAACAGTGgtttaagagctatg
ctggaaacagcatagcaagtttaaataaggctagtccgttatcaacttgaaaaagtggcaccgagtcggtg
cttttttttctagactgcagagggccctgcgtatgagtgcaagtgggttttaggaccaggatgaggcgcgg
tg
U6-sgAtp1a3-1(FE) sequence
(SEQ ID NO: 24)
agtggccaactccatcactaggggttcctgcggccggcacgcgtaaggtcgggcaggaagagggcctattt
cccatgattccttcatatttgcatatacgatacaaggctgttagagagataattagaattaatttgactgt
aaacacaaagatattagtacaaaatacgtgacgtagaaagtaataatttcttgggtagtttgcagttttaa
aattatgttttaaaatggactatcatatgcttaccgtaacttgaaagtatttcgatttcttggctttatat
atcttgtgggaaaggacgaaacaccgAGGAGATCCTAGCCCGGGATgtttaagagcctatgctggaaacag
catagcaagtttaaataaggctagtccgttatcaacttgaaaaagtggcaccgagtcggtgctttttttcc
cagtcacgacgttgtaaaacgacggccagtgagcgcgcgtaatacgactcactatagg
U6-sgAtp1a3-2(FE) sequence
(SEQ ID NO: 25)
cccagtcacgacgttgtaaaacgacggccagtgagcgcgcgtaatacgactcactatagcAaggtgggcag
gaagagggcctatttcccatgattccttcatatttgcatatacgatacaaggctgttagagagataattag
aattaatttgactgtaaacacaaagatattagtacaaaatacgtgacgtagaaagtaataatttcttgggt
agtttgcagttttaaaattatgttttaaaatggactatcatatgcttaccgtaacttgaaagtatttcgat
ttcttggctttatatatcttgtggaaaggacgaaacaccgGCCGGTGATGATCACGACTGgtttaagagct
atgctggaaacagcatagcaagtttaaataaggctagtccgttatcaacttgaaaaagtggcaccgagtcg
gtgctttttttagcggataacaatttcacacaggaaacagctatgaccatgattacgccaagcgcgc
U6-sgAtp1a3-3(FE) sequence
(SEQ ID NO: 26)
agcggataacaatttcacacaggaaacagctatgaccatgattacgccaagcgcggcaaggtcgggcagga
agagggcctatttcccatgattccttcatatttgcatatacgatacaaggctgttagagagataattagaa
ttaatttgactgtaaacacaaagatattagtacaaaatacgtgacgtagaaagtaataatttcttgggtag
tttgcagttttaaaattatgttttaaaatggactatcatatgcttaccgtaacttgaaagtatttcgattt
cttgcctttatatatcttgtggaaaggacgaaacaccgCGGGGTGTGGTGGTAGCCACgtttaagagctat
gctggaaacagcatagcaagtttaaataaggctagtccgttatcaacttgaaaaagtggcaccgagtcggt
gcttttttttctagactgcagagggccctgcgtatgagtgcaagtgggttttaggaccaggatgaggcgcg
gctg
U6-sgMtor-1(FE) sequence
(SEQ ID NO: 27)
agtggccaactccatcactaggggttcctgcggccggcacgcgtaaggtcgggcaggaagagggcctattt
cccatgattccttcatatttgcatatacgatacaaggctgttagagagataattagaattaatttgactgt
aaacacaaagatattagtacaaaatacgtgacgtagaaagtaataatttcttgggtagtttgcagttttaa
aattatgttttaaaatggactatcatatgcttaccgtaacttgaaagtatttcgatttcttgcctttatat
atcttgtggaaaggacgaaacacccgAAGTGTCCCCTGCCATCGCCgtttaagagctatgctggaaacagc
atagcaagtttaaataaggctagtccgttatcaacttgaaaaagtggcaccgagtcggtgctttttttccc
agtcacgacgttgtaaaacgacggccagtgagcgcgcgtaatacgactcactatagg
U6-sgMtor-2(FE) sequence
(SEQ ID NO: 28)
cccagtcacgacgttgtaaaacgacggccagtgagcgcgcgtaatacgactcactatagcAaggtcgggca
ggaagagggcctatttcccatgattccttcatatttgcatatacgatacaaggctgttagagagataatta
gaattaatttgactgtaaacacaaagatattagtacaaaatacgtgacgtagaaagtaataatttcttggg
tagtttgcagttttaaaattatgttttaaaatggactatcatatgcttaccgtaacttgaaagtatttcga
tttcttggctttatatatcttgtggaaaggacgaaacaccgCTGCGTGGTGAGAATCAGACgtttaagagc
tatgctggaaacagcatagcaagtttaaataaggctagtccgttatcaacttgaaaaagtggcaccgagtc
ggtgctttttttagcggataacaatttcacacaggaaacagctatgaccatgattacgccaagcgcgc
U6-sgMtor-3(FE) sequence
(SEQ ID NO: 29)
agcggataacaatttcacacaggaaacagctatgaccatgattacgccaagcgcgcaaggtcgggcaggaa
gagggcctatttcccatgattccttcatatttgcatatacgatacaaggctgttagagagataattagaat
taatttgactgtaaacacaaagatattagtacaaaatacctgacgtagaaagtaataatttcttgggtagt
ttgcagttttaaaattatgttttaaaatggactatcatatgcttaccgtaacttgaaagtatttcgatttc
ttgcctttatatatcttgtggaaaggacgaaacaccgGACGTTGATGCAGAAGGTAGgtttaagagctatg
ctggaaacagcatagcaagtttaaataaggctagtccgttatcaacttgaaaaagtggcaccgagtcggtg
cttttttttctagactgcagagggccctgcgtatgagtgcaagtgggttttaggaccaggatgaggcggcg
gtg
U6-sgCantnap2-1(FE) sequence
(SEQ ID NO: 30)
agtggccaactccatcactaggggttcctgcggccgcacgcgtaaggtcgggcaggaagagggcctatttc
ccatgattccttcatatttgcatatacgatacaaggctgttagagagataattagaattaatttgactgta
aacacaaagatattagtacaaaatacgtgacgtagaaagtaataatttcttgggtagtttgcagttttaaa
attatgttttaaaatggactatcatatgcttaccgtaacttgaaagtatttcgatttcttggctttatata
tcttgtggaaaggacgaaacaccgGAGCTGCTGAAGGACACATGgtttaagagctatgctggaaacagcat
agcaagtttaaataaggctagtccgttatcaacttgaaaaagtggcaccgagtcggtgctttttttcccag
tcacgacgttgtaaaacgacggccagtgagcgcgcgtaatacgactcactatagg
U6-sgCntnap2-2(FE) sequence
(SEQ ID NO: 31)
cccagtcacgacgttgtaaaacgacggccagtgagcgcgcgtaatacgactcactatagcAaggtcgggca
ggaagagggcctatttcccatgattccttcatatttgcatatacgatacaaggctgttagagagataatta
gaattaatttgactgtaaacacaaagatattagtacaaaatacgtgacgtagaaagtaataatttcttggg
tagtttgcagttttaaaattatgttttaaaatggactatcatatgcttaccgtaacttgaaagtatttcga
tttcttggctttatatatcttgtggaaaggacgaaacaccgATCAGTGCCATTGCAACCCAgtttaagagc
tatgctgggaaacagcatagcaagtttaaataaggctagtccgttatcaacttgaaaaagtggcaccgagt
ccggtgctttttttagcggataacaatttcacacaggaaacagctatgaccatgattacgccaagcgcgc
U6-sgCatnap2-3(FE) sequence
(SEQ ID NO: 32)
agcggataacaatttcacacaggaaacagctatgaccatgattacgccaagcgcgcgcaaggtcgggcagg
aagagggcctatttcccatgattccttcatatttgcatatacgatacaaggctgttagagagataattaga
attaatttgactgtaaacacaaagatattagtacaaaatacctgacgtagaaagtaataatttcttgggta
gtttgcagttttaaaattatgttttaaaatggactatcatatgcttaccgtaacttgaaagtatttcgatt
tcttgcctttatatatcttgtggaaaggacgaaacaccgAGGTCACATCGGGCTGCGTGgtttaagagcta
tgctggaaacagcatagcaagtttaaataaggctagtccgttatcaacttgaaaaagtggcaccgagtcgg
tgcttttttttctagactgcagagggccctgcgtatgagtgcaagtgggttttaggaccaggatgaggcgg
cgctg

Mouse surgery and optic fiber implants. Animals were anesthetized with isoflurane for stereotaxic injections, and were given 1 mg kg⁻¹ meloxicam as analgesic prior to incisions. Injections were targeted to PreSub (−3.8 mm AP, +/−1.75 mm ML, −1.7 mm DV), RSC (−2.46 mm AP, +/−0.25 mm ML, −0.9 mm DV), ATN (−0.55 mm AP, +/−0.9 mm ML, −3.15 mm DV), AD (−0.7 mm AP, +/−0.75 mm ML, −2.75 mm DV), AV (−0.58 mm AP, +/−1.1 mm ML, −3.25 mm DV), EC (−4.75 mm AP, +/−3.35 mm ML, −3 mm DV), hippocampal CA1 (−2.1 mm AP, +/−1.5 mm ML, −1.4 mm DV), RE (−0.58 mm AP, +/−0.25 mm ML, −4.25 mm DV), and PFC (+1.94 mm AP, +/−0.4 mm ML, −2.9 mm DV). Standard injection volumes were 200 nl for PreSub and RSC, 300 nl for ATN, 125 nl for AD and AV, 300 nl for EC, 400 nl for CA1, 250 nl for RE, and 300 nl for PFC. Except for certain retrograde tracing experiments (listed in the rabies virus sub-heading), all other experiments employed these standard injection volumes. CTB/viruses were injected at 70 nl min⁻¹ using a glass micropipette attached to a 10 ml Hamilton microsyringe. The needle was lowered to the target site and remained for 5 min before beginning the injection. After the injection, the needle stayed for 10 min before it was withdrawn. For behavioral manipulation experiments using optogenetics, single mono-fiber implants (200 μm core diameter, Newdoon) were lowered either above injection sites or terminals bilaterally (AV, −0.58 mm AP, +/−1.1 mm ML, −3.1 mm DV: RSC, −2.46 mm AP, +/−0.25 mm ML, −0.7 mm DV: PreSub (−3.8 mm AP, +/−1.75 mm ML, −1.85 mm DV): EC, −4.65 mm AP, +/−3.35 mm ML, −2.25 mm DV). The implant was secured to the skull with two jewelry screws, adhesive cement (C&B Metabond), and dental cement. Mice were given 1−2 mg kg⁻¹ sustained-release buprenorphine as analgesic after surgeries and allowed to recover for at least 2 weeks before behavioral experiments. All injection sites were verified histologically. As criteria, the inventors only included mice with virus expression limited to the targeted regions.

Immunohistochemistry. Mice were dispatched using an overdose of isoflurane and transcardially perfused with PBS, followed by 4% paraformaldehyde (PFA). Brains were extracted and incubated in 4% PFA at room temperature overnight. Brains were transferred to PBS and 50 μm coronal slices were prepared using a vibratome. For immunostaining, each slice was placed in PBS+0.2% Triton X−100 (PBS-T), with 5% normal goat serum for 1 hr and then incubated with primary antibody at 4° C. for 24 hr. Slices then underwent three wash steps for 10 min each in PBS-T, followed by a 2 hr incubation with secondary antibody. After three more wash steps of 10 min each in PBS-T, slices were mounted on microscope slides. Antibodies used for staining were as follows: rabbit anti-CIQL2 (1:500, Thermo Fisher) and anti-rabbit Alexa-488 (1:500), chicken anti-GFP (1:1000, Life Technologies) and anti-chicken Alexa-488 (1:1000), rabbit anti-RFP (1:1000, Rockland) and anti-rabbit Alexa-555 (1:500), rabbit anti-cFos (1:500, Cell Signaling Technology) and anti-rabbit Alexa-488 or Alexa-555 (1:300), and nuclei were stained with DAPI (1:3000, Sigma). To visualize rabies virus starter cells, GFP antibody staining was performed. To visualize ChR2-expressing terminals in ATN, both GFP and RFP antibody staining was performed. To visualize AD hM4Di-mCherry terminals in RSC, RFP antibody staining was performed. To visualize

ChR2-eYFP terminals in ATN, GFP antibody staining was performed. To visualize AD and AV cell body labeling in ATN, both GFP and RFP antibody staining was performed. All analyses were performed blind to the experimental conditions.

Fos-TRAP activity-dependent labeling. For activity-dependent labeling experiments, as mentioned above FosTRAP mice crossed to Ai14 reporter mice were employed. 4-hydroxytamoxifen (4-OHT, Sigma-Aldrich) was dissolved in 100% ethanol solution by shaking at 37° C. for 20−30 min. One-part castor oil to four parts sunflower oil was combined to prepare the oil mixture that would eventually be injected intraperitoneally (IP) into the mouse. Dissolved 4-OHT was combined with the oil mixture, followed by ethanol evaporation using a centrifuge. The final concentration of 4-OHT dissolved in the oil mixture was 10 mg ml−1. For each mouse, optimal activity-dependent labeling was achieved using a target concentration of 30−40 mg kg−1. One hour prior to the behavioral epoch of interest, mice were injected with 4-OHT. Following behavior experiments, mice were returned to their home cages and remained undisturbed for at least 72 hours, after which they were perfused for histological analyses.

Chemogenetic and optogenetic experiments. For chemogenetic (i.e., hM4Di or hM3Dq) neuronal activity manipulation experiments, we used the second-generation agonist known as compound 21 (C21). This agonist was purchased in a water-soluble dihydrochloride form (Hello Bio). For each mouse, optimal chemogenetic activity was achieved using a target concentration of 2 mg kg⁻¹ (injected IP), 45 min before the behavioral epoch of interest. The exception to this target concentration was for low (0.6 mg kg⁻¹) vs. regular (2 mg kg⁻¹) dose experiments in PTCHD1 KD mice, and low dose experiments in YWHAG and HERC1 KD mice. For optogenetic neuronal activity manipulation experiments, ChR2 was activated at 20 Hz (15 ms pulse width) with a 473 nm laser (10−15 mW, blue light), eArch and NpHR was activated with a 570 nm laser (10 mW, constant green light), CIVI was activated at 20 Hz (15 ms pulse width) with a 570 nm laser (10 mW, green light), and ChETA was activated at 20 Hz (15 ms pulse width) with a 410 nm laser (10 mW, blue light).

Cell counting. For details regarding quantification of RV tracing experiments, please refer to the rabies virus sub-heading. Unless specified, brain sections were imaged with a 20× magnification objective on a Leica confocal microscope. Images were processed using ImageJ, and quantifications were performed manually from 3−5 sections per animal. All counting experiments were conducted blind to experimental group. Researcher 1 trained the animals, prepared slices, and randomized images, while Researcher 2 performed cell counting. Percentage of PreSub-projecting (CTB555) AD neurons that send collaterals to RSC (CTB488) was calculated as ((CTB488⁺ CTB555⁺)/(Total CTB555⁺))×100. The percentage of retrogradely-labeled (by RV) AD neurons that express the marker C1ql2 was calculated as ((RV⁺ C1ql2⁺)/(RV⁺))×100. To quantify the number of neurons in each brain region projecting to AD or AV neurons, RV-mCherry⁺ neurons in each upstream target structure were counted from all coronal slices containing the structure per mouse. To quantify the number of activated (cFos⁺) neurons in Fos-TRAP/Ail4 mice, tdTomato neurons in AD thalamus were manually counted from home cage, CFC training, and immediate shock groups. To quantify neuronal activity in RSC/CA1, cFos⁺ neurons were manually counted from specified behavioral groups. These active neuron cell counts were normalized to the number of DAPI⁺ cells in the field of view. Percentage of retrogradely-labeled (CTB⁺) neurons that are activated (cFos⁺) was calculated as ((CTB⁺cFos⁺)/(Total CTB555))×100. Percentage of PV-Cre or VIP-Cre (eYFP) neurons that are activated (cFos) was calculated as ((eYFP⁺cFos)/(Total eYFP⁺))×100. To quantify the number of neurons in AV thalamus that project to PV⁺, SST⁺, or VIP⁺ inhibitory neurons in RSC, RV-mCherry⁺ neurons in AV were manually counted. Percentage of Sle17a6⁺ neurons in AD that express the marker C1ql2 was calculated as ((Slc17a6 Clql2)/(Total Slc17a6⁺))×100. Percentage of RSC neurons that receive both AD input (eYFP⁺) and AV input (cFos⁺) was calculated as ((eYFP⁺cFos⁺)/(eYFP⁺))×100. Similarly, for risk gene KD experiments, fluorescence intensity was measured in AD or CA1 using ImageJ. These values were averaged and compared between mCh control and KD mice. Data were analyzed using Microsoft Excel with the Statplus plug-in or Prism 6 software.

### Ex Vivo Electrophysiology

Slice preparation. All ex vivo experiments were conducted blind to experimental group. Researcher I trained the animals and administered drug, while Researcher 2 dispatched the animals and conducted physiological recordings. Mice (8−12 weeks old) were anesthetized with isoflurane, decapitated, and brains were quickly removed. For AMPA/NMDA ratio recordings, coronal slices (300 μm thick) were prepared in an oxygenated cutting solution at 4° C. by using a vibratome (Leica). The cutting solution contained (in mM): 30 NaCl, 4.5 KCI, 1.2 NaH₂PO₄, 194 sucrose, 26 NaHCO₃, 10 D-glucose, 0.2 CaCl₂, 8 MgSO₄,and saturated with 95% O₂-5% CO₂(pH 7.3, osmolarity of 350 mOsm). Slices were recovered in ACSF at 33° C. (+/−0.5° C.) for 15 min and then kept at room temperature for 1 hr before recordings. The ACSF contained (in mM): 119 NaCl, 2.3 KCl, 2.5 CaCl₂, 1.3 MgSO₄, 26.2 NaHCO₃, 1 NaH₂PO₄, 11 D-glucose, and saturated with 95% O₂-5% CO₂ (pH 7.3, osmolarity of 300 ). For all other recordings, the brain was quickly removed and placed in ice-cold ACSF consisting of (in mM): 125 NaCl, 3 KCl, 1.25 NaH₂PO₄, 2 MgSO₄, 2 CaCl₂, 25 NaHCO₃, and 10 D-glucose. Slices were stored for 30 min at 33° C. (+/−0.5° C.) and then kept at room temperature until recording.

Electrophysiological recordings. Whole cell recordings in current clamp- or voltage clamp-mode were performed using an IR-DIC microscope (Olympus) with a water immersion 40× objective (NA 0.8), equipped with four automatic manipulators (Luigs and Neumann) and a CCD camera (Hamamatsu Co). For all recordings, borosilicate glass pipettes were fabricated (Sutter Instrument) with resistances of 3.5 to 5 MSΩ. The AMPA/NMDA ratio measurements were performed by adding 100 μM picrotoxin (Tocris) in the extracellular solution, and voltage clamp recordings were performed using the following intracellular solution (in mM): 120 cesium methansulfonate. 10 HEPES, 1.1 EGTA, 5 NaCl, 1.1 TEA-Cl, 4 Mg-ATP, 0.3 Na-GTP. 4 QX314, and 0.5% biocytin. The osmolarity of this intracellular solution was 298 mOsm and the pH was 7.2. AMPA/NMDA ratio is defined as the ratio of the EPSC peak at -70 mV to the EPSC magnitude at +40 mV (50 ms following stimulation). To measure calcium currents (('aV2.1, CaV2.2), patch pipettes were filled with a solution containing the following (in mM): 103 CsCl, 12 CsOH, 12 methanesulfonic acid, 4 NaCl. 5 TEA-C1. 10 HEPES. 0.5 EGTA. 10 phosphocreatine, 5 lidocaine N-ethyl chloride, 4 ATP magnesium salt, and 0.3 GTP sodium salt. pH was adjusted to 7.2−7.4 with KOH, and osmolarity was adjusted to 298−300 mOsm with K₂SO₄. Neurons were held at −80 mV and stepped from −60 mV to +20 mV in 10 mV increments, in the presence of TTX (1 μM), picrotoxin (100 μM), 4AP (1 mM), tetraethylammonium chloride (10 mM), and cesium chloride (2 mM). Calcium currents were recorded before and after application of ω-Conotoxin GVIA (200 nM), and further addition of ω-Agatoxin IVA (100 nM), CaV2.1 currents were the component blocked by @-Agatoxin IVA, while (aV2.2 currents were the component blocked by @-Conotoxin GVIA. For other recordings, pipettes were filled with the following intracellular solution (in mM): 110 K-gluconate, 40 KC;, 10 HEPES. 3 ATP. 0.5 GTP. 0.2 EGTA, and 0.5% biocytin. The osmolarity of this intracellular solution was 290 mOsm and the pH was 7.25. To measure potassium current (Kir 2.2), neurons were held at −60 mV and stepped to −140 mV in 10 mV increments, in the presence of TTX (1 μM), before and after Ba2 (10 μM) application. Kir 2.2 currents were the component blocked by Ba²⁺. For mEPSC recordings, neurons were clamped at −70 mV in the presence of TTX (1 μM) and picrotoxin (100 μM). Synaptic currents were analyzed with the Mini Analysis Program (Synaptosoft). A series of 500 ms suprathreshold currents of 50−300 pA were used to quantify the excitability with holding at −55 mV. Membrane time constant (tau) was measured with a single exponential fit of the voltage deflection produced by a small hyperpolarizing current injection from the holding potential (−70 mV). Input resistance (Rᵢₙ) was calculated as the slope of linear fits of current-voltage plots generated from a series of increasing current injection steps. Shape parameters were measured from the first action potential with 200 ms current injection (from the holding potential of −70 mV). In-induced sag currents were evoked by brief injections of hyperpolarizing currents in current clamp mode. To compare sag amplitudes between different groups,amplitudes of the current injections were adjusted in each cell to result in the same peak hyperpolarization, and the sag amplitude was determined as the repolarization from the peak to a steady state, during the entire length of current injection. Recordings were amplified using up to two dual channel amplifiers (Molecular Devices), filtered at 2 KHz. digitized (20 kHz), and acquired through an ADC/DAC data acquisition unit (Instrutech) using custom software running on Igor Pro (Wavemetrics). Access resistance (RA) was monitored throughout the duration of the experiment and data acquisition was suspended whenever RA was beyond 20 MSΩ. For recordings after CFC training, anesthesia and slice preparation was initiated 45 min after the behavioral epoch. For recordings related to the excitability rescue in KD mice. C21 was injected IP 1 hr prior to anesthesia and slice preparation.

Optogenetic stimulation during recordings. Optogenetic stimulation was achieved through Polygon400 (Mightex) with built-in LED sources (470) nm or 590 nm). Light power on the sample was 20 mW/mm². To test ChR2 expression, slices were stimulated with 5 Hz blue light pulses. To test NpHR function. continuous green light was delivered to the slices. To test synaptic connections, slices were stimulated with a single light pulse of 1 s, repeated 10 times every 5 s, and the average response was computed. The monosynaptic glutamatergic nature of a connection was confirmed by sequential bath application of I uM TTX (Tocris). 100 μM 4AP (Tocris), and 10 μM CNQX (Tocris). Paired-pulse ratio refers to the ratio of the peak of the second EPSC to the peak of the first EPSC using a 50 ms interstimulus interval. Post-hoc immunohistochemistry. Recorded cells were filled with biocytin and subsequently recovered for brain region and/or cell type verification. Slices were first incubated with 4% PFA for 16 hr at 4° C. After washing with 0.5% Triton X−100 in PBS, slices were incubated in 5% normal goat serum for 2 hr. Following serum, slices were incubated in streptavidin CF555 (1:200. Biotium) for 2 hr at room temperature. Before mounting, slices were incubated with DAPI (1:3000) for 30 min.

### In Vivo LFP Recordings

Surgical procedure. C57BL/6J male mice (25−35 g, 10−16 weeks of age. Jackson Laboratory) were used for LFP recordings using chronically implanted electrodes. Mice were group housed before implantation surgeries but housed individually after in order to minimize damage to the implants. Animals were initially anesthetized with 5% isoflurane and maintained under anesthesia with 1−2% isoflurane during surgery. Implantable LFP electrodes made by teflon-coated tungsten microwires (50 μm, A-M Systems) were targeted to AD (−0.7mm AP, +/−0.75 mm ML, −2.75 mm DV), RSC (−2 mm AP, +/−0.25 mm ML, −1.1 mm DV), and PreSub (−3.8 mm AP, +/−1.75 mm ML, −1.7 mm DV). LFP electrodes were coated with Dil555 (Thermo Fisher Scientific) prior to implantation, which provided a fluorescent track for post-hoc electrode tip verification in brain sections. The reference and ground screws with wire lead (Pinnacle Technology) were targeted to the occipital skull. All electrodes were secured with dental cement and connected to a headmount (Pinnacle Technology) in combination with EMG leads for detecting sleep-wake status during LFP recordings. Animals recovered for at least 10 days post-surgery before LFP recordings during behavior. For pre vs. post CFC training LFP recordings, mice that were previously being recorded in their home cages were carefully unplugged, followed by behavioral procedures. Immediately after behavior (˜5 min later), mice were plugged back into the LFP recording system for data collection. For circuit recordings, electrodes were targeted to AD and RSC or AD and PreSub on one hemisphere. As criteria, the inventors only included mice with DiI555 electrode tip staining limited to the targeted regions.

Data acquisition and processing. LFP signals were amplified, digitized continuously at 1 kHz using a tethered recording system with a differential amplifier (Pinnacle Technology) in awake, freely moving mice, and acquired (Pinnacle Sirenia acquisition software) for offline analysis using MATLAB (MathWorks). Spectral power was calculated in 0.5 Hz bins (fast Fourier transform with Hamming windows) with artifact-free LFP signals based on the following frequency bands: delta (1−4 Hz), theta (6−10 Hz), beta (12−30 Hz), and gamma (30)-100 Hz). The coherence between two signals x(t) and y(t) were calculated as a function of the power spectral density of x and y (Pₓₓ and P_{yy}), and the cross power spectral density of x and y (P_{xy}) with values between 0 and 1 for verifying x and y correspondence at each frequency. Inter-regional (AD→RSC) cross-frequency phase-amplitude coupling was calculated as previously described (Tort et al., 2010). The modulation index (MI) is a measure of the magnitude with which the phase of low-frequency rhythms (1−12 Hz) modulates the amplitude of high-frequency rhythms (20−100 Hz). MI was evaluated in 1 Hz frequency bins. Instantaneous phase and amplitude time series data were calculated by Hilbert transformation of band-pass-filtered LFP signals (zero phase filtering with a finite impulse response (FIR) filter of order 60, 3- and 2-Hz bandwidths for phase and amplitude frequencies, respectively).

### Behavior Assays

Experiments were conducted during the light cycle (7 am to 7 pm). Mice were randomly assigned to experimental groups for specific behavioral assays immediately after surgery. Mice were habituated to investigator handling for 1−2 minutes on three consecutive days. Handling took place in the holding room where the mice were housed. Prior to each handling session, mice were transported by wheeled cart to and from the vicinity of the behavior rooms to habituate them to the journey. All behavior experiments were analyzed blind to experimental group. Unpaired student's t-tests were used for independent group comparisons. with Welch's correction when group variances were significantly different, or ANOVA followed by Bonferroni post-hoc tests were used. Given behavioral variability, assays were performed using a minimum of 6−10 mice per group to ensure adequate power for any observed differences. Following behavioral protocols, brain sections were prepared to confirm efficient viral labeling in target areas. Animals lacking adequate labeling were excluded prior to behavior quantification.

Open field exploration. Spontaneous motor activity was measured in an open field arena (40×40×30 cm) for 20 min. Mice were transferred to the testing room and acclimated for 30 min before the test session. During the testing period, lighting in the room was turned off. The apparatus was cleaned with quatricide before and between runs. Total movement (distance traveled) in the arena was quantified using an automated infrared (IR) detection system (Omnitech Digiscan. AccuScan Instruments). To test the effect of different optogenetic manipulations on locomotion, mice were plugged into the laser source and light was turned on once the animals were placed into the arena. Recordings were performed for 10 min. Raw data were extracted and analyzed using Microsoft Excel. Rotarod motor coordination. Controlled motor coordination was measured in a rotarod apparatus (Med Associates). Mice were transferred to the testing room and acclimated for 15 min before the test session. Mice were placed on the rod, which accelerated from 4−40 r.p.m., until they fell (this time was provided by the apparatus and recorded as latency to fall for each trial). Each mouse was tested for three trials in a single day, with about 15 min between trials. Raw data were recorded and analyzed using Microsoft Excel.

Contextual fear conditioning. Two distinct contexts were employed. The conditioning context was a 29×25×22 cm chamber with grid floors, dim white lighting, and scented with 0.25% benzaldehyde. The neutral context consisted of a 29×25×22 cm chamber with white perspex floors, red lighting, and scented with 1% acetic acid. All mice were conditioned (120) s exploration, one 0.65 mA shock of 2 s duration at 120 s, 60 s post-shock period., second 0.65 mA shock of 2 s duration at 180 s, 60 s post-shock period), and tested (3 min) one day later. Twenty-four hours after the recall test on day 2, the neutral context test (3 min) was performed (i.e., neutral context tests were always on day 3). Experiments showed no generalization in the neutral context for wild type/control mice. Floors of chambers were cleaned with quatricide before and between runs. Mice were transported to and from the experimental room in their home cages using a wheeled cart. For immediate shock controls, animals were placed in the conditioning chamber, received a 2 s foot shock after the first 5 s and then were immediately removed from the chamber. For experiments that included optogenetic manipulations, the behavior chamber ceilings were customized to hold a rotary joint (Doric Lenses) connected to two 0.3 m optic fibers. All mice had optic fibers attached to their optic fiber implants prior to training and recall tests. Since optogenetic manipulations (i.e., optic fibers) interfered with automated motion detection, freezing behavior was manually quantified for all experiments.

Inhibitory avoidance. A 29×25×22 cm unscented chamber with square ceilings and intermediate lighting was used. The chamber consisted of two sections, one with grid flooring and the other with a white platform. During the training session (1 min), mice were placed on the white platform, which is the less preferred section of the chamber (relative to the grid section). Once mice entered the grid section of the chamber (all four feet), 0.65 mA shocks of 2 s duration were delivered. On average, each mouse received 2−3 shocks per training session. After 1 min, mice were returned to their home cage. The next day, total time on the white platform was manually quantified (3 min test).

Innate avoidance. Innate avoidance behavior in response to 2,3,5-trimethyl−3-thiazoline (TMT), a component of fox feces, was measured. Mice were placed in the center of a 40×30 cm Plexiglass arena, which contained four small dishes (3 cm diameter) in each of the corners. Mice were first habituated to the arena for 10 min. During trial 1, mice were allowed to explore the arena in which all four dishes contained 1× PBS (0.5 ml each) for 15 min. The preferred corner was recorded for the subsequent trial for each mouse. Approximately 30 min after trial 1, mice were returned to the arena in which their preferred corner now had 5% TMT (colorless) instead of 1× PBS (trial 2). Mice were once again allowed to explore the area for 15 min, after which they were returned to their home cages. Relative to the time spent in their preferred corner during trial 1, time spent in this same corner during trial 2 was manually quantified (i.e., avoidance behavior). The arena was rotated between mice, and to make sure that the TMT odor did not persist between mice these tests were performed in the fume hood.

Tone fear conditioning. The conditioning context was a 29×25×22 cm chamber with grid floors, bright white lighting, and scented with 1% acetic acid. The recall test context consisted of a 30×25×33 cm chamber with white perspex floors, red lighting, and scented with 0.25% benzaldehyde. Mice were conditioned (120 s exploration. 10 s tone co-terminating with a 0.65 mA shock of 2 s duration. 60 s post-shock period, repeated 2 more times). Memory recall was tested (1 min exploration. 60 s ton, 60 s post-tone period. repeated 2 more times) one day later. The tone was calibrated to 75 dB SPL, with a frequency of 5 kHz. Experiments showed no generalization in the recall test context during the initial exploration period. Freezing behavior was manually quantified.

Spatial working memory: T-maze. For spatial working memory behavior, the inventors used the delayed non-match-to-place (DNMP) T-maze protocol. Mice selected for this paradigm were food deprived until they reached 85% of their initial body weight. During food deprivation. mice were habituated to the sugar pellets (20 mg), which would subsequently be used as a reward in the T-maze. Mice were habituated to the T-maze for 10 min. During habituation to the maze, sugar pellet rewards were placed in the reward cups (2.5 cm diameter) at the end of each arm, and were replaced as they were consumed. The behavioral training consisted of ten trials per day with each trial having two separate runs (Sample and Choice runs). The first run in each trial was the Sample run, in which mice were placed in the stem of the T-maze and allowed to run to the end of one arm of the maze (the other arm was closed off). This open arm was rewarded. After reward consumption. mice were returned to their home cage for ˜30 s when the T-maze was quickly cleaned and both arms were opened. Mice were once again placed in the stem of the T-maze and during this Choice run mice were allowed to choose which of the two arms to visit. The opposite arm from the one visited during the previous Sample run was rewarded. If the mouse chose the incorrect arm (i.e., the previous arm), it was blocked in that arm for a 30 s punishment. Following this Sample and Choice run procedure for a single trial, each mouse performed nine more trials per day with an inter-trial interval of 20 min. Mice were manually scored on the percentage of time that they made a successful alternation and how many days until they reached a daily success rate of over 70% for two consecutive days (referred to as days to criterion). Once they reached criterion, the next two days were used for testing animals' success rate when the delay between Sample and Choice runs was 10 s (ten trials per day). Their performance in the 10 s delay condition was an average of these two test days. Similarly, the following two days were used for testing animals' success rate when the delay between Sample and Choice runs was 60 s (ten trials per day), which was a more demanding version of this task.

Cocaine-induced conditioned place preference. The conditioned place preference (CPP) behavior chamber was a rectangular arena (42×15 cm), divided into three quadrants (left. middle, right). The left and right quadrants were 15 cm long. while the middle quadrant was 12 cm long. The left quadrant had wide grid floors and a pattern (series of parallel lines) on the wall. The right quadrant had white smooth polypropylene floors and a pattern (series of circles) on the wall. On day 1 (pre-exposure), mice were allowed to explore the entire arena for 30 min. Experiments showed no preference to any one quadrant. On day 2 (training), mice were confined to the left or right quadrants for 10 min following cocaine (20 mg kg⁻¹) or saline intraperitoneal administration in addition to receiving optogenetic light activation for the entire session. This 10 min session was repeated twice with an inter-trial interval of 3 hr. On days 3-7 (training continued), mice were conditioned in opposite quadrants in an alternating manner (i.e., cocaine left-saline right-cocaine left, etc) until every mouse received 3 cocaine- and 3 saline-pairing days. For every behavioral cohort, half the mice were conditioned with cocaine in the left quadrant, while the remaining mice received cocaine in the right quadrant. On day 8, memory recall was measured by preference to the left or right quadrant (10 min), without optogenetic light activation. All sessions were performed with dim white lighting. On day 9, generalization behavior was measured using a modified CPP chamber (10 min), without optogenetic light activation. Specifically, in this modified chamber, the left quadrant had thin grid floors and a pattern (series of squares) on the wall. while the right quadrant had smooth black floors and a pattern (series of triangles) on the wall, and the room had bright white lighting. Mouse behavior, specifically position tracking and duration, was recorded using an automated infrared (IR) detection system (EthoVision XT. Noldus). Raw data were extracted and analyzed using Microsoft Excel.

### QUANTIFICATION AND STATISTICAL ANALYSIS

Data are presented as mean values accompanied by SEM. No statistical methods were used to predetermine sample sizes. Data analysis was performed blind to the conditions of the experiments. Data were analyzed using Microsoft Excel with the Statplus plug-in and Prism 6 software. One-way ANOVA followed by Bonferroni post-hoc tests, two-way ANOVA with repeated measures followed by Bonferroni post-hoc tests, two-tailed unpaired/tests, and paired/tests were used to test for statistical significance when appropriate. Statistical parameters including the exact value of n, precision measures (mean ±SEM), and statistical significance are reported in each main and supplementary FIG. legend. The significance threshold was placed at a=0.05 (NS, p→0.05; * p<0.05; ** p<0.01; *** p<0.001).

### REFERENCES

Aida, T., Chiyo, K., Usami, T., Ishikubo, H., Imahashi, R., Wada, Y., Tanaka, K. F., Sakuma, T., Yamamoto, T., and Tanaka, K. (2015). Cloning-free CRISPR/Cas system facilitates functional cassette knock-in in mice. Genome Biol. 16, 87.
Aggleton, J. P., Keith, A. B., and Sahgal, A. (1991). Both fornix and anterior thalamic, but not mammillary, lesions disrupt delayed non-matching-to-position memory in rats. Behav. Brain Res. 44, 151-161.
Challis, R. C., Kumar, S. R., Chan, K. Y., Challis, C., Beadle, K., Jang, M. J., Kim, H. M., Rajendran, P. S., Tompkins, J. D., Shivkumar, K., et al. (2019). Systemic AAV vectors for widespread and targeted delivery in rodents. Nat. Protoc. 14, 379-414.
Chatterjee, S., Sullivan, H. A., MacLennan, B. J., Xu, R., Hou, Y., Lavin, T. K., Lea, N. E., Michalski, J. E., Babcock, K. R., Dietrich, S., et al. (2018). Nontoxic, double-deletion-mutant rabies viral vectors for retrograde targeting of projection neurons. Nat. Neurosci. 21, 638-646.
Chaudhry, A., Noor, A., Begagne, B., Baker, K., Bok, L. A., Brady, A. F., Chitayat, D., Chung, B. H., Cytrynbaum, C., Dyment, D., et al. (2015). Phenotypic spectrum associated with PTCHD1 deletions and truncating mutations includes intellectual disability and autism spectrum disorder. Clin. Genet. 88, 224-233.
Colgin, L. L. (2015). Theta-gamma coupling in the entorhinal-hippocampal system. Curr. Opin. Neurobiol. 31, 45-50.
Doench, J. G., Fusi, N., Sullender, M., Hegde, M., Vaimberg, E. W., Donovan, K. F., Smith, I., Tothova, Z., Wilen, C., Orchard, R., et al. (2016). Optimized sgRNA design to maximize activity and minimize off-target effects of CRISPR-Cas9. Nat. Biotechnol. 34, 184-191.
Golden, C. E. M., Buxbaum, J. D., and De Rubeis, S. (2018). Disrupted circuits in mouse models of autism spectrum disorder and intellectual disability. Curr. Opin. Neurobiol. 48, 106-112.
Gruter, T., Wiescholleck, V., Dubovyk, V., Aliane, V., and Manahan-Vaughan, D. (2015). Altered neuronal excitability underlies impaired hippocampal function in an animal model of psychosis. Front. Behav. Neurosci. 9, 117.
Guenthner, C. J., Miyamichi, K., Yang, H. H., Heller, H. C., and Luo, L. (2013). Permanent genetic access to transiently active neurons via TRAP: targeted recombination in active populations. Neuron 78, 773-784.
Jankowski, M. M., Ronnqvist, K. C., Tsanov, M., Vann, S. D., Wright, N. F., Erichsen, J. T., Aggleton, J. P., and O'Mara, S.M . (2013). The anterior thalamus provides a subcortical circuit supporting memory and spatial navigation. Front. Syst. Neurosci. 7, 45.
Kasten, M. R., Rudy, B., and Anderson, M. P. (2007). Differential regulation of action potential firing in adult murine thalamocortical neurons by Kv3.2, Kvl, and SK potassium and N-type calcium channels. J. Physiol. 584, 565-82.
Kvajo, M., Mckellar, H., Arguello, P. A., Drew, L. J., Moore, H., MacDermott, A. B., Karayiorgou, M., and Gogos, J. A. (2008). A mutation in mouse Discl that models a schizophrenia risk allele leads to specific alterations in neuronal architecture and cognition. Proc. Natl. Acad. Sci. 105, 7076-7081.
Lein, E. S., Hawrylycz, M. J., Ao, N., Ayres, M., Bensinger, A., Bernard, A., Boe, A. F., Boguski, M. S., Brockway, K. S., Byrnes, E. J., et al. (2007). Genome-wide atlas of gene expression in the adult mouse brain. Nature 445, 168-176.
Li, Y., Lopez-Huerta, V. G., Adiconis, X., Levandowski, K., Choi, S., Simmons, S.K., Arias-Garcia, M. A., Guo, B., Yao, A. Y., Blosser, T. B., et al. (2020). Distinct subnetworks of the thalamic reticular nucleus. Nature 583, 819-824.
Matson, J. L., and Shoemaker, M. (2009). Intellectual disability and its relationship to autism spectrum disorders. Res. Dev. Disabil. 30, 1107-1114.
Mitchell, A. S., and Dalrymple-Alford, J. C. (2006). Lateral and anterior thalamic lesions impair independent memory systems. Learn. Mem. 13, 388-396.
Morgan, V. A., Leonard, H., Bourke, J., and Jablensky, A. (2008). Intellectual disability co-occurring with schizophrenia and other psychiatric illness: population-based study. Br. J. Psychiatry 193, 364-372.
Nakajima, M., Schmitt, L. I., Feng. G., and Halassa, M. (2019). Combinatorial targeting of distributed forebrain networks reverses noise hypersensitivity in a model of autism spectrum disorder. Neuron 104, 488-500.
O'Tuathaigh, C. M., Babovic, D., O'Sullivan, G. J., Clifford, J. J., Tighe, O., Croke, D. T., Harvey, R., and Waddington, J. L. (2007). Phenotypic characterization of spatial cognition and social behavior in mice with ‘knockout’ of the schizophrenia risk gene neuregulin 1. Neuroscience 147, 18-27.
Saunders, A., Johnson, C. A., and Sabatini, B. L. (2012). Novel recombinant adeno-associated viruses for Cre activated and inactivated transgene expression in neurons. Front. Neural Circuits 6, 47.
Saunders, A., Macosko, E. Z., Wysoker, A., Goldman, M., Krienen, F. M., de Rivera, H., Bien, E., Baum, M., Bortolin, L., Wang. S., et al. (2018). Molecular diversity and specializations among the cells of the adult mouse brain. Cell 174, 1015-1030.
Savage, L. M., Hall, J. M., and Vetreno, R. P. (2011). Anterior thalamic lesions alter both hippocampal-dependent behavior and hippocampal acetylcholine release in the rat. Learn. Mem. 18. 751-758.
Singh, T., Neale, B. M., Daly, M., and the Schizophrenia Exome Meta-Analysis (SCHEMA) Consortium. (2020). Exome sequencing identifies rare coding variants in 10 genes which confer substantial risk for schizophrenia. medRxiv 20192815 [Preprint]. 18 September 2020.
Speca, D. J., Ogata, G., Mandikian, D., Bishop, H. I., Wiler, S. W., Eum, K., Wenzel. H.J ., Doisy, E. T., Matt. L., Campi, K. L., et al. (2014). Deletion of the Kv2.1 delayed rectifier potassium channel leads to neuronal and behavioral hyperexcitability. Genes Brain Behav. 13, 394-408.
Tervo, D. G. R., Hwang. B. Y., Viswanathan, S., Gaj, T., Lavzin, M., Ritola, K. D., Lindo, S., Michael. S., Kuleshova, E., Ojala. D., et al. (2016). A designer AAV variant permits efficient retrograde access to projection neurons. Neuron 92, 372-382. 20)
Tort, A. B., Komorowski, R., Eichenbaum, H., and Kopell, N. (2010). Measuring phase-amplitude coupling between neuronal oscillations of different frequencies. J. Neurophysiol. 104,1195-1210.
Vertes, R. P., Linley, S. B., Groenewegen, H. J., and Witter, M. P. (2015). “Thalamus” in The Rat Nervous System, Paxinos, G., Ed. (Academic Press).
Vetere, G., Xia. F., Ramsaran, A. I., Tran, L. M., Josselyn, S. A., and Frankland, P. W. (2021). An inhibitory hippocampal-thalamic pathway modulates remote memory retrieval. Nat. Neurosci. 24, 685-693.
Warburton, E. C., and Aggleton, J. P. (1999). Differential deficits in the Morris water maze following cytotoxic lesions of the anterior thalamus and fornix transection. Behav. Brain Sci. 98. 27-38. 35
Wells, M. F., Wimmer, R. D., Schmitt, L. I., Feng, G., and Halassa, M. (2016). Thalamic reticular impairment underlies attention deficit in Ptchd1Y/- mice. Nature 532, 58-63.
Wickersham, I. R., Lyon, D. C., Barnard, R. J. O., Mori, T., Finke, S., Conzelmann, K. K., Young, J. A. T., and Callaway, E. M. (2007). Monosynaptic restriction of transsynaptic tracing from single, genetically targeted neurons. Neuron 53, 639-647.
Winter, S. S., Clark, B. J., and Taube, J. S. (2015). Disruption of the head direction cell network impairs the parahippocampal grid cell signal. Science 347, 870-874.
Witter, M. P., Doan, T. P., Jacobsen, B., Nilssen, E.S ., and O'Mara, S. (2017). Architecture of the entorhinal cortex: a review of entorhinal anatomy in rodents with some comparative notes. Front. Syst. Neurosci. 11, 46.
Xu, W., and Sudhof, T. C. (2013). A neural circuit for memory specificity and generalization. Science 339, 1290-1295.
Xu, J., Bartolome, C. L., Low, C. S., Yi, X., Chien, C. H., Wang, P., and Kong, D. (2018) Genetic identification of leptin neural circuits in energy and glucose homeostasis. Nature 556, 505-509.
Yamawaki, N., Li, X., Lambot, L., Ren, L. Y., Radulovic, J., and Shepherd, G. (2019). Long-range inhibitory intersection of a retrosplenial thalamocortical circuit by apical tuft-targeting CA1 neurons. Nat. Neurosci. 22, 618-626.
Ye, L., Allen, W. E., Thompson, K. R., Tian, Q., Hsueh, B., Ramakrishnan, C., Wang, A. C., Jennings, J. H., Adhikari, A., Halpern, C., et al. (2016). Wiring and molecular features of prefrontal ensembles representing distinct experiences. Cell 165, 1776-1788.
Young, K. A., Manaye, K. F., Liang, C., Hicks, P. B., and German, D. C. (2000). Reduced number of mediodorsal and anterior thalamic neurons in schizophrenia. Biol. Psychiatry 47, 944-953.
Zingg, B., Chou, X. L., Zhang, Z. G., Mesik, L., Liang, F., Tao, H. W., and Zhang, L. I. (2017). AAV-mediated anterograde transsynaptic tagging: mapping corticocollicular input-defined neural pathways for defense behaviors. Neuron 93, 33-47.

### OTHER EMBODIMENTS

From the foregoing description, it will be apparent that variations and modifications may be made to embodiments of the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for increasing cognitive performance, the method comprising:
administering to a subject a recombinant adeno-associated virus (rAAV) comprising a sequence encoding an engineered M4 muscarinic acetylcholine receptor, and expressing the receptor in neurons of the subject that inhibit anterodorsal thalamus hyperexcitability,
thereby increasing cognitive performance in the subject.

2. A method for reducing cognitive impairment associated with a neuropsychiatric disorder in a subject, the method comprising:
administering to a subject a recombinant adeno-associated virus (rAAV) comprising a sequence encoding an engineered M4 muscarinic acetylcholine receptor, and expressing the receptor in neurons of the subject that inhibit anterodorsal thalamus hyperexcitability,
thereby reducing cognitive impairment in the subject.

3. The method of claim 1, wherein the neurons project to the retrosplenial cortex.

4. The method of claim 1, wherein the neurons are neurons of the anterodorsal (AD) thalamus, anteroventral (AV) thalamus, or entorhinal cortex neurons.

5. The method of claim 1, wherein the receptor is expressed in neurons present in the anterodorsal thalamus →retrosplenial cortex (RSC) circuit, thereby enhancing memory encoding.

6. The method of claim 1, wherein the receptor is expressed in neurons present in the AV→RSC circuit, thereby enhancing memory specificity.

7. The method of claim 1, wherein the subject has, is suspected of having, or is at risk of developing a neuropsychiatric disorder.

8. The method of claim 7, wherein neuropsychiatric disorder is autism, schizophrenia, or a disorder associated with an alteration in the sequence, activity, or expression of PTCHD1, YWHAG, or HERC1.

9. The method of claim 1, further comprising administering to the individual an agonist of the receptor.

10. A method for increasing cognitive performance, the method comprising:
administering to a subject a recombinant adeno-associated virus (rAAV) comprising a promoter expressed in a neuron selected from the group consisting of neurons of the anterodorsal (AD) thalamus, anteroventral (AV) thalamus, entorhinal cortex, neurons present in the anterodorsal thalamus →retrosplenial cortex (RSC) circuit, and neurons present in the AV→RSC circuit,
wherein the promoter is operatively linked to a polynucleotide encoding an hM4Di polypeptide, and
administering to the subject a ligand of hM4Di polypeptide,
such that the hM4Di polypeptide expression inhibits anterodorsal thalamus hyperexcitability,
thereby increasing cognitive performance in the subject.

11. A method for reducing cognitive impairment associated with a neuropsychiatric disorder in a subject, the method comprising:
administering to a subject a recombinant adeno-associated virus (rAAV) comprising a promoter expressed in a neuron selected from the group consisting of neurons of the anterodorsal thalamus, anteroventral thalamus, entorhinal cortex, neurons present in the anterodorsal thalamus →retrosplenial cortex (RSC) circuit, and neurons present in the AV→RSC circuit,
wherein the promoter is operatively linked to a polynucleotide encoding an hM4Di polypeptide, and
administering to the subject a ligand of hM4Di polypeptide,
such that the hM4Di polypeptide expression inhibits anterodorsal thalamus hyperexcitability,
thereby reducing cognitive impairment in the subject.

12. The method of claim 1, wherein the rAAV comprises a polynucleotide encoding a Cre-dependent inhibitory Designer Receptors Exclusively Activated by Designer Drugs (DREADD) hM4Di.

13. The method of claim 1, wherein the ligand is compound 21 (C21).


SEQUENCE LISTING (32 sequences)

SEQ ID NO: 1 - protein, 552 aa, synthetic construct
  REGION 1..552: note = Description of Artificial Sequence: Synthetic polypeptide
MANFTPVNGSSGNQSVRLVTSSSHNRYETVEMVFIATVTGSLSLVTVVGNILVMLSIKVNRQLQTVNNYFLFSLACADLIIGAFSMNLYTVYIIKGYWPLGAVVCDLWLALDCVVSNASVMNLLIISFDRYFCVTKPLTYPARRTTKMAGLMIAAAWVLSFVLWAPAILFWQFVVGKRTVPDNQCFIQFLSNPAVTFGTAIAGFYLPVVIMTVLYIHISLASRSRVHKHRPEGPKEKKAKTLAFLKSPLMKQSVKKPPPGEAAREELRNGKLEEAPPPALPPPPRPVADKDTSNESSSGSATQNTKERPATELSTTEATTPAMPAPPLQPRALNPASRWSKIQIVTKQTGNECVTAIEIVPATPAGMRPAANVARKFASIARNQVRKKRQMAARERKVTRTIFAILLAFILTWTPYNVMVLVNTFCQSCIPDTVWSIGYWLCYVNSTINPACYALCNATFKKTFRHLLLCQYRNIGTARYPYDVPDYAYPYDVPDYAYKYRNRDEGSYHVDESRNYISNSAQSNGAVVKEKQPSSAKSANKNKKNKDKEYYV

SEQ ID NO: 2 - DNA, 1659 bp, synthetic construct
  misc_feature 1..1659: note = Description of Artificial Sequence: Synthetic polynucleotide
  source 1..1659: mol_type = other DNA
atggcgaacttcacgcctgtcaatgggagctccggtaatcagtcggtacggctcgtgacgtcgtcctcgcacaatcgctacgaaacagtagaaatggtattcatcgccacagtaactggaagcctttcacttgtgactgtcgtgggaaacattctggtcatgctctcgattaaggtcaacaggcaactccagaccgtaaacaactatttcttgttctccctggcttgcgcggatctgatcattggggctttctcgatgaacctctatacagtatacatcatcaaagggtactggcctctcggagccgtcgtgtgtgacctctggcttgcacttgactgtgtggtgagcaatgcttcagtcatgaaccttctgattatctcatttgatcgctatttctgtgtaaccaaacccttgacctaccccgcaaggagaacgaccaaaatggcagggctgatgatcgcggctgcatgggtcctctcgttcgtactttgggctcctgccattctgttctggcagtttgtcgtgggtaaacgcacagtcccggacaaccaatgtttcatccaattcctttcgaaccccgctgtgactttcgggactgcgattgcgggattctaccttccggtcgtcattatgaccgtgttgtacattcacatctcgcttgcgtcaagaagccgcgtccacaagcacaggcccgaggggcctaaggagaagaaagccaagacattggcttttctcaaatcaccgcttatgaaacagtccgtgaaaaagcctcctcccggtgaagcagcgcgcgaggagctgaggaatggtaaactggaagaggcgccacctccggcgctgcctcctccaccgcgacccgtggcggataaggacacttccaacgagtcgtcgtcggggagcgcgacccagaatacgaaagagcgacctgctaccgagttgagcacaacagaagcgacgacgccggcaatgccggcaccgcctcttcagcccagggcgctgaaccccgcgtcgcgatggtcaaagattcagattgtgacgaaacaaacgggtaatgagtgcgtaacagccatcgagatcgtacccgcaacgcccgctggtatgaggccagccgccaacgtcgccagaaagttcgcgtcaatcgcgcggaatcaggtgcggaagaaaagacaaatggccgcgagagagagaaaggtgacaagaacgattttcgccatcctcctggcgtttatcttgacgtggactccgtataatgtcatggtgttggtgaatacattttgtcaatcgtgcatcccagacacggtctggtcgatcgggtattggctctgctacgtcaactccactatcaatccggcatgctacgcattgtgtaacgccacgttcaagaaaacgttccgccatctcctgttgtgccagtaccgaaatatcgggacagcgagatacccatatgatgtccccgattacgcctatccctatgacgtcccggattacgcttacaagtatcggaaccgggacgaaggctcgtatcatgtggacgagtcacggaactatatctcgaactccgcccagtcaaacggagcggtggtgaaggaaaagcagccctcatcggcgaagtcagcgaataagaacaagaagaataaggacaaagagtactatgtgtga

SEQ ID NO: 3 - protein, 888 aa, Homo sapiens
MLRQVLHRGLRTCFSRLGHFIASHPVFFASAPVLISILLGASFSRYQVEESVEHLLAPQHSLAKIERNLVNSLFPVNRSKHRLYSDLQTPGRYGRVIVTSFQKANMLDQHHTDLILKLHAAVTKIQVPRPGFNYTFAHICILNNDKTCIVDDIVHVLEELKNARATNRTNFAITYPITHLKDGRAVYNGHQLGGVTVHSKDRVKSAEAIQLTYYLQSINSLNDMVAERWESSFCDTVRLFQKSNSKVKMYPYTSSSLREDFQKTSRVSERYLVTSLILVVTMAILCCSMQDCVRSKPWLGLLGLVTISLATLTAAGIINLTGGKYNSTFLGVPFVMLGHGLYGTFEMLSSWRKTREDQHVKERTAAVYADSMLSFSLTTAMYLVTFGIGASPFTNIEAARIFCCNSCIAIFFNYLYVLSFYGSSLVFTGYIENNYQHSIFCRKVPKPEALQEKPAWYRFLLTARFSEDTAEGEEANTYESHLLVCFLKRYYCDWITNTYVKPFVVLFYLIYISFALMGYLQVSEGSDLSNIVATATQTIEYTTAQQKYFSNYSPVIGFYIYESIEYWNTSVQEDVLEYTKGFVRISWFESYLNYLRKLNVSTGLPKKNFTDMLRNSFLKAPQFSHFQEDIIFSKKYNDEVDVVASRMFLVAKTMETNREELYDLLETLRRLSVTSKVKFIVFNPSFVYMDRYASSLGAPLHNSCISALFLLFFSAFLVADSLINVWITLTVVSVEFGVIGFMTLWKVELDCISVLCLIYGINYTIDNCAPMLSTFVLGKDFTRTKWVKNALEVHGVAILQSYLCYIVGLIPLAAVPSNLTCTLFRCLFLIAFVTFFHCFAILPVILTFLPPSKKKRKEKKNPENREEIECVEMVDIDSTRVVDQITTV

SEQ ID NO: 4 - DNA, 12883 bp, Homo sapiens
  source 1..12883: mol_type = genomic DNA
ctcctcccgcgcccgctctgctctaggatgctgcggcaggttctgcacaggggcttgaggacgtgtttctcccggctcggccacttcattgccagtcaccctgtcttcttcgcctcggcgccggtgctcatctccatcctgctcggcgccagcttcagccgctaccaggtcgaggagagcgtggagcacctgctggcgccccagcacagcctggccaagatcgagcgcaacctcgttaacagcctcttcccggtcaaccgctccaagcaccgtctctactcggacctgcagacccccgggcgctacggccgggtcatcgtcacctccttccagaaagccaacatgctggaccagcatcacaccgacctgatcttaaagttgcatgctgctgtcaccaagatccaggttccaaggcctggttttaattacacgtttgcccatatatgtatcctgaataatgataagacttgcatcgtggatgacatagtgcacgtcctggaagagctaaagaatgctcgggccaccaatcggaccaattttgctatcacatacccaatcactcacttaaaggacgggagggctgtgtacaatgggcaccagcttgggggcgtcactgtgcacagcaaagaccgggtgaaatctgcagaggccatccagctcacctactacctgcagtcaatcaacagtctcaatgacatggtggctgagaggtgggagtccagcttctgcgacactgtcagactgtttcagaaatccaacagcaaagtcaaaatgtacccttacacgtcctcctcactgagggaagatttccagaagaccagccgcgtatcagaacgttacctggtcaccagcctgattctggtggttaccatggccatcctgtgttgctctatgcaggactgcgtccgcagcaaaccctggctaggcctgctcggattggtgaccataagcctggccactctcactgcagccgggatcatcaatcttactggtgggaaatataattccaccttcctgggagtccctttcgtcatgctaggtcatggattatatgggacttttgaaatgttatcctcctggaggaaaactagagaagaccaacatgttaaagagagaactgcagcagtctatgcagactccatgctctccttttctctcaccactgccatgtacctggtcacctttggcataggggccagccctttcacgaacattgaggcagccaggattttctgctgcaattcctgtattgcaatcttcttcaactacctctatgtactctcgttttatggttccagcctagtgttcactggctacatagaaaacaattaccagcatagtatcttctgtagaaaagtcccaaagcctgaggcattgcaggagaagccggcatggtacaggtttctcctgacggccagattcagtgaggacacagctgaaggcgaggaagcgaacacttacgagagtcacctattggtatgtttcctcaaacgctattactgtgactggataaccaacacctatgtcaagccttttgtagttctcttttaccttatttatatttcctttgccttaatgggctatctgcaggtcagtgaagggtcagaccttagtaacattgtagcaaccgcgacacaaaccattgagtacactactgcccagcaaaagtacttcagcaactacagtcctgtgattgggttttacatatatgagtctatagaatactggaacactagtgtccaagaagatgttctagaatacaccaaggggtttgtgcggatatcctggtttgagagctatttaaattaccttcggaaactcaatgtatccactggcttgcctaagaaaaatttcacagacatgttgaggaattcctttctgaaagcccctcaattttcacattttcaagaggacatcatcttctctaaaaaatacaatgatgaggtcgatgtagtggcctccagaatgtttttggtggccaagaccatggaaacaaacagagaagaactctatgatctcttggaaaccctgaggagactttctgtcacctccaaggtgaagttcatcgtcttcaatccgtcctttgtatacatggatcgatatgcctcctctctgggagcccccctgcacaactcctgcatcagtgctttgttcctgctcttcttctcggcattcctggtggcagattcactgattaacgtctggatcactctcacagttgtgtccgtggagtttggagtgataggtttcatgacattatggaaagtagaactggactgcatttctgtgctatgcttaatttatggaattaattacacaattgacaattgtgctccaatgttatccacatttgttctgggcaaggatttcacaagaactaaatgggtaaaaaatgccctggaagtgcatggggtagctattttacagagttacctctgctatattgttggtctgattcctcttgcagctgtgccttcaaatctgacctgtacactgttcaggtgcttgtttttaatagcatttgtcaccttctttcactgctttgccattttacctgtgatactgactttcctgccaccctctaagaaaaaaaggaaagagaagaaaaatcctgagaaccgggaggaaattgagtgtgtagaaatggtagatatcgatagtacccgtgtggttgaccaaattacaacagtgtgataatgtctgcttggcatattttcaccttaggtcttatcaagaccaaagagattatgttaatgaaacaattaaattcaaagttcttcccttttttaaagataggaaacaggcattgccaaaaaaaaaaaaaaaaaaaaaaggaaaggacagtggggagaaatgggcctggcatattttcagtctttaaaacaaaggagttgttatgagaattcacacacacatagacacacacacacacacacacacacacacacacacacacacaccctgggagacctatagtctcttaaactaagatcaagtagaagaaagcttattaacaagcaggatcctgccttatccaaactgcagatgttgctggcattgtgacaaaacccactgattgaaaggtcaactgccaaggcagaaacacctttaagcattgttcaaacaataaggcttccagaacttctgtagagcagtagctccagtcatggtctgtggtttgaggttttagctgtctcacctagctccctaacactgaaggagatacttgtgaaagttctgaccagcaaaagcaagccagagccttggaaactgatatgtggtagagtggccatcactcatggactaaaattgattcaccgctaaatttacccaggtgaagcagtttcgttgtctagaatgaaattatcatattccgccattggtatgcctttaacatttgtatagtttggtttgcttaaaacaccttaaaaccaatgacagctccagcactgcagaattggtgtgattctactttggaatagcttgtcacttgtcaccaaatgggtctgctttattagttacagctcttggcaggaggatccagggacccaaaaccacagggccaaacccaaatacctggcatgatggagcaaaagcaggtgtctacttggacccagatatagtgtctccattttaacaacaacaacaaaatagccagctggtacagctgtttgcattggccctacatgcattttttgcatggatatccagaaacatctgcccacacaaaactgcggggaaaaaaaatgaacactgaaatagttatttgctgttgcttccaacttgtagtgccagtctgcctttgctgtgaaacacacctgctcagagacagagaggggaagaagatctttggtaagtctaagtcctgacgctgagaagctttgtaaaagtgcagggagataaagggccaaaagggagatagatggaaaacactggaaaaagtattcactgatacaaatctatcaatgatggcagtccaattctcttgctaaagtggctgcacctcaccttgctggtcccccccacaccttttttgatgtccttctgcgtcatcatagcaaggcccttctgtaaattaacaagcctagatatttatactcttgacttccagtatctacagaagaatggttcatagatctaaacagaaatggtttagatctaaaaaggctgtatacgttgcccaggcccctgcatttctttaaatttataaaaatgaagctaaaacctggttacatttgaagcaaatatctacagtatttttcccttttagagatgtagcttccttagacatctgtagtggtaagcatttcccaaaagcatcttacctttctgaaccttagcagacatactgtgcagcttacctatcttctgcagaggaggaaactgagacctaggagaataaagtgactcactcaggtcacaccactaaagggttttcatcatttcagcatacctaagacagggcagtccaattttcagtattctcataagatggctattactcctctcaaaatgcatttccaaagtaggaacataggacttcgttggccacagggcagacatttttttagtgtctggaattaaaatgtttgaggtttaggtttgccattgtctttccaaaaggccaaataattcagatgtaaccacaccaagtgcaaacctgtgctttctatttcacgtactgttgtccatacagttctaaatacatgtgcaggggattgtagctaatgcattacacagtcgttcagtcttctctgcagacacactaagtgatcataccaacgtgttatacactcaactagaagataataagctttaatctgagggcaagtacagtcctgacaaaagggcaagtttgcataatagatcttcgatcaattctctctccaaggggcccgcaactaggctattattcataaaacacaactgaagaggggattggttttactgttaaatcatgtgttgctaaatcattttctgaacagtgtgttctaaatcagtcattgatttagtgtcagccacgtggagcacctcggcttaaagcagctccacaaaacctgacacaacacacacaccaattaaatggattttgttgagaatttaatcattcaatttggtcaaccagaatgacttcctgtggaactctgttttatgacagataatagttttccaacttgattgagtctctgtataccctgggatattgtattttttaatgaagggcattttcaaacttgtcaacttctcttttcagcacttgaaatgaaggcttatggaattctgactgtgaaatgaatttttctattgggagattatgcatgccaagatttattatttattgttagataattattatttatttgttaatactgattattgattattgctctgctgcattggtttagccccgctatggagaagacactttagaagtaactcagaatcacatccccataaaatctaagtctttacttcatcaagttttactttttgaattgctataaagacggaaaaattgatccctgcaaacacttgaagaagctgtgttttttttctttcaattaattccaaccaagacattaacgtccactttaccaaagaaacttacttggttggtttttagttggtgtttgttgtttgtttaccaaagaaaatattcgattcatcaaaaatattgctcctggtcaactaaaaaactgtgatactgggacttttttggccattcataaggaaaatttttaaaagtcaaggtaagggtgatattctctcatttagtggaagaagaagatgtcaattcacagggaatgtggtgttcgcaagaaagaaaatgatacttgaaatagggcattattgtttagcctacagcaacatttcaacaagcagcatcagggctcctcaggttaaaagaagtacagtttgtttcagagaaaaaggaagacaaaccctcgtgaaatgtttctccattgttccagctcataagttttactttgttttgggttttggagtttttagtttctttgatgtttgttttgctttaatttggtttgtggtttgtgtttgcttgtgtttgcttctgcctttcctaccttctgccttttactccctcagttttcagtcagaagtgtgataatcagatgccatcctccatggccacgcttatgtatgcaatataccgctccaaatgaggaccttggtaacccttgaccctttaattctaactaaccatcacccaatcgtaagtggcattatgggaccatgataccacttttctccagtgtcaatgacagagctcagctgtgtccatcacaagaaaaggtggaatgggcagctgtcttgggggtgtggcaagaggcagaacctcttggacacaaatgttatcttcctcataatggctttattcttttggatcctattaaagtacccctgtagcttatagtagtgtttgacctctgtgactctgttttctcatctgtaaaatggggataatatcagtaaatgtctttaaattcagcaaatacttatcgagtgccaaatatcgcccagatactgttcgagaggctggggacacaacagtcaacaaatcagattttttaaaatctgctcttgtagagtctacctttgagtgacattgcaggtatattgtggggattgaatgagatgagagatgacatttggaaagcactcttggaaaggcctggcaaagaaaagagactcaagacattatttgtaattcagcaatctcgcacagtagcattagggctcgggatgtaaagctactgagaagtagatttgagcctgagaattagggctacaaatacaatgagttgccacagaaggtagttaattaccagtcattttaggacatcaaaatgaagctgatcaccacatagtggggccactgtaaaggggctgcaagtcctggatgttggcatcatactatgatctttgaaaaaaacagatacaggcacttgcttttggggatggggaagggacaccactgattactgaataactgtggctggcttgtctgagggaatgtaaacattcccttgaggtaatttgcttttaattttgtgtaaaaagcaccaggtatttagagacataaagttcccgttttttaaaagaatagtaaatctgcaatacaaagaaaaatattaagttagtggattcgctggcaaagaactaagaatggctgcagccaacacactgcagagttcctccctgtggtggttacaactgttatttctgtttcatgccagtccaggagaaagcaaagaggaaagtggatgagctataatccgttcacctgaataggcagtgccaggatgtcccccccagcttgcctctcacccgccccgctgctgcccagctgatttgtttgaagtcctctttgatatctatgagagcaccgtggaaggaagaagaagggaggcgaccggagagctatggcctctgtgcagatttttgagttagattaaaattagtaccacctgattaggagcctacctggtgaggaaaaaagccttcttttcaaagtacaaaaaaaagcgatgtgtaccaaggagaaaaccagcagagaaagcttggtggaagaagtgatgttcttgtggaatgtgccagcagcttctccttccagaatgctgacaccagatgaatctttggttagaattaagcctacccctttgtttttcacatgagaaaactgaaccctcgagtaatgaaattatattcacagagctaattagaaacaggagactataattgaaatctagtttttagcctcaattctttctccattttgccactttactgccaaattctattcatctaaattattggggtcttaacctgggggccaaatgagttcatgggaggggtctctgagtttcctacaattaagttacatgcaaaaatgtgtgtatatgttcaacatgtgcattttcctaaactgagggctcagattccaaaagaggtctatgaccgaaaaagggtgaagaaccactgtcctaaattaatacagacctcattgggagtgtgatcctggtgtcctcattttggctaatgagcaatcaagaacttttgaactatcttttctctaaggatccaaaaatgcttcaaaatattggtttcttcatcccaagccaagtgcctacttctattcatttattcttcattcattctttcattcattcatttctttattctgcaaatgtattttcagtgccaattcatgttctagcagcagctaaatatgccctccacagcaggaccaggaaaggagccctctgtactgtgctcttgttggcaagaggaacaccaggttgctaccacattctcactgttgcaacaaattatatatagcctcaaattttcagtaaccatcccacacacacatacacacataaaggcagcagccaacaaagccaacatatagaagtgtgccctgtcaaagaaccaggaagagcagcctatttccaggttatatacagtgatatgctgtttacaactgcataccacatccttagaaggcctgctcccaaatgagccctactctgagatgtctacaccaggcatgctttcacacacgtgcagtaccctactctcaaaacaagtatggagagaataagtctcattcatctcgttaatatttttagtgggcaaagaagcaaccgtggccatggacagatgggtgcatttaaggacaggtgtggccgggcacagtggctcaagcctgtaatcccagcactttgggaggcggaggcgggcagatcacgaggttaagagattgagaccatcctggccagcatggtgaaaccccgcctctactaaaaaatacaaaaaaattagctgggcgtggtggcacgtgcctgtagtcccagctactcaggaggcaggagaatcccttgaacccgggaggtagaagttgcagtgagccgagatttcaccactgccctccagactggcaacagagtgagacttcgtctcaaaaaaataaaggacaagtgtgttttatacacaaatatgcagaagcccagttctcttgactccaagtcagaagctagataaaatagaggttgctgggggaagttggaaaataaatgactatactgtacttgttggctctgctgctgctgatataacttcaggcacaacggaagccgcctcggttggcctatgggaagggctgggtgaatcactgcttggccttgcatgaagcacaaaggacacaccaatggctgatcagtggcccttccatcatcttctgcccagccatgataaggaaagccttctattgagatggaggaatattagctcaactttgtagcatctgatgtaaagtttgaggggtttgtcttttttgtttgtttgtttgtttttttttgagatggagtctcactctgtcacccaggctggagtgcagtggcgccatctcagctcactgcaagctccacctcccaggttcacgacattctcctgcctcagcctcctgagtagctgggactacaggtgcccgccaccgcgcccggctaattgtgtgtgtgtgtgtgtgtgtgtgtgtgtgtgtgtttagtagagatggggtttcaccgtgttacccatgatagtctcgatctcctaacctcgtgatccacccgcctcggcctcccaaagtgctgggattacaggcgtgagccaccgtgcccggcaagggctttgttcattaggatcaacaaagtgctttgtctaattcctgcaaaatggcaaagacccagcccatttccattactcattccaggctcatgctcaggaaaactaataacgttctctttgacaaaacaatttccttaattttctaggatgaatcttgaacatgtttagctcagcctttcagatatttattctttaaattaccatcctgtcaaaattgcctttacgcactgatccagaatcaccaaggcagttgccacgatgcctcccaggactgtgggtgactgggcaaactggtaatgacccgttggctctgcttcacacacacacatacacattgacacacataaatgtacacatgcatatatgtgtgtacaacacaactataaaacatagaaacatatacctgtacatatgcacatgcttacatacacacatatcacagatgtgcatgcacacatacacatgcacaccaccttttacaatatgcctgacacatttgaggcacattaaaatgccttggcaggcccagccctatagttctgtaaggtttggcaggccaaaggcttcggatgtctgcaggataccattgaaaaaatgttatcttccccttctatatgtttcttatgcacgagcacagctaagagcagccaaatagtaagctttaaagaaaagaaacctggaaaagtgaatgtgccttgcaatttaaaaattgctaacagtttcacagtgggaaaattgcatttgttggaattttccaagtcataagcaagaagaatagcatagacccaaaacgacagagatctcttccaaggtatggaattgtggaaatttatttagaatatttgcttggtgatccagagaagggggaaagaagtggtgaggaagaatatatggggtgatccctccaaattgccaactcctcgtcttaggaagaagtgggcagtttccattgatccctagcatcagtttgacgggaaaaccggtgcatttcatcaacctcaaggaaaggaagtgccatgcatggaggcctggtgataggcaacatccatttagaagaacagtcacacaagcaacagaagaactatccaagactagaagcttagaagcttataccaaactctctgtctctctctctctctctcacacacacacacacagagtggatgggaggggacattaagaggaaccctagtgcagttcccccaaaaaaccttggcatagagtataacagggaaaaagtaattactgggaagctgaaaacaatcaaatatgtgaggaatgactgaatatgagctgaggataatgtaaattgttctggattgtctataagataaagatacagagcctgaatctatgtttttctgaataaagagaccccatattctcttttgaattattgacctataattcctcttcagcagctttgaatacactcaagttctcttcctattgtttatgatcccataatacaaccacccactatttgttgtggtttacaatccagcttagaacccttgagcaaaattattgaattattgaatgctaggcccaaacaaaagaaatttcagcttggttttagtaatttcttgatcaaacttctctcattcctctaaatgtacaaaggtttcaaagttgtaaaaaatatgtctacatagccaatgctcttagtatttttaacagcaaagataatctctgccaataattattaatgacaaaatagtgataactgttcaatgttgctttaacagtccaattgtagtaggtgagaaaacaaaaaagaagagaagctatgttgaatatttagtaatgttggaaaagatcctcaccctagacataaaatagtttaaacatttaaacatggcccctctcttattgtcattgctagtgaaagaagttcctgaaattgcatattacattgtaaagttttgggctaaatcacaatacatttttaagttgcttccatggcaatagatcgtaagaccacagcttgtcaaagtaaccactaattcagtattcagaaaccttatcatctgttcctccaaaaagaccccatagttaatgttgtgtttcccagtattttagttgggtttcttcacacttaggcaagtgggtgattctggtcataataaactattttcaaactaggaaaaagtatccaaatgctagagcacataatgagttcagcttggtcagttttgagacagataagcataaaaccctactagaaaaaaataatagtcaaacctaaagctttaaaaaatgtgagtcacatcacttttctccaggaaaggcagaacgtgctcagggtggaatttaatcaaaaatgtacccagaacaatttcccacaaagagctgtaatactttgcagcaaggggcattaataaactctaatttttaccgaaagtcaagagacacacagatcttttttggtaccctctacatcatcatagcaaggcccttctgaacattaacaagcctagatgtttctactgcttatttgcagtatctacaggggaatggttcgtggatataaacagaaatgttttatctgaagaggctgtgtatgccaccccagacccctgcatttcttaatatctataaaaatatgactaaacttgttacatttgaggcaaatatgtacagtatttttcccttttaaagatagtttaaaaaatcaaggtaataaaaaacggccaactctttcaggaaaaaaaaaatccggtattttgtactacttgagtttctctaatgcctaaaatatgcatttaaatcatcccacacctacacagaaacaaaatcagcagggaaaaccatcccactgatgattggcaattccaccgttcctctccatctgaccagctttcaccagagggcgatctttcatgcttgcacatcgagtttatacaatgtagaagcccccaagtcacctgggaaatggctcatgccaaatacatggagtccttagttgactatcttttcatattaaaatcccatcttccaatgaagcatcctgttttcaatactgatatcaaatttcgaatgtaaaggagatttcaaatgtattgctttcaccttttcttttttattcatttatttttaattgacataaaaattgtatatatttatgggatacaatgtgatgttttgatatatgtatgcattgtggaatgattaaatcaagctaattaacata

SEQ ID NO: 5 - protein, 247 aa, Homo sapiens
MVDREQLVQKARLAEQAERYDDMAAAMKNVTELNEPLSNEERNLLSVAYKNVVGARRSSWRVISSIEQKTSADGNEKKIEMVRAYREKIEKELEAVCQDVLSLLDNYLIKNCSETQYESKVFYLKMKGDYYRYLAEVATGEKRATVVESSEKAYSEAHEISKEHMQPTHPIRLGLALNYSVFYYEIQNAPEQACHLAKTAFDDAIAELDTLNEDSYKDSTLIMQLLRDNLTLWTSDQQDDDGGEGNN

SEQ ID NO: 6 - DNA, 3705 bp, Homo sapiens
  source 1..3705: mol_type = genomic DNA
gcagccgcctcgcgcccggtcccgcggtcgcagctccagccgcctcctccgcgcagccgccgcctcagctgctcgctctgtgggtcggtcctctccggcacttgggctccagtcgcgccctccaagcccttcaggccgccccagtgtcctcctccttctccggccagacccagccccgcgaagatggtggaccgcgagcaactggtgcagaaagcccggctggccgagcaggcggagcgctacgacgacatggccgcggccatgaagaacgtgacagagctgaatgagccactgtcgaatgaggaacgaaaccttctgtctgtggcctacaagaacgttgtgggggcacgccgctcttcctggagggtcatcagtagcattgagcagaagacatctgcagacggcaatgagaagaagattgagatggtccgtgcgtaccgggagaagatagagaaggagttggaggctgtgtgccaggatgtgctgagcctgctggataactacctgatcaagaattgcagcgagacccagtacgagagcaaagtgttctacctgaagatgaaaggggactactaccgctacctggctgaagtggccaccggagagaaaagggcgacggtggtggagtcctccgagaaggcctacagcgaagcccacgagatcagcaaagagcacatgcagcccacccaccccatccgattaggcctggctcttaactactccgtcttctactatgagatccagaacgccccagagcaagcgtgccacttggccaagaccgcgttcgacgacgccatcgccgagcttgacaccctcaacgaggactcctacaaggactccacgctcatcatgcagctcctccgcgacaacctcacgctctggacgagcgaccagcaggacgacgatggcggcgaaggcaacaattaaggccccaggggaactggcagcgcacgcggatgctactactgcagtctttatttttttcccatgagttgggggtcgggtgggggagggaaagggagggatgaccttcccagggagaaacccacgacctgtcctgtctttgatcgcctctttgacatttttgccaaaataccactagtggaaagtcaggctagctgtgctggtattggaatagcagcctcacactggcgtctggactgttctgtagattcatgcaagtggagctgtctgtctctaatttaacttattgctagataatagggttttcagatgaaaagaaaacttaaagaggaatggccctcattcagtaagttctgtggttccagtaaggatttttatgtacatacgctctcgtctctcgttttgggtactttctatctcatctgtctcggctctgcatgttttccagggtgtagcctacagacatggaacagtgtaaatcccagactgacagacttagaacctgaggtctcattcatccttatggtttaggccttgccagttttccgaagtctctgattagttgacagtattaacactaaattgcagtttacagtatttctacattacagccatatgtaacatcaagccatcgattgtgtacttttcctttgctagttgtttgggctttaacatccttattcagccttatccaggttggttttgctgttgatcggtctcctaggctaaatgagaatgaaagcgacttcaggtcaggtggctgtgggattttttttttttggtccttctttcctcttaacgtaaatccaccaccaaaattattaatcctcttgagagaaacgtgaaacgccacaaaaatagagaaaattcaggtctgtatgtcatggatcgtgttggtattttcagagaacatcccgcttctgaagctgctgcagctccctcctcagggatcacactgccgtcacccactctgcactggggcgtttcctactgcgcctcgtgctggcggacgcagctgggtgcagaagctgtggggtcggagaggcgtttggagaaggtctgtggtgcagtgtgtgaaaattcaggtgctagaagcctactggtagaaaaacccaaaaggaagagctatatccttaaccattctgtccaatttcgggagccttgtcagtgtgtcagtttttcctccccgaagacactccttccccaagtaattgtaggaagataaaaaaactgttaccagataacaaacactgaactcctatttgaccagaactttttcctctcgagatagttttttctttttaatgaaaaaagcataggaattggagattggcttgtctcacgcagccagtgcacatttggaattgacggaaacaacgttgctatttccacccatttgttttcggcagccttaaggccctcattctcatttcgggtgaatctgtctatctgtgaacgtggcccgcatgtgcattcttttttttatatatataaagtcagtgacgaggaactcccgagacgtgtaatgacaccacacttgttttctttgtttctttgttttatttaggcaagaagaggtgtgagtaattgaggaaaaactgacagatgcttttgctaataccaaaattgagcttacaattaggaactgagtatgtgtaacaggatacaggtgacagtgaagatagaagaaccacgatgaccacagactcaatgtgctctgtaacatcgcacagtttacccagcatgactttccttaggaggccccctcctcacgctagagtaaaagtcccagttaagtgaagcctaccagaagaactagtagaagaagctttgccgcttttgtgcctctcacaggcgcctaaagtcattgccatgggaggaagacgatttggggggggaggggggggggggcagggtaggtggggctttccctaatttatcttcatgtccagtgagcagtgttgcgtttttccttgtagcatttggaaatgatttactggaattacaaaacctatttttcctttaaatttcagctttggctctggctgctttttagaataatgcaagataaaaatcacacctgagggctgaaaacggagagggaatgggagacttgatatttaagcagcttgaatggtttttcttttctttatttttaaagaaatgcacttgcctatgatactgtctctccagtgaaatgattactcctccattactctattgatacaatattgtgcatgctagtgttgtatttctatacagtagcttgaaattgattaacttatactgtaggtgttatgtattcctatgacaaaaaaaattaagtcttcaaattttttaaaggttttttttttttaatttaatttttccttttgggggtaaagtttgctctaccaaatagtgattgtaacaaattgatctgttttggatgttgctatagtgacatgcagttatatattttgtttttaaaagggggggagcaaaagaaacaccagtgttagcttaatcttaatgtctggtgtttgtcatggtgaaattataactattacagtgttggagaacaacaaatatgttctctgaatgagcctttgtgctttttgtcatgttatgcagtgaactatttttaaggtctaatcagtgattatttttccagctccgtgtttctctaaggaattatttcacacacggaccatctttagcagtttcctcagtgatggaatatcatgaatgtgagtcattatgtagctgtcgtacattgagcaaataaacttacagatctga

SEQ ID NO: 7 - protein, 4861 aa, Homo sapiens
MATMIPPVKLKWLEHLNSSWITEDSESIATREGVAVLYSKLVSNKEVVPLPQQVLCLKGPQLPDFERESLSSDEQDHYLDALLSSQLALAKMVCSDSPFAGALRKRLLVLQRVFYALSNKYHDKGKVKQQQHSPESSSGSADVHSVSERPRSSTDALIEMGVRTGLSLLFALLRQSWMMPVSGPGLSLCNDVIHTAIEVVSSLPPLSLANESKIPPMGLDCLSQVTTFLKGVTIPNSGADTLGRRLASELLLGLAAQRGSLRYLLEWIEMALGASAVVHTMEKGKLLSSQEGMISFDCFMTILMQMRRSLGSSADRSQWREPTRTSDGLCSLYEAALCLFEEVCRMASDYSRTCASPDSIQTGDAPIVSETCEVYVWGSNSSHQLVEGTQEKILQPKLAPSFSDAQTIEAGQYCTFVISTDGSVRACGKGSYGRLGLGDSNNQSTLKKLTFEPHRSIKKVSSSKGSDGHTLAFTTEGEVFSWGDGDYGKLGHGNSSTQKYPKLIQGPLQGKVVVCVSAGYRHSAAVTEDGELYTWGEGDFGRLGHGDSNSRNIPTLVKDISNVGEVSCGSSHTIALSKDGRTVWSFGGGDNGKLGHGDTNRVYKPKVIEALQGMFIRKVCAGSQSSLALTSTGQVYAWGCGACLGCGSSEATALRPKLIEELAATRIVDVSIGDSHCLALSHDNEVYAWGNNSMGQCGQGNSTGPITKPKKVSGLDGIAIQQISAGTSHSLAWTALPRDRQVVAWHRPYCVDLEESTFSHLRSFLERYCDKINSEIPPLPFPSSREHHSFLKLCLKLLSNHLALALAGGVATSILGRQAGPLRNLLFRLMDSTVPDEIQEVVIETLSVGATMLLPPLRERMELLHSLLPQGPDRWESLSKGQRMQLDIILTSLQDHTHVASLLGYSSPSDAADLSSVCTGYGNLSDQPYGTQSCHPDTHLAEILMKTLLRNLGFYTDQAFGELEKNSDKFLLGTSSSENSQPAHLHELLCSLQKQLLAFCHINNISENSSSVALLHKHLQLLLPHATDIYSRSANLLKESPWNGSVGEKLRDVIYVSAAGSMLCQIVNSLLLLPVSVARPLLSYLLDLLPPLDCLNRLLPAADLLEDQELQWPLHGGPELIDPAGLPLPQPAQSWVWLVDLERTIALLIGRCLGGMLQGSPVSPEEQDTAYWMKTPLFSDGVEMDTPQLDKCMSCLLEVALSGNEEQKPFDYKLRPEIAVYVDLALGCSKEPARSLWISMQDYAVSKDWDSATLSNESLLDTVSRFVLAALLKHTNLLSQACGESRYQPGKHLSEVYRCVYKVRSRLLACKNLELIQTRSSSRDRWISENQDSADVDPQEHSFTRTIDEEAEMEEQAERDREEGHPEPEDEEEEREHEVMTAGKIFQCFLSAREVARSRDRDRMNSGAGSGARADDPPPQSQQERRVSTDLPEGQDVYTAACNSVIHRCALLILGVSPVIDELQKRREEGQLQQPSTSASEGGGLMTRSESLTAESRLVHTSPNYRLIKSRSESDLSQPESDEEGYALSGRRNVDLDLAASHRKRGPMHSQLESLSDSWARLKHSRDWLCNSSYSFESDFDLTKSLGVHTLIENVVSFVSGDVGNAPGFKEPEESMSTSPQASIIAMEQQQLRAELRLEALHQILVLLSGMEEKGSISLAGSRLSSGFQSSTLLTSVRLQFLAGCFGLGTVGHTGGKGESGRLHHYQDGIRAAKRNIQIEIQVAVHKIYQQLSATLERALQANKHHIEAQQRLLLVTVFALSVHYQPVDVSLAISTGLLNVLSQLCGTDTMLGQPLQLLPKTGVSQLSTALKVASTRLLQILAITTGTYADKLSPKVVQSLLDLLCSQLKNLLSQTGVLHMASFGEGEQEDGEEEEKKVDSSGETEKKDFRAALRKQHAAELHLGDFLVFLRRVVSSKAIQSKMASPKWTEVLLNIASQKCSSGIPLVGNLRTRLLALHVLEAVLPACESGVEDDQMAQIVERLFSLLSDCMWETPIAQAKHAIQIKEKEQEIKLQKQGELEEEDENLPIQEVSFDPEKAQCCLVENGQILTHGSGGKGYGLASTGVTSGCYQWKFYIVKENRGNEGTCVGVSRWPVHDFNHRTTSDMWLYRAYSGNLYHNGEQTLTLSSFTQGDFITCVLDMEARTISFGKNGEEPKLAFEDVDAAELYPCVMFYSSNPGEKVKICDMQMRGTPRDLLPGDPICSPVAAVLAEATIQLIRILHRTDRWTYCINKKMMERLHKIKICIKESGQKLKKSRSVQSREENEMREEKESKEEEKGKHTRHGLADLSELQLRTLCIEVWPVLAVIGGVDAGLRVGGRCVHKQTGRHATLLGVVKEGSTSAKVQWDEAEITISFPTFWSPSDTPLYNLEPCEPLPFDVARFRGLTASVLLDLTYLTGVHEDMGKQSTKRHEKKHRHESEEKGDVEQKPESESALDMRTGLTSDDVKSQSTTSSKSENEIASFSLDPTLPSVESQHQITEGKRKNHEHMSKNHDVAQSEIRAVQLSYLYLGAMKSLSALLGCSKYAELLLIPKVLAENGHNSDCASSPVVHEDVEMRAALQFLMRHMVKRAVMRSPIKRALGLADLERAQAMIYKLVVHGLLEDQFGGKIKQEIDQQAEESDPAQQAQTPVTTSPSASSTTSFMSSSLEDTTTATTPVTDTETVPASESPGVMPLSLLRQMFSSYPTTTVLPTRRAQTPPISSLPTSPSDEVGRRQSLTSPDSQSARPANRTALSDPSSRLSTSPPPPAIAVPLLEMGFSLRQIAKAMEATGARGEADAQNITVLAMWMIEHPGHEDEEEPQSGSTADSRPGAAVLGSGGKSNDPCYLQSPGDIPSADAAEMEEGFSESPDNLDHTENAASGSGPSARGRSAVTRRHKFDLAARTLLARAAGLYRSVQAHRNQSRREGISLQQDPGALYDFNLDEELEIDLDDEAMEAMFGQDLTSDNDILGMWIPEVLDWPTWHVCESEDREEVVVCELCECSVVSFNQHMKRNHPGCGRSANRQGYRSNGSYVDGWFGGECGSGNPYYLLCGTCREKYLAMKTKSKSTSSERYKGQAPDLIGKQDSVYEEDWDMLDVDEDEKLTGEEEFELLAGPLGLNDRRIVPEPVQFPDSDPLGASVAMVTATNSMEETLMQIGCHGSVEKSSSGRITLGEQAAALANPHDRVVALRRVTAAAQVLLARTMVMRALSLLSVSGSSCSLAAGLESLGLTDIRTLVRLMCLAAAGRAGLSTSPSAMASTSERSRGGHSKANKPISCLAYLSTAVGCLASNAPSAAKLLVQLCTQNLISAATGVNLTTVDDSIQRKFLPSFLRGIAEENKLVTSPNFVVTQALVALLADKGAKLRPNYDKSEVEKKGPLELANALAACCLSSRLSSQHRQWAAQQLVRTLAAHDRDNQTTLQTLADMGGDLRKCSFIKLEAHQNRVMTCVWCNKKGLLATSGNDGTIRVWNVTKKQYSLQQTCVFNRLEGDAEESLGSPSDPSFSPVSWSISGKYLAGALEKMVNIWQVNGGKGLVDIQPHWVSALAWPEEGPATAWSGESPELLLVGRMDGSLGLIEVVDVSTMHRRELEHCYRKDVSVTCIAWFSEDRPFAVGYFDGKLLLGTKEPLEKGGIVLIDAHKDTLISMKWDPTGHILMTCAKEDSVKLWGSISGCWCCLHSLCHPSIVNGIAWCRLPGKGSKLQLLMATGCQSGLVCVWRIPQDTTQTNVTSAEGWWEQESNCQDGYRKSSGAKCVYQLRGHITPVRTVAFSSDGLALVSGGLGGLMNIWSLRDGSVLQTVVIGSGAIQTTVWIPEVGVAACSNRSKDVLVVNCTAEWAAANHVLATCRTALKQQGVLGLNMAPCMRAFLERLPMMLQEQYAYEKPHVVCGDQLVHSPYMQCLASLAVGLHLDQLLCNPPVPPHHQNCLPDPASWNPNEWAWLECFSTTIKAAEALTNGAQFPESFTVPDLEPVPEDELVFLMDNSKWINGMDEQIMSWATSRPEDWHLGGKCDVYLWGAGRHGQLAEAGRNVMVPAAAPSFSQAQQVICGQNCTFVIQANGTVLACGEGSYGRLGQGNSDDLHVLTVISALQGFVVTQLVTSCGSDGHSMALTESGEVFSWGDGDYGKLGHGNSDRQRRPRQIEALQGEEVVQMSCGFKHSAVVTSDGKLFTFGNGDYGRLGLGNTSNKKLPERVTALEGYQIGQVACGLNHTLAVSADGSMVWAFGDGDYGKLGLGNSTAKSSPQKIDVLCGIGIKKVACGTQFSVALTKDGHVYTFGQDRLIGLPEGRARNHNRPQQIPVLAGVIIEDVAVGAEHTLALASNGDVYAWGSNSEGQLGLGHTNHVREPTLVTGLQGKNVRQISAGRCHSAAWTAPPVPPRAPGVSVPLQLGLPDTVPPQYGALREVSIHTVRARLRLLYHFSDLMYSSWRLLNLSPNNQNSTSHYNAGTWGIVQGQLRPLLAPRVYTLPMVRSIGKTMVQGKNYGPQITVKRISTRGRKCKPIFVQIARQVVKLNASDLRLPSRAWKVKLVGEGADDAGGVFDDTITEMCQELETGIVDLLIPSPNATAEVGYNRDRFLFNPSACLDEHLMQFKFLGILMGVAIRTKKPLDLHLAPLVWKQLCCVPLTLEDLEEVDLLYVQTLNSILHIEDSGITEESFHEMIPLDSFVGQSADGKMVPIIPGGNSIPLTFSNRKEYVERAIEYRLHEMDRQVAAVREGMSWIVPVPLLSLLTAKQLEQMVCGMPEISVEVLKKVVRYREVDEQHQLVQWFWHTLEEFSNEERVLFMRFVSGRSRLPANTADISQRFQIMKVDRPYDSLPTSQTCFFQLRLPPYSSQLVMAERLRYAINNCRSIDMDNYMLSRNVDNAEGSDTDY

SEQ ID NO: 8 - DNA, 15197 bp, Homo sapiens
  source 1..15197: mol_type = genomic DNA
gtctttccctttgacaagtcgctgctgctgcagcaaaaataaaggacgcgaccgccgcagcctctgcggagccgggctcgggtcgccggagccgcgccccaccccgccagctccagagccacgactaatggctgaaggataaatcaacatggcaactatgattccaccagtgaagctgaaatggcttgaacacttgaacagctcctggattacagaggacagtgaatctattgctacaagagagggagttgctgttctgtattctaaactggttagcaataaggaagtagtacctttgccccaacaagttttatgcctcaaaggaccacagttgccagactttgaacgtgagtctctttcaagtgatgagcaggaccactatttggatgcccttcttagcagccagctagcattggcaaagatggtatgttcagattccccatttgccggggcacttagaaaacgactgcttgtactccagcgtgtcttttatgcactttctaataaataccatgacaaaggcaaggtgaagcagcagcagcattctccggagagcagttctggttcagcagatgtccattctgttagtgaacgcccccggtcaagcactgatgcacttatagaaatgggtgttcgaactggtctaagtttattatttgcgcttctaagacaaagttggatgatgcctgtgtcaggacctggtctcagtctttgcaacgatgtcattcatactgcaattgaagttgtgagctctttgccaccattatcattagcaaatgaaagcaagattcctcctatgggcttggactgcttatcgcaagtaacaacatttcttaaaggagtcactattcctaattctggggcagacactttaggtcgtagattagcttctgagttgctgcttggtttggcagctcaacgaggctcattgcgatatcttcttgaatggatagaaatggctttgggggcttcggcagttgtacacaccatggagaaaggcaaactactctcaagccaggaaggaatgatcagctttgactgctttatgaccatattaatgcagatgaggcgttctttgggttcatctgctgatcggagtcagtggagagaaccaaccagaacatcggatggcttgtgctccctttacgaggcagcattatgtctctttgaagaggtttgcagaatggcttctgattattcgagaacatgtgctagcccagatagcattcagactggtgatgctcccattgtctccgaaacctgtgaggtttatgtttgggggagcaatagcagccatcagttggtagaaggtacacaggagaaaatactgcaacccaaactggctcctagtttctctgatgcacagaccattgaagctggacagtactgcacttttgtcatttctacggatggctctgttagagcttgcgggaaaggcagctatgggagactgggccttggagactccaataatcagtcaactttaaaaaagttaacattcgagcctcacagatccattaaaaaggtttcatcttctaaaggatctgatggtcacactttagcctttacgacagaaggagaagtcttcagttggggagatggtgattatgggaaactggggcatggaaatagttcaacacagaaatatcccaagcttattcagggacctctacaaggaaaggtagttgtttgtgtgtcagctggatacagacatagtgctgctgtcacagaggatggggaattatacacatggggtgaaggagactttggaagattaggtcatggtgacagcaatagtcgtaacattccaacattagtaaaagacatcagcaatgtaggagaggtttcttgtggcagttcacatactattgctctgtctaaagatgggagaactgtatggtcttttggaggaggagacaatggtaaacttggtcatggtgataccaacagagtgtataaacctaaagttattgaagctttacaaggaatgttcattcgcaaagtttgtgctgggagccagtcttcacttgctttgacatcaacagggcaggtctatgcttggggctgtggagcttgtctaggttgtggttcttcagaagctactgctttgagacccaagcttattgaagaactggctgccacaagaatagttgatgtttctattggagacagtcattgtttggctctttctcatgataatgaagtttatgcctggggcaataactcaatggggcaatgtggtcagggaaattccacaggtcctattactaaaccaaagaaagtgagtggcttagatggcatagctattcagcagatttcggctggaacatcacatagtctggcatggactgctcttcctagggacagacaagttgttgcatggcaccgaccttattgtgtagatcttgaagagagtaccttctcacacctgcgttcttttcttgagagatactgtgataaaataaacagtgagattcccccactccctttcccttcatcaagagaacaccacagttttctcaagctgtgcctgaagctactttcaaatcaccttgctcttgcacttgcgggaggggtagctaccagcattctcgggaggcaggcaggtccacttcgaaatttgctcttcagactgatggactcaactgtcccagatgaaatccaagaggtggtaattgaaactttatcagtgggagcaaccatgctgttacctccattacgagaacggatggaattacttcattctcttttacctcaaggacctgatagatgggaaagcttatctaaaggacagagaatgcaactggatatcatcctgacaagtttgcaagatcatacccacgtagcctccctacttggctatagttcaccctctgatgctgctgacctatcttctgtgtgtactggctacggaaatctgtcagatcaaccttacggcactcagagctgccatccagatacccacctggctgaaattttgatgaagaccctcttaagaaatttaggattttatacagatcaagcatttggagagctagaaaagaatagtgataaatttctacttggaacatcatcatcagaaaacagtcagcctgctcatcttcatgaactgctatgttcactacagaaacagctgctggcattttgccatatcaataacattagtgagaactcaagcagtgtggcattgcttcataaacatcttcagcttttgttgcctcatgccacagatatttattcacgttctgcaaatttgctcaaagaaagtccttggaatggcagtgttggagaaaaattaagagatgtgatatacgtctcagctgctggcagtatgctctgccagattgttaactccctgctgttactccctgtgtcagtggctcggcctttattgagttacctcctcgacttgttgccacctcttgattgccttaatagactcctgccagctgctgatcttttagaagaccaggagttacagtggcctcttcatggagggccagaactaattgatcctgctggtctgccattacctcagccagctcagtcctgggtatggcttgtggatctagaaagaacaattgctctccttattgggcggtgtcttggtggcatgcttcagggctcccctgtgtctccagaggaacaggacactgcatattggatgaaaacgccactgttcagtgacggtgtagaaatggacactcctcaattggataaatgtatgagttgcctgttagaagtagcactttctggaaatgaagaacagaagccttttgattataaattgcggcctgaaattgctgtctatgtagacttggcattgggttgttctaaagagcctgcccgaagcctttggatcagcatgcaggactatgctgttagtaaagattgggacagtgcaactttaagtaatgagtcactcttggacactgtgtctagatttgttcttgcagctcttctgaaacacacaaatttacttagtcaagcatgtggagaaagccgatatcaacctggtaaacacttatcagaagtgtaccgttgtgtatacaaagttcgaagtcgtttacttgcttgcaagaaccttgaacttattcaaacaaggtcatcatcacgggacagatggatatcagaaaaccaggactctgcagatgttgatcctcaggagcattcatttactcgaactattgatgaagaagctgaaatggaagaacaggctgagagagaccgggaagaggggcatccggagccagaggatgaagaggaggaacgggaacatgaagtgatgacagctggcaaaatctttcagtgtttcctctcagcccgtgaagtagctcgtagccgagaccgagatagaatgaacagtggggcagggtctggggctcgagctgatgatccacctcctcagtctcagcaagagcgaagggtcagcacagaccttcctgagggtcaggatgtgtacactgctgcatgcaactccgtgatccatcggtgtgccctgttaatattaggagtaagtcctgtgatagatgagcttcagaagcgaagagaagaaggacagttgcagcaaccttcaacaagtgcctctgaagggggtggacttatgaccaggagtgaaagtcttactgcagagagccggctagtccacacaagcccaaattatagactgatcaaatcgaggagtgaatctgatttgtctcagcctgaatcagatgaagagggttacgcactgagtggcagacgaaatgttgatttggatttggcagcatctcacagaaagagaggtcctatgcacagtcaattggaatccctgagtgactcttgggctcgcctgaaacatagcagagactggttatgcaactcctcctattcctttgagtcagattttgatcttaccaagtctttgggagttcacactttgattgaaaatgttgtaagctttgtgagtggagatgtggggaatgccccaggttttaaagagccagaggaaagtatgtctacaagtccccaggcctccatcattgcaatggaacagcagcagttaagggcagaacttcgtttagaggcacttcatcagatcctcgttctattgtctgggatggaagaaaaaggtagcatctcactggcaggaagcagattgagttcaggcttccagtcctccacactactcacgtctgtgaggctgcagttcctagcagggtgttttggtttaggcactgttggacacacaggaggcaagggagagagtggccgattgcatcactatcaggatgggatcagagcagctaagagaaatattcagattgaaatccaggtagctgtgcataaaatttatcaacagttgtctgctaccctggaaagagccctgcaagcaaacaagcatcacattgaagcccagcaacgtctgcttctggttacagtttttgccctaagtgttcattatcaaccagtagatgtttctttggcaatttccactggtctgctaaacgtattgtcacagttgtgtggtacagacaccatgctaggacagcccctgcagttgttgccaaagacgggtgtttcccagcttagcacagctttgaaagtggccagtacaaggttgctccagattctagccatcactactgggacctatgctgataaactgagtcccaaagtagttcaatccttgttggatctactctgtagtcagttgaagaatttattgtcccaaactggtgtactacatatggcctctttcggagaaggggagcaagaagacggtgaagaagaagaaaaaaaagttgactccagtggagaaactgagaagaaagatttcagagctgctcttaggaaacaacatgcagccgaactccatctaggggattttttagtttttcttcgcagagttgtatcttcaaaagcaattcaatcaaaaatggcttccccaaagtggaccgaagtgcttctaaatatagcatctcagaaatgttcttcaggtatccctctggttggtaacttaagaacaaggctccttgcacttcatgtccttgaagctgtgctgccagcttgtgaatctggtgtagaagatgatcaaatggcccagattgttgagcgcttattttcccttctctctgattgtatgtgggagacacccattgctcaggccaaacatgctattcagataaaggaaaaagaacaagaaataaaactacagaagcagggcgagttggaagaagaagatgagaatcttcctatccaagaagtatcctttgacccggagaaagctcagtgttgcctagtggagaatggacagattttaactcacggcagtggagggaaaggatatggattggcatctacaggagtaacttctgggtgctatcagtggaagttttatattgtgaaggaaaacagaggtaatgaaggcacgtgtgttggagtttctcgctggccagtacatgactttaatcaccgcactacctcggatatgtggctctatagggcctacagtggtaacctctatcacaatggagaacagactctcacattgtccagctttactcaaggagatttcattacctgtgtgttagacatggaagccaggaccatttcttttgggaaaaatggagaggaacccaaattagcttttgaagatgtggatgcagcagagttgtacccatgtgtgatgttctatagtagcaatccaggggaaaaggtgaaaatttgtgatatgcagatgcgtggcacaccccgagacttacttccaggagaccctatttgtagtccagtagcagcagtgctggctgaggccactattcagctcatccgtatccttcaccgaacagaccgttggacttactgcattaacaaaaaaatgatggaaaggcttcacaaaattaagatatgtattaaagagtcaggtcagaagctaaagaaaagccgctcggttcagagccgagaggaaaatgaaatgagagaggagaaggagagcaaagaggaagagaaaggtaaacatactaggcatggcctcgctgacctctcagagctgcagctgaggactctttgcatagaggtgtggcccgtgctggctgtgataggaggagttgatgctggtcttagagttggaggtcggtgtgttcacaagcaaactgggcgccatgccacgctgctgggagtggtcaaagagggcagcacgtctgccaaggtccaatgggatgaagcagaaattactatcagcttcccaactttttggtcgcctagtgatactccattgtataatctggaaccctgtgaaccattgccgtttgatgtggcgcgattccgaggcctgacggcttctgtgctgctggacctaacatatctcactggcgttcatgaagacatgggcaaacagagcaccaaacgacatgaaaagaaacaccgacatgaatccgaggagaaaggggatgttgagcagaaacctgagagtgaatccgctttagatatgcgaacaggcctaacatctgatgacgtcaaaagtcagagtaccacaagctccaaatcagaaaatgaaatcgcttcattttctttagatccaacactgccaagtgtggaatcccaacatcaaataacagaagggaaaagaaaaaatcatgaacacatgtccaaaaaccatgatgtagcccagtcagaaatcagagcagtccagctgtcctatctttacctcggtgctatgaagtcacttagtgcccttcttggctgtagtaaatatgctgagctgttgctgataccaaaagttctggctgaaaatggccacaactcagactgtgcaagttctccagttgttcatgaagacgtggagatgcgagcagccctgcagttcttgatgcgacacatggtgaagcgagcagtcatgcggtcacccataaagagagcattgggattagctgatctggaacgagcgcaagccatgatctataaattagtggttcatgggcttttggaagaccagtttgggggcaaaattaagcaagagattgatcaacaagctgaagaaagtgaccctgcccagcaggcacagacaccagttactactagcccatcagcctcaagcacgacctcctttatgagcagctctctggaggacaccacaactgccaccactccagtcactgacacagaaacagtgcctgcatccgagtccccgggagtgatgcctcttagtcttctcaggcaaatgttctctagttacccaactaccactgtacttcccacacgtcgggcacagactcctccaatatcttcgttaccaacctctccttctgatgaagtaggaaggaggcaaagtttaacttctcctgattcccagtcagcaaggccagctaaccgcacagccttgtcagacccaagcagtagactttcaacttctcctcctcctccagcaattgcagttcccttgctggaaatggggttctctcttcggcagattgccaaagccatggaagctacaggtgctaggggagaggctgatgcccagaatatcactgtccttgccatgtggatgatagagcaccctgggcatgaggatgaagaggagccccagtcgggcagcacagcagactctaggcctggagcagccgttctaggcagtggcgggaagtcaaatgatccctgttatttgcagtcacctggagacataccatcagctgatgctgctgaaatggaggaaggttttagtgaaagccctgataatttggatcatacagagaatgcagcttctggaagtggaccatcagctagaggtcgctcagcggtaacaagaagacacaagtttgacttagctgctcgcacactgctagcaagagcagcgggattataccgctctgtgcaggcccacaggaatcaaagtcggagagaaggaatatctttgcagcaagacccaggggcgttgtatgactttaatttagatgaggaattggaaattgatcttgatgatgaggcgatggaagctatgtttggacaagacctgaccagtgacaatgatattctgggaatgtggatcccagaggtactggattggcctacctggcatgtttgtgagtctgaagacagggaagaagtggtggtgtgtgaactgtgtgaatgcagcgtcgtcagcttcaatcagcacatgaagagaaaccatccaggctgtgggcgcagtgcaaaccgccagggctatcgcagcaatggttcctatgtggatggctggtttggcggtgaatgtgggagtggaaatccgtactacctgttatgtggcacctgcagggagaagtacttagccatgaagaccaaatctaagtcaacaagttctgaaaggtacaagggacaagctccagatctaattggcaagcaagacagtgtgtatgaagaagactgggacatgttggatgttgatgaagatgaaaagctaactggtgaagaagaatttgaattacttgctggaccgcttggtttaaatgaccggcgcattgtaccagaaccagttcagttccctgacagcgatccactgggagcatcagtagcaatggtcacagccaccaacagtatggaagagactctgatgcaaataggttgccatggctccgtagaaaagagctcctctgggagaataacgttaggagagcaggcagctgccctagcaaaccctcatgaccgtgtggtggctttaaggagagtgactgctgctgctcaggttcttctggccagaaccatggtcatgagagcgctgtctcttctctcagtcagtggttccagttgtagcctggctgctggtcttgagtctctggggctaacagatatccgaacgctagttcgattaatgtgcttggcagcagcagggagagctggcctctccaccagcccttctgccatggctagcacctcagaacgatcacgaggtgggcatagcaaggctaacaagcctatctcttgcctggcctatttgagcacagcagtgggatgtctggcatcaaatgctcctagtgctgccaaactgcttgtacagttgtgtacacagaacttgatttctgctgcaacaggtgtaaatctaaccacagttgatgactcaattcagcgaaagtttctacccagctttctccgaggaattgctgaagagaacaagcttgtgacctccccaaactttgttgtaacacaggcccttgtggcattgctagcagacaaaggggccaaactaagacctaactatgataagtcagaagttgaaaagaaaggccctctggagttggctaatgccctggcagcctgctgcctctcctccaggctgtcctcacagcatcggcaatgggcagctcagcaactcgtgcgcactcttgctgcacacgaccgtgacaaccaaactactctgcagacacttgctgatatgggaggagatcttagaaaatgctcctttatcaaattggaggctcatcagaacagagtaatgacatgtgtttggtgtaataaaaaaggtcttttggctacaagtggcaatgatggcaccatccgcgtatggaatgttaccaagaagcaatattcactgcaacagacctgtgtgttcaacagattggaaggggatgctgaggaaagcctgggatcacccagtgatccaagtttctcaccagtttcctggagtatcagtggcaaatatctagcaggcgctttggaaaagatggtgaatatctggcaagttaatggaggaaaaggattagtagatattcagcctcattgggtatctgccctggcttggccagaagagggtccggctacagcctggtcaggagagtctccagaattgttgttggtgggacggatggatggatctctgggactgattgaagttgttgatgtgtccaccatgcaccgtcgagaattggagcattgctatcgaaaggatgtgtctgttacttgcattgcatggttcagtgaagacagaccatttgcagtgggatattttgatggaaaactgttactgggaacaaaggaaccacttgagaaaggaggcattgttctaattgatgcacataaggatactcttattagcatgaagtgggaccctacaggtcatattcttatgacatgtgccaaagaagacagtgtgaaactctggggctctatttcgggatgctggtgctgtctacattcactctgccatccatctattgtaaatggcattgcttggtgccgccttccagggaaaggatccaagttgcagttactgatggctactggctgtcagagtggcttagtatgtgtttggcgcattcctcaagatactacacagaccaatgtgactagtgcagaaggatggtgggagcaggaatcaaattgccaggatggatataggaaatcatcaggagccaagtgtgtttatcagctgcggggacacatcactcctgttcggactgttgcctttagttctgatgggttggccctggtgtctggtggactaggtgggctcatgaacatttggtctttaagggatggctctgtcttgcaaactgttgtgataggctctggagctattcagaccacagtatggattccagaagttggagtagctgcttgctcaaatagatcaaaggatgttttggtcgtgaattgtacagcagaatgggcagctgccaatcatgttttggcaacctgtaggacagcattgaaacagcagggtgttctgggattgaacatggctccctgcatgagagcatttttggagcggctccccatgatgcttcaggagcagtatgcctatgaaaagcctcatgtggtttgtggtgaccaacttgttcatagcccctatatgcaatgcttggcttcccttgctgtgggacttcatctggatcagctgttgtgtaaccctccagtgccaccacaccaccagaactgtctccctgaccctgcatcctggaatccaaatgaatgggcctggttagaatgtttctcaaccactataaaagctgccgaagccctgaccaatggagcccagtttccagaatcttttaccgttccagatctagaacctgttccagaggatgaacttgtatttctaatggataacagtaaatggattaacggcatggatgaacaaattatgtcttgggcaacttccagacctgaggactggcacctgggaggtaaatgtgatgtctacttatggggtgctggtaggcatggacagctggcagaagctggaagaaatgtaatggtacctgcagcagctccctcattctcacaggcccaacaggtcatttgtggtcagaattgtacctttgtcatccaggccaatggcacagtgttggcttgtggggaaggaagttatggcagattaggacaaggaaattcagatgaccttcatgtgctgacagttatttcagccttacaaggctttgtggtgacccagctggtgacttcctgtggttctgatgggcactctatggccctaactgaaagtggtgaggtctttagctggggagatggtgactatggtaaacttggccatgggaacagcgacaggcagcggcggcccaggcagatcgaggccttacaaggagaagaagtggtgcagatgtcttgtggcttcaagcactcagcagtggtcacttcagatggcaaactgttcacctttgggaatggtgactatggtcgtctgggtcttggaaatacctctaacaaaaaacttccagagagagtgactgcactggagggatatcagattggacaggtggcctgtggattaaaccacactttggcagtgtcagcagatggttccatggtgtgggcttttggagatggagactatggaaaactaggcttaggaaattccactgcaaaatcttcacctcagaaaattgacgtcctttgtggaattggaataaaaaaggttgcttgtggaactcagttttctgttgctttgaccaaagatggtcatgtgtatacctttggtcaagatcgcctgataggcttgccagaggggcgtgctcgcaatcacaatcgaccgcaacaaatccctgtcctggctggagtaatcatagaagatgtggcagttggagctgaacacacacttgctttggcatcaaatggagatgtgtatgcctgggggagcaattcagaagggcagctcggcttaggccataccaaccatgttcgagaaccaaccctggtaacaggtctgcaagggaaaaatgttcggcagatctcggctggccgctgccacagtgctgcatggacagcaccacctgtcccaccaagagcaccaggtgtgtcagtacctctgcagctgggcctgcctgacacagtgcccccccagtatggggcgctgagagaagtcagcattcacacggtgcgggccaggctccggctgctctaccacttctctgacctcatgtactcatcctggagactgctgaaccttagccccaacaaccagaacagcacatcccattataatgctggaacttggggcattgtacagggacaacttcggcctttgttagccccaagagtctacactctgccaatggtgcgctccataggaaaaaccatggttcaaggcaaaaactatggacctcagataactgtaaagaggatatcaaccagaggacggaagtgtaagcctatttttgtccaaatagcgagacaagtagttaagctgaatgcttcagacctccgcctgccttcccgagcgtggaaggttaagctggttggagaaggggctgatgatgctggaggagtgtttgatgacaccatcacagagatgtgccaggaacttgaaactggtattgttgaccttcttataccctctcccaatgccaccgcagaagtgggttacaatagggacaggttcctttttaacccttctgcctgcctcgatgaacacttaatgcagtttaagtttttaggaattttaatgggggttgccattcgcacaaagaagcctctggacctccacttggcccctctggtgtggaagcagctgtgctgtgtcccactcaccctagaggacctggaggaggtggatctgctctacgtgcagactctcaacagcattcttcacattgaagacagtgggattaccgaggagagtttccatgagatgattcctcttgattcttttgttggccagagtgctgatggcaaaatggttcctataatccctggtggaaatagtatcccactcacattttccaacaggaaggaatatgtggagagggccattgaatatcgacttcatgagatggacagacaggtggctgcagtccgagaagggatgtcctggattgttcctgtgccgctgctgtccctcctcacagcaaaacaactggagcagatggtgtgtgggatgcccgagatctctgtggaagtcttgaagaaagtggtgcggtaccgtgaggtggatgagcagcatcagctggtgcagtggttctggcacacgctggaagagttctccaatgaggagcgggtgcttttcatgaggtttgtgtcaggaagatctcgactaccagccaacactgctgacatttctcagagatttcaaatcatgaaggttgataggccttacgacagtctgcctacctcacagacctgcttcttccagctgaggctgcccccgtactccagccagctggtcatggccgagcgcctgcgctatgccatcaacaactgccgctcaatcgacatggacaactacatgctctcgagaaacgtggacaacgccgagggctccgacactgactactgaccgtgcgggtgctctcaccctcccttctctccctcaataatgctcacttctgatttgatgttgatatacttttatggtaactacatagatgttataagaacataaaccaacattataaacaatggccacatttagttactctaaatgtaacaaagaaattagatgtttttatttttctgtgattgtacaaaaacaacaaaaacgaagtgctctcagtcaggtttttccctccatatttttggtcacttttgataagtttgcatgaaaccattttggtgcatttttagttgggaatggtacatttttgtaaatccacccagtgaacatgaaattgtacattgtgtataattgttcattagaaaggacagttttacatgaatattcatatatttattttgttttaatttgaattgcctgttcagggttccttatgcagagaaataaagcagattcaggaattgga

SEQ ID NO: 9 - RNA, 20 nt, synthetic construct
  misc_feature 1..20: note = Description of Artificial Sequence: Synthetic oligonucleotide
  source 1..20: mol_type = other RNA
cgccctctaggcccctaatc

SEQ ID NO: 10 - DNA, 27 bp, synthetic construct
  misc_feature 1..27: note = Description of Artificial Sequence: Synthetic primer
  source 1..27: mol_type = other DNA
gagttcatctacaaggtgaagctgcgc

SEQ ID NO: 11 - DNA, 27 bp, synthetic construct
  misc_feature 1..27: note = Description of Artificial Sequence: Synthetic primer
  source 1..27: mol_type = other DNA
cttgtaggtggtcttgacctcagcgtc

SEQ ID NO: 12 - DNA, 481 bp, synthetic construct
  misc_feature 1..481: note = Description of Artificial Sequence: Synthetic polynucleotide
  source 1..481: mol_type = other DNA
agtggccaactccatcactaggggttcctgcggccgcacgcgtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctaccaggtcgaagagagcggtttaagagctatgctggaaacagcatagcaagtttaaataaggctagtccgttatcaacttgaaaaagtggcaccgagtcggtgctttttttcccagtcacgacgttgtaaaacgacggccagtgagcgcgcgtaatacgactcactatagg

SEQ ID NO: 13 - DNA, 494 bp, synthetic construct
  misc_feature 1..494: note = Description of Artificial Sequence: Synthetic polynucleotide
  source 1..494: mol_type = other DNA
cccagtcacgacgttgtaaaacgacggccagtgagcgcgcgtaatacgactcactataggaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtgcttggagcggttgaccgtttaagagctatgctggaaacagcatagcaagtttaaataaggctagtccgttatcaacttgaaaaagtggcaccgagtcggtgctttttttagcggataacaatttcacacaggaaacagctatgaccatgattacgccaagcgcgc

SEQ ID NO: 14 - DNA, 499 bp, synthetic construct
  misc_feature 1..499: note = Description of Artificial Sequence: Synthetic polynucleotide
  source 1..499: mol_type = other DNA
agcggataacaatttcacacaggaaacagctatgaccatgattacgccaagcgcgcaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtataatgggcaccaactcgggtttaagagctatgctggaaacagcatagcaagtttaaataaggctagtccgttatcaacttgaaaaagtggcaccgagtcggtgcttttttttctagactgcagagggccctgcgtatgagtgcaagtgggttttaggaccaggatgaggcggggtg

SEQ ID NO: 15 - DNA, 481 bp, synthetic construct
  misc_feature 1..481: note = Description of Artificial Sequence: Synthetic polynucleotide
  source 1..481: mol_type = other DNA
agtggccaactccatcactaggggttcctgcggccgcacgcgtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggatggtggaccgcgagcaacgtttaagagctatgctggaaacagcatagcaagtttaaataaggctagtccgttatcaacttgaaaaagtggcaccgagtcggtgctttttttcccagtcacgacgttgtaaaacgacggccagtgagcgcgcgtaatacgactcactatagg

SEQ ID NO: 16 - DNA, 494 bp, synthetic construct
  misc_feature 1..494: note = Description of Artificial Sequence: Synthetic polynucleotide
  source 1..494: mol_type = other DNA
cccagtcacgacgttgtaaaacgacggccagtgagcgcgcgtaatacgactcactataggaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaggaggttccgttcctcatgtttaagagctatgctggaaacagcatagcaagtttaaataaggctagtccgttatcaacttgaaaaagtggcaccgagtcggtgctttttttagcggataacaatttcacacaggaaacagctatgaccatgattacgccaagcgcgc

SEQ ID NO: 17 - DNA, 499 bp, synthetic construct
  misc_feature 1..499: note = Description of Artificial Sequence: Synthetic polynucleotide
  source 1..499: mol_type = other DNA
agcggataacaatttcacacaggaaacagctatgaccatgattacgccaagcgcgcaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagcagaagacgtctgcggagtttaagagctatgctggaaacagcatagcaagtttaaataaggctagtccgttatcaacttgaaaaagtggcaccgagtcggtgcttttttttctagactgcagagggccctgcgtatgagtgcaagtgggttttaggaccaggatgaggcggggtg

SEQ ID NO: 18 - DNA, 481 bp, synthetic construct
  misc_feature 1..481: note = Description of Artificial Sequence: Synthetic polynucleotide
  source 1..481: mol_type = other DNA
agtggccaactccatcactaggggttcctgcggccgcacgcgtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacgtggtagttcaaatggaagtttaagagctatgctggaaacagcatagcaagtttaaataaggctagtccgttatcaacttgaaaaagtggcaccgagtcggtgctttttttcccagtcacgacgttgtaaaacgacggccagtgagcgcgcgtaatacgactcactatagg

SEQ ID NO: 19 - DNA, 494 bp, synthetic construct
  misc_feature 1..494: note = Description of Artificial Sequence: Synthetic polynucleotide
  source 1..494: mol_type = other DNA
cccagtcacgacgttgtaaaacgacggccagtgagcgcgcgtaatacgactcactataggaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcctttaaggctgggcgcatcgtttaagagctatgctggaaacagcatagcaagtttaaataaggctagtccgttatcaacttgaaaaagtggcaccgagtcggtgctttttttagcggataacaatttcacacaggaaacagctatgaccatgattacgccaagcgcgc

SEQ ID NO: 20 - DNA, 499 bp, synthetic construct
  misc_feature 1..499: note = Description of Artificial Sequence: Synthetic polynucleotide
  source 1..499: mol_type = other DNA
agcggataacaatttcacacaggaaacagctatgaccatgattacgccaagcgcgcaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaaagccatttatgagcctggtttaagagctatgctggaaacagcatagcaagtttaaataaggctagtccgttatcaacttgaaaaagtggcaccgagtcggtgcttttttttctagactgcagagggccctgcgtatgagtgcaagtgggttttaggaccaggatgaggcggggtg

SEQ ID NO: 21 - DNA, 481 bp, synthetic construct
  misc_feature 1..481: note = Description of Artificial Sequence: Synthetic polynucleotide
  source 1..481: mol_type = other DNA
agtggccaactccatcactaggggttcctgcggccgcacgcgtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcatctgctgatcggagtcaggtttaagagctatgctggaaacagcatagcaagtttaaataaggctagtccgttatcaacttgaaaaagtggcaccgagtcggtgctttttttcccagtcacgacgttgtaaaacgacggccagtgagcgcgcgtaatacgactcactatagg

SEQ ID NO: 22 - DNA, 494 bp, synthetic construct
  misc_feature 1..494: note = Description of Artificial Sequence: Synthetic polynucleotide
  source 1..494: mol_type = other DNA
cccagtcacgacgttgtaaaacgacggccagtgagcgcgcgtaatacgactcactataggaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactatgggaaactaggacatgtttaagagctatgctggaaacagcatagcaagtttaaataaggctagtccgttatcaacttgaaaaagtggcaccgagtcggtgctttttttagcggataacaatttcacacaggaaacagctatgaccatgattacgccaagcgcgc

SEQ ID NO: 23 - DNA, 499 bp, synthetic construct
  misc_feature 1..499: note = Description of Artificial Sequence: Synthetic polynucleotide
  source 1..499: mol_type = other DNA
agcggataacaatttcacacaggaaacagctatgaccatgattacgccaagcgcgcaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccgagatagaatgaacagtggtttaagagctatgctggaaacagcatagcaagtttaaataaggctagtccgttatcaacttgaaaaagtggcaccgagtcggtgcttttttttctagactgcagagggccctgcgtatgagtgcaagtgggttttaggaccaggatgaggcggggtg

SEQ ID NO: 24 - DNA, 481 bp, synthetic construct
  misc_feature 1..481: note = Description of Artificial Sequence: Synthetic polynucleotide
  source 1..481: mol_type = other DNA
agtggccaactccatcactaggggttcctgcggccgcacgcgtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaggagatcctagcccgggatgtttaagagctatgctggaaacagcatagcaagtttaaataaggctagtccgttatcaacttgaaaaagtggcaccgagtcggtgctttttttcccagtcacgacgttgtaaaacgacggccagtgagcgcgcgtaatacgactcactatagg

SEQ ID NO: 25 - DNA, 494 bp, synthetic construct
  misc_feature 1..494: note = Description of Artificial Sequence: Synthetic polynucleotide
  source 1..494: mol_type = other DNA
cccagtcacgacgttgtaaaacgacggccagtgagcgcgcgtaatacgactcactataggaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggccggtgatgatcacgactggtttaagagctatgctggaaacagcatagcaagtttaaataaggctagtccgttatcaacttgaaaaagtggcaccgagtcggtgctttttttagcggataacaatttcacacaggaaacagctatgaccatgattacgccaagcgcgc

SEQ ID NO: 26 - DNA, 499 bp, synthetic construct
  misc_feature 1..499: note = Description of Artificial Sequence: Synthetic polynucleotide
  source 1..499: mol_type = other DNA
agcggataacaatttcacacaggaaacagctatgaccatgattacgccaagcgcgcaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcggggtgtggtggtagccacgtttaagagctatgctggaaacagcatagcaagtttaaataaggctagtccgttatcaacttgaaaaagtggcaccgagtcggtgcttttttttctagactgcagagggccctgcgtatgagtgcaagtgggttttaggaccaggatgaggcggggtg

SEQ ID NO: 27 - DNA, 481 bp, synthetic construct
  misc_feature 1..481: note = Description of Artificial Sequence: Synthetic polynucleotide
  source 1..481: mol_type = other DNA
agtggccaactccatcactaggggttcctgcggccgcacgcgtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaagtgtcccctgccatcgccgtttaagagctatgctggaaacagcatagcaagtttaaataaggctagtccgttatcaacttgaaaaagtggcaccgagtcggtgctttttttcccagtcacgacgttgtaaaacgacggccagtgagcgcgcgtaatacgactcactatagg

SEQ ID NO: 28 - DNA, 494 bp, synthetic construct
  misc_feature 1..494: note = Description of Artificial Sequence: Synthetic polynucleotide
  source 1..494: mol_type = other DNA
cccagtcacgacgttgtaaaacgacggccagtgagcgcgcgtaatacgactcactataggaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgcgtggtgagaatcagacgtttaagagctatgctggaaacagcatagcaagtttaaataaggctagtccgttatcaacttgaaaaagtggcaccgagtcggtgctttttttagcggataacaatttcacacaggaaacagctatgaccatgattacgccaagcgcgc

SEQ ID NO: 29 - DNA, 499 bp, synthetic construct
  misc_feature 1..499: note = Description of Artificial Sequence: Synthetic polynucleotide
  source 1..499: mol_type = other DNA
agcggataacaatttcacacaggaaacagctatgaccatgattacgccaagcgcgcaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggacgttgatgcagaaggtaggtttaagagctatgctggaaacagcatagcaagtttaaataaggctagtccgttatcaacttgaaaaagtggcaccgagtcggtgcttttttttctagactgcagagggccctgcgtatgagtgcaagtgggttttaggaccaggatgaggcggggtg

SEQ ID NO: 30 - DNA, 481 bp, synthetic construct
  misc_feature 1..481: note = Description of Artificial Sequence: Synthetic polynucleotide
  source 1..481: mol_type = other DNA
agtggccaactccatcactaggggttcctgcggccgcacgcgtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagctgctgaaggacacatggtttaagagctatgctggaaacagcatagcaagtttaaataaggctagtccgttatcaacttgaaaaagtggcaccgagtcggtgctttttttcccagtcacgacgttgtaaaacgacggccagtgagcgcgcgtaatacgactcactatagg

SEQ ID NO: 31 - DNA, 494 bp, synthetic construct
  misc_feature 1..494: note = Description of Artificial Sequence: Synthetic polynucleotide
  source 1..494: mol_type = other DNA
cccagtcacgacgttgtaaaacgacggccagtgagcgcgcgtaatacgactcactataggaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatcagtgccattgcaacccagtttaagagctatgctggaaacagcatagcaagtttaaataaggctagtccgttatcaacttgaaaaagtggcaccgagtcggtgctttttttagcggataacaatttcacacaggaaacagctatgaccatgattacgccaagcgcgc

SEQ ID NO: 32 - DNA, 499 bp, synthetic construct
  misc_feature 1..499: note = Description of Artificial Sequence: Synthetic polynucleotide
  source 1..499: mol_type = other DNA
agcggataacaatttcacacaggaaacagctatgaccatgattacgccaagcgcgcaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaggtcacatcgggctgcgtggtttaagagctatgctggaaacagcatagcaagtttaaataaggctagtccgttatcaacttgaaaaagtggcaccgagtcggtgcttttttttctagactgcagagggccctgcgtatgagtgcaagtgggttttaggaccaggatgaggcggggtg